(19) United States
(12) Patent Application Publication
(10) Pub. No.: US 2012/0302609 A1
(43) Pub. Date: Nov. 29, 2012

(54) Pyrazolines for the Modulation of PKM2

(75) Inventors: Oren M. Becker, Mevasseret Zion (IL); Alina Shitrit, Tel-Aviv (IL); Nili Schutz, Tel-Aviv (IL); Efrat Ben-Zeev, Kiryat Motzkin (IL); Avihai Yacovan, Gedera (IL); Rachel Ozeri, Gimzo (IL); Daniel Sherman, Jerusalem (IL); Vered Behar, Moshav Bet Zayit (IL); Osnat Kashtan, Kadima (IL); Alexander Konson, Beersheba (IL)

(73) Assignee: Dynamix Pharmaceuticals LTD., Rehovot (IL)

(21) Appl. No.: 13/479,131

(22) Filed: May 23, 2012

Related U.S. Application Data
(60) Provisional application No. 61/519,650, filed on May 25, 2011.

(51) Int. Cl. A61K 31/4155; A61K 31/415; C07D 405/10; C07D 405/14; C07D 401/06; A61P 35/00; C07D 409/04; A61P 37/00; A61P 37/06; A61P 29/00; A61P 3/00; C07D 231/06; A61K 31/4439

(52) U.S. Cl. 514/341; 548/379.4; 514/403; 548/364.4; 548/364.7; 548/364.1; 546/275.4; 548/365.7

(57) ABSTRACT

The invention relates to pyrazoline substituted compounds and methods for activating PKM2. The compounds and methods are useful in treating or preventing a disease or disorder selected from cancer, cell proliferative disorder, inflammatory disorder, metabolic disorder, and immune system disorder.

### BACKGROUND OF THE INVENTION

Tumor development is associated with major metabolic changes. In the 1920s, Otto Warburg observed that cancer cells have high glucose consumption and lactate production even in the presence of oxygen (a process termed aerobic glycolysis). Recent research has demonstrated that these metabolic differences drive tumor growth. By modulating their metabolic processes, cancer cells are able to divert sugars, fats and other energy sources away from energy production to satisfy the ever growing demands of uncontrolled proliferation.

Pyruvate Kinase (PK) is a metabolic enzyme that catalyzes the transfer of a phosphate group from phosphoenol pyruvate to ADP, to produce pyruvate and ATP during glycolysis. There are four PK isozymes—the L and the R isozymes are expressed in liver and red blood cells, respectively; the M1 isozyme is expressed in most adult cells, and the M2 isoenzyme—an M2 splice variant (PKM2)—is exclusively expressed during embryonic development and in cancer cells.

While PKM1 is a constitutively active enzyme, PKM2 undergoes a transformation from an energy efficient tetrametic form to an ‘energy inefficient’ dimer form. The main effector that balances the dimer-tetramer ratio of PKM2 in tissues is fructose 1,6-bisphosphate (FBP), a glycolysis intermediate product upstream of PKM2.

PKM2 is a key mediator of the Warburg effect in cancer cells leading to lower energy production and an abundance of building blocks for tumor replication and growth. There is thus a need in the art for, inter alia, modulators of the metabolism of proliferating cells.

### SUMMARY OF THE INVENTION

The invention is directed to compounds that modulate the metabolism of proliferating cells (e.g., cancer cells or lymphocytes, such as B or T cells). For example, in some embodiments, compounds of the invention are useful in the modulation (e.g., activation) of PKM2. Compounds of the invention are useful as pharmaceutical agents. The compounds may be used without limitation, for example, as anti-cancer, anti-proliferative, anti-inflammatory and/or immunosuppressive agents, for treating mammals, such as for treating humans. Compounds of the invention may be useful for modulating the metabolism of proliferating cells in a disease or disorder. Such diseases and disorders include, without limitation, cancers, inflammatory disorders, autoimmune disorders, immune system dysfunction, immune disease, metabolic disorders and transplant rejection. Compounds of the invention may be useful for regulating (e.g., activating) a PKM2 involved in a disease or disorder, such as a cancer.

In one aspect, a compound of the invention includes a compound of Formula I:

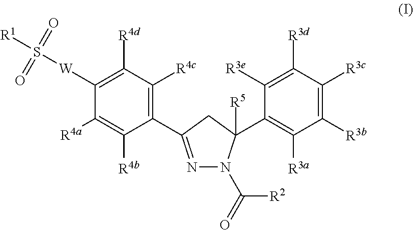

or a salt, solvate, hydrate or prodrug thereof, wherein W, R¹, R², R^{3a}, R^{3b}, R^{3c}, R^{3d}, R^{3e}, R^{4a}, R^{4b},
R^{4c}, R^{4d} and R⁵ are as described herein.

In one aspect, embodiments of the invention include compounds that activate PKM2 by at least about 10%, by at least about 20%, by at least about 25%, by at least about 30%, by at least about 40%, by at least about 50%, by at least about 60%, by at least about 70%, by at least about 75%, by at least about 80%, by at least about 90%, or by about 95% or more.

In one aspect, a compound of the invention may be used as a pharmaceutical agent. For example, a compound of the invention is useful as an anti-cancer, anti-proliferative, anti-inflammatory and/or immunosuppressive agent, for treating humans and/or animals, such as for treating humans or other mammals, preferably humans.

In one aspect, a compound of the invention is used in the manufacture of a medicament to treat or prevent a disease or disorder that is modulated by PKM2 activaction. For example, a compound of the invention may be used in the manufacture of a medicament to be used as an anti-cancer, anti-proliferative, anti-inflammatory and/or immunosuppressive agent.

In one aspect, embodiments of the invention are drawn to methods of treating or preventing cancers and/or cell proliferation disorders in a subject by administering a pharmaceutical composition that includes an effective amount of a compound of the invention or a salt, solvate, hydrate, or prodrug thereof. For example, the cancer or cell proliferation disorder is cancer, pre-cancer or a hyperproliferative disorder. In some embodiments, the foregoing methods are monotherapies for preventing or treating cancer and/or cell proliferation disorder. In some embodiments, the foregoing methods are part of a combination therapy with other therapeutic agents (e.g., a cancer metabolism modulators or a cytotoxic agent) and/or non-drug therapies (e.g., surgery, immunotherapy or radiation treatment). In some embodiments of the combination therapy, the additional therapy is conducted substantially simultaneously or concurrently with the pharmaceutical composition's administration. In some embodiments, the administration of the pharmaceutical composition is conducted prior to the additional therapy of the combination therapy. In some embodiments, the administration of the pharmaceutical composition is conducted subsequent to the additional therapy. In some embodiments, the pharmaceutical composition is administered chronically (e.g., as part of a maintenance therapy). In some embodiments, the cancer and/or cell proliferation disorder is a cell proliferative disorder of the hematologic system (e.g., leukemia or lymphoma). In some embodiments, the cancer and/or cell proliferation disorder is a cell proliferative disorder of the lung (e.g., lung cancer). In some embodiments, the cancer and/or cell proliferation disorder is a cell proliferative disorder of the colon (e.g., colon cancer). In some embodiments, the cancer and/or cell proliferation disorder is a cell proliferative disorder of the pancreas (e.g., pancreatic cancer). In some embodiments, the cancer and/or cell proliferation disorder is a cell proliferative disorder of the prostate (e.g., prostate cancer). In some embodiments, the cancer and/or cell proliferation disorder is a cell proliferative disorder of the skin (e.g., a skin cancer). In some embodiments, the cancer and/or cell proliferation disorder is a cell proliferative disorder of the ovary (e.g., ovarian cancer). In some embodiments, the cancer and/or cell proliferation disorder is a cell proliferative disorder of the breast (e.g., breast cancer).

In certain embodiments, the administration of the compound is carried out orally, parentally, subcutaneously, intravenously, intramuscularly, intraperitoneally, by intranasal instillation, by intracavitary or intravesical instillation, topically, intraarterially, intralesionally, by metering pump, or by application to mucous membranes. In some embodiments, the compound is administered with a pharmaceutically acceptable carrier.

Another aspect of the invention includes a method of regulating immune system activity in a subject comprising administering a compound of the invention or a salt, solvate, hydrate, or prodrug thereof. For example, modulating immune system activity includes modulating autoimmune diseases such as transplant rejection (e.g., kidney, heart, lung, liver, pancreas, skin, host versus graft reaction (HVGR), etc.), rheumatoid arthritis, and amyotrophic lateral sclerosis. Another aspect of the invention includes use of a compound of the invention in the manufacture of a medicament to regulate immune system activity.

In some embodiments, the administration of the compound is carried out orally, parentally, subcutaneously, intravenously, intramuscularly, intraperitoneally, by intranasal instillation, by intracavitary or intravesical instillation, topically, intraarterially, intralesionally, by metering pump, or by application to mucous membranes. In some embodiments, the compound is administered with a pharmaceutically acceptable carrier. In some embodiments, the compound is administered before the onset of immune system irregularity. In other embodiments, the compound is administered after the onset of immune system irregularity.

In one aspect, embodiments of the invention are drawn to methods of activating PKM2 by contacting PKM2 with a compound of the invention or a salt, solvate, hydrate, or prodrug thereof. In some embodiments, PKM2 is activated by at least about 10%, by at least about 20%, by at least about 25%, by at least about 30%, by at least about 40%, by at least about 50%, by at least about 60%, by at least about 70%, by at least about 75%, by at least about 80%, by at least about 90%, or by about 95% or more. In some embodiments, the PKM2 activated is in a cell, e.g., a human cell.

A compound of the invention may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

It is contemplated that whenever appropriate, any embodiment of the invention can be combined with one or more other embodiments of the invention, even though the embodiments are described under different aspects of the invention.

The above description sets forth rather broadly the more important features of the invention in order that the detailed description thereof that follows may be understood, and in order that the present contributions to the art may be better appreciated. Other objects and features of the invention will become apparent from the following detailed description considered in conjunction with the examples and figures.

### BRIEF DESCRIPTION OF THE DRAWINGS

FIG. 1 shows HPLC chromatograms of N-(4-(1-acetyl-5-(4-methoxyphenyl)-4,5-dihydro-1H-pyrazol-3-yl)phenyl)ethanesulfonamide (Compound 1A). The chromatogram for the starting racemate is shown in FIG. 1A, and the chromatograms for each of the resolved individual enantiomers are shown in FIGS. 1B (S-enantiomer) and 1C (R-enantiomer), respectively.

FIG. 2 shows HPLC chromatograms of N-(4-(1-acetyl-5-(4-methoxyphenyl)-4,5-dihydro-1H-pyrazol-3-yl)phenyl)-4-fluorobenzenesulfonamide (Compound 6A). The chromatogram for the starting racemate is shown in FIG. 2A, and the chromagtograms for each of the resolved individual enantiomers are shown in FIGS. 2B (R-enantiomer) and 2C (S-enantiomer), respectively.

FIGS. 3A and 3B show ¹H-NMR analyses for the individual R- (FIG. 3A) and S- (FIG. 3B) enantiomers, respectively, of N-(4-(1-acetyl-5-(4-methoxyphenyl)-4,5-dihydro-1H-pyrazol-3-yl)phenyl)-4-fluorobenzenesulfonamide (Compound 6A).

FIG. 4 shows the efficacy and safety results using N-(4-(1-acetyl-5-(4-methoxyphenyl)-4,5-dihydro-1H-pyrazol-3-yl)phenyl)ethanesulfonamide (Compound 1A) in a HT-29 xenograft mouse model as described in Example 10. FIG. 4A is a plot of tumor size at different time points after tumor inoculation, for a dosing schedule of 400 mg/kg QD, the vehicle, and a positive control (Irinotecan). FIG. 4B is a plot of Tumor Growth Inhibition (TGI) as a function of time for two dosing schedules, i.e., 400 mg/kg QD and 200 mg/kg QD. FIG. 4C is a plot of body weight as a function of time for the each of the cohorts in the study. FIG. 4D shows blood pharmacokinetic values for each of the cohorts in the study.

FIG. 5 shows the results of treating HT-29 cells (a colorectal cancer cell line) with a combination of N-(4-(1-acetyl-5-(4-methoxyphenyl)-4,5-dihydro-1H-pyrazol-3-yl)phenyl)ethanesulfonamide (Compound 1A) and 5-FU (a standard of care first line chemotherapeutic for colorectal cancer).

FIG. 6 shows as a function of concentration of compound a bar graph of % cells in the indicated stage of the cell cycle (left panel) and a plot of cell count (right panel) after H1299 cells (a non-small cell lung carcinoma cell line) were treated for 48 hours with the indicated concentration of (S)—N-(4-(1-acetyl-5-(4-methoxyphenyl)-4,5-dihydro-1H-pyrazol-3-yl)phenyl)-4-fluorobenzenesulfonamide (Compound 13A).

FIG. 7 shows a Western blot of PKM2. Recombinant PKM2 was incubated with various compounds or FBP for 60 min., followed by 0.025% glutaraldehyde cross-linking (5 min @ 4° C.) before SDS-PAGE (4-20%) and Western blot analysis with anti-PKM2 antibody (CST). The tetrameric and monomeric forms of PKM2. Compound 6A was N-(4-(1-acetyl-5-(4-methoxyphenyl)-4,5-dihydro-1H-pyrazol-3-yl)phenyl)-4-fluorobenzenesulfonamide and Compound 1A was N-(4-(1-acetyl-5-(4-methoxyphenyl)-4,5-dihydro-1H-pyrazol-3-yl)phenyl)ethanesulfonamide.

FIGS. 8A and 8B show proliferation changes of H460 cells affected by media conditions or Compounds 13A, 35A, 39A, 40A, 25A, and 19A.

FIGS. 9A and 9B show proliferation changes of HT-29 cells affected by media conditions or Compounds 13A, 35A, 39A, 40A, 25A, and 19A. FIGS. 9C and 9D show proliferation changes of H460 cells affected by Compounds 1A, 2A, and 3A; 6A, 12A, and 13A; 18A, 29A, and 30A; 19A, 39A, and 40A; 25A and 35A; 26A, 37A, and 38A and control compound (Compound 13A) in BME media (FIG. 9C) and lipoprotein-free media (FIG. 9D).

FIG. 10 shows individual times to endpoint for mice in the H1299 nude mouse xenograft model.

FIG. 11A and FIG. 11B show median tumor growth and Kaplan-Meier plot in the H1299 nude mouse xenograft model.

FIG. 12 shows percent group mean body weight changes from Day 1 for mice in the H1299 nude mouse xenograft model.

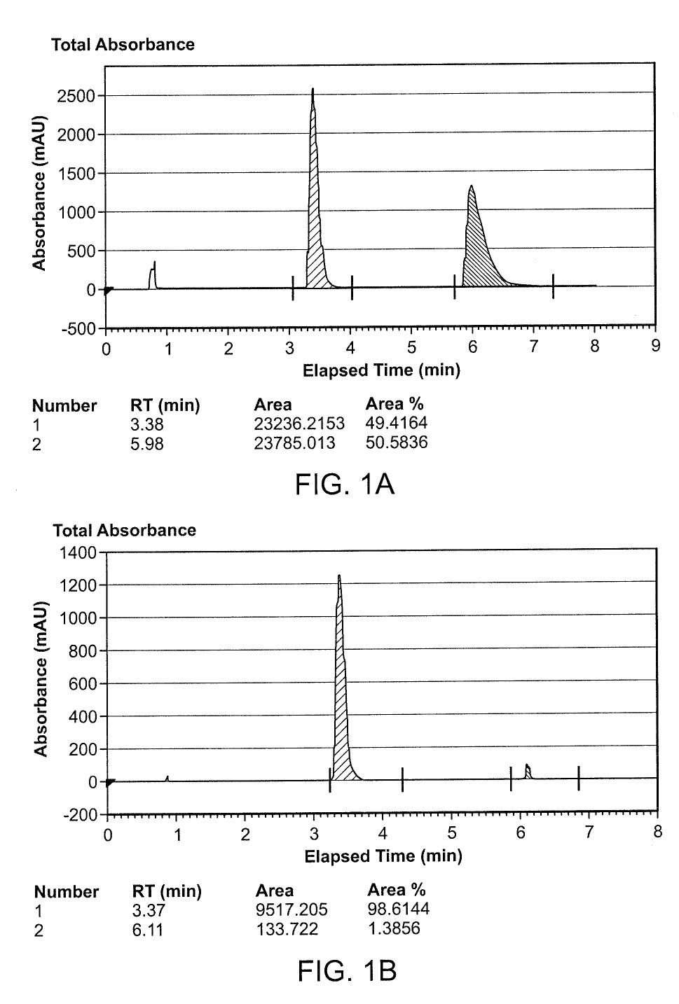

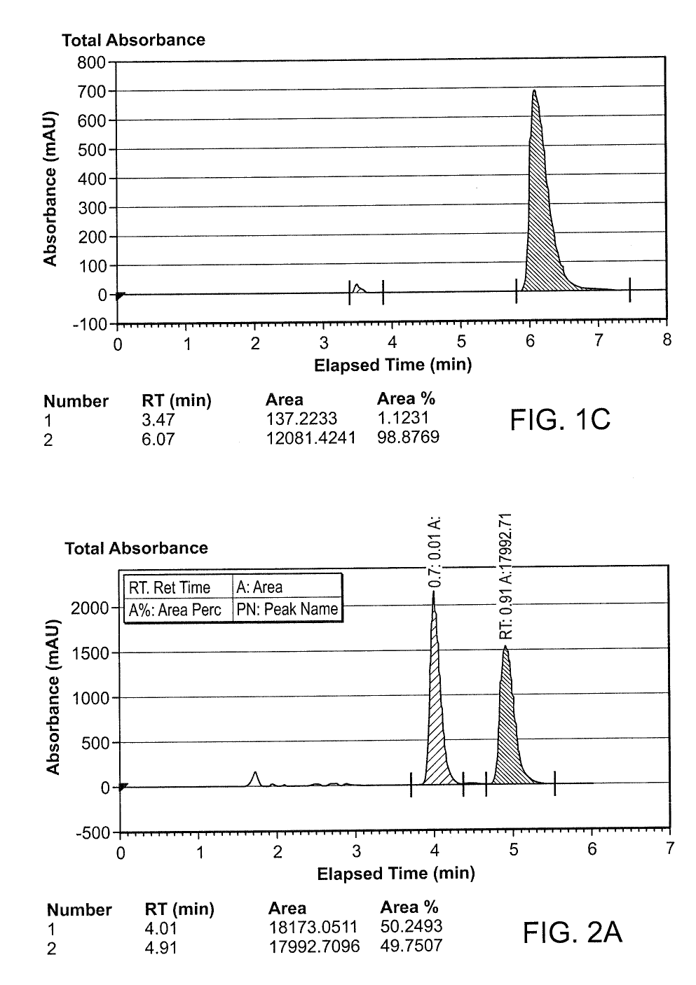

### DETAILED DESCRIPTION OF THE INVENTION

The details of one or more embodiments of the invention are set forth in the accompanying description below. Although any methods and materials similar or equivalent to those described herein can be used in the practice or testing of the invention, the preferred methods and materials are now described. Other features, objects, and advantages of the invention will be apparent from the description. In the specification, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present specification will control.

In one aspect, a compound of the invention modulates the metabolism of proliferating cells (e.g., cancer cells or lymphocytes, such as B or T cells). Compounds of the invention may be useful as pharmaceutical agents for modulating the metabolism of proliferating cells in a disease or disorder. Such diseases and disorders include, without limitation, cancers, inflammatory disorders, autoimmune disorders, immune system dysfunction, immune disease, metabolic disorders, and transplant rejection. For example, the compounds may be useful as anti-cancer, anti-proliferative, anti-inflammatory and/or immunosuppressive agents, for treating mammals, such as for treating humans.

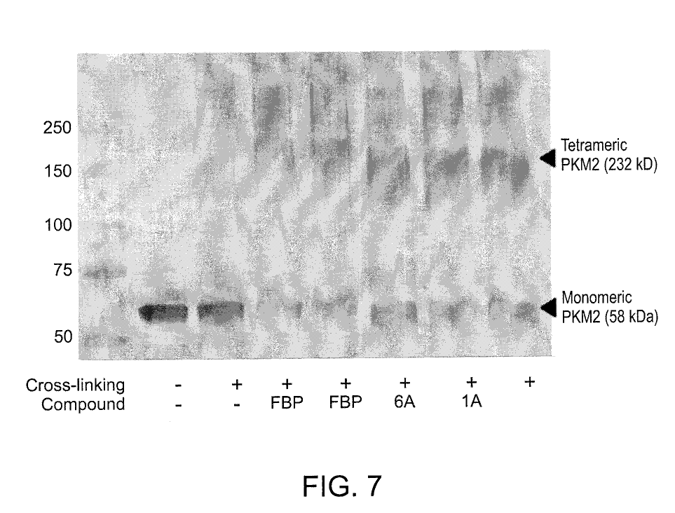

Without wishing to be bound by theory, PKM2 is a key mediator of the Warburg effect in cancer cells, leading to lower energy production and an abundance of building blocks for tumor replication and growth. Thus, it is believed that activating PKM2 may be an important way to treat or prevent cancers and proliferative diseases. Compounds of the invention are useful in the activation of PKM2. For example, and without wishing to be bound by theory, coumpounds according to certain embodiments of the invention stabilize the active tetrameric form of PKM2, as shown in FIG. 7. The compounds of the invention are useful as pharmaceutical agents, for example, as therapeutic agents for treating humans and animals. The compounds may be used without limitation, for example, as anti-cancer or other cell proliferation-related disorders.

In one aspect, the invention provides a compound according to Formula I:

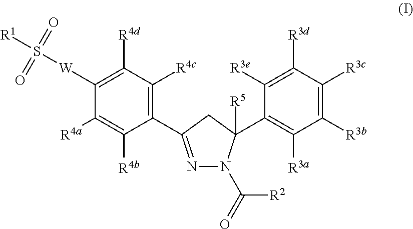

or a salt, solvate, hydrate, or prodrug thereof,
wherein:
W is NR^{a} or is absent,
wherein R^{a} is (i) a group selected from the group consisting of a hydrogen, a linear or branched, saturated or unsaturated C₁-C₆ alkyl, C(O)R^{c}, and a linear or branched, saturated or unsaturated C₁-C₆ alkoxy; or (ii) R^{a} and R^{4a} together with the atoms to which they are attached, form a five to seven membered heterocycloalkyl or heteroaromatic ring;
   R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,
wherein said alkyl, heteroalkyl, cycloalkyl, heterocycloalkyl, aryl or heteroaryl is unsubstituted or substituted with one or more R^{b},
wherein R^{b} is independently at each occurrence selected from the group consisting of:
   a) a linear or branched, saturated or unsaturated C₁-C₆ alkyl,
   b) a linear or branched, saturated or unsaturated C₁-C₆ haloalkyl,
   c) a linear or branched, saturated or unsaturated C₁-C₆ alkoxy or aryloxy,
   d) a linear or branched, saturated or unsaturated C₁-C₆ haloalkoxy,
   e) a linear or branched, saturated or unsaturated C₁-C₆ alkylsulfonyl,
   f) a linear or branched, saturated or unsaturated C₁-C₆ thioalkyl or thioaryl,
   g) a saturated or unsaturated C₃-C₈ cycloalkyl,
   h) an aryl,
   i) a heteroaryl,
   j) a heterocycloalkyl,
   k) hydroxyl,
   l) cyano,
   m) amino,
   n) nitro,
   o) halogen,
   p) COR^{c},
   q) COOR^{c},
   r) CONR^{c}R^{d},
   s) NHCOR^{b}, and
   t) NR^{c}R^{d}
      wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      or wherein two R^{b} on adjacent carbon atoms form a heteroaryl ring or heterocycloalkyl ring;
R² and R⁵ are, each independently, selected from hydrogen, a linear or branched, saturated or unsaturated C₁-C₆ alkyl or heteroalkyl, heteroaryl, and aryl,
   wherein said alkyl, heteroalkyl, heteroaryl, or aryl is unsubstituted or substituted with one or more R^{e},
      wherein R^{e} is independently at each occurrence selected from the group consisting of COR^{f}, COOR^{f}, CONR^{f}R^{g}, NHCOR^{f}, and NR^{f}R^{g},
      wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
         R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, or
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring;
   R^{4b}, R^{4c} and R^{4d}, and when R^{4a} does not form a five to seven membered ring with R^{a}, R^{4a}, are each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{j},
r) COOR^{j},
s) CONR^{j}R^{k},
t) NHCOR^{j}, and
u) NR^{j}R^{k}
   wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{4a}, R^{4b}, R^{4c} and R^{4d}, together with the atoms to which they are attached, form a C₅-C₇ cycloalkyl, heterocycloalkyl, aromatic or heteroaromatic ring.

In one aspect, compounds according to embodiments of the invention include a compounds of Formula I or a salt, solvate, hydrate or prodrug thereof wherein:

W is NR^{a} or is absent,
wherein R^{a} is (i) a group selected from the group consisting of a hydrogen, a linear or branched, saturated or unsaturated C₁-C₆ alkyl, and a linear or branched, saturated or unsaturated C₁-C₆ alkoxy; or (ii) R^{a} and R^{4a} together with the atoms to which they are attached, form a five to seven membered heterocycloalkyl or heteroaromatic ring;
   R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, heteroalkyl, cycloalkyl, heterocycloalkyl, aryl or heteroaryl is unsubstituted or substituted with one or more R^{b},

wherein R^{b} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R² and R⁵ are, each independently, selected from hydrogen, or a linear or branched, saturated or unsaturated C₁-C₆ alkyl or heteroalkyl,
wherein said alkyl or heteroalkyl is unsubstituted or substituted with one or more R^{e},
   wherein R^{e} is independently at each occurrence selected from the group consisting of COR^{f}, COOR^{f}, CONR^{f}R^{g}, NHCOR^{f}, and NR^{f}R^{g},
   wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring;
   R^{4b}, R^{4c} and R^{4d}, and when R^{4a}, does not form a five to seven membered ring with R^{a}, R^{4a}, are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{j},
r) COOR^{j},
s) CONR^{j}R^{k},
t) NHCOR^{j}, and
u) NR^{j}R^{k}
   wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{4a}, R^{4b}, R^{4c} and R^{4d}, together with the atoms to which they are attached, form a C₅-C₇ cycloalkyl, heterocycloalkyl, aromatic or heteroaromatic ring.

In one aspect, embodiments of the invention include a compound of Formula Ia:

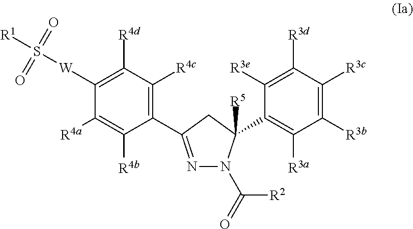

or a salt, solvate, hydrate, or prodrug thereof.

In one aspect, embodiments of the invention include a compound of Formula Ib:

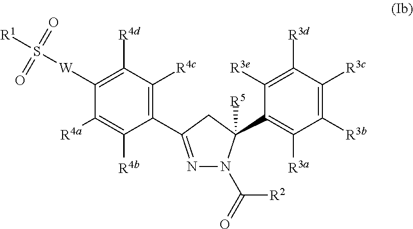

In one aspect, embodiments of the invention include a mixture of a compound of Formula Ia and a compound of Formula Ib.

Some embodiments of classes of Formulae I, Ia, and Ib are discussed below.

In some embodiments, W is absent. In some embodiments, W is NH. In some embodiments, W is NR^{a}, where R^{a} is not a hydrogen. In some embodiments, R^{a} and R^{4a} together with the atoms to which they are attached, form a five to seven membered heterocycloalkyl. For example, R^{a} and R^{4a} together with the atoms to which they are attached, form a five membered heterocycloalkyl ring, where R^{a} and R^{4a} collectively are (CH₂)₂.

In some embodiments, R¹ is C₁-C₆ alkyl, such as C₁-C₃ alkyl (e.g., methyl or ethyl).

In some embodiments, R¹ is an unsubstituted or substituted, aryl or heteroaryl group. For example, R¹ is a substituted or unsubstituted benzyl group. In some embodiments, the aryl or heteroaryl is substituted with one or more halogen (e.g., fluorine). In some cases, the aryl or heteroaryl may suibtably be substituted with a single halogen (e.g., R¹ is 4-fluorophenyl). In some cases, the aryl or heteroaryl may suibtably be substituted with two or more halogen (e.g., R¹ is 2,6-difluorophenyl). In some embodiments, the aryl or heteroaryl is substituted with one or more alkoxy (e.g., methoxy). In some cases, the aryl or heteroaryl may suibtably be substituted with a single alkoxy. In some cases, the aryl or heteroaryl may suibtably be substituted with two or more alkoxy. In some embodiments, the aryl or heteroaryl is substituted with two adjacent substituents that together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)ₚ—O—, where p is 1 to 3 (e.g., 2).

In some embodiments, R² is C₁-C₆ alkyl, such as C₁-C₃ alkyl (e.g., methyl or ethyl).

In some embodiments, R^{3a}, R^{3b}, R^{3c}, R^{3d} and R^{3e} are all hydrogen. In some embodiments, one or more of R^{3a}, R^{3b}, R^{3c}, R^{3d} and R^{3e} are not hydrogen. In some embodiments, only one of R^{3a}, R^{3b}, R_{3c}, R^{3d} and R^{3e} is not hydrogen, and the remaining values are hydrogen. In some embodiments, one or more of R^{3a}, R^{3b}, R^{3c}, R^{3d} and R^{3e} are halogen (e.g., fluorine). In some embodiments, only one of R^{3a}, R^{3b}, R^{3c}, R^{3d} and R^{3e} is halogen (e.g., either R^{3a} or R^{3c} is fluorine), and the remaining values are hydrogen. In some embodiments, one or more of R^{3a}, R^{3b}, R^{3c}, R^{3d} and R^{3e} are alkoxy (e.g., methoxy). In some embodiments, only one of R^{3a}, R^{3b}, R^{3c}, R^{3d} and R^{3e} is alkoxy (e.g., R^{3e} is methoxy), and the remaining values are hydrogen. In some embodiments, two or more of R^{3a}, R^{3b}, R^{3c}, R^{3d} and R^{3e} is alkoxy (e.g., methoxy), and the remaining values are hydrogen.

In some embodiments, two adjacent of R^{3a}, R^{3b}, R^{3c}, R^{3d} and R^{3e}, together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)_{q}—O—, where q is 1 to 3 (e.g., 1 or 2).

In some embodiments, R^{4a}, R^{4b}, r^{4c} and R^{4d} are all hydrogen. In some embodiments, one or more of R^{4a}, R^{4b}, R^{3c} and R^{4d} are not hydrogen. In some embodiments, only one of R^{4a}, R^{4b}, R^{4c} and R^{4d} is not hydrogen, and the remaining values are hydrogen.

In some embodiments, R⁵ is hydrogen. In some embodiments, R⁵ is not hydrogen.

In one aspect, the invention provides a compound of Formula II:

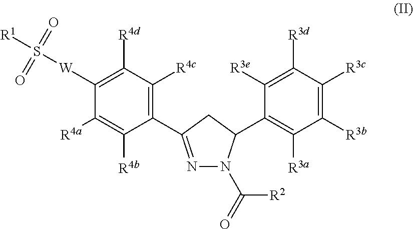

or a salt, solvate, hydrate, or prodrug thereof:
W is NR^{a} or is absent,
wherein R^{a} is (i) a group selected from the group consisting of a hydrogen, a linear or branched, saturated or unsaturated C₁-C₆ alkyl, C(O)R^{c}, and a linear or branched, saturated or unsaturated C₁-C₆ alkoxy; or (ii) R^{a} and R^{4a} together with the atoms to which they are attached, form a five to seven membered heterocycloalkyl or heteroaromatic ring;
   R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, heteroalkyl, cycloalkyl, heterocycloalkyl, aryl or heteroaryl is unsubstituted or substituted with one or more R^{b},

wherein R^{b} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
   wherein two R^{b} on adjacent carbon atoms form a heteroaryl ring or heterocycloalkyl ring;
      R² is selected from hydrogen, a linear or branched, saturated or unsaturated C₁-C₆ alkyl, heteroalkyl, heteroaryl, or aryl,
wherein said alkyl, heteroalkyl, heteroaryl, or aryl is unsubstituted or substituted with one or more R^{e},
   wherein R^{e} is independently at each occurrence selected from the group consisting of COR^{f}, COOR^{f}, CONR^{f}R^{g}, NHCOR^{f}, and NR^{f}R^{g},
   wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring;
   R^{4b}, R^{4c} and R^{4d}, and when R^{a4} does not form a five to seven membered ring with R^{a}, R^{4a}, are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
1) hydroxyl,
m) cyano,
n) amino,
o) nitro,
p) halogen,
q) COR^{j},
r) COORⱼ,
s) CONR^{j}R^{k},
t) NHCOR^{j}, and
u) NRⱼR^{k}
   wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{4a}, R^{4b}, R^{4c}, and R^{4d}, together with the atoms to which they are attached, form a C₅-C₇ cycloalkyl, heterocycloalkyl, aromatic or heteroaromatic ring.

In one aspect, compounds according to embodiments of the invention include a compound of Formula II, wherein:

W is NR^{a} or is absent,
wherein R^{a} is (i) a group selected from the group consisting of a hydrogen, a linear or branched, saturated or unsaturated C₁-C₆ alkyl, and a linear or branched, saturated or unsaturated C₁-C₆ alkoxy; or (ii) R^{a} and R^{4a} together with the atoms to which they are attached, form a five to seven membered heterocycloalkyl or heteroaromatic ring;
   R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, heteroalkyl, cycloalkyl, heterocycloalkyl, aryl or heteroaryl is unsubstituted or substituted with one or more R^{b},

wherein R^{b} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R² is selected from hydrogen, or a linear or branched, saturated or unsaturated C₁-C₆ alkyl and heteroalkyl,

wherein said alkyl or heteroalkyl is unsubstituted or substituted with one or more R^{e},
wherein R^{e} is independently at each occurrence selected from the group consisting of COR^{f}, COOR^{f}, CONR^{f}R^{g}, NHCOR^{f}, and NR^{f}R^{g},
wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
   R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d}, and R^{3e}, together with the atoms to which they are attached, form a C₅-C₇ cycloalkyl, heterocycloalkyl, aromatic or heteroaromatic ring;
   R^{4b}, R^{4c} and R^{4d}, and when R^{4a} does not form a five to seven membered ring with R^{a}, R^{4a}, are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{j},
r) COOR^{j},
s) CONR^{k}R^{k},
t) NHCOR^{j}, and
u) NRⱼR^{k}
   wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{4a}, R^{4b}, R^{4c} and R^{4d}, together with the atoms to which they are attached, form a C₅-C₇ cycloalkyl, heterocycloalkyl, aromatic or heteroaromatic ring.

In one aspect, embodiments of the invention include a compound of Formula IIa:

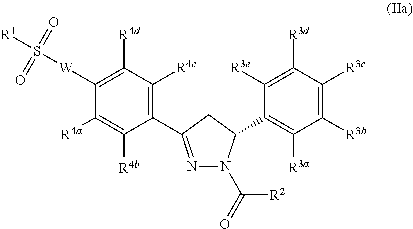

or a salt, solvate, hydrate, or prodrug thereof.

In one aspect, embodiments of the invention include a compound of Formula IIb:

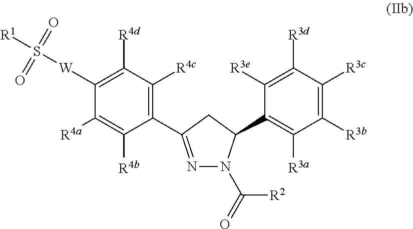

or a salt, solvate, hydrate, or prodrug thereof.

In one aspect, embodiments of the invention include a mixture of a compound of Formula IIa and a compound of Formula IIb.

Some embodiments of classes of formulae II, IIa, or IIb are described below.

In some embodiments, W is absent. In some embodiments, W is NH. In some embodiments, W is NR^{a}, where R^{a} is not hydrogen. In some embodiments, R^{a} and R^{4a} together with the atoms to which they are attached, form a five to seven membered heterocycloalkyl. For example, R^{a} and R^{4a} together with the atoms to which they are attached, form a five membered heterocycloalkyl ring, where R^{a} and R^{4a} collectively are (CH₂)₂.

In some embodiments, R¹ is C₁-C₆ alkyl, such as C₁-C₃ alkyl (e.g., methyl or ethyl).

In some embodiments, R¹ is an unsubstituted or substituted aryl or heteroaryl group. For example, R¹ is a substituted or unsubstituted benzyl group. In some embodiments, the aryl or heteroaryl is substituted with one or more halogen (e.g., fluorine). In some cases, the aryl or heteroaryl may suibtably be substituted with a single halogen (e.g., R¹ is 4-fluorophenyl). In some cases, the aryl or heteroaryl may suibtably be substituted with two or more halogen (e.g., R¹ is 2,6-difluorophenyl). In some embodiments, the aryl or heteroaryl is substituted with one or more alkoxy (e.g., methoxy). In some cases, the aryl or heteroaryl may suibtably be substituted with a single alkoxy. In some cases, the aryl or heteroaryl may suibtably be substituted with two or more alkoxy. In some embodiments, the aryl or heteroaryl is substituted with two adjacent substituents that together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)ₚ—O—, where p is 1 to 3 (e.g., 2).

In some embodiments, R² is C₁-C₆ alkyl, such as C₁-C₃ alkyl (e.g., methyl or ethyl).

In some embodiments, R^{3a}, R^{3b}, R^{3c}, R^{3d} and R^{3e} are all hydrogen. In some embodiments, one or more of R^{3a}, R^{3b}, R^{3c}, R^{3d} and R^{3e} are not hydrogen. In some embodiments, only one of R^{3a}, R^{3b}, R^{3c}, R^{3d} and R^{3e} is not hydrogen, and the remaining values are hydrogen. In some embodiments, one or more of R^{3a}, R^{3b}, R^{3c}, R^{3d} and R^{3e} are halogen (e.g., fluorine). In some embodiments, only one of R^{3a}, R^{3b}, R^{3c}, R^{3d} and R^{3e} is halogen (e.g., either R^{3a} or R^{3c} is fluorine), and the remaining values are hydrogen. In some embodiments, one or more of R^{3a}, R^{3b}, R^{3c}, R^{3d} and R^{3e} are alkoxy (e.g., methoxy). In some embodiments, only one of R^{3a}, R^{3b}, R^{3c}, R^{3d} and R^{3e} is alkoxy (e.g., R^{3e} is methoxy), and the remaining values are hydrogen. In some embodiments, two or more of R^{3a}, R^{3b}, R^{3c}, R^{3d} and R^{3e} is alkoxy (e.g., methoxy), and the remaining values are hydrogen.

In some embodiments, two adjacent of R^{3a}, R^{3b}, R^{3c}, R^{3d} and R^{3e}, together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)_{q}—O—, where q is 1 to 3 (e.g., 1 or 2).

In some embodiments, R^{4a}, R^{4b}, R_{4c} and R^{4d} are all hydrogen. In some embodiments, one or more of R^{4a}, R^{4b}, R_{4c} and R^{4e} are not hydrogen. In some embodiments, only one of R^{4a}, R^{4b}, R^{4c} and R^{4d} is not hydrogen, and the remaining values are hydrogen.

In one aspect, the invention provides a compound according to Formula III:

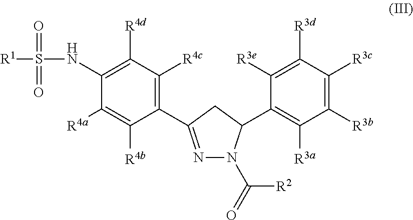

or a salt, solvate, hydrate or prodrug, wherein:
R¹ is selected from the group consisting of a hydrogen, a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, cycloalkyl, heterocycloalkyl, aryl or heteroaryl is unsubstituted or substituted with one or more R^{b},

wherein R^{b} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
   wherein two R^{b} on adjacent carbon atoms form a heteroaryl ring or a heterocycloalkyl ring;
      R² is selected from hydrogen, a linear or branched, saturated or unsaturated C₁-C₆ alkyl, heteroalkyl, heteroaryl, or aryl,
wherein said alkyl, heteroalkyl, heteroaryl, or aryl is unsubstituted or substituted with one or more R^{e},
   wherein R^{e} is independently at each occurrence selected from the group consisting of COR^{f}, COOR^{f}, CONR^{f}R^{g}, NHCOR^{f}, and NR^{f}R^{g},
   wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring;
   R^{4a}, R^{4b}, R^{4c} and R^{4d} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{j},
r) COORⱼ,
s) CONR^{j}R^{h},
t) NHCOR^{j}, and
u) NR^{j}R^{k}
   wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
or
two adjacent of R^{4a}, R^{4b}, R^{3c} and R^{4d}, together with the atoms to which they are attached, form a C₅-C₇ cycloalkyl, heterocycloalkyl, aromatic or heteroaromatic ring.

In one aspect, compounds according to embodiments of the invention include a compound of Formula III, wherein:

R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, heteroalkyl, cycloalkyl, heterocycloalkyl, aryl or heteroaryl is unsubstituted or substituted with one or more R^{b},

wherein R^{b} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R² is selected from hydrogen, or a linear or branched, saturated or unsaturated C₁-C₆ alkyl or heteroalkyl,

wherein said alkyl or heteroalkyl is unsubstituted or substituted with one or more R^{e},
wherein R^{e} is independently at each occurrence selected from the group consisting of COR^{f}, COOR^{f}, CONR^{f}R^{g}, NHCOR^{f}, and NR^{f}R^{g},
wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
   R^{3a}, R^{3b}, R^{3c}, R^{4d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring;
   R^{4a}, R^{4b}, R^{4c} and R^{4d} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{j},
r) COORⱼ,
s) CONR^{j}R^{k},
t) NHCOR^{j}, and
u) NRⱼR^{k}
   wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{4a}, R^{4b}, R^{4c} and R^{4d}, together with the atoms to which they are attached, form a C₅-C₇ cycloalkyl, heterocycloalkyl, aromatic or heteroaromatic ring.

In one aspect, embodiments of the invention include a compound of Formula Ma:

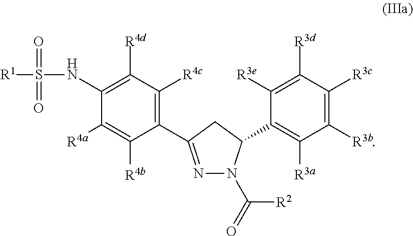

In one aspect, embodiments of the invention include a compound of Formula Mb:

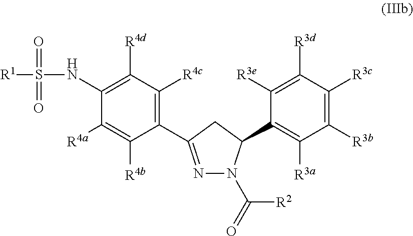

In one aspect, embodiments of the invention include a mixture of a compound of Formula IIIa and a compound of Formula Mb.

Some embodiments of classes of formulae III, IIIa, or IIIb are described below.

In some embodiments, R¹ is C₁-C₆ alkyl, such as C₁-C₃ alkyl (e.g., methyl or ethyl).

In some embodiments, R¹ is an unsubstituted or substituted aryl or heteroaryl group. For example, R¹ is a substituted or unsubstituted benzyl group. In some embodiments, the aryl or heteroaryl is substituted with one or more halogen (e.g., fluorine). In some cases, the aryl or heteroaryl may suibtably be substituted with a single halogen (e.g., R¹ is 4-fluorophenyl). In some cases, the aryl or heteroaryl may suibtably be substituted with two or more halogen (e.g., R¹ is 2,6-difluorophenyl). In some embodiments, the aryl or heteroaryl is substituted with one or more alkoxy (e.g., methoxy). In some cases, the aryl or heteroaryl may suibtably be substituted with a single alkoxy. In some cases, the aryl or heteroaryl may suibtably be substituted with two or more alkoxy. In some embodiments, the aryl or heteroaryl is substituted with two adjacent substituents that together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)ₚ—O—, where p is 1 to 3 (e.g., 2).

In some embodiments, R² is C₁-C₆ alkyl, such as C₁-C₃ alkyl (e.g., methyl or ethyl).

In some embodiments, R^{3a}, R^{3b}, R^{3c}, R^{3d} and R^{3e} are all hydrogen. In some embodiments, one or more of R^{3a}, R^{3b}, R^{3c}, R^{3d} and R^{3e} are not hydrogen. In some embodiments, only one of R^{3a}, R^{3b}, R^{3c}, R^{3d} and R^{3e} is not hydrogen, and the remaining values are hydrogen. In some embodiments, one or more of R^{3a}, R^{3b}, R^{3c}, R^{3d} and R^{3e} are halogen (e.g., fluorine). In some embodiments, only one of R^{3a}, R^{3b}, R^{3c}, R^{3d} and R^{3e} is halogen (e.g., either R^{3a} or R^{3e} is fluorine), and the remaining values are hydrogen. In some embodiments, one or more of R^{3a}, R^{3b}, R^{3c}, R^{3d} and R^{3e} are alkoxy (e.g., methoxy). In some embodiments, only one of R^{3a}, R^{3b}, R^{3c}, R^{3d} and R^{3e} is alkoxy (e.g., R^{3c} is methoxy), and the remaining values are hydrogen. In some embodiments, two or more of R^{3a}, R^{3b}, R^{3c}, R^{3d} and R^{3e} is alkoxy (e.g., methoxy), and the remaining values are hydrogen.

In some embodiments, two adjacent of R^{3a}, R^{3b}, R^{3c}, R^{3d} and R^{3e}, together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)_{q}—O—, where q is 1 to 3 (e.g., 1 or 2).

In some embodiments, R^{4a}, R^{4b}, R^{4c} and R^{4d} are all hydrogen. In some embodiments, one or more of R^{4a}, R^{3b}, R^{4c} and R^{4d} are not hydrogen. In some embodiments, only one of R^{4a}, R^{4b}, R^{4c} and R_{4d} is not hydrogen, and the remaining values are hydrogen.

In one aspect, the invention provides a compound according to Formula IV:

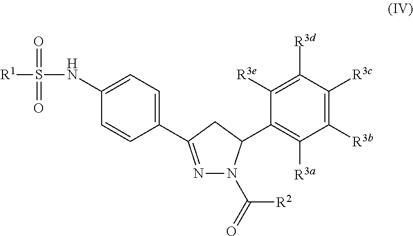

or a salt, solvate, hydrate, or prodrug thereof,
wherein:
R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, cycloalkyl, heterocycloalkyl, aryl or heteroaryl is unsubstituted or substituted with one or more R^{b},

wherein R^{b} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
   wherein two R^{b} on adjacent carbon atoms form a heteroaryl ring or a heterocycloalkyl ring;
      R² is selected from hydrogen, a linear or branched, saturated or unsaturated C₁-C₆ alkyl, heteroalkyl, heteroaryl, and aryl,
wherein said alkyl, heteroalkyl, heteroaryl, or aryl is unsubstituted or substituted with one or more R^{e},
   wherein R^{e} is independently at each occurrence selected from the group consisting of COR^{f}, COOR^{f}, CONR^{f}R^{g}, NHCOR^{f}, and NR^{f}R^{g},
   wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring.

In one aspect, compounds according to embodiments of the invention include a compound of Formula IV, wherein:

R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,

wherein said alkyl, heteroalkyl, cycloalkyl, heterocycloalkyl, aryl or heteroaryl is unsubstituted or substituted with one or more R^{h},

wherein R^{h} is independently at each occurrence selected from the group consisting of:
a) a linear or branched, saturated or unsaturated C₁-C₆ alkyl,
b) a linear or branched, saturated or unsaturated C₁-C₆ haloalkyl,
c) a linear or branched, saturated or unsaturated C₁-C₆ alkoxy or aryloxy,
d) a linear or branched, saturated or unsaturated C₁-C₆ haloalkoxy,
e) a linear or branched, saturated or unsaturated C₁-C₆ alkylsulfonyl,
f) a linear or branched, saturated or unsaturated C₁-C₆ thioalkyl or thioaryl,
g) a saturated or unsaturated C₃-C₈ cycloalkyl,
h) an aryl,
i) a heteroaryl,
j) a heterocycloalkyl,
k) hydroxyl,
l) cyano,
m) amino,
n) nitro,
o) halogen,
p) COR^{c},
q) COOR^{c},
r) CONR^{c}R^{d},
s) NHCOR^{c}, and
t) NR^{c}R^{d}
   wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R² is selected from hydrogen, or a linear or branched, saturated or unsaturated C₁-C₆ alkyl or heteroalkyl,
wherein said alkyl or heteroalkyl is unsubstituted or substituted with one or more R^{e},
   wherein R^{e} is independently at each occurrence selected from the group consisting of COR^{f}, COOR^{f}, CONR^{f}R^{g}, NHCOR^{f}, and NR^{f}R^{g},
   wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
      R^{3a}, R^{3b}, R^{3c}, R^{3d} and R^{3e} are, each independently, selected from:
a) hydrogen
b) a linear or branched, saturated or unsaturated C₁-C₆ alkyl,
c) a linear or branched, saturated or unsaturated C₁-C₆ haloalkyl,
d) a linear or branched, saturated or unsaturated C₁-C₆ alkoxy or aryloxy,
e) a linear or branched, saturated or unsaturated C₁-C₆ haloalkoxy,
f) a linear or branched, saturated or unsaturated C₁-C₆ alkylsulfonyl,
g) a linear or branched, saturated or unsaturated C₁-C₆ thioalkyl or thioaryl,
h) a saturated or unsaturated C₃-C₈ cycloalkyl,
i) an aryl,
j) a heteroaryl,
k) a heterocycloalkyl,
l) hydroxyl,
m) cyano,
n) amino,
o) nitro,
p) halogen,
q) COR^{h},
r) COOR^{h},
s) CONR^{h}Rⁱ,
t) NHCOR^{h}, and
u) NR^{h}Rⁱ
   wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;

or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring.

In one aspect, embodiments of the invention include a compound of Formula IVa:

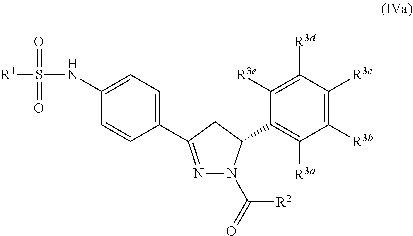

or a salt, solvate, hydrate, or prodrug thereof.

In one aspect, embodiments of the invention include a compound of Formula IVb:

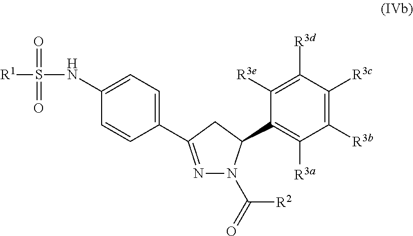

or a salt, solvate, hydrate, or prodrug thereof.

In one aspect, embodiments of the invention include a mixture of a compound of Formula IVa and a compound of Formula IVb.

Some embodiments of preferred classes of formulae IV, IVa, and IVb are described below.

In some embodiments, R¹ is C₁-C₆ alkyl, such as a C₁-C₃ alkyl (e.g., methyl or ethyl).

In some embodiments, R¹ is an unsubstituted or substituted aryl or heteroaryl group. For example, R¹ is a substituted or unsubstituted benzyl group. In some embodiments, the aryl or heteroaryl is substituted with one or more halogen (e.g., fluorine). In some cases, the aryl or heteroaryl may suibtably be substituted with a single halogen (e.g., R¹ is 4-fluorophenyl). In some cases, the aryl or heteroaryl may suibtably be substituted with two or more halogen (e.g., R¹ is 2,6-difluorophenyl). In some embodiments, the aryl or heteroaryl is substituted with one or more alkoxy (e.g., methoxy). In some cases, the aryl or heteroaryl may suibtably be substituted with a single alkoxy. In some cases, the aryl or heteroaryl may suibtably be substituted with two or more alkoxy. In some embodiments, the aryl or heteroaryl is substituted with two adjacent substituents that together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)ₚ—O—, where p is 1 to 3 (e.g., 2).

In some embodiments, R² is C₁-C₆ alkyl, such as C₁-C₃ alkyl (e.g., methyl or ethyl).

In some embodiments, R^{3a}, R^{3b}, R^{3c}, R^{3d} and R^{3e} are all hydrogen. In some embodiments, one or more of R^{3a}, R^{3b}, R^{3c}, R^{3d} and R^{3e} are not hydrogen. In some embodiments, only one of R^{3a}, R^{3b}, R^{3c}, R^{3d} and R^{3e} is not hydrogen, and the remainding values are hydrogen.

In some embodiments, one or more of R^{3a}, R^{3b}, R^{3c}, R^{3d} and R^{3e} are halogen (e.g., fluorine). In some embodiments, only one of R^{3a}, R^{3b}, R^{3c}, R^{3d} and R^{3e} is halogen (e.g., either R^{3a} or R^{3c} is fluorine), and the remaining values are hydrogen. In some embodiments, one or more of R^{3a}, R^{3b}, R^{3c}, R^{3d} and R^{3e} are alkoxy (e.g., methoxy). In some embodiments, only one of R^{3a}, R^{3b}, R^{3c}, R^{3d} and R^{3c} is alkoxy (e.g., R^{3c} is methoxy), and the remaining values are hydrogen. In some embodiments, two or more of R^{3a}, R^{3b}, R^{3c}, R^{3d} and R^{3e} is alkoxy (e.g., methoxy), and the remaining values are hydrogen.

In some embodiments, two adjacent of R^{3a}, R^{3b}, R^{3c}, R^{3d} and R^{3e}, together with the atoms to which they are attached, form a five to seven membered ring. In some embodiments, the ring is a six membered ring. In some embodiments, the ring is a five membered ring. In some embodiments, the two adjacent substituents, together with the atoms to which they are attached, form a heterocycloalkyl ring, where the two adjacent substituents collectively are —O—(CH₂)_{q}—O—, where q is 1 to 3 (e.g., 1 or 2).

In one aspect, the invention provides a compound of Formula V:

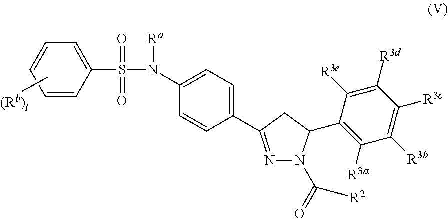

or a salt, solvate, hydrate, or prodrug thereof, wherein R^{a}, R², R^{3a}, R^{3b}, R^{3c}, R^{3d}, R^{3e}, and R^{b} are as described for Formula I and t is 0, 1, 2, 3, 4 or 5.

In one aspect, embodiments of the invention include a compound of Formula Va:

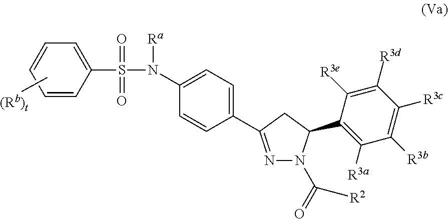

or a salt, solvate, hydrate, or prodrug thereof, wherein R^{a}, R², R^{3a}, R^{3b}, R^{3c}, R^{3d}, R^{3e}, and R^{b} are as described for Formula I and t is 0, 1, 2, 3, 4 or 5.

In one aspect, embodiments of the invention include a compound of Formula Vb:

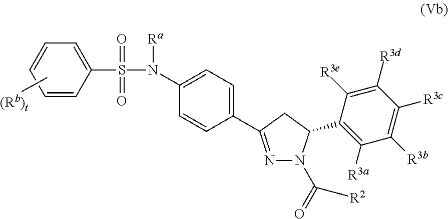

or a salt, solvate, hydrate, or prodrug thereof, wherein R^{a}, R², R^{3a}, R^{3b}, R^{3c}, R^{3d}, R^{3e}, and R^{b} are as described for Formula I and t is 0, 1, 2, 3, 4 or 5.

In one aspect, the invention provides a compound of Formula VI:

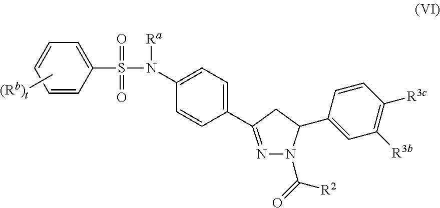

or a salt, solvate, hydrate, or prodrug thereof, wherein R^{a}, R², R^{3b}, R^{3c}, and R^{b} are as described for Formula I and t is 0, 1, 2, 3, 4 or 5.

In one aspect, embodiments of the invention include a compound of Formula VIa:

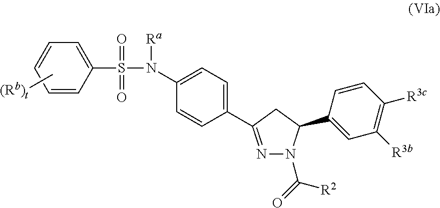

or a salt, solvate, hydrate, or prodrug thereof, wherein R^{a}, R², R^{3b}, R^{3c}, and R^{b} are as described for Formula I and t is 0, 1, 2, 3, 4 or 5.

In one aspect, embodiments of the invention include a compound of Formula VIb:

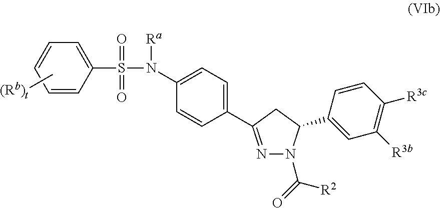

or a salt, solvate, hydrate, or prodrug thereof, wherein R^{a}, R², R^{3b}, R_{3c}, and R^{b} are as described for Formula I and t is 0, 1, 2, 3, 4 or 5.

Some embodiments of preferred classes are described below for formulae of the invention.

In one aspect, embodiments of the invention include a compound of Formulae I, Ia, Ib, II, IIa, IIb, III, IIIa, IIIb, IV, IVa, or IVb, wherein R¹ is a C₁-C₃ alkyl. In one aspect, embodiments of the invention include a compound of Formulae I, Ia, Ib, II, IIa, IIb, III, IIIa, IIIb, IV, IVa, or IVb, wherein R¹ is methyl. In one aspect, embodiments of the invention include a compound of Formulae I, Ia, Ib, II, IIa, IIb, III, IIIa, IIIb, IV, IVa, or IVb, wherein R¹ is ethyl.

In one aspect, embodiments of the invention include a compound of Formulae I, Ia, Ib, II, IIa, IIb, III, IIIa, IIIb, IV, IVa, or IVb), wherein R¹ is a substituted or unsubstituted aryl. In one aspect, embodiments of the invention include a compound of Formulae I, Ia, Ib, II, IIa, IIb, III, IIIa, IIIb, IV, IVa, or IVb, wherein R¹ is aryl substituted with one or more halogen. In one aspect, embodiments of the invention include a compound of Formulae I, Ia, Ib, II, IIa, IIb, III, IIIa, IIIb, IV, IVa, or IVb, wherein R¹ is an unsubstituted phenyl. In one aspect, embodiments of the invention include a compound of Formulae I, Ia, Ib, II, IIa, IIb, III, IIIa, IIIb, IV, IVa, or IVb, wherein R¹ is a phenyl substituted with one R^{b}.

In one aspect, embodiments of the invention include a compound of Formulae I, Ia, Ib, II, IIa, IIb, III, IIIa, IIIb, IV, IVa, or IVb, wherein R¹ is:

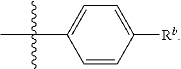

In one aspect, embodiments of the invention include a compound of Formulae I, Ia, Ib, II, IIa, IIb, III, IIIa, IIIb, IV, IVa, or IVb) wherein R¹ is:

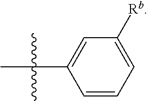

In one aspect, embodiments of the invention include a compound of Formulae I, Ia, Ib, II, IIa, IIb, III, IIIa, IIIb, IV, IVa, or IVb), wherein R¹ is:

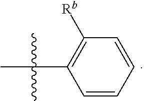

In one aspect, embodiments of the invention include a compound of Formulae I, Ia, Ib, II, IIa, IIb, III, IIIa, IIIb, IV, IVa, or IVb, wherein R¹ is a phenyl substituted with two R^{b}.

In one aspect, embodiments of the invention include a compound of Formulae I, Ia, Ib, II, IIa, IIb, III, IIIa, IIIb, IV, IVa, or IVb, wherein R¹ is:

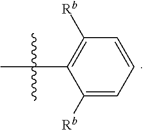

In one aspect, embodiments of the invention include a compound of Formulae I, Ia, Ib, II, IIa, IIb, III, IIIa, IIIb, IV, IVa, or IVb, wherein R¹ is a phenyl substituted with one or more R^{b} and R^{b} is selected from halogen, C₁-C₃ alkoxy, CF₃, NHC(O)R^{c}, and NR^{c}R^{d} or two R^{b} on adjacent carbon atoms form a heterocycloalkyl ring and R^{c} and R^{d} are, each independently, hydrogen or linear or branched, saturated or unsaturated C₁-C₃ alkyl.

In one aspect, embodiments of the invention include a compound of Formulae I, Ia, Ib, II, IIa, IIb, III, IIIa, IIIb, IV, IVa, or IVb, wherein R¹ is a phenyl substituted with one or more R^{b} and R^{b} is selected from fluorine, chlorine, methoxy, CF₃, NHC(O)CH₃, and NH₂ or two R^{b} on adjacent carbon atoms form a heterocycloalkyl ring.

In one aspect, embodiments of the invention include a compound of Formulae I, Ia, Ib, II, IIa, IIb, III, IIIa, IIIb, IV, IVa, or IVb, wherein R¹ is a phenyl substituted with one or more R^{b} and R^{b} is selected from fluorine, CF₃, NHC(O)CH₃, and NH₂ or two R^{b} on adjacent carbon atoms form a heterocycloalkyl ring.

In one aspect, embodiments of the invention include a compound of Formulae I, Ia, Ib, II, IIa, IIb, III, IIIa, IIIb, IV, IVa, or IVb, wherein R¹ is a phenyl substituted with one or more fluorine. In one aspect, embodiments of the invention include a compound of Formulae I, Ia, Ib, II, IIa, IIb, III, IIIa, IIIb, IV, IVa, or IVb, wherein R¹ is a phenyl substituted with one fluorine. In one aspect, embodiments of the invention include a compound of Formulae I, Ia, Ib, II, IIa, IIb, III, IIIa, IIIb, IV, IVa, or IVb, wherein R¹ is a phenyl substituted with two fluorine.

In one aspect, embodiments of the invention include a compound of Formulae I, Ia, Ib, II, IIa, IIb, III, IIIa, IIIb, IV, IVa, or IVb, wherein R¹ is a phenyl substituted with one or more methoxy. In one aspect, embodiments of the invention include a compound of Formulae I, Ia, Ib, II, IIa, IIb, III, IIIa, IIIb, IV, IVa, or IVb, wherein R¹ is a phenyl substituted with one methoxy.

In one aspect, embodiments of the invention include a compound of Formulae I, Ia, Ib, II, IIa, IIb, III, IIIa, IIIb, IV, IVa, or IVb, wherein R¹ is a phenyl substituted with one or more CF₃. In one aspect, embodiments of the invention include a compound of Formulae I, Ia, Ib, II, IIa, IIb, III, IIIa, IIIb, IV, IVa, or IVb, wherein R¹ is a phenyl substituted with one CF₃.

In one aspect, embodiments of the invention include a compound of Formulae I, Ia, Ib, II, IIa, IIb, III, IIIa, IIIb, IV, IVa, or IVb, wherein R¹ is a phenyl substituted two R^{b} to form the heterocycloalkyl ring:

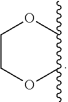

In one aspect, embodiments of the invention include a compound of Formulae V, Va, Vb, VI, VIa, or VIb), wherein R^{b} is selected from halogen, C₁-C₃ alkoxy, CF₃, NHC(O)R^{c}, and NR^{c}R^{d} or two R^{b} on adjacent carbon atoms form a heterocycloalkyl ring and R^{c} and R^{d} are, each independently, hydrogen or linear or branched, saturated or unsaturated C₁-C₃ alkyl.

In one aspect, embodiments of the invention include a compound of FIn one aspect, embodiments of the invention include a compound of Formulae V, Va, Vb, VI, VIa, or VIb), wherein R^{b} is selected from fluorine, chlorine, methoxy, CF₃, NHC(O)CH₃, and NH₂ or two R^{b} on adjacent carbon atoms form a heterocycloalkyl ring.

In one aspect, embodiments of the invention include a compound of Formulae V, Va, Vb, VI, VIa, or VIb, wherein R^{b} is selected from fluorine, CF₃, NHC(O)CH₃, and NH₂ or two R_{b} on adjacent carbon atoms form a heterocycloalkyl ring.

In one aspect, embodiments of the invention include a compound of Formulae V, Va, Vb, VI, VIa, or VIb, wherein R^{b} is fluorine.

In one aspect, embodiments of the invention include a compound of Formulae V, Va, Vb, VI, VIa, or VIb, wherein R^{b} is methoxy.

In one aspect, embodiments of the invention include a compound of Formulae V, Va, Vb, VI, VIa, or VIb, wherein R^{b} is CF₃.

In one aspect, embodiments of the invention include a compound of Formulae I, Ia, Ib, II, IIa, IIb, III, IIIa, IIIb, IV, IVa, IVb, V, Va, Vb, VI, VIa, or VIb, wherein R² is selected from C₁-C₃ alkyl, heteroaryl, and aryl.

In one aspect, embodiments of the invention include a compound of Formulae I, Ia, Ib, II, IIa, IIb, III, IIIa, IIIb, IV, IVa, IVb, V, Va, Vb, VI, VIa, or VIb, wherein R² is C₁-C₃ alkyl. In one aspect, embodiments of the invention include a compound of Formulae I, Ia, Ib, II, IIa, IIb, III, IIIa, IIIb, IV, IVa, IVb, V, Va, Vb, VI, VIa, or VIb, wherein R² is methyl or ethyl. In one aspect, embodiments of the invention include a compound of Formulae I, Ia, Ib, II, IIa, lib, III, IIIa, IIIb, IV, IVa, IVb, V, Va, Vb, VI, VIa, or VIb, wherein R² is methyl.

In one aspect, embodiments of the invention include a compound of Formulae I, Ia, Ib, II, IIa, IIb, III, IIIa, IIIb, IV, IVa, IVb, V, Va, Vb, VI, VIa, or VIb), wherein R² is heteroaryl. In one aspect, embodiments of the invention include a compound of Formulae I, Ia, Ib, II, IIa, IIb, III, IIIa, IIIb, IV, IVa, IVb, V, Va, Vb, VI, VIa, or VIb, wherein R² is furan or pyridine.

In one aspect, embodiments of the invention include a compound of Formulae I, Ia, Ib, II, IIa, IIb, III, IIIa, IIIb, IV, IVa, IVb, V, Va, or Vb, wherein one of R^{3a}, R^{3b}, R^{3c}, R^{3d} and R^{3e} is not hydrogen and the remaining R^{3a}, R^{3b}, R^{3c}, R^{3d} and R^{3e} are hydrogen. In one aspect, embodiments of the invention include a compound of Formulae I, Ia, Ib, II, IIa, IIb, III, IIIa, TIM, IV, IVa, IVb, V, Va, or Vb, wherein R^{3c} is not hydrogen. In one aspect, embodiments of the invention include a compound of Formulae I, Ia, Ib, II, IIa, IIb, III, IIIa, IIIb, IV, IVa, IVb, V, Va, or Vb), wherein two of R^{3a}, R^{3b}, R^{3c}, R^{3d} and R^{3e} are not hydrogen and the remaining R^{3a}, R^{3b}, R^{3c}, R^{3d}, and R^{3e} are hydrogen. In one aspect, embodiments of the invention include a compound of Formulae I, Ia, Ib, II, IIa, IIb, III, TIM, TIM, IV, IVa, IVb, V, Va, or Vb, wherein R^{3b} and R^{3c} are not hydrogen and the remaining R^{3a}, R^{3d} and R^{3e} are hydrogen. In one aspect, embodiments of the invention include a compound of Formulae I, Ia, Ib, II, IIa, IIb, III, IIIa, TIM, IV, IVa, IVb, V, Va, or Vb, wherein three of R^{3a}, R^{3b}, R^{3c}, R^{3d} and R^{3e} are not hydrogen and the remaining R^{3a}, R^{3b}, R^{3c}, R^{3d} and R^{3e} are hydrogen.

In one aspect, embodiments of the invention include a compound of Formulae I, Ia, Ib, II, IIa, IIb, III, IIIa, IIIb, IV, IVa, IVb, V, Va, or Vb, wherein one or more of R^{3a}, R^{3b}, R^{3c}, R^{3d} and R^{3e} is C₁-C₃ alkoxy, halogen, C₁-C₃ alkyl, or NR^{h}Rⁱ, wherein R^{h} and TV are each independently, hydrogen or C₁-C₃ alkyl or two adjacent of R^{3a}, R^{3b}, R_{3c}, R^{3d}, and R^{3e}, together with the atoms to which they are attached, form a heterocycloalkyl or heteroaromatic ring.

In one aspect, embodiments of the invention include a compound of Formulae I, Ia, Ib, II, IIa, IIb, III, IIIa, IIIb, IV, IVa, IVb, V, Va, or Vb, wherein one or more of R^{3a}, R^{3b}, R^{3c}, R^{3d} and R^{3e} is methoxy, fluorine, chlorine, methyl, or NH₂.

In one aspect, embodiments of the invention include a compound of Formulae I, Ia, Ib, II, IIa, IIb, III, TIM, TIM, IV, IVa, IVb, V, Va, or Vb, wherein one of R^{3a}, R^{3b}, R^{3c}, R^{3d} and R^{3e} is methoxy and the remaining R^{3a}, R^{3b}, R^{3c}, R^{3d} and R^{3e} are hydrogen.

In one aspect, embodiments of the invention include a compound of Formulae I, Ia, Ib, II, IIa, IIb, III, IIIa, IIIb, IV, IVa, IVb, V, Va, or Vb, wherein two adjacent of R^{3a}, R^{3b}, R^{3c}, R^{3d}, and R^{3e}, together with the atoms to which they are attached, form a ring selected from:

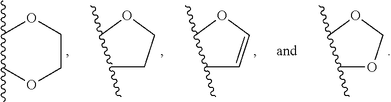

In one aspect, embodiments of the invention include a compound of Formulae I, Ia, Ib, II, IIa, IIb, III, IIIa, IIIb, IV, IVa, IVb, V, Va, or Vb, wherein two adjacent of R^{3b} and R^{3c} together with the atoms to which they are attached, form a ring selected from:

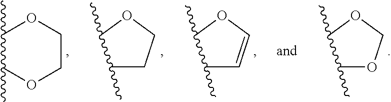

In one aspect, embodiments of the invention include a compound of Formulae VI, VIa, or VIb), wherein one of R^{3b} or R^{3c} is C₁-C₃ alkoxy, halogen, C₁-C₃ alkyl, or NR^{h}Rⁱ, wherein R^{h} and Rⁱ are each independently, hydrogen or C₁-C₃ alkyl and the remaining R^{3b} or R^{3c} is hydrogen.

In one aspect, embodiments of the invention include a compound of Formulae VI, VIa, or VIb, wherein one of R^{3b} or R^{3c} is methoxy, fluorine, chlorine, methyl, or NH₂ and the remaining R^{3b} or R^{3c} is hydrogen.

In one aspect, embodiments of the invention include a compound of Formulae I, R^{3b}, R^{3c}, Ia, Ib, II, IIa, IIb, III, IIIa, IIIb, IV, IVa, IVb, V, Va, or Vb, wherein one or more of R^{3a}, R^{3d} and R^{3e} is C₁-C₃ alkoxy.

In one aspect, embodiments of the invention include a compound of Formulae I, Ia, Ib, II, IIa, IIb, III, IIIa, IIIb, IV, IVa, IVb, V, Va, or Vb, wherein one or more of R^{3a}, R^{3b}, R^{3c}, R^{3d} and R^{3e} is methoxy.

In one aspect, embodiments of the invention include a compound of Formulae I, Ia, Ib, II, IIa, IIb, III, IIIa, or IIIb, wherein R^{4a}, R^{4b}, R^{4c}, and R^{4d} are each hydrogen.

In one aspect, embodiments of the invention include a compound of Formulae I, Ia, or Ib, wherein R⁵ is hydrogen.

In one aspect, embodiments of the invention include a compound of Formula V, Va, Vb, VI, VIa or VIb, wherein t is 0.

In one aspect, embodiments of the invention include a compound of Formula V, Va, Vb, VI, VIa or VIb, wherein t is 1, 2, or 3.

In one aspect, embodiments of the invention include a compound of Formula V, Va, Vb, VI, VIa or VIb, wherein t is 1

In one aspect, embodiments of the invention include a compound of Formula V, Va, Vb, VI, VIa or VIb, wherein t is 2.

While all enantiomers and mixtures thereof are useful as PKM2 modulators, in one aspect, single enantiomers are preferred. Furthermore, while all of the compounds of the invention are useful as PKM2 modulators, certain classes are as described above. It will be understood that the above classes may be combined to form additional classes, as for example the combination of certain selections for two or more substituents. Some illustrative combinations are below for Formula V, Va, Vb, VI, VIa, and VIb.

In one aspect, embodiments of the invention include a compound of Formula V, Va, Vb, wherein R^{a} is hydrogen; R² is methyl or heteroaryl; R^{b} is fluorine, CF₃, NHC(O)CH₃, NH₂ or two R^{b} on adjacent carbon atoms form a heterocycloalkyl ring; t is 0, 1, or 2; R^{3a}, R^{3d}, and R^{3c} are each hydrogen and one of R^{3b} or R^{3c} is methoxy, fluorine, chlorine, methyl, or NH₂ and the remaining R^{3b} or R^{3c} is hydrogen or R^{3b} and R^{3c} form a ring selected from:

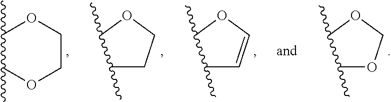

In one aspect, embodiments of the invention include a compound of Formula V, Va, Vb, wherein R^{a} is hydrogen; R² is methyl; R^{b} is fluorine, CF₃, NHC(O)CH₃, NH₂ or two R^{b} on adjacent carbon atoms form a heterocycloalkyl ring; t is 0, 1, or 2; R^{3a}, R^{3d}, and R^{3c} are each hydrogen and one of R^{3b} or R^{3c} is methoxy, fluorine, chlorine, methyl, or NH₂ and the remaining R^{3b} or R^{3c} is hydrogen or R^{3b} and R^{3c} form a ring selected from:

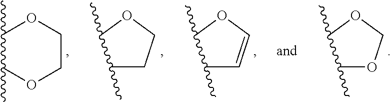

In one aspect, embodiments of the invention include a compound of Formula V, Va, Vb, wherein R^{a} is hydrogen; R² is methyl or heteroaryl; R^{b} is fluorine, CF₃, NHC(O)CH₃, NH₂; t is 0, 1, or 2; R^{3a}, R^{3d}, and R^{3e} are each hydrogen and one of R^{3b} or R^{3c} is methoxy, fluorine, chlorine, methyl, or NH₂ and the remaining R^{3b} or R^{3c} is hydrogen or R^{3b} and R^{3c} form a ring selected from:

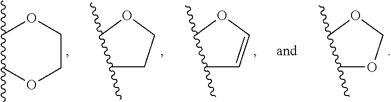

In one aspect, embodiments of the invention include a compound of Formula V, Va, Vb, wherein R^{a} is hydrogen; R² is methyl; R^{b} is fluorine; t is 1, or 2; R^{3a}, R^{3b}, and R^{3c} are each hydrogen and one of R^{3b} or R^{3c} is methoxy, fluorine, chlorine, methyl, or NH₂ and the remaining R^{3b} or R^{3c} is hydrogen or R^{3b} and R^{3c} form a ring selected from:

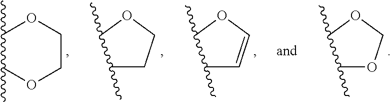

In one aspect, embodiments of the invention include a compound of Formula V, Va, Vb, wherein R^{a} is hydrogen; R² is methyl; R^{b} is fluorine; t is 1; R^{3a}, R^{3d}, and R^{3c} are each hydrogen and one of R^{3b} or R^{3c} is methoxy, fluorine, chlorine, methyl, or NH₂ and the remaining R^{3b} or R^{3c} is hydrogen or R^{3b} and R^{3c} form a ring selected from:

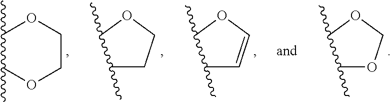

In one aspect, embodiments of the invention include a compound of Formula V, Va, Vb, wherein R^{a} is hydrogen; R² is methyl; R^{b} is fluorine; t is 2; R^{3a}, R^{3d}, and R^{3c} are each hydrogen and one of R^{3b} or R^{3c} is methoxy, fluorine, chlorine, methyl, or NH₂ and the remaining R^{3b} or R^{3c} is hydrogen or R^{3b} and R^{3c} form a ring selected from:

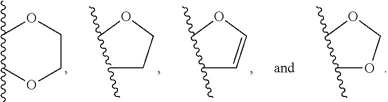

In one aspect, embodiments of the invention include a compound of Formula V, Va, Vb, wherein R^{a} is hydrogen; R² is methyl; R^{b} is fluorine, CF₃, NHC(O)CH₃, NH₂ or two Rb on adjacent carbon atoms form a heterocycloalkyl ring; t is 0, 1, or 2; R^{3a}, R^{3d}, and R^{3e} are each hydrogen and one of R^{3b} or R^{3c} is methoxy, fluorine, chlorine, methyl, or NH₂.

In one aspect, embodiments of the invention include a compound of Formula V, Va, Vb, wherein R^{a} is hydrogen; R² is methyl; R^{b} is fluorine, CF₃, NHC(O)CH₃, NH₂ or two R^{b} on adjacent carbon atoms form a heterocycloalkyl ring; t is 0, 1, or 2; R^{3a}, R^{3d}, and R^{3e} are each hydrogen and R^{3b} and R^{3c} form a ring selected from:

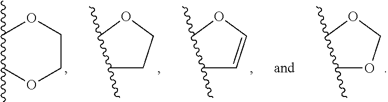

In one aspect, embodiments of the invention include a compound of Formula V, Va, Vb, wherein R^{a} is hydrogen; R² is methyl; R^{b} is fluorine, CF₃, NHC(O)CH₃, NH₂ or two R^{b} on adjacent carbon atoms form a heterocycloalkyl ring; t is 0, 1, or 2; R^{3a}, R^{3d}, and R^{3e} are each hydrogen and one of R^{3b} or R^{3c} is methoxy.

In one aspect, embodiments of the invention include a compound of Formula V, Va, Vb, wherein R^{a} is hydrogen; R² is methyl; R^{b} is fluorine, CF₃, NHC(O)CH₃, or NH₂; t is 0, 1, or 2; R^{3a}, R^{3d}, and R^{3e} are each hydrogen and one of R^{3b} or R^{3c} is methoxy.

In one aspect, embodiments of the invention include a compound of Formula VI, VIa, VIb, wherein R^{a} is hydrogen; R² is methyl or heteroaryl; R^{b} is fluorine, CF₃, NHC(O)CH₃, or NH₂ or two R^{b} on adjacent carbon atoms form a heterocycloalkyl ring; t is 0, 1, or 2; one of R^{3b} or R^{3c} is methoxy, fluorine, chlorine, methyl, or NH₂ and the remaining R^{3b} or R^{3c} is hydrogen or R^{3b} and R^{3c} form a ring selected from:

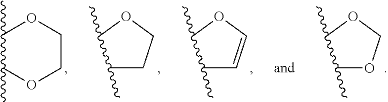

In one aspect, embodiments of the invention include a compound of Formula VI, VIa, VIb, wherein R^{a} is hydrogen; R² is methyl; R^{b} is fluorine, CF₃, NHC(O)CH₃, or NH₂ or two R^{b} on adjacent carbon atoms form a heterocycloalkyl ring; t is 0, 1, or 2; one of R^{3b} or R^{3c} is methoxy, fluorine, chlorine, methyl, or NH₂ and the remaining R^{3b} or R^{3c} is hydrogen or R^{3b} and R^{3c} form a ring selected from:

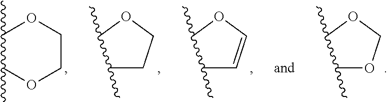

In one aspect, embodiments of the invention include a compound of Formula VI, VIa, VIb, wherein R^{a} is hydrogen; R² is methyl or heteroaryl; R^{b} is fluorine, CF₃, NHC(O)CH₃, or NH₂; t is 0, 1, or 2; one of R^{3b} or R^{3c} is methoxy, fluorine, chlorine, methyl, or NH₂ and the remaining R^{3b} or R^{3c} is hydrogen or R^{3b} and R^{3c} form a ring selected from:

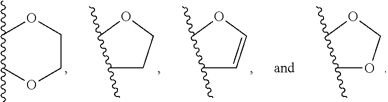

In one aspect, embodiments of the invention include a compound of Formula V, Va, Vb, wherein R^{a} is hydrogen; R² is methyl; R^{b} is fluorine; t is 1, or 2; one of R^{3b} or R^{3c} is methoxy, fluorine, chlorine, methyl, or NH₂ and the remaining R^{3b} or R^{3c} is hydrogen or R^{3b} and R^{3c} form a ring selected from:

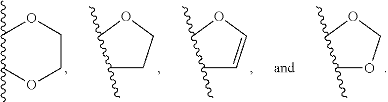

In one aspect, embodiments of the invention include a compound of Formula VI, VIa, VIb, wherein R^{a} is hydrogen; R² is methyl; R^{b} is fluorine; t is 1; one of R^{3b} or R^{3c} is methoxy, fluorine, chlorine, methyl, or NH₂ and the remaining R^{3b} or R^{3c} is hydrogen or R^{3b} and R^{3c} form a ring selected from:

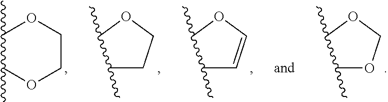

In one aspect, embodiments of the invention include a compound of Formula VI, VIa, VIb, wherein R^{a} is hydrogen; R² is methyl; R^{b} is fluorine; t is 2; one of R^{3b} or R^{3c} is methoxy, fluorine, chlorine, methyl, or NH₂ and the remaining R^{3b} or R^{3c} is hydrogen or R^{3b} and R^{3c} form a ring selected from:

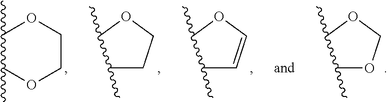

In one aspect, embodiments of the invention include a compound of Formula VI, VIa, VIb, wherein R^{a} is hydrogen; R² is methyl; R^{b} is fluorine, CF₃, NHC(O)CH₃, or NH₂ or two R^{b} on adjacent carbon atoms form a heterocycloalkyl ring; t is 0, 1, or 2; one of R^{3b} or R^{3c} is methoxy, fluorine, chlorine, methyl, or NH₂.

In one aspect, embodiments of the invention include a compound of Formula VI, VIa, VIb, wherein R^{a} is hydrogen; R² is methyl; R^{b} is fluorine, CF₃, NHC(O)CH₃, NH₂ or two R^{b} on adjacent carbon atoms form a heterocycloalkyl ring; t is 0, 1, or 2; and R^{3b} and R^{3c} form a ring selected from:

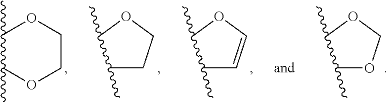

In one aspect, embodiments of the invention include a compound of Formula VI, VIa, VIb, wherein R^{a} is hydrogen; R² is methyl; R^{b} is fluorine, CF₃, NHC(O)CH₃, or NH₂ or two R^{b} on adjacent carbon atoms form a heterocycloalkyl ring; t is 0, 1, or 2; one of R^{3b} or R^{3c} is methoxy and the remaining R^{3b} or R^{3c} is hydrogen.

In one aspect, embodiments of the invention include a compound of Formula VI, VIa, VIb, wherein R^{a} is hydrogen; R² is methyl; R^{b} is fluorine, CF₃, NHC(O)CH₃, or NH₂; t is 0, 1, or 2; and one of R^{3b} or R^{3c} is methoxy and the remaining R^{3b} or R^{3c} is hydrogen.

In one aspect, the invention provides a compound of Table 1.

| Comp. # | Structure |
|---|---|
| 1A | 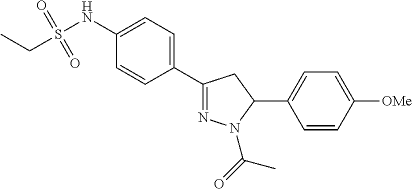 |
| 2A | 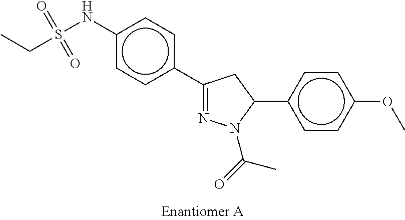 |
| 3A | 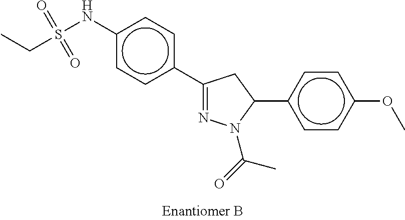 |
| 4A | 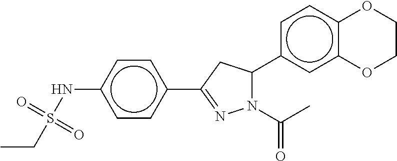 |
| 5A | 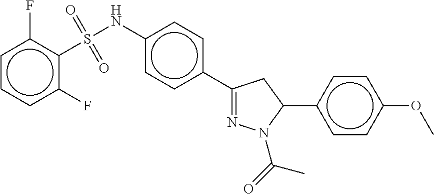 |
| 6A | 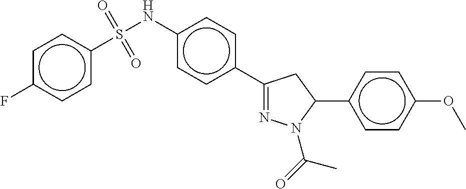 |
| 7A | 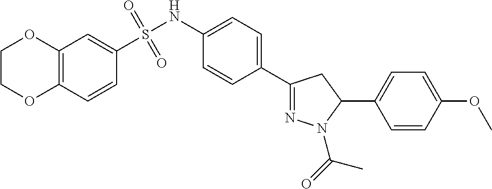 |
| 8A | 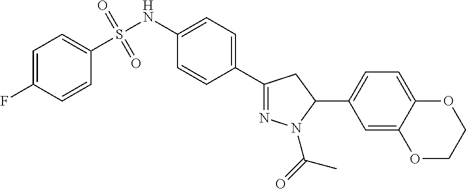 |
| 9A | 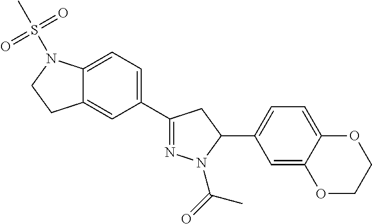 |
| 10A | 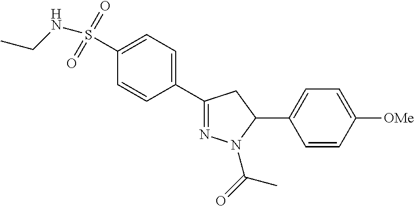 |
| 11A | 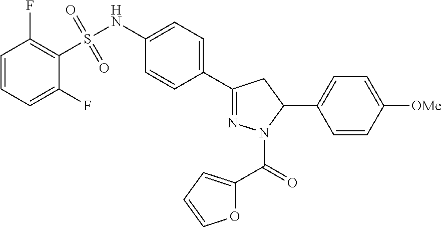 |
| 12A | 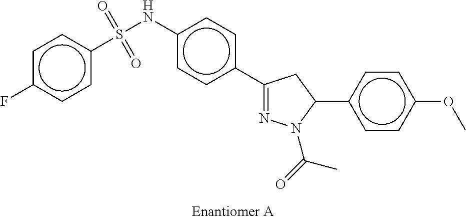 |
| 13A | 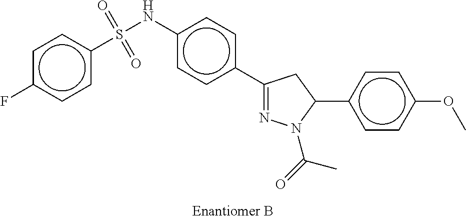 |
| 14A | 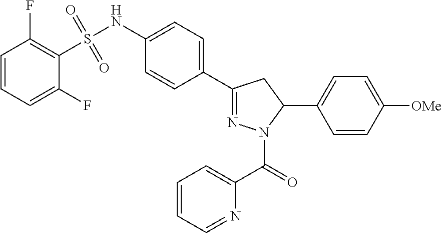 |
| 15A | 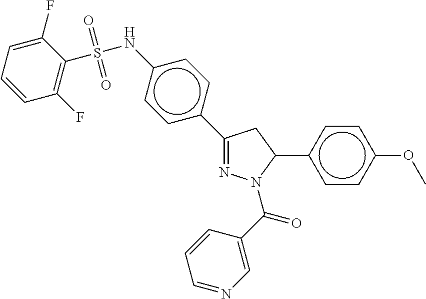 |
| 16A | 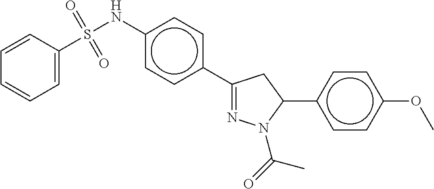 |
| 17A | 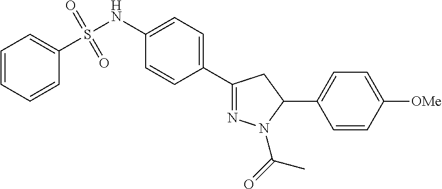 |
| 18A | 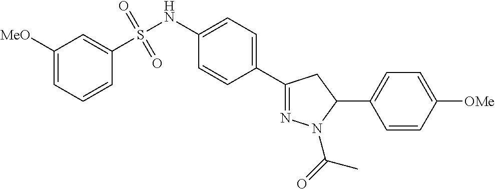 |
| 19A | 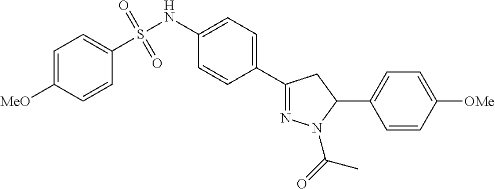 |
| 20A | 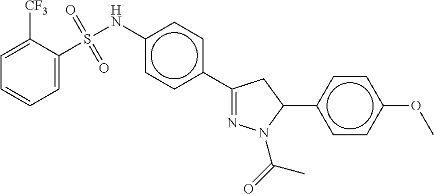 |
| 21A | 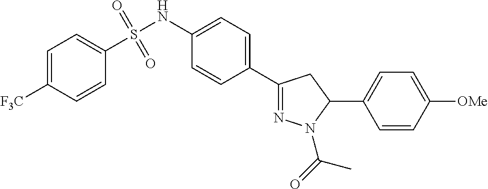 |
| 22A | 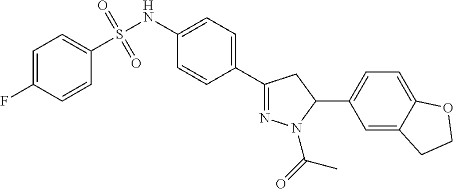 |
| 23A | 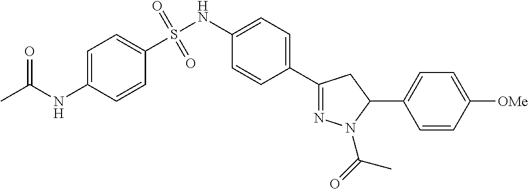 |
| 24A | 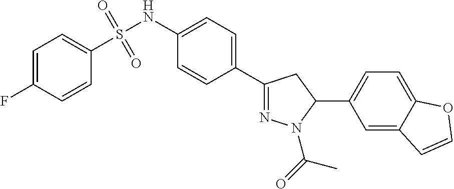 |
| 25A | 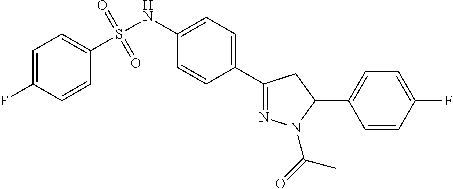 |
| 26A | 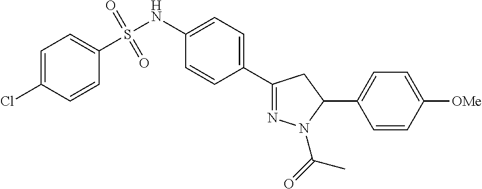 |
| 27A | 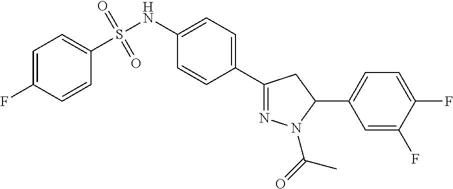 |
| 28A | 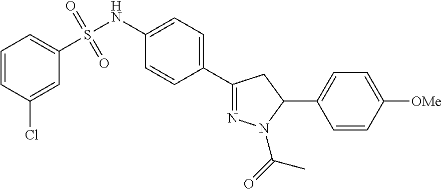 |
| 29A | 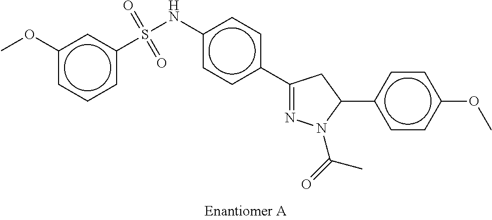 |
| 30A | 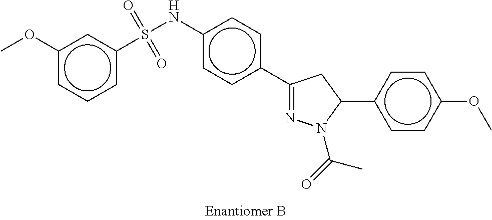 |
| 31A | 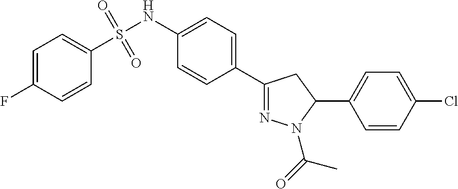 |
| 32A | 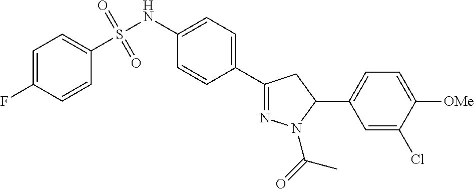 |
| 33A | 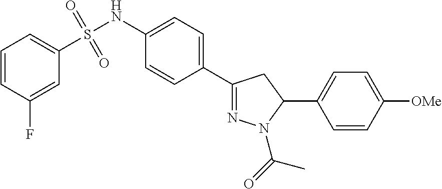 |
| 34A | 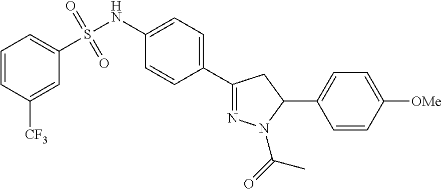 |
| 35A | 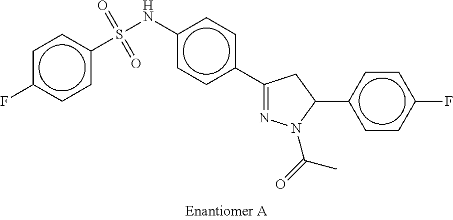 |
| 36A | 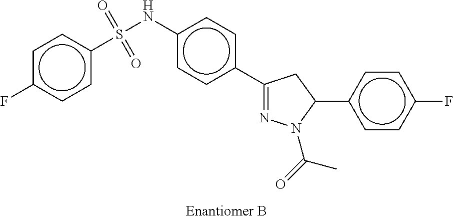 |
| 37A | 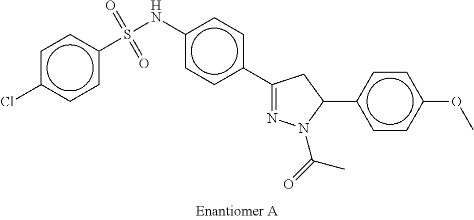 |
| 38A | 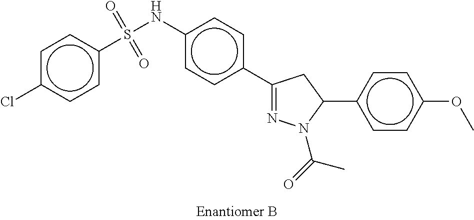 |
| 39A | 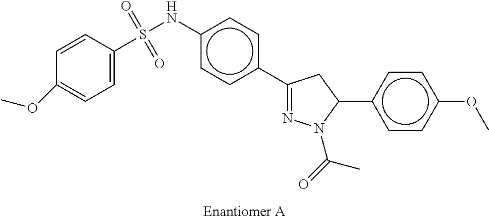 |
| 40A | 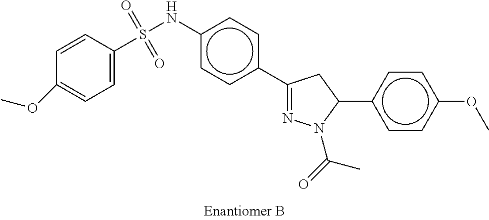 |
| 41A | 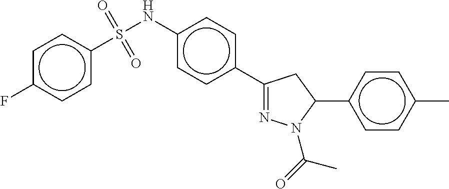 |
| 42A | 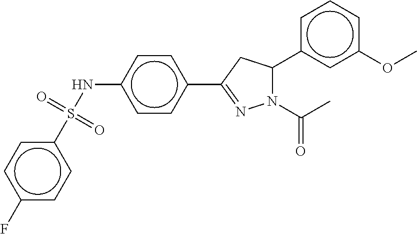 |
| 43A | 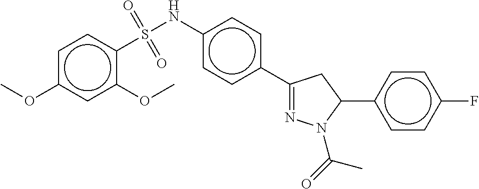 |
| 44A | 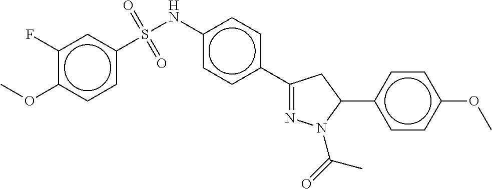 |
| 45A | 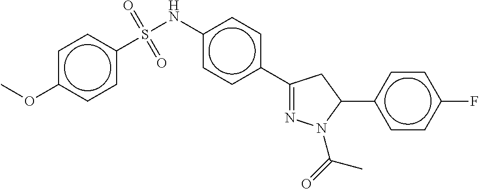 |
| 46A | 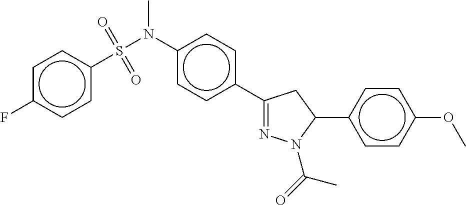 |
| 47A | 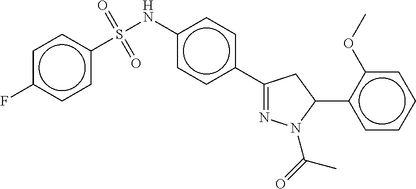 |
| 48A | 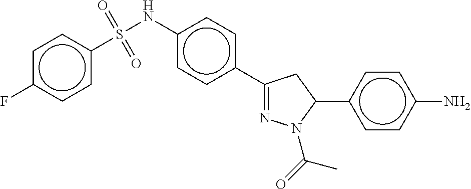 |
| 49A | 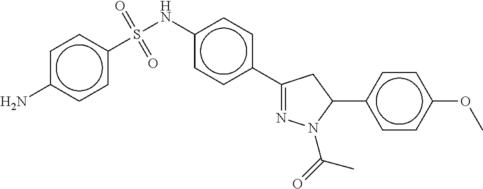 |
| 50A | 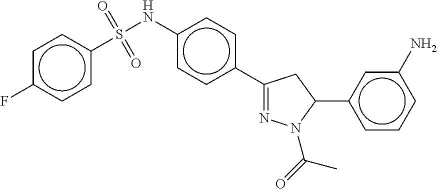 |
| 51A | 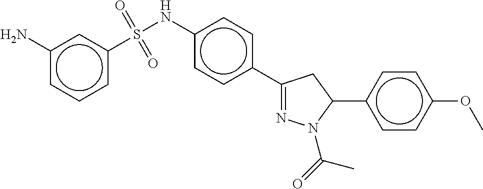 |
| 52A | 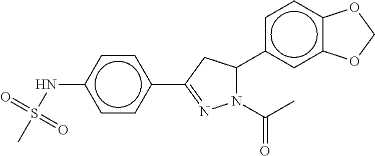 |
| 53A | 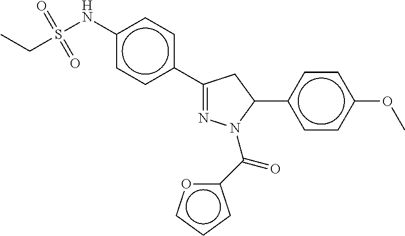 |
| 54A | 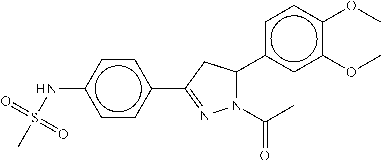 |
| 55A | 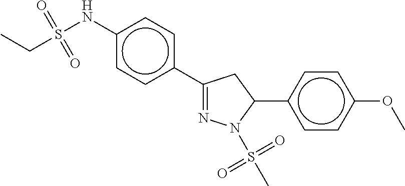 |
| 56A | 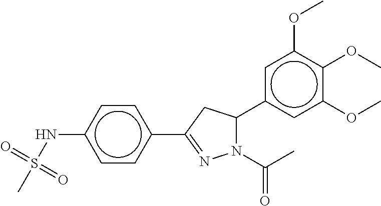 |
| 57A | 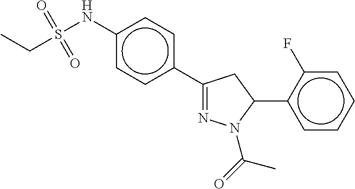 |
| 58A | 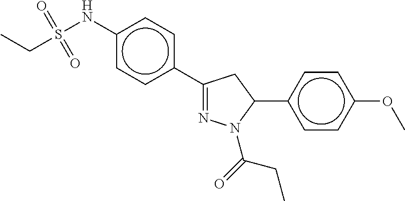 |
| 59A | 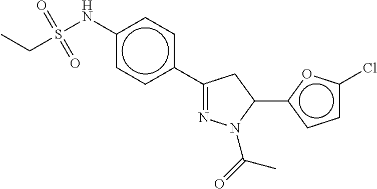 |
| 60A | 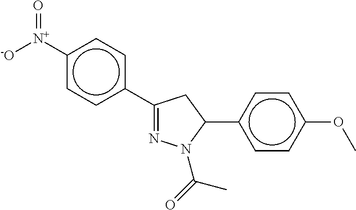 |
| 61A | 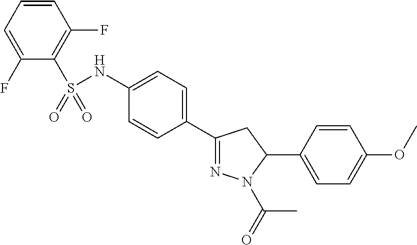 |
| 62A | 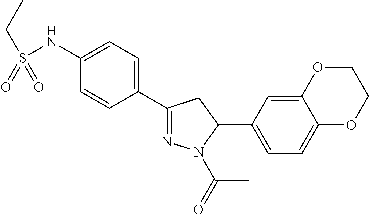 |
| 63A | 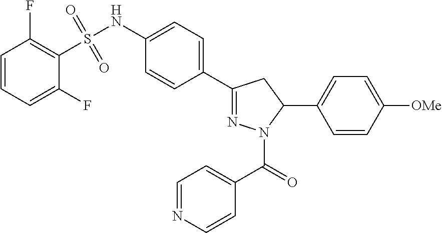 |
| 64A | 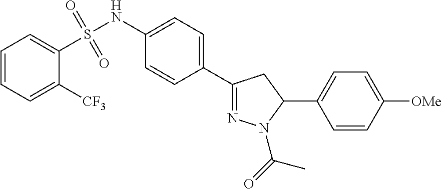 |
| 65A | 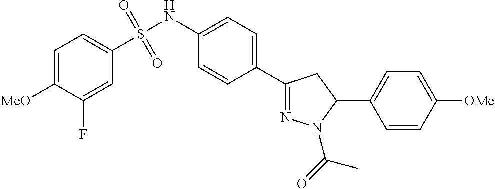 |
| 66A | 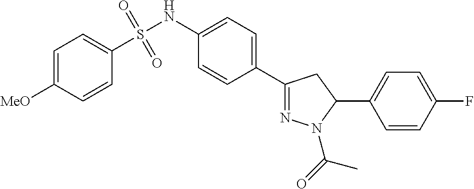 |
| 67A | 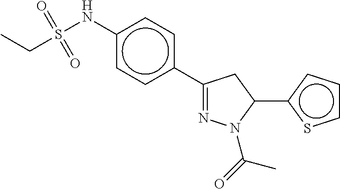 |

In one aspect, the compounds of Table 1A and Table 1B are useful in the methods of treating or preventing the disorders and diseases described herein.

TABLE 1A

| | |
|---|---|
| 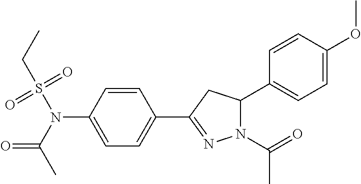 | 101A |
| 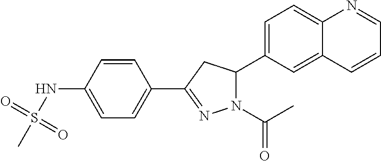 | 102A |
| 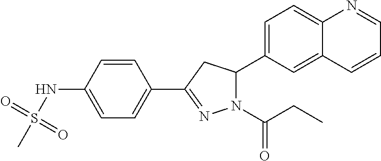 | 103A |
| 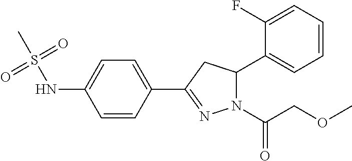 | 104A |
| 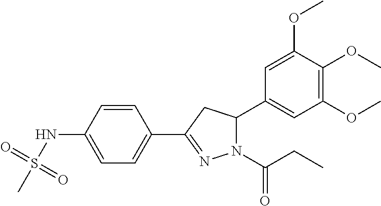 | 105A |
| 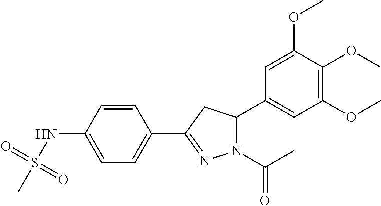 | 106A |
| 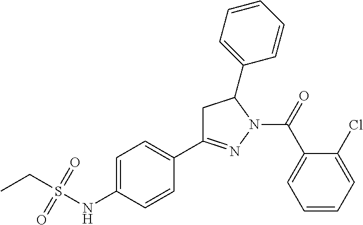 | 107A |
| 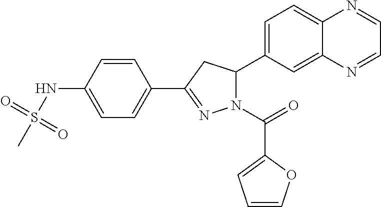 | 108A |
| 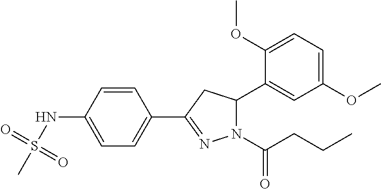 | 109A |
| 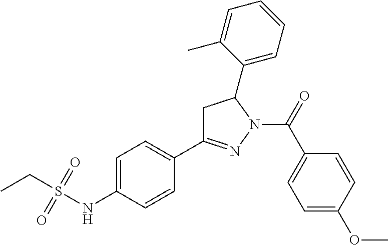 | 110A |
| 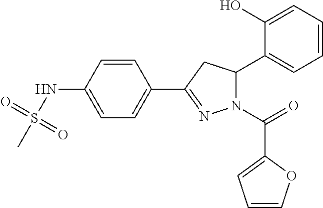 | 111A |
| 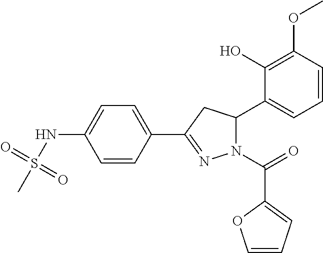 | 112A |
| 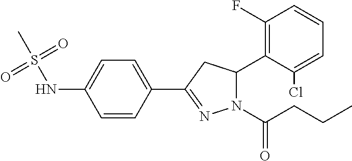 | 113A |
| 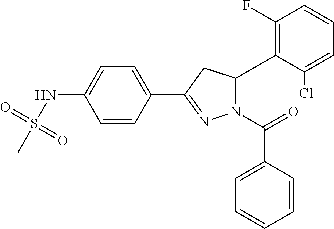 | 114A |
| 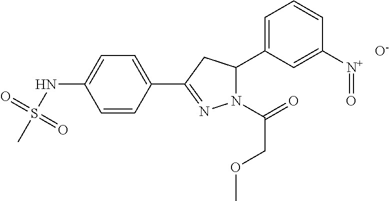 | 115A |
| 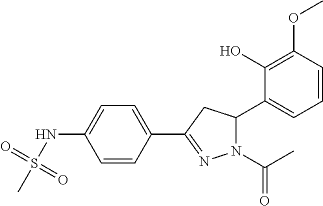 | 116A |
| 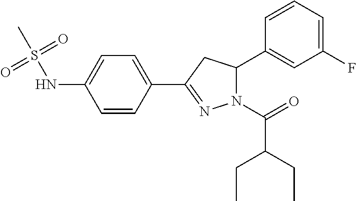 | 117A |
| 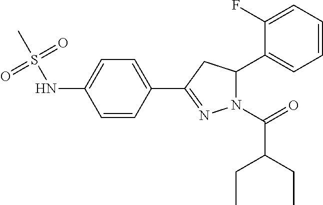 | 118A |
| 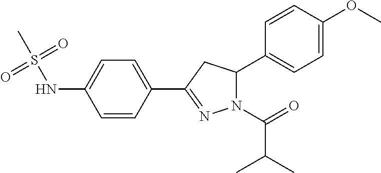 | 119A |
| 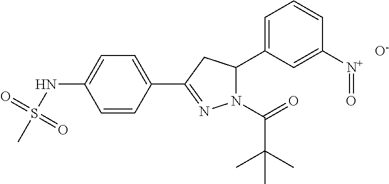 | 120A |
| 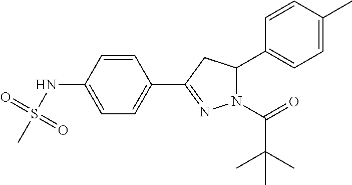 | 121A |
| 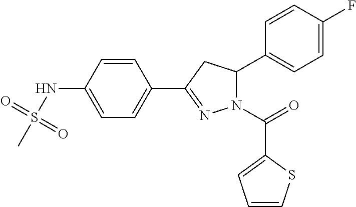 | 122A |
| 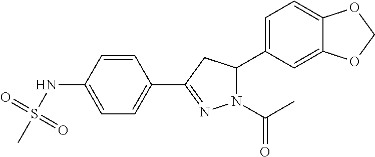 | 123A |
| 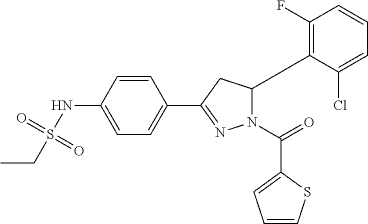 | 124A |
| 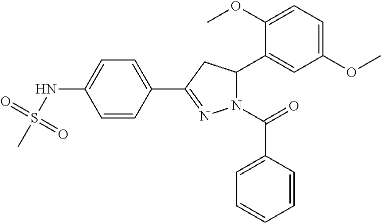 | 125A |
| 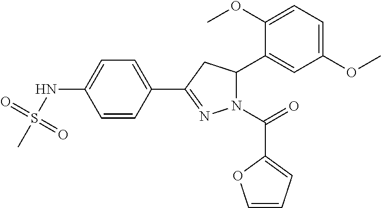 | 126A |
| 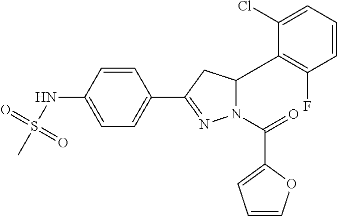 | 127A |
| 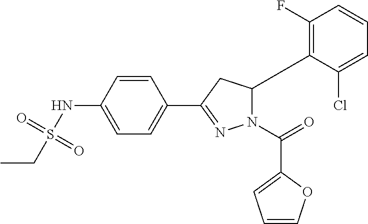 | 128A |
| 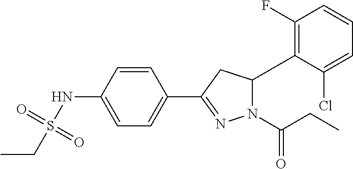 | 129A |
| 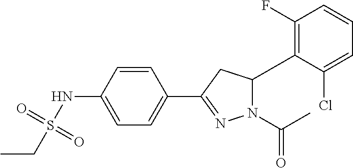 | 130A |
| 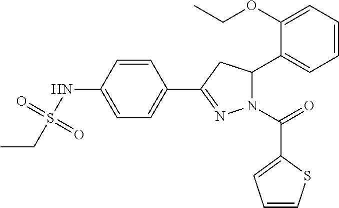 | 131A |
| 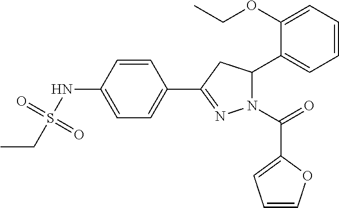 | 132A |
| 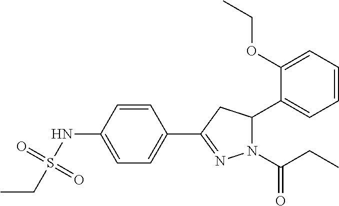 | 133A |
| 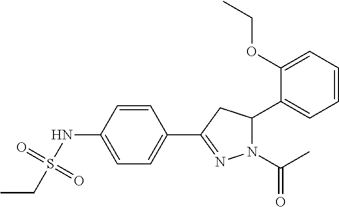 | 134A |
| 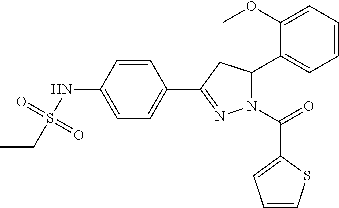 | 135A |
| 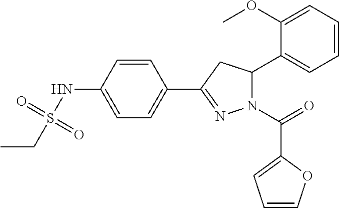 | 136A |
| 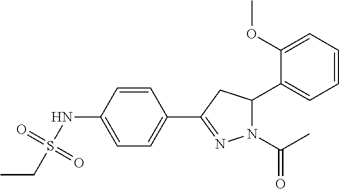 | 137A |
| 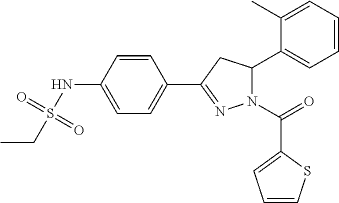 | 138A |
| 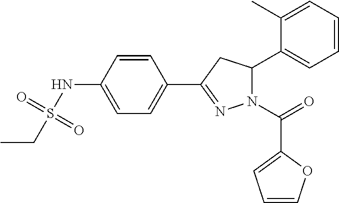 | 139A |
| 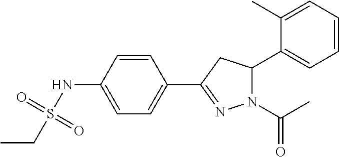 | 140A |
| 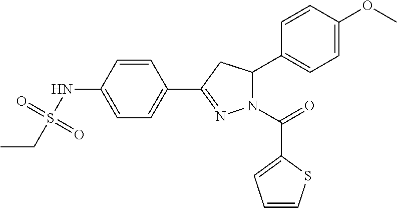 | 141A |
| 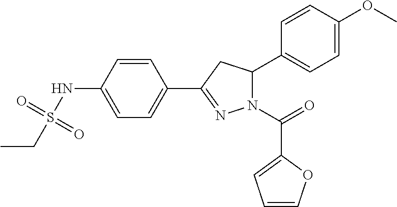 | 142A |
| 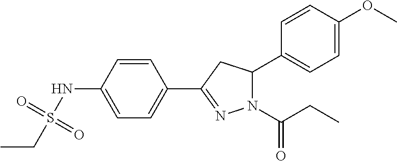 | 143A |
| 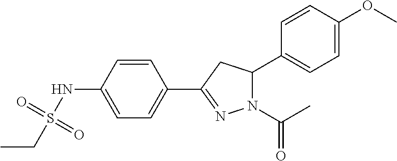 | 144A |
| 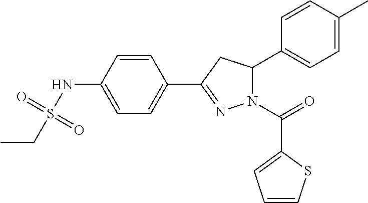 | 145A |
| 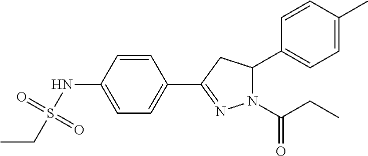 | 146A |
| 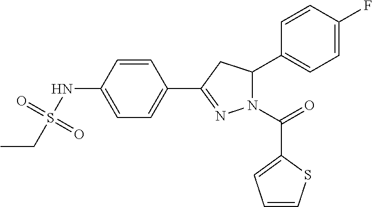 | 147A |
| 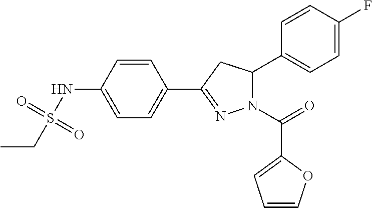 | 148A |
| 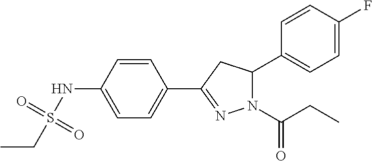 | 149A |
| 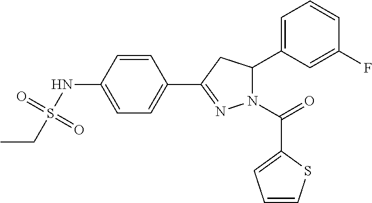 | 150A |
| 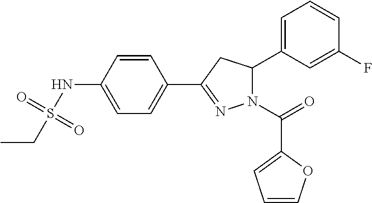 | 151A |
| 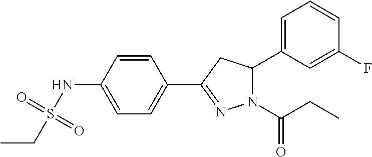 | 152A |
| 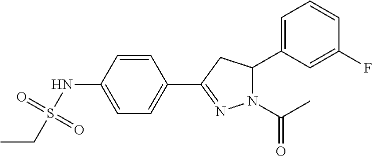 | 153A |
| 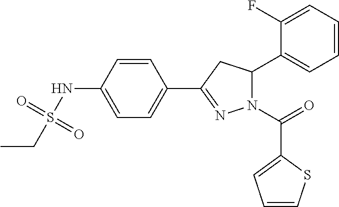 | 154A |
| 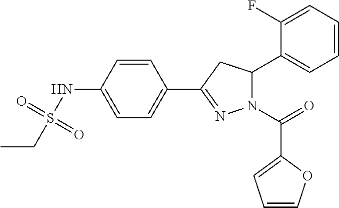 | 155A |
| 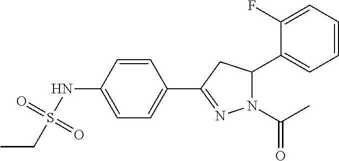 | 156A |
| 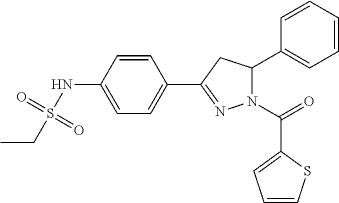 | 157A |
| 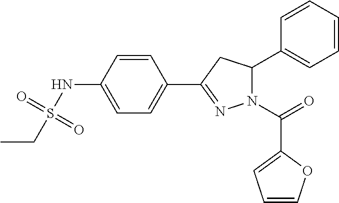 | 158A |
| 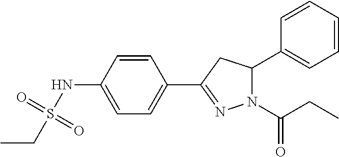 | 159A |
| 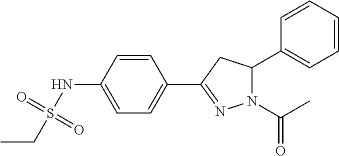 | 160A |
| 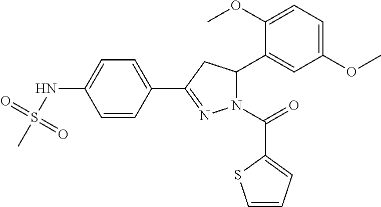 | 161A |
| 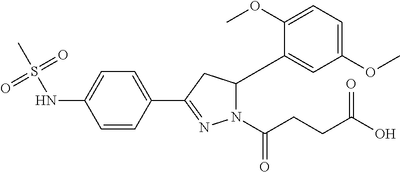 | 162A |
| 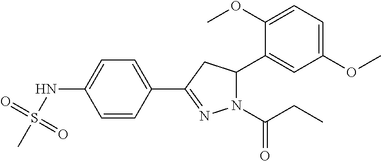 | 163A |
| 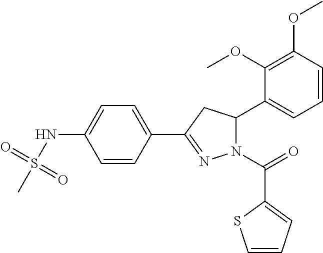 | 164A |
| 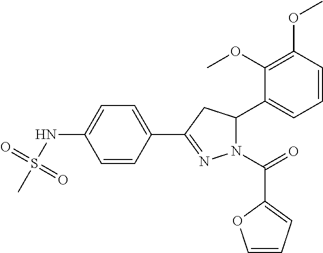 | 165A |
| 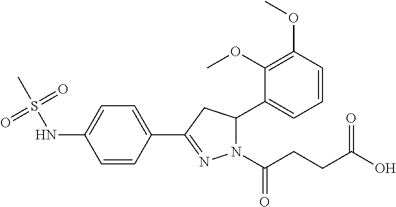 | 166A |
| 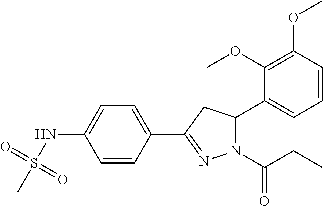 | 167A |
| 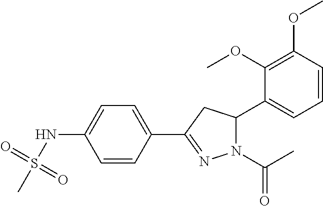 | 168A |
| 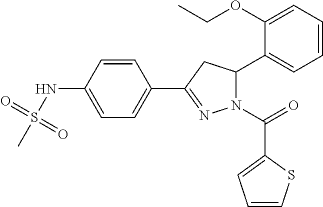 | 169A |
| 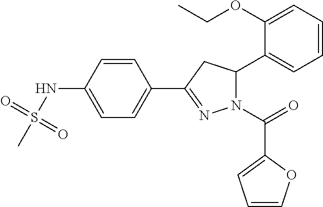 | 170A |
| 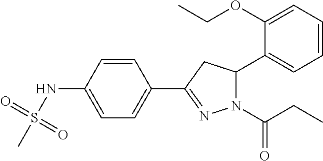 | 171A |
| 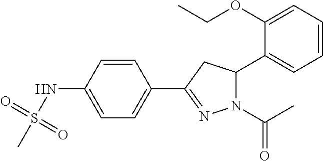 | 172A |
| 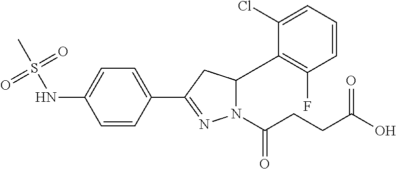 | 173A |
| 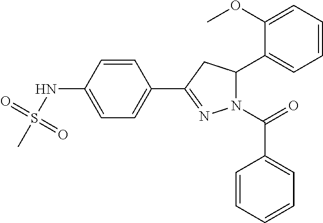 | 174A |
| 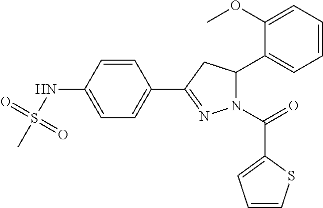 | 175A |
| 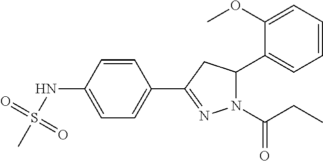 | 176A |
| 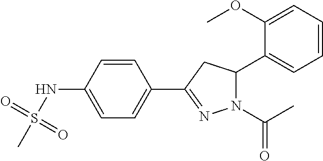 | 177A |
| 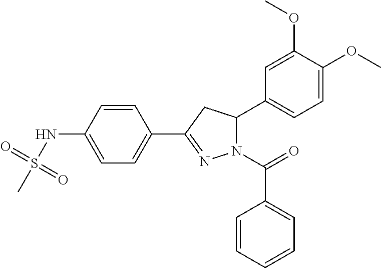 | 178A |
| 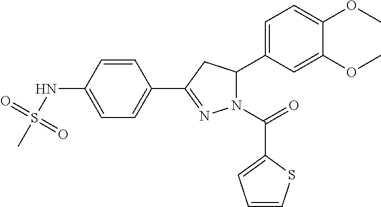 | 179A |
| 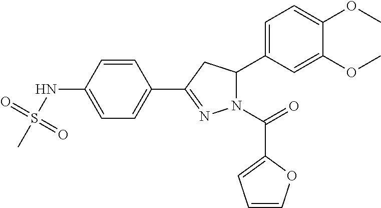 | 180A |
| 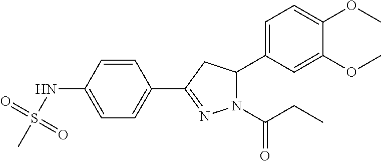 | 181A |
| 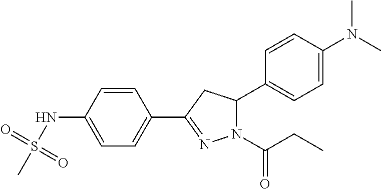 | 182A |
| 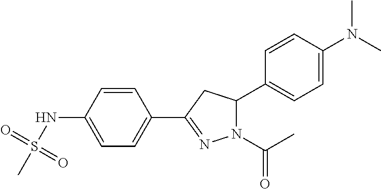 | 183A |
| 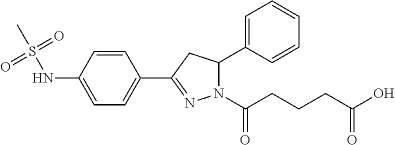 | 184A |
| 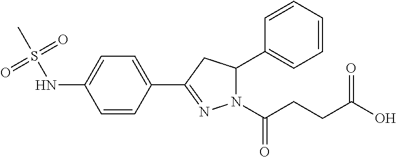 | 185A |
| 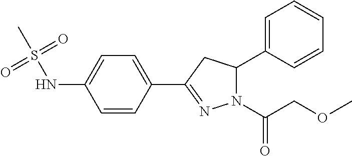 | 186A |
| 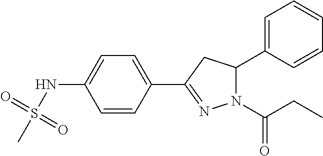 | 187A |
| 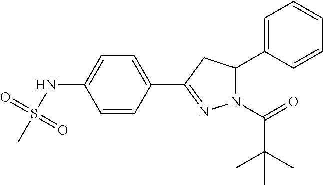 | 188A |
| 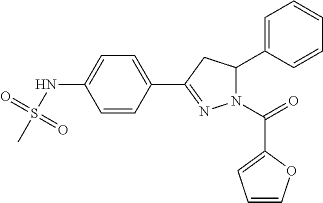 | 189A |
| 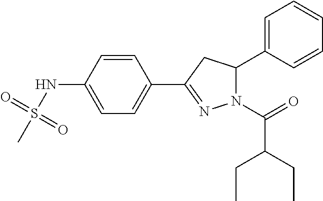 | 190A |
| 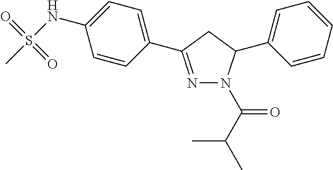 | 191A |
| 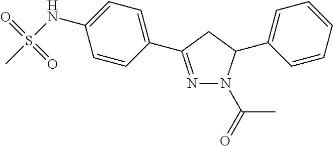 | 192A |
| 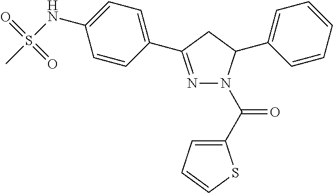 | 193A |
| 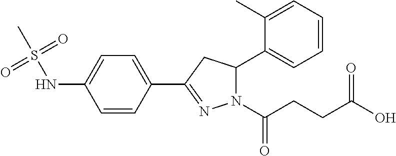 | 194A |
| 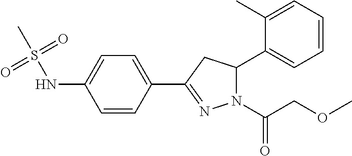 | 195A |
| 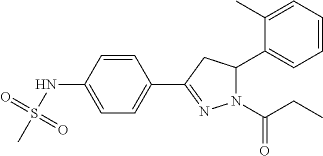 | 196A |
| 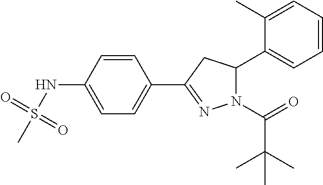 | 197A |
| 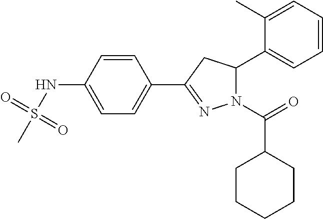 | 198A |
| 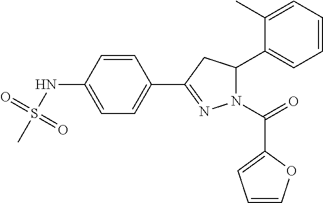 | 199A |
| 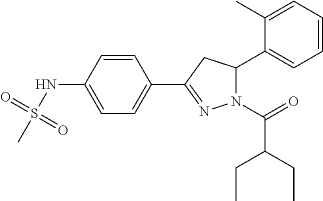 | 200A |
| 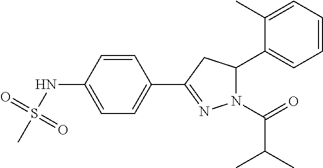 | 201A |
| 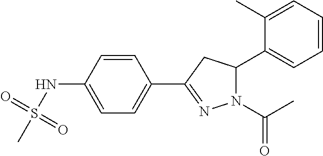 | 202A |
| 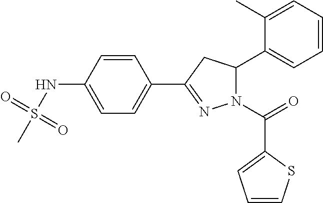 | 203A |
| 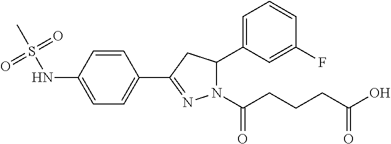 | 204A |
| 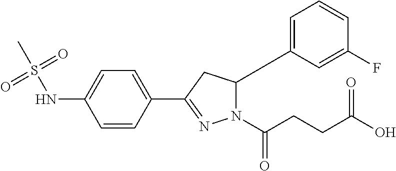 | 205A |
| 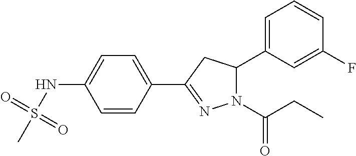 | 206A |
| 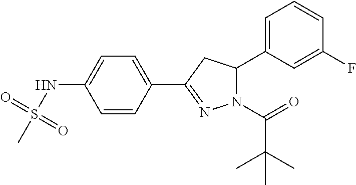 | 207A |
| 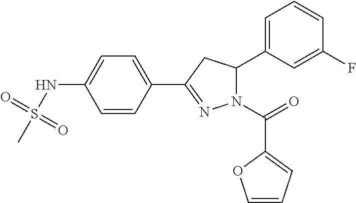 | 208A |
| 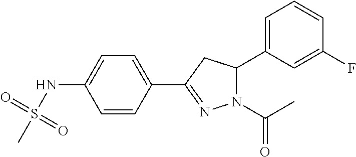 | 209A |
| 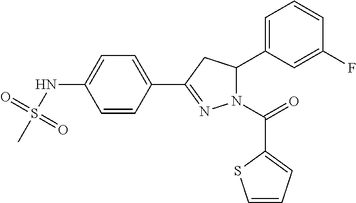 | 210A |
| 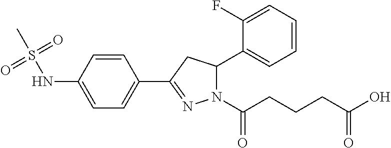 | 211A |
| 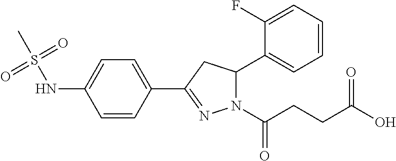 | 212A |
| 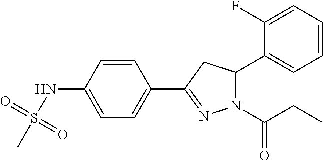 | 213A |
| 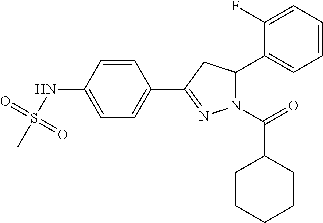 | 214A |
| 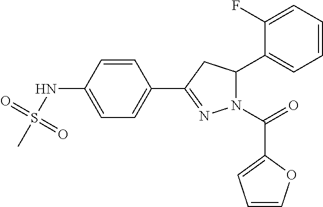 | 215A |
| 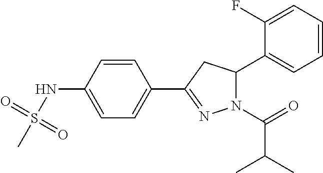 | 216A |
| 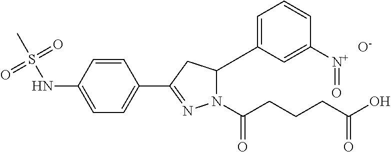 | 217A |
| 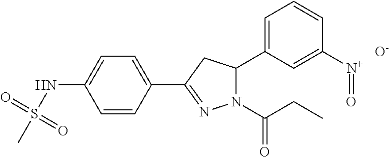 | 218A |
| 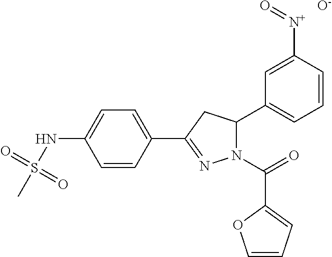 | 219A |
| 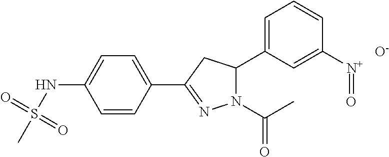 | 220A |
| 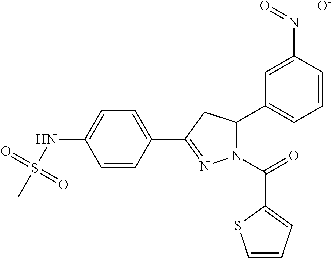 | 221A |
| 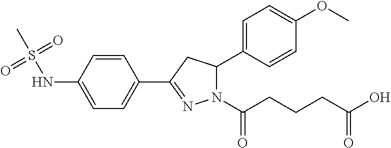 | 222A |
| 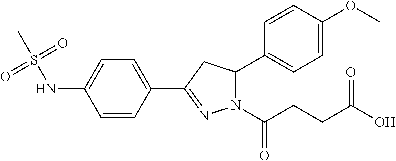 | 223A |
| 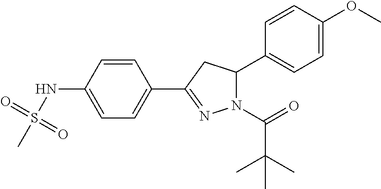 | 224A |
| 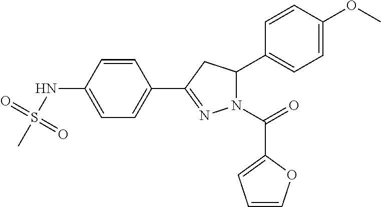 | 225A |
| 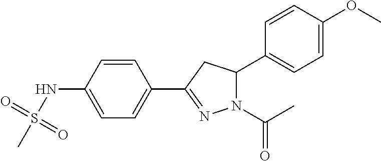 | 226A |
| 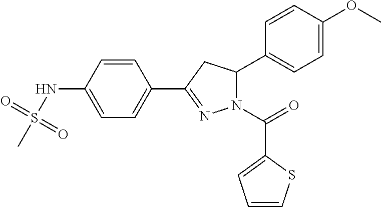 | 227A |
| 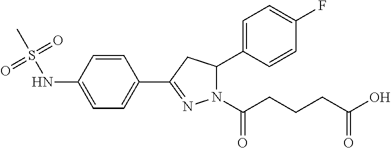 | 228A |
| 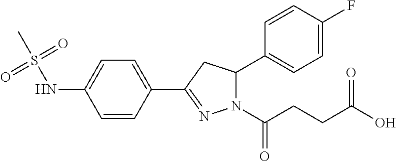 | 229A |
| 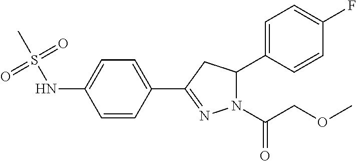 | 230A |
| 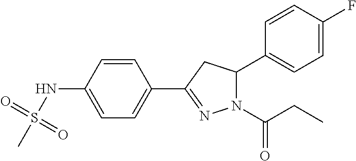 | 231A |
| 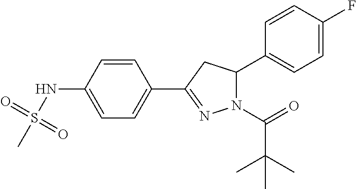 | 232A |
| 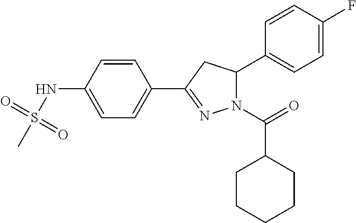 | 233A |
| 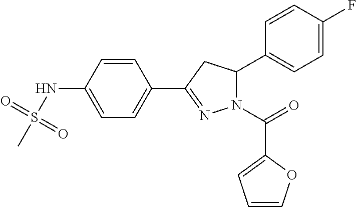 | 234A |
| 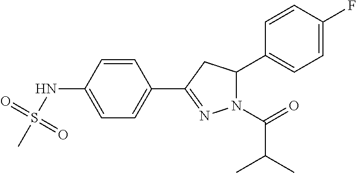 | 235A |
| 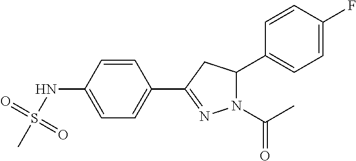 | 236A |
| 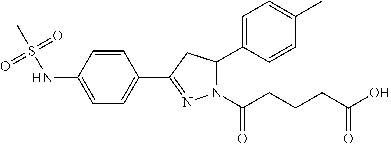 | 237A |
| 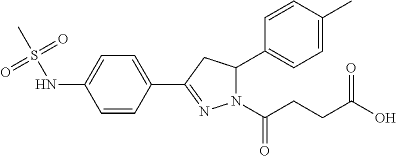 | 238A |
| 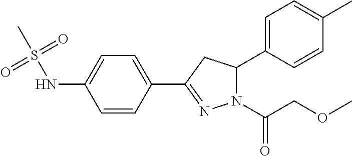 | 239A |
| 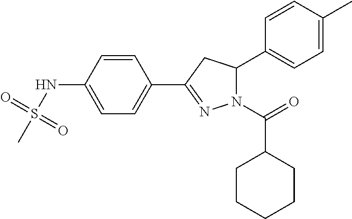 | 240A |
| 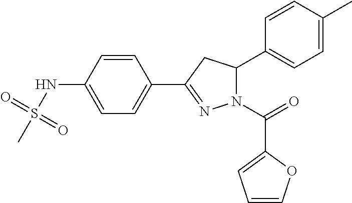 | 241A |
| 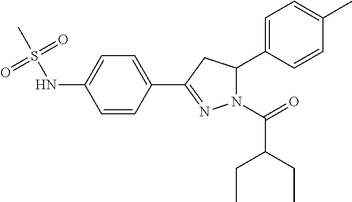 | 242A |
| 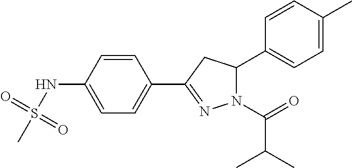 | 243A |
| 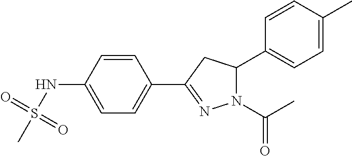 | 244A |
| 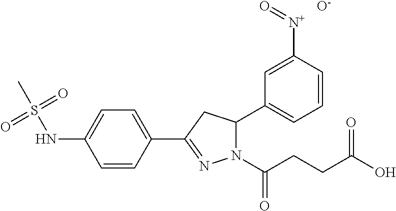 | 245A |
| 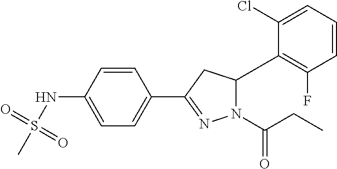 | 246A |
| 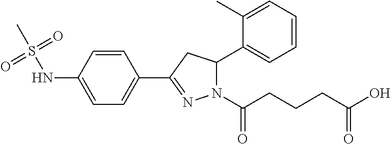 | 247A |
| 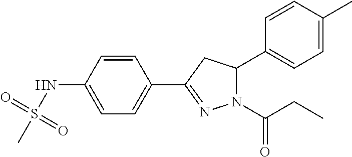 | 248A |
| 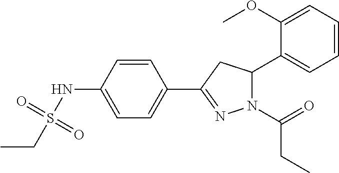 | 249A |
| 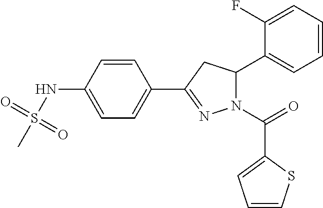 | 250A |
| 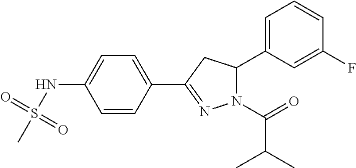 | 251A |
| 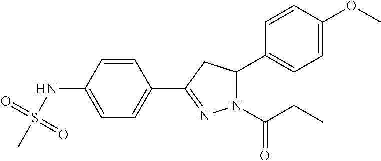 | 252A |
| 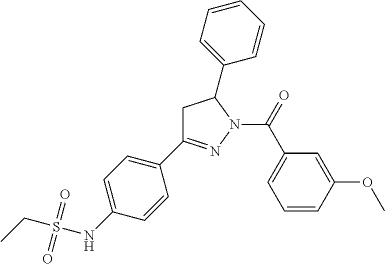 | 253A |
| 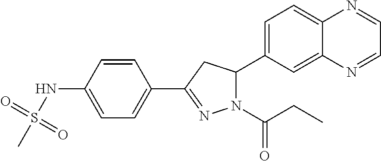 | 254A |
| 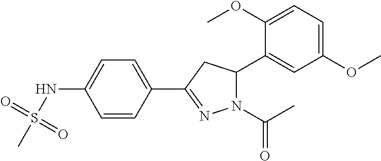 | 255A |
| 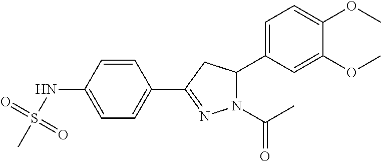 | 256A |
| 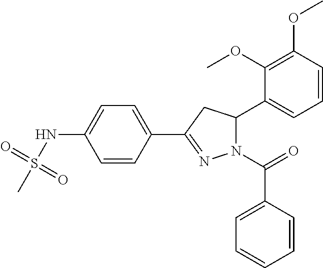 | 257A |
| 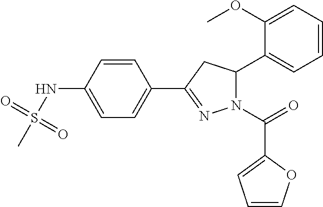 | 258A |
| 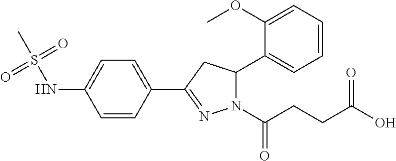 | 259A |
| 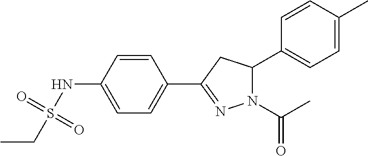 | 260A |
| 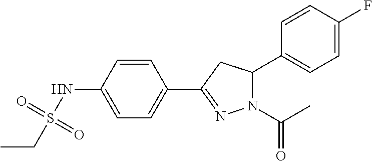 | 261A |
| 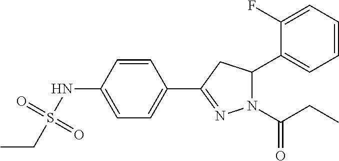 | 262A |
| 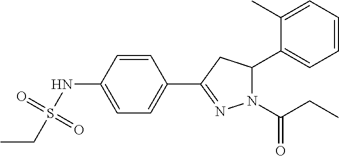 | 263A |
| 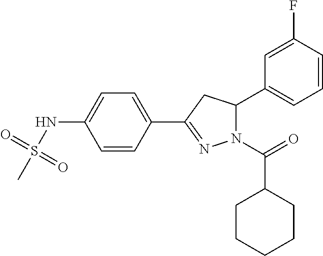 | 264A |
| 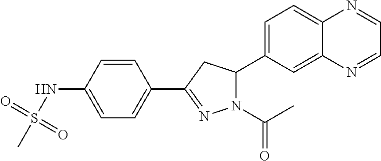 | 265A |
| 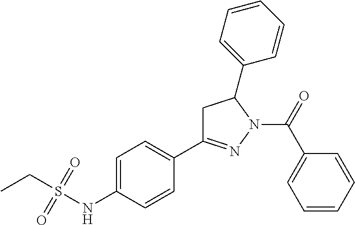 | 266A |
| 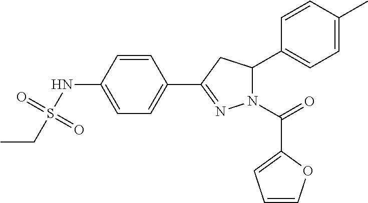 | 267A |
| 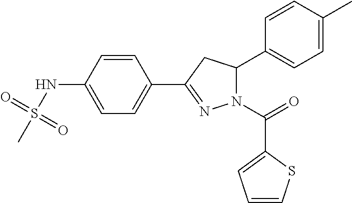 | 268A |
| 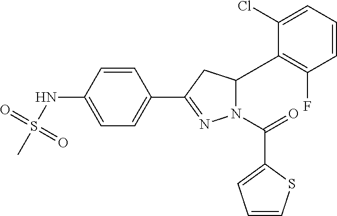 | 269A |
| 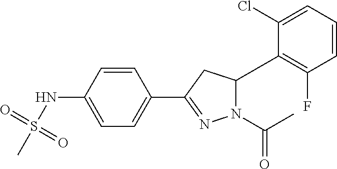 | 270A |

TABLE 1B

| | |
|---|---|
| 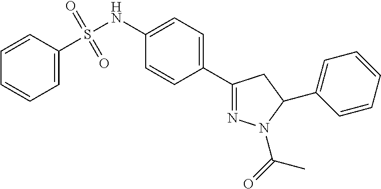 | 301A |
| 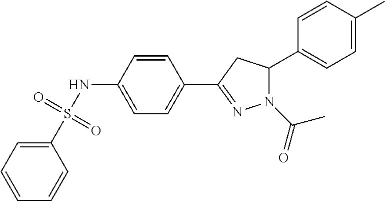 | 302A |
| 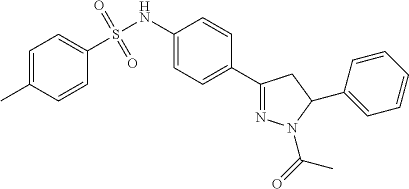 | 303A |
| 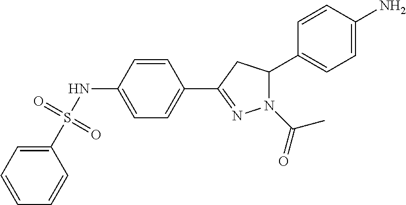 | 304A |
| 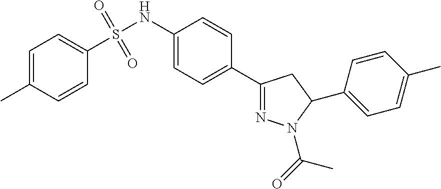 | 305A |
| 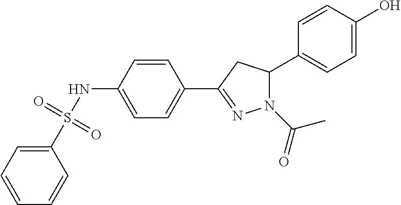 | 306A |
| 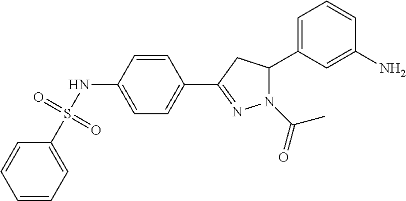 | 307A |
| 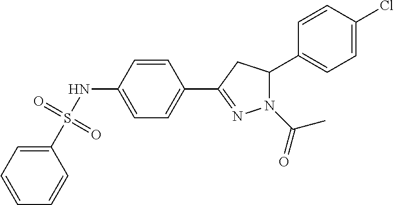 | 308A |
| 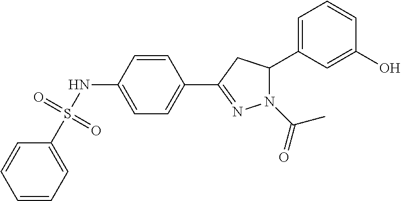 | 309A |
| 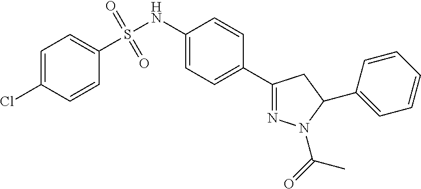 | 310A |
| 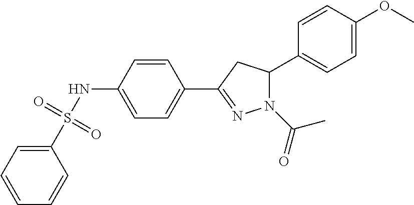 | 311A |
| 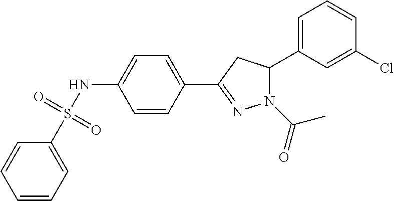 | 312A |
| 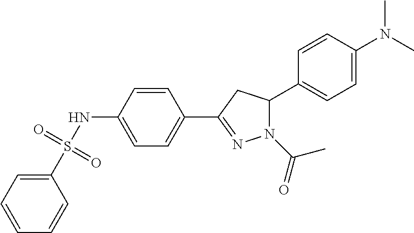 | 313A |
| 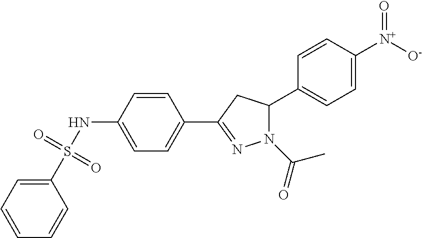 | 314A |
| 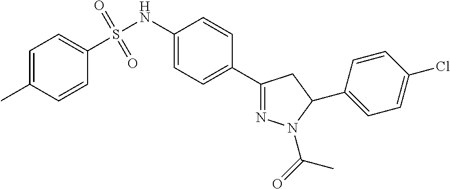 | 315A |
| 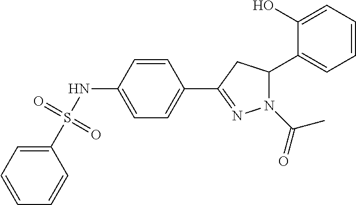 | 316A |
| 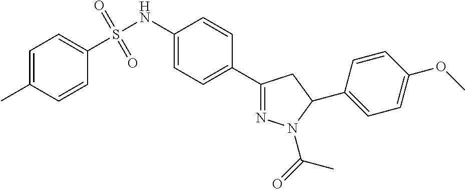 | 317A |
| 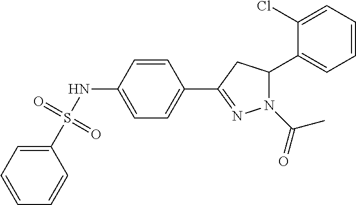 | 318A |
| 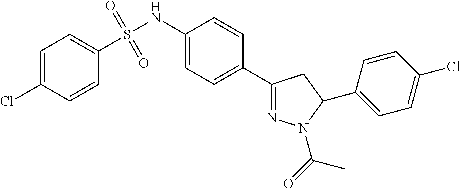 | 319A |
| 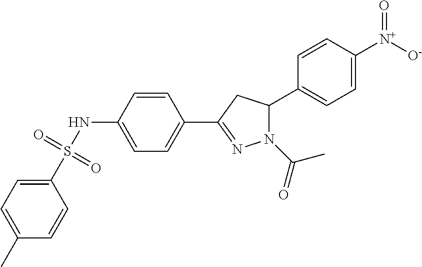 | 320A |
| 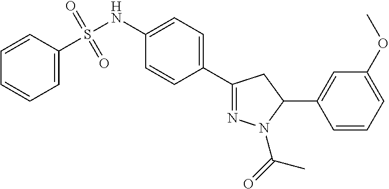 | 321A |
| 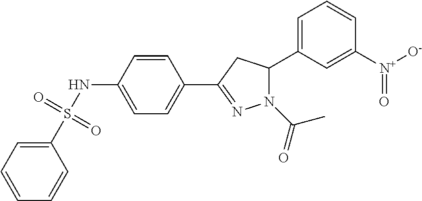 | 322A |
| 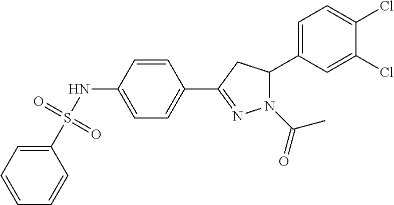 | 323A |
| 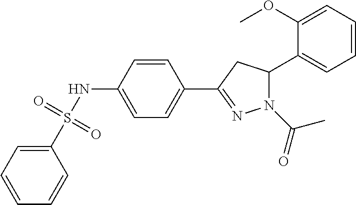 | 324A |
| 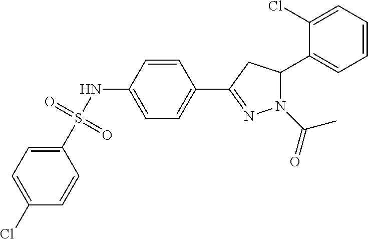 | 325A |
| 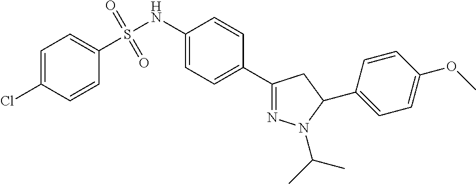 | 326A |
| 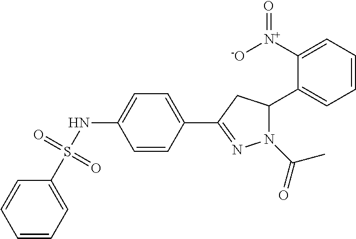 | 327A |
| 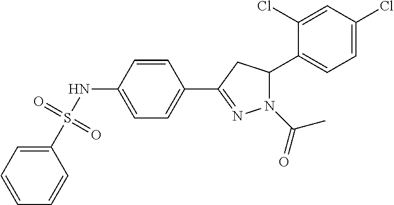 | 328A |
| 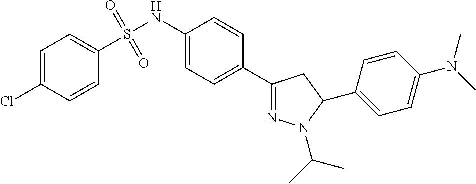 | 329A |
| 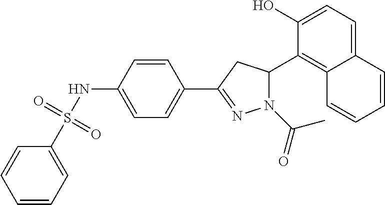 | 330A |
| 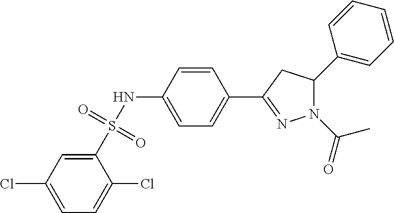 | 331A |
| 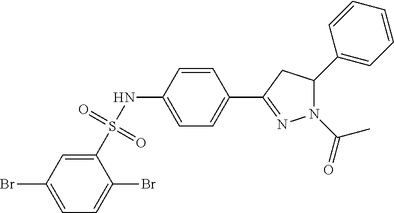 | 332A |
| 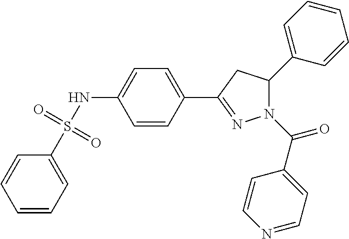 | 333A |
| 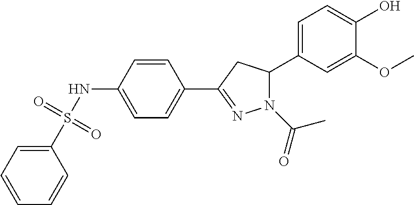 | 334A |
| 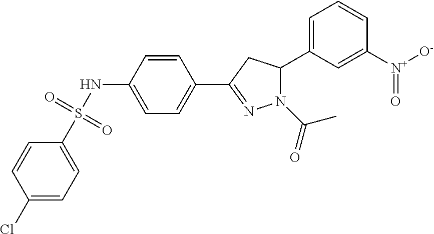 | 335A |
| 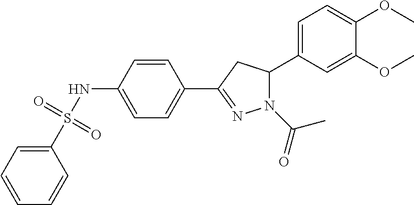 | 336A |
| 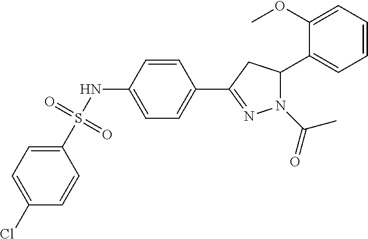 | 337A |
| 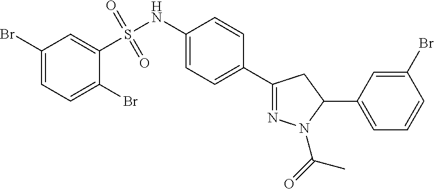 | 338A |
| 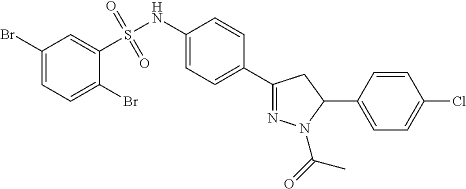 | 339A |
| 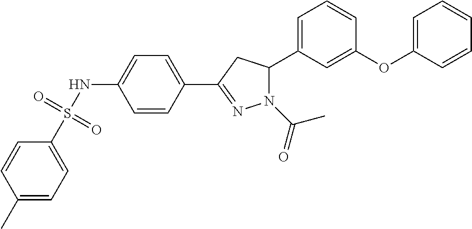 | 340A |
| 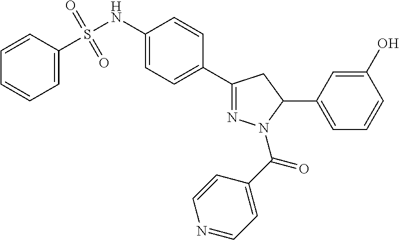 | 341A |
| 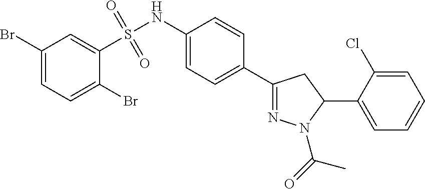 | 342A |
| 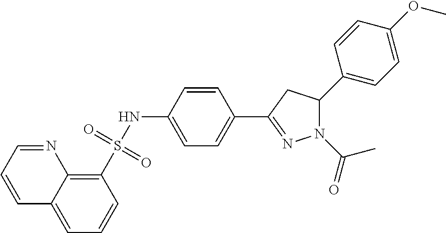 | 343A |
| 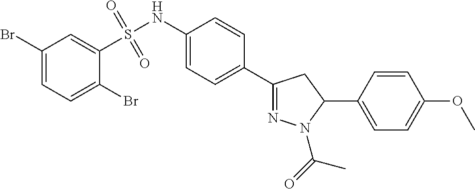 | 344A |
| 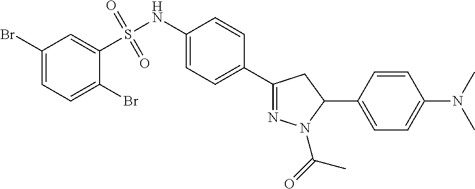 | 345A |
| 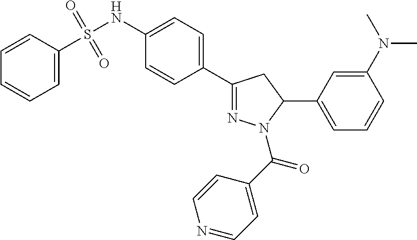 | 346A |
| 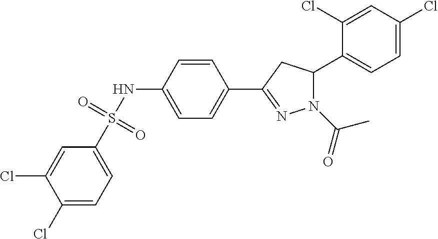 | 347A |
| 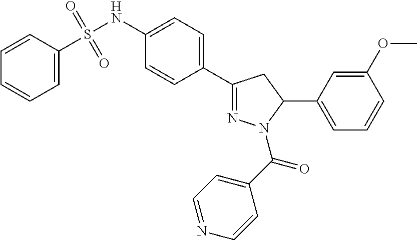 | 348A |
| 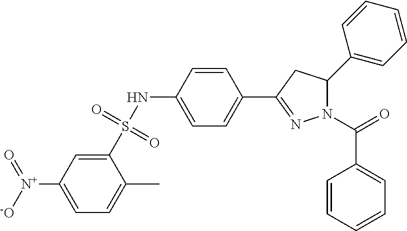 | 349A |
| 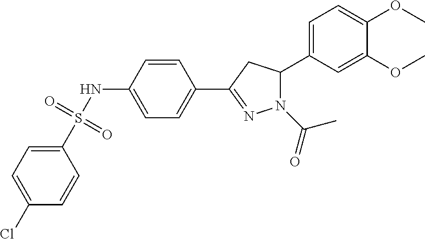 | 350A |
| 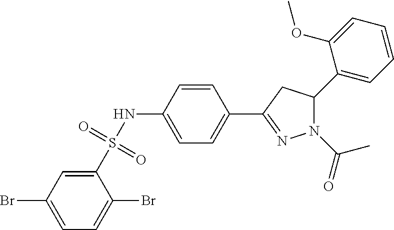 | 351A |
| 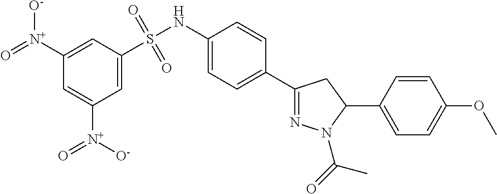 | 352A |
| 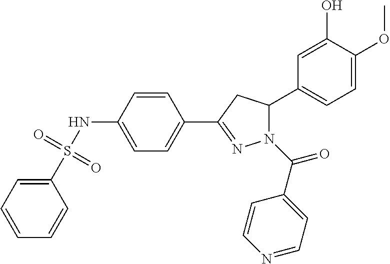 | 353A |
| 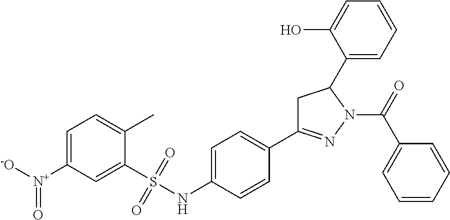 | 354A |
| 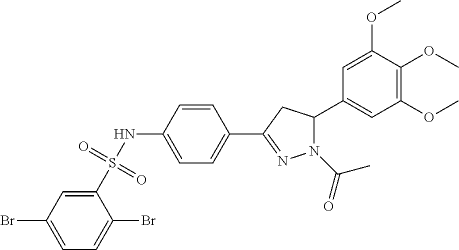 | 355A |
| 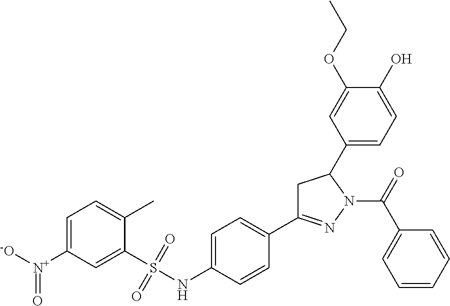 | 356A |

Embodiments of the invention include a compound or a pharmaceutically acceptable salt of a compound according to any one of formulae of the invention. Embodiments of the invention include a pharmaceutically acceptable salt of a compound according to any one of formulae of the invention. Embodiments of the invention include a solvate of a compound according to any one of formulae of the invention. Embodiments of the invention include a hydrate of a compound according to any one of the formulae of the invention. Embodiments of the invention include an acid addition salt (e.g., a pharmaceutically acceptable salt) of a compound according to any one of the formulae of the invention. For example, the salt is a hydrochloride salt.

Embodiments of the invention include a compound of any one of formulae I, II, III, IV, V, or VI or a pharmaceutically acceptable salt thereof, wherein the compound is a racemate. Embodiments of the invention include a compound of any one of formulae I, II, III, IV, V, or VI, wherein the compound is a single enantiomer or a pharmaceutically acceptable salt thereof. Embodiments of the invention include a compound of any one of formulae I, II, III, IV, or VI or a pharmaceutically acceptable salt thereof, wherein the compound is a single enantiomer that rotates plane-polarized light in the clockwise direction (+). Embodiments of the invention include a compound of any one of formulae I, II, III, IV, V, or VI or a pharmaceutically acceptable salt thereof, wherein the compound is a single enantiomer that rotates plane-polarized light in the counterclockwise direction (−). Embodiments of the invention include a compound of any one of formulae I, II, III, IV, V, or VI or a pharmaceutically acceptable salt thereof, wherein the stereogenic center attached to R⁵ is in the R-configuration. Embodiments of the invention include a compound of any one of formulae I, II, III, IV, V, or VI or a pharmaceutically acceptable salt thereof, wherein the stereogenic center attached to R⁵ is in the S-configuration.

Embodiments of the invention also include a composition comprising a compound according to any one of the formulae of the invention and at least one pharmaceutically acceptable excipient. When the compound according to any one of the formulae of the invention has more than one stereoisomeric form, the pharmaceutical composition may be prepared with a pure or an essentially pure enantiomeric form of the compound, having an enantiopurity of at least 90% enantiomeric excess (EE), preferably at least 95% EE, more preferably at least 98% EE, and most preferably at least 99% EE. Alternatively, the pharmaceutical composition may be prepared as mixture of enantiomeric forms of the compounds (e.g., as a racemic mixture or as a mixture with a ratio of 60:40, 70:30, 80:20 or 90:10 between the enantiomeric forms).

In one aspect, compounds of the invention include any of the compounds listed in Tables 1A and 1B or a stereoisomer, salt, solvate, hydrate, or prodrug thereof, wherein the compound activates PKM2 by at least about 10%, by at least about 20%, by at least about 25%, by at least about 30%, by at least about 40%, by at least about 50%, by at least about 60%, by at least about 70%, by at least about 75%, by at least about 80%, by at least about 90%, or by about 95% or more.

In one aspect, embodiments of the invention include the foregoing compounds that activate PKM2 by at least about 10%, by at least about 20%, by at least about 25%, by at least about 30%, by at least about 40%, by at least about 50%, by at least about 60%, by at least about 70%, by at least about 75%, by at least about 80%, by at least about 90%, or by about 95% or more.

In one aspect, compounds of the invention are prepared using methods known in the art. For example, compounds of the invention can be prepared based on the procedures of Patel, P. et al. (1996) IL FARMACO 51(1), 59-63 and Jamode, V. S. et al. (2003) Indian J. Heterocyclic Chem. 12, 323-326. As shown in the scheme below, compounds of the invention can be prepared as follows. In the first step, a substituted acetophenone (1) can be condensed with an arylcarbonyl moiety (2) under acidic or basic conditions to afford α, β-unsaturated ketone (3). In the next step, the unsaturated ketone (3) can be treated with hydrazine and substituted carboxylic acid or anhydride (4) concurrently or sequentially, to yield the desired 2-pyrazoline product (5). The transformation from (3) to (5) can be performed as one step or as two separate steps.

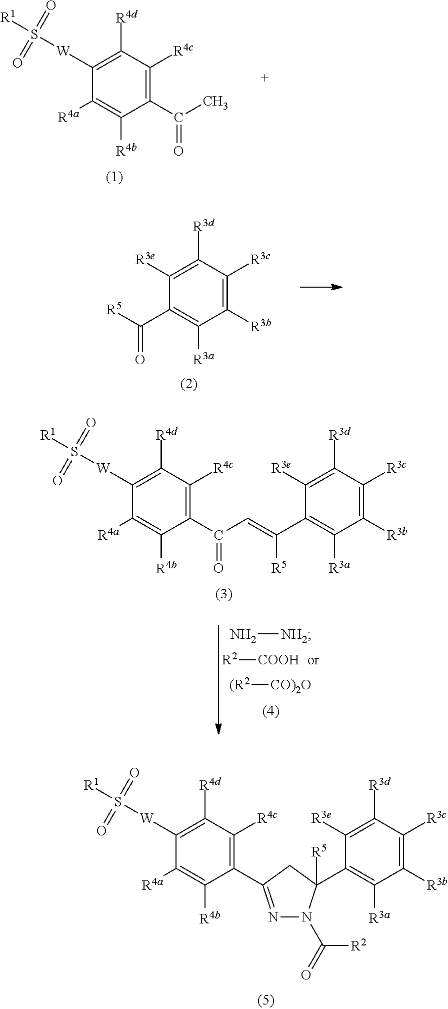

In one aspect, embodiments of the invention are drawn to methods of activating PKM2 by contacting PKM2 with a compound of the invention, e.g., a compound according to any one of formulae of the invention, or a salt, solvate, hydrate, or prodrug thereof. In some embodiments, PKM2 is activated by at least about 10%, by at least about 20%, by at least about 25%, by at least about 30%, by at least about 40%, by at least about 50%, by at least about 60%, by at least about 70%, by at least about 75%, by at least about 80%, by at least about 90%, or by about 95% or more. In some embodiments, the PKM2 activated is in a cell, e.g., a human cell.

Embodiments of the invention include methods of preventing or treating a cell proliferation-related disorder, a cancer, an inflammatory disorder, a metabolic disorder or an autoimmune disorder by administering to a subject in need thereof a pharmaceutical composition that includes a compound according to embodiments of the invention, e.g., a compound according to any one of the formulae of the invention, or a salt, solvate, hydrate, or prodrug thereof, and at least one pharmaceutically acceptable excipient to a subject in need thereof.

When the pharmaceutical composition includes a compound of the invention that has more than one stereoisomeric form, the pharmaceutical composition may be prepared with a pure or an essentially pure enantiomeric form of the compound, with an enantiopurity of at least 90% enantiomeric excess (EE), preferably at least 95% EE, more preferably at least 98% EE, and most preferably at least 99% EE. Alternatively, the pharmaceutical composition may be prepared as mixture of enantiomeric forms of the compounds (e.g., as a racemic mixture or as a mixture with a ratio of 60:40, 70:30, 80:20 or 90:10 between the enantiomeric forms). The invention also includes use of a compound of the invention in the manufacture of a medicament to prevent or treat a cell proliferation-related disorder, a cancer, an inflammatory disorder, metabolic disorder, or an autoimmune disorder. When the medicament includes a compound of the invention that has more than one stereoisomeric form, the medicament may be prepared with a pure or an an essentially pure enantiomeric form of the compound, with an enantiopurity of at least 90% enantiomeric excess (EE), preferably at least 95% EE, more preferably at least 98% EE, and most preferably at least 99% EE. Alternatively, the medicament may be prepared as mixture of enantiomeric forms of the compounds (e.g., as a racemic mixture or as a mixture with a ratio of 60:40, 70:30, 80:20 or 90:10 between the enantiomeric forms).

The invention relates to a method of treating or preventing a disease or disorder that is modulated by PKM2 activation, by administering a pharmaceutical composition that includes a compound of the invention and at least one pharmaceutically acceptable excipient. When the pharmaceutical composition includes a compound of the invention that has more than one stereoisomeric form, the pharmaceutical composition may be prepared with a pure or an essentially pure enantiomeric form of the compound, with an enantiopurity of at least 90% enantiomeric excess (EE), preferably at least 95% EE, more preferably at least 98% EE, and most preferably at least 99% EE. Alternatively, the pharmaceutical composition may be prepared as mixture of enantiomeric forms of the compounds (e.g., as a racemic mixture or as a mixture with a ratio of 60:40, 70:30, 80:20 or 90:10 between the enantiomeric forms). For example, the disease or disorder that is modulated by PKM2 activation is cancer, pre-cancer or a hyperproliferative disorder.

One aspect of the invention includes methods of regulating immune system activity in a subject comprising administering a compound of the invention. Embodiments of the invention also include use of a compound of the invention in the manufacture of a medicament to regulate immune system activity. Examples of diseases that may be treated or prevented according to the foregoing methods include, but are not limited to, allergies, asthma, autoimmune diseases such as transplant rejection (e.g., kidney, heart, lung, liver, pancreas, skin, host versus graft reaction (HVGR), etc.), rheumatoid arthritis, and amyotrophic lateral sclerosis, multiple sclerosis, psoriasis and Sjogren' syndrome, Type II inflammatory disease such as vascular inflammation (including vasculitis, ateritis, atherosclerosis and coronary artery disease), diseases of the central nervous system such as stroke, pulmonary diseases such as bronchitis obliterous and primary and primary pulmonary hypertension, delayed or cell-mediated, Type IV hypersensitivity and solid and hematologic malignancies such as leukemias and lymphomas.

In one aspect, a compound of the invention may be used as a pharmaceutical agent. The compounds may be used without limitation, for example, as anti-cancer, anti-proliferative, anti-inflammatory and/or immunosuppressive agents, for treating humans and/or animals, such as for treating humans and/or other mammals.

In some embodiments, the administration of the compound is carried out orally, parentally, subcutaneously, intravenously, intramuscularly, intraperitoneally, by intranasal instillation, by intracavitary or intravesical instillation, topically, intraarterially, intralesionally, by metering pump, or by application to mucous membranes. In some embodiments, the compound is administered with a pharmaceutically acceptable carrier.

In certain embodiments, the cell proliferation disorder includes any type of cancer including solid tumors and non-solid tumors. In specific embodiments the solid tumors are selected from tumors in the CNS (central nervous system), liver cancer, colorectal carcinoma, breast cancer, gastric cancer, pancreatic cancer, bladder carcinoma, cervical carcinoma, head and neck tumors, vulvar cancer and dermatological neoplasms including melanoma, squamous cell carcinoma and basal cell carcinomas. In other embodiments, non-solid tumors include lymphoproliferative disorders including leukemias and lymphomas. In other embodiments, the disorder is metastatic disease.

The compounds of the invention also may be used in the treatment of a cancer or cell proliferation disorder in a combination therapy with one or more of anti-cancer treatments such as surgery, radiation therapy, immunotherapy and/or one or more anti-cancer agents selected from the group consisting of anti-proliferative agents, other agents that modulate the metabolism of cancer cells, cytotoxic agents, cytostatic agents, and chemotherapeutic agents and salts and derivatives thereof. According to certain embodiments, the compounds of the invention may be used in the treatment of a cancer or cell proliferation disorder in combination therapy with any one of the drugs selected from a group consisting of an alkaloid, an alkylating agent, an antitumor antibiotic, an antimetabolite, a Bcr-Abl tyrosine kinase inhibitor, a nucleoside analogue, a multidrug resistance reversing agent, a DNA binding agent, microtubule binding drug, a toxin and a DNA antagonist. Those of skill in the art will recognize the chemotherapeutic agents classified into one or more particular classes of chemotherapeutic agents described above.

When used in combination with additional anti-proliferation agents, the compounds of the invention may enhance (e.g., synergize) the activity of these agents. Further, such synergism would permit the compounds of the invention, additional anti-proliferation agents, or both to be administered at lower dosages, and/or may significantly enhance the anti-proliferation properties of the compounds at any given dose.

### DEFINITIONS

For convenience, certain terms used in the specification, examples and appended claims are collected here.

A compound “activates PKM2” if the compound stimulates the enzymatic activity by PKM2 of reaction 1 by at least 10% relative to the activity of PKM2 under the same conditions but lacking only the presence of the compound.

Phosphoenolpyruvate+ADP→Pyruvate+ATP (1)

The activity of PKM2 may be measured by any reproducible means. The activity of PKM2 may be measured in vitro or in vivo.

For the avoidance of doubt, the term “a compound of the invention” or “compounds of the invention” refers to any compound or compounds disclosed herein e.g., a compound of the invention includes a compound of formulae I, Ia, Ib, II, IIa, IIb, III, IIIc, IIIb, IV, IVa, IVb, V, Va, Vb, VIa, and VIb and/or a compound in Tables 1, 1A and/or 1B. In some aspects, a compound of the invention does not include a compound in Tables 1A and 1B. Whenever the term is used in the context of the invention it is to be understood that the reference is being made to both the free base and the corresponding salts, solvates, and prodrugs provided that such is possible and/or appropriate under the circumstances.

The term “formula of the invention” or “formulae of the invention” refers any one or more of formulae I, Ia, Ib, II, IIa, IIb, III, IIIa, IIIb, IV, IVa, IVb, V, Va, Vb, VIa, and VIb.

“Treating”, includes any effect, e.g., lessening, reducing, modulating, or eliminating, that results in the improvement of the condition, disease, disorder, etc. “Treating” or “treatment” of a disease state includes: (1) inhibiting the disease state, i.e., arresting the development of the disease state or its clinical symptoms or (2) relieving the disease state, i.e., causing temporary or permanent regression of the disease state or its clinical symptoms.

“Preventing” means causing the clinical symptoms of the disease state not to develop, i.e., inhibiting the onset of disease, in a subject that may be exposed to or predisposed to the disease state, but does not yet experience or display symptoms of the disease state.

“Disease state” means any disease, disorder, condition, symptom, or indication.

In some embodiments, the cell proliferation disorder is cancer. As used herein, the term “cancer” includes solid tumors, such as lung, breast, colon, ovarian, brain, liver, pancreas, prostate, malignant melanoma, non-melanoma skin cancers, as well as hematologic tumors and/or malignancies, such as childhood leukemia and lymphomas, multiple myeloma, Hodgkin's disease, lymphomas of lymphocytic and cutaneous origin, acute and chronic leukemia such as acute lymphoblastic, acute myelocytic or chronic myelocytic leukemia, plasma cell neoplasm, lymphoid neoplasm and cancers associated with AIDS.

An “effective amount” of a compound is the quantity which, when administered to a subject having a disease or disorder, results in regression of the disease or disorder in the subject. Thus, for example, for a cell proliferation disorder an effective amount of a compound of the disclosed invention is the quantity which, when administered to a subject having a cell proliferation disorder, results in regression of cell growth in the subject. The amount of the compound to be administered to a subject will depend on the particular disorder, the mode of administration, co-administered compounds, if any, and the characteristics of the subject, such as general health, other diseases, age, sex, genotype, body weight and tolerance to drugs. The skilled artisan will be able to determine appropriate dosages depending on these and other factors.

“A therapeutically effective amount” means the amount of a compound that, when administered to a mammal, e.g., a human, for treating a disease, is sufficient to effect such treatment for the disease. The “therapeutically effective amount” will vary depending on the compound, the disease and its severity and the age, weight, etc., of the mammal to be treated.

A therapeutically effective amount of one or more of the compounds can be formulated with a pharmaceutically acceptable carrier for administration to a human or an animal. Accordingly, the compounds or the formulations can be administered, for example, via oral, parenteral, or topical routes, to provide an effective amount of the compound. In alternative embodiments, the compounds prepared in accordance with the invention can be used to coat or impregnate a medical device, e.g., a stent.

The term “prophylactically effective amount” means an effective amount of a compound or compounds, of the invention that is administered to prevent or reduce the risk of a disease state.

“Pharmacological effect” as used herein encompasses effects produced in the subject that achieve the intended purpose of a therapy.

With respect to the compounds useful in the invention, the following terms can be applicable:

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N).

The invention is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

The compounds described herein may have asymmetric centers. Compounds of the invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the invention. Cis and trans geometric isomers may be isolated as a mixture of isomers or as separated isomeric forms. All chiral, diastereomeric, racemic, and geometric isomeric forms of a structure are intended, unless the specific stereochemistry or isomeric form is specifically indicated. All tautomers of shown or described compounds are also considered to be part of the invention.

When any variable (e.g., R^{b}) occurs more than one time in any constituent or formula for a compound, its definition at each occurrence is independent of its definition at every other occurrence. Thus, for example, if a group is shown to be substituted with 0-2 R^{b} moieties, then the group may optionally be substituted with up to two R^{b} moieties and R^{b} at each occurrence is selected independently from the definition of R^{b}. Also, combinations of substituents and/or variables are permissible, but only if such combinations result in stable compounds.

When an atom or chemical moiety is followed by a subscripted numeric range (e.g., C₁₋₆), the invention is meant to encompass each number within the range as well as all intermediate ranges. For example, “C₁₋₆ alkyl” is meant to include alkyl groups with 1, 2, 3, 4, 5, 6, 1-6, 1-5, 1-4, 1-3, 1-2, 2-6, 2-5, 2-4, 2-3, 3-6, 3-5, 3-4, 4-6, 4-5, and 5-6 carbons.

As used herein, “alkyl” is intended to include both branched and straight-chain saturated aliphatic hydrocarbon groups having the specified number of carbon atoms. For example, C₁₋₆ alkyl is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, s-pentyl, and n-hexyl. In certain embodiments, a straight chain or branched chain alkyl has six or fewer carbon atoms in its backbone (e.g., C₁-C₆ for straight chain, C₃-C₆ for branched chain), and in another embodiment, a straight chain or branched chain alkyl has four or fewer carbon atoms. Likewise, cycloalkyls have from three to eight carbon atoms in their ring structure, and in other embodiments, cycloalkyls have five or six carbons in the ring structure.

The term “heteroalkyl” refers to an alkyl group in which one or more skeletal atoms of the alkyl are selected from an atom other than carbon, e.g., oxygen, nitrogen, sulfur, phosphorus or combinations thereof.

Unless the number of carbons is otherwise specified, “lower alkyl” includes an alkyl group, as defined above, but having from one to ten, or in other embodiments from one to six, carbon atoms in its backbone structure. “Lower alkenyl” and “lower alkynyl” have chain lengths of, for example, 2-6 carbon atoms.

The term “substituted alkyls” refers to alkyl moieties having substituents replacing hydrogen on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)).

“Alkenyl” includes unsaturated aliphatic groups analogous in length and possible substitution to the alkyls described above, but that contain at least one double bond. For example, the term “alkenyl” includes straight-chain alkenyl groups (e.g., ethenyl, propenyl, butenyl, pentenyl, hexenyl, heptenyl, octenyl, nonenyl, decenyl), branched-chain alkenyl groups, cycloalkenyl (e.g., alicyclic) groups (e.g., cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. In certain embodiments, a straight chain or branched chain alkenyl group has six or fewer carbon atoms in its backbone (e.g., C₂-C₆ for straight chain, C₃-C₆ for branched chain). Likewise, cycloalkenyl groups may have from three to eight carbon atoms in their ring structure, and in some embodiments, cycloalkenyl groups have five or six carbons in the ring structure. The term “C₂-C₆” includes alkenyl groups containing two to six carbon atoms. The term “C₃-C₆” includes alkenyl groups containing three to six carbon atoms.

The term “substituted alkenyls” refers to alkenyl moieties having substituents replacing hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight-chain alkynyl groups (e.g., ethynyl, propynyl, butynyl, pentynyl, hexynyl, heptynyl, octynyl, nonynyl, decynyl), branched-chain alkynyl groups, and cycloalkyl or cycloalkenyl substituted alkynyl groups. In certain embodiments, a straight chain or branched chain alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “substituted alkynyls” refers to alkynyl moieties having substituents replacing hydrogen on one or more hydrocarbon backbone carbon atoms. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Aryl” includes groups with aromaticity, including 5- and 6-membered “unconjugated”, or single-ring, aromatic groups that may include from zero to four heteroatoms, as well as “conjugated”, or multicyclic, systems with at least one aromatic ring. Examples of aryl groups include benzene, phenyl, pyrrole, furan, thiophene, thiazole, isothiazole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthyridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings, which are not aromatic so as to form a multicyclic system (e.g., tetralin, methylenedioxyphenyl).

As used herein, “halo” or “halogen” refers to fluoro, chloro, bromo, and iodo. The term “perhalogenated” generally refers to a moiety wherein all hydrogens are replaced by halogen atoms.

“Counterion” is used to represent a small, negatively charged species such as chloride, bromide, hydroxide, acetate, and sulfate.

The term “non-hydrogen substituent” refers to substituents other than hydrogen. Non-limiting examples include alkyl groups, alkoxy groups, halogen groups, hydroxyl groups, aryl groups, etc.

As used herein, “carbocycle” or “carbocyclic ring” is intended to mean any stable monocyclic, bicyclic, or tricyclic ring having the specified number of carbons, any of which may be saturated, unsaturated, or aromatic. For example a C₃₋₁₄ carbocycle is intended to mean a mono-, bi-, or tricyclic ring having 3, 4, 5, 6, 7, 8, 9, 10, 11, 12, 13, or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl, and tetrahydronaphthyl. Bridged rings are also included in the definition of carbocycle, including, for example, [3.3.0]bicyclooctane, [4.3.0]bicyclononane, [4.4.0]bicyclodecane, and [2.2.2]bicyclooctane. A bridged ring occurs when one or more carbon atoms link two non-adjacent carbon atoms. In some embodiments, bridge rings are one or two carbon atoms. It is noted that a bridge always converts a monocyclic ring into a tricyclic ring. When a ring is bridged, the substituents recited for the ring may also be present on the bridge. Fused (e.g., naphthyl and tetrahydronaphthyl) and spiro rings are also included.

As used herein, the term “heterocycle” or “heterocyclic” is intended to mean any stable monocyclic, bicyclic, or tricyclic ring which is saturated, unsaturated, or aromatic and comprises carbon atoms and one or more ring heteroatoms, e.g., 1 or 1-2 or 1-3 or 1-4 or 1-5 or 1-6 heteroatoms, independently selected from the group consisting of nitrogen, oxygen, and sulfur. A bicyclic or tricyclic heterocycle may have one or more heteroatoms located in one ring, or the heteroatoms may be located in more than one ring. The nitrogen and sulfur heteroatoms may optionally be oxidized (i.e., N→O and S(O)ₚ, where p=1 or 2). When a nitrogen atom is included in the ring it is either N or NH, depending on whether or not it is attached to a double bond in the ring (i.e., a hydrogen is present if needed to maintain the tri-valency of the nitrogen atom). The nitrogen atom may be substituted or unsubstituted (i.e., N or NR wherein R is H or another substituent, as defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocycle may optionally be quaternized. In some embodiments, when the total number of S and O atoms in the heterocycle exceeds 1, then these heteroatoms are not adjacent to one another. Bridged rings are also included in the definition of heterocycle. A bridged ring occurs when one or more atoms (i.e., C, O, N, or S) link two non-adjacent carbon or nitrogen atoms. Bridges include, but are not limited to, one carbon atom, two carbon atoms, one nitrogen atom, two nitrogen atoms, and a carbon-nitrogen group. It is noted that a bridge always converts a monocyclic ring into a tricyclic ring. When a ring is bridged, the substituents recited for the ring may also be present on the bridge. Spiro and fused rings are also included.

As used herein, the term “aromatic heterocycle” or “heteroaryl” is intended to mean a stable 5, 6, or 7-membered monocyclic or bicyclic aromatic heterocyclic ring or 7, 8, 9, 10, 11, or 12-membered bicyclic aromatic heterocyclic ring which consists of carbon atoms and one or more heteroatoms, e.g., 1 or 1-2 or 1-3 or 1-4 or 1-5 or 1-6 heteroatoms, independently selected from the group consisting of nitrogen, oxygen, and sulfur. In the case of bicyclic heterocyclic aromatic rings, only one of the two rings needs to be aromatic (e.g., 2,3-dihydroindole), though both may be (e.g., quinoline). The second ring can also be fused or bridged as defined above for heterocycles. The nitrogen atom may be substituted or unsubstituted (i.e., N or NR wherein R is H or another substituent, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl.

“Acyl” includes compounds and moieties that contain the acyl radical (CH₃CO—) or a carbonyl group. “Substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

“Acylamino” includes moieties wherein an acyl moiety is bonded to an amino group. For example, the term includes alkylcarbonylamino, arylcarbonylamino, carbamoyl and ureido groups.

“Aroyl” includes compounds and moieties with an aryl or heteroaromatic moiety bound to a carbonyl group. Examples of aroyl groups include phenylcarboxy, naphthyl carboxy, etc.

“Alkoxyalkyl”, “alkylaminoalkyl” and “thioalkoxyalkyl” include alkyl groups, as described above, which further include oxygen, nitrogen or sulfur atoms replacing one or more hydrocarbon backbone carbon atoms, e.g., oxygen, nitrogen or sulfur atoms.

The term “alkoxy” or “alkoxyl” includes substituted and unsubstituted alkyl, alkenyl, and alkynyl groups covalently linked to an oxygen atom. Examples of alkoxy groups (or alkoxyl radicals) include methoxy, ethoxy, isopropyloxy, propoxy, butoxy, and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, and trichloromethoxy.

The term “thiocarbonyl” or “thiocarboxy” includes compounds and moieties which contain a carbon connected with a double bond to a sulfur atom.

The term “ether” or “alkoxy” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or heteroatoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group.

The term “hydroxy” or “hydroxyl” includes groups with an —OH or —O⁻.

“Polycyclyl” or “polycyclic radical” refers to two or more cyclic rings (e.g., cycloalkyls, cycloalkenyls, cycloalkynyls, aryls and/or heterocyclyls) in which two or more carbons are common to two adjoining rings. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminocarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

An “anionic group,” as used herein, refers to a group that is negatively charged at physiological pH. Anionic groups include carboxylate, sulfate, sulfonate, sulfinate, sulfamate, tetrazolyl, phosphate, phosphonate, phosphinate, or phosphorothioate or functional equivalents thereof “Functional equivalents” of anionic groups are intended to include bioisosteres, e.g., bioisosteres of a carboxylate group. Bioisosteres encompass both classical bioisosteric equivalents and non-classical bioisosteric equivalents. Classical and non-classical bioisosteres are known in the art (see, e.g., Silverman, R. B. The Organic Chemistry of Drug Design and Drug Action, Academic Press, Inc.: San Diego, Calif., 1992, pp. 19-23). In some embodiments, an anionic group is a carboxylate.

In the specification, the structural formula of the compound represents a certain isomer for convenience in some cases, but the invention includes all isomers such as geometrical isomer, optical isomer based on an asymmetrical carbon, stereoisomer, tautomer and the like which occur structurally and an isomer mixture and is not limited to the description of the formula for convenience, and may be any one isomer or a mixture. Therefore, an asymmetrical carbon atom may be present in the molecule and an optically active compound and a racemic compound may be present in the compound, but the invention is not limited to them and includes any one. In addition, a crystal polymorphism may be present but is not limiting, but any crystal form may be single or a crystal form mixture, or an anhydride or hydrate. Further, so-called metabolite which is produced by degradation of the compound in vivo is included in the scope of the invention.

“Isomerism” means compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

“Chiral isomer” means a compound with at least one chiral center. It has two enantiomeric forms of opposite chirality and may exist either as an individual enantiomer or as a mixture of enantiomers. A mixture containing equal amounts of individual enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al, Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J., Chem. Educ. 1964, 41, 116).

“Geometric Isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Further, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

The terms “crystal polymorph” or “polymorph” or “crystal form” means crystal structures in which a compound (or salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate.

“Tautomer” refers to compounds whose structures differ markedly in arrangement of atoms, but which exist in easy and rapid equilibrium. It is to be understood that the compounds of the invention may be depicted as different tautomers. It should also be understood that when compounds have tautomeric forms, all tautomeric forms are intended to be within the scope of the invention, and the naming of the compounds does not exclude any tautomer form.

Some compounds of the invention can exist in tautomeric forms, which are also intended to be encompassed within the scope of the invention.

The compounds, salts and prodrugs of the invention can exist in several tautomeric forms, including the enol and imine form, and the keto and enamine form and geometric isomers and mixtures thereof. All such tautomeric forms are included within the scope of the invention. Tautomers exist as mixtures of a tautomeric set in solution. In solid form, usually one tautomer predominates. Even though one tautomer may be described, the invention includes all tautomers of the compounds.

A tautomer is one of two or more structural isomers that exist in equilibrium and are readily converted from one isomeric form to another. This reaction results in the formal migration of a hydrogen atom accompanied by a switch of adjacent conjugated double bonds. In solutions where tautomerization is possible, a chemical equilibrium of the tautomers will be reached. The exact ratio of the tautomers depends on several factors, including temperature, solvent, and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism, is exhibited by glucose. It arises as a result of the aldehyde group (—CHO) in a sugar chain molecule reacting with one of the hydroxy groups (—OH) in the same molecule to give it a cyclic (ring-shaped) form.

Tautomerizations are catalyzed by: Base: 1. deprotonation; 2. formation of a delocalized anion (e.g. an enolate); 3. protonation at a different position of the anion; Acid: 1. protonation; 2. formation of a delocalized cation; 3. deprotonation at a different position adjacent to the cation.

Common tautomeric pairs are: ketone-enol, amide-nitrile, lactam-lactim, amide-imidic acid tautomerism in heterocyclic rings (e.g. in the nucleobases guanine, thymine, and cytosine), amine-enamine and enamine-enamine.

It will be noted that the structure of some of the compounds of the invention include asymmetric carbon atoms. It is to be understood accordingly that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate. The compounds of this invention may exist in stereoisomeric form, therefore can be produced as individual stereoisomers or as mixtures.

As used herein, the term “analog” refers to a compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative”, refers to compounds that have a common core structure, and are substituted with various groups as described herein. For example, all of the compounds represented by Formula I are pyrazoline derivatives, and have pyrazoline as a common core.

A “pharmaceutical composition” is a formulation containing the compounds in a form suitable for administration to a subject. In some embodiments, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a formulation of the disclosed compound or salt, hydrate, solvate, or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

The term “immediate release” is defined as a release of compound from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a compound from a dosage form over a prolonged period.

A “subject” includes mammals, e.g., humans, companion animals (e.g., dogs, cats, birds, and the like), farm animals (e.g., cows, sheep, pigs, horses, fowl, and the like) and laboratory animals (e.g., rats, mice, guinea pigs, birds, and the like). In some embodiments, the subject is human.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used herein includes both one and more than one such excipient.

The compounds of the invention are capable of further forming salts. All of these forms are also contemplated within the scope of the invention.

“Pharmaceutically acceptable salt” of a compound means a salt that is pharmaceutically acceptable and that possesses the desired pharmacological activity of the parent compound.

As used herein, “pharmaceutically acceptable salts” refer to derivatives of the compounds wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The pharmaceutically acceptable salts of the invention can be synthesized from a parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; non-aqueous media like ether, ethyl acetate, ethanol, isopropanol, or acetonitrile can be used. Lists of suitable salts are found in Remington's Pharmaceutical Sciences, 18th ed. (Mack Publishing Company, 1990). For example, salts can include, but are not limited to, the hydrochloride and acetate salts of the aliphatic amine-containing, hydroxyl amine-containing, and imine-containing compounds of the invention.

Compounds of the invention can also be prepared as esters, for example pharmaceutically acceptable esters. For example a carboxylic acid function group in a compound can be converted to its corresponding ester, e.g., a methyl, ethyl, or other ester. Also, an alcohol group in a compound can be converted to its corresponding ester, e.g., an acetate, propionate, or other ester.

Compounds of the invention can also be prepared as prodrugs, for example pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.) the compounds of the invention can be delivered in prodrug form. Thus, the invention is intended to cover prodrugs of the presently claimed compounds, methods of delivering the same and compositions containing the same. “Prodrugs” are intended to include any covalently bonded carriers that release an active parent drug of the invention in vivo when such prodrug is administered to a subject. Prodrugs may be prepared by modifying functional groups present in the compound in such a way that the modifications are cleaved, either in routine manipulation or in vivo, to the parent compound. Prodrugs include compounds of the invention wherein a hydroxy, amino, sulfhydryl, carboxy, or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates, and benzoate derivatives) and carbamates (e.g., N,N-dimethylaminocarbonyl) of hydroxy functional groups, esters groups (e.g. ethyl esters, morpholinoethanol esters) of carboxyl functional groups, N-acyl derivatives (e.g. N-acetyl) N-Mannich bases, Schiff bases and enaminones of amino functional groups, oximes, acetals, ketals and enol esters of ketone and aldehyde functional groups in compounds of Formula I, and the like, See Bundegaard, H. “Design of Prodrugs” p1-92, Elesevier, New York-Oxford (1985).

“Stable compound” and “stable structure” are meant to indicate a compound that is sufficiently robust to survive isolation to a useful degree of purity from a reaction mixture, and formulation into an efficacious therapeutic agent.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the specification will control.

All percentages and ratios used herein, unless otherwise indicated, are by weight.

The invention provides methods for the treatment of a cell proliferative disorder in a subject in need thereof by administering to a subject in need of such treatment, a therapeutically effective amount of a compound of the invention, or a pharmaceutically acceptable salt, prodrug, metabolite, polymorph or solvate thereof. The cell proliferative disorder can be cancer or a precancerous condition. The invention further provides the use of a compound of the invention, or a pharmaceutically acceptable salt, prodrug, metabolite, polymorph or solvate thereof, for the preparation of a medicament useful for the treatment of a cell proliferative disorder.

The invention also provides methods of protecting against a cell proliferative disorder in a subject in need thereof by administering a therapeutically effective amount of compound of the invention, or a pharmaceutically acceptable salt, prodrug, metabolite, polymorph or solvate thereof, to a subject in need of such treatment. The cell proliferative disorder can be cancer or a precancerous condition. The invention also provides the use of compound of the invention, or a pharmaceutically acceptable salt, prodrug, metabolite, polymorph or solvate thereof, for the preparation of a medicament useful for the prevention of a cell proliferative disorder.

As used herein, a “subject in need thereof” is a subject having a cell proliferative disorder, or a subject having an increased risk of developing a cell proliferative disorder relative to the population at large. A subject in need thereof can have a precancerous condition. Preferably, a subject in need thereof has cancer. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

As used herein, the term “cell proliferative disorder” refers to conditions in which unregulated or abnormal growth, or both, of cells can lead to the development of an unwanted condition or disease, which may or may not be cancerous. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. In one aspect, the methods provided herein are used to treat or alleviate a symptom of cancer. The term “cancer” includes solid tumors, as well as, hematologic tumors and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; psoriasis; eczema; ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; Multiple Sclerosis; lupus; fibromyalgia; AIDS and other viral diseases such as Herpes Zoster, Herpes Simplex I or II, influenza virus and cytomegalovirus; metabolic disorders, and diabetes mellitus.

Exemplary cancers include, but are not limited to, adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, uringary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

A “cell proliferative disorder of the hematologic system” is a cell proliferative disorder involving cells of the hematologic system. A cell proliferative disorder of the hematologic system can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. In one aspect, compositions of the invention may be used to treat a cancer selected from the group consisting of a hematologic cancer of the invention or a hematologic cell proliferative disorder of the invention. A hematologic cancer of the t invention can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

A “cell proliferative disorder of the lung” is a cell proliferative disorder involving cells of the lung. Cell proliferative disorders of the lung can include all forms of cell proliferative disorders affecting lung cells. Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. Compositions of the invention may be used to treat lung cancer or cell proliferative disorders of the lung. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma”, bronchioalveolar carcinoma, giant cell carcinoma, spindle cell carcinoma, and large cell neuroendocrine carcinoma. Lung cancer can include lung neoplasms having histologic and ultrastructual heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

A “cell proliferative disorder of the colon” is a cell proliferative disorder involving cells of the colon. In one aspect, the cell proliferative disorder of the colon is colon cancer. In one aspect, compositions of the invention may be used to treat colon cancer or cell proliferative disorders of the colon. Colon cancer can include all forms of cancer of the colon. Colon cancer can include sporadic and hereditary colon cancers. Colon cancer can include malignant colon neoplasms, carcinoma in situ, typical carcinoid tumors, and atypical carcinoid tumors. Colon cancer can include adenocarcinoma, squamous cell carcinoma, and adenosquamous cell carcinoma. Colon cancer can be associated with a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis. Colon cancer can be caused by a hereditary syndrome selected from the group consisting of hereditary nonpolyposis colorectal cancer, familial adenomatous polyposis, Gardner's syndrome, Peutz-Jeghers syndrome, Turcot's syndrome and juvenile polyposis.

Cell proliferative disorders of the colon can include all forms of cell proliferative disorders affecting colon cells. Cell proliferative disorders of the colon can include colon cancer, precancerous conditions of the colon, adenomatous polyps of the colon and metachronous lesions of the colon. A cell proliferative disorder of the colon can include adenoma. Cell proliferative disorders of the colon can be characterized by hyperplasia, metaplasia, and dysplasia of the colon. Prior colon diseases that may predispose individuals to development of cell proliferative disorders of the colon can include prior colon cancer. Current disease that may predispose individuals to development of cell proliferative disorders of the colon can include Crohn's disease and ulcerative colitis. A cell proliferative disorder of the colon can be associated with a mutation in a gene selected from the group consisting of p53, ras, FAP and DCC. An individual can have an elevated risk of developing a cell proliferative disorder of the colon due to the presence of a mutation in a gene selected from the group consisting of p53, ras, FAP and DCC.

A “cell proliferative disorder of the pancreas” is a cell proliferative disorder involving cells of the pancreas. Cell proliferative disorders of the pancreas can include all forms of cell proliferative disorders affecting pancreatic cells. Cell proliferative disorders of the pancreas can include pancreas cancer, a precancer or precancerous condition of the pancreas, hyperplasia of the pancreas, and dysaplasia of the pancreas, benign growths or lesions of the pancreas, and malignant growths or lesions of the pancreas, and metastatic lesions in tissue and organs in the body other than the pancreas. Pancreatic cancer includes all forms of cancer of the pancreas. Pancreatic cancer can include ductal adenocarcinoma, adenosquamous carcinoma, pleomorphic giant cell carcinoma, mucinous adenocarcinoma, osteoclast-like giant cell carcinoma, mucinous cystadenocarcinoma, acinar carcinoma, unclassified large cell carcinoma, small cell carcinoma, pancreatoblastoma, papillary neoplasm, mucinous cystadenoma, papillary cystic neoplasm, and serous cystadenoma. Pancreatic cancer can also include pancreatic neoplasms having histologic and ultrastructual heterogeneity (e.g., mixed cell types).

A “cell proliferative disorder of the prostate” is a cell proliferative disorder involving cells of the prostate. Cell proliferative disorders of the prostate can include all forms of cell proliferative disorders affecting prostate cells. Cell proliferative disorders of the prostate can include prostate cancer, a precancer or precancerous condition of the prostate, benign growths or lesions of the prostate, and malignant growths or lesions of the prostate, and metastatic lesions in tissue and organs in the body other than the prostate. Cell proliferative disorders of the prostate can include hyperplasia, metaplasia, and dysplasia of the prostate.

A “cell proliferative disorder of the skin” is a cell proliferative disorder involving cells of the skin. Cell proliferative disorders of the skin can include all forms of cell proliferative disorders affecting skin cells. Cell proliferative disorders of the skin can include a precancer or precancerous condition of the skin, benign growths or lesions of the skin, melanoma, malignant melanoma and other malignant growths or lesions of the skin, and metastatic lesions in tissue and organs in the body other than the skin. Cell proliferative disorders of the skin can include hyperplasia, metaplasia, and dysplasia of the skin.

A “cell proliferative disorder of the ovary” is a cell proliferative disorder involving cells of the ovary. Cell proliferative disorders of the ovary can include all forms of cell proliferative disorders affecting cells of the ovary. Cell proliferative disorders of the ovary can include a precancer or precancerous condition of the ovary, benign growths or lesions of the ovary, ovarian cancer, malignant growths or lesions of the ovary, and metastatic lesions in tissue and organs in the body other than the ovary. Cell proliferative disorders of the skin can include hyperplasia, metaplasia, and dysplasia of cells of the ovary.

A “cell proliferative disorder of the breast” is a cell proliferative disorder involving cells of the breast. Cell proliferative disorders of the breast can include all forms of cell proliferative disorders affecting breast cells. Cell proliferative disorders of the breast can include breast cancer, a precancer or precancerous condition of the breast, benign growths or lesions of the breast, and malignant growths or lesions of the breast, and metastatic lesions in tissue and organs in the body other than the breast. Cell proliferative disorders of the breast can include hyperplasia, metaplasia, and dysplasia of the breast.

A cell proliferative disorder of the breast can be a precancerous condition of the breast. Compositions of the invention may be used to treat a precancerous condition of the breast. A precancerous condition of the breast can include atypical hyperplasia of the breast, ductal carcinoma in situ (DCIS), intraductal carcinoma, lobular carcinoma in situ (LCIS), lobular neoplasia, and stage 0 or grade 0 growth or lesion of the breast (e.g., stage 0 or grade 0 breast cancer, or carcinoma in situ). A precancerous condition of the breast can be staged according to the TNM classification scheme as accepted by the American Joint Committee on Cancer (AJCC), where the primary tumor (T) has been assigned a stage of T0 or Tis; and where the regional lymph nodes (N) have been assigned a stage of N0; and where distant metastasis (M) has been assigned a stage of M0.

The cell proliferative disorder of the breast can be breast cancer. Breast cancer includes all forms of cancer of the breast. Breast cancer can include primary epithelial breast cancers. Breast cancer can include cancers in which the breast is involved by other tumors such as lymphoma, sarcoma or melanoma. Breast cancer can include carcinoma of the breast, ductal carcinoma of the breast, lobular carcinoma of the breast, undifferentiated carcinoma of the breast, cystosarcoma phyllodes of the breast, angiosarcoma of the breast, and primary lymphoma of the breast. Breast cancer can include Stage I, II, IIIA, IIIB, IIIC and IV breast cancer. Ductal carcinoma of the breast can include invasive carcinoma, invasive carcinoma in situ with predominant intraductal component, inflammatory breast cancer, and a ductal carcinoma of the breast with a histologic type selected from the group consisting of comedo, mucinous (colloid), medullary, medullary with lymphcytic infiltrate, papillary, scirrhous, and tubular. Lobular carcinoma of the breast can include invasive lobular carcinoma with predominant in situ component, invasive lobular carcinoma, and infiltrating lobular carcinoma. Breast cancer can include Paget's disease, Paget's disease with intraductal carcinoma, and Paget's disease with invasive ductal carcinoma. Breast cancer can include breast neoplasms having histologic and ultrastructual heterogeneity (e.g., mixed cell types).

A breast cancer that is to be treated can include familial breast cancer. A breast cancer that is to be treated can include sporadic breast cancer. A breast cancer that is to be treated can arise in a male subject. A breast cancer that is to be treated can arise in a female subject. A breast cancer that is to be treated can arise in a premenopausal female subject or a postmenopausal female subject. A breast cancer that is to be treated can arise in a subject equal to or older than 30 years old, or a subject younger than 30 years old. A breast cancer that is to be treated has arisen in a subject equal to or older than 50 years old, or a subject younger than 50 years old. A breast cancer that is to be treated can arise in a subject equal to or older than 70 years old, or a subject younger than 70 years old.

A breast cancer that is to be treated can be typed to identify a familial or spontaneous mutation in BRCA1, BRCA2, or p53. A breast cancer that is to be treated can be typed as having a HER₂/neu gene amplification, as overexpressing HER₂/neu, or as having a low, intermediate or high level of HER₂/neu expression. A breast cancer that is to be treated can be typed as HER₂-negative or HER₂-positive. HER₂-typing of a breast cancer may be performed by any reproducible means. A breast cancer that is to be treated can be typed for a marker selected from the group consisting of estrogen receptor (ER), progesterone receptor (PR), human epidermal growth factor receptor-2, Ki-67, CA15-3, CA 27-29, and c-Met. A breast cancer that is to be treated can be typed as ER-unknown, ER-rich or ER-poor. A breast cancer that is to be treated can be typed as ER-negative or ER-positive. ER-typing of a breast cancer may be performed by any reproducible means. ER-typing of a breast cancer may be performed as set forth in Onkologie 27: 175-179 (2004). A breast cancer that is to be treated can be typed as PR-unknown, PR-rich or PR-poor. A breast cancer that is to be treated can be typed as PR-negative or PR-positive. PR-typing of a breast cancer may be performed by any reproducible means. A breast cancer that is to be treated can be typed as receptor positive or receptor negative. A breast cancer that is to be treated can have multiple receptors each independently typed as receptor positive or receptor negative. For example, a breast cancer that can be treated can be “a triple negative breast cancer” (i.e., typed as ER-negative, PR-negative, and HER₂-negative). A breast cancer that is to be treated can be typed as being associated with elevated blood levels of CA 15-3, or CA 27-29, or both.

A breast cancer that is to be treated can include a localized tumor of the breast. A breast cancer that is to be treated can include a tumor of the breast that is associated with a negative sentinel lymph node (SLN) biopsy. A breast cancer that is to be treated can include a tumor of the breast that is associated with a positive sentinel lymph node (SLN) biopsy. A breast cancer that is to be treated can include a tumor of the breast that is associated with one or more positive axillary lymph nodes, where the axillary lymph nodes have been staged by any applicable method. A breast cancer that is to be treated can include a tumor of the breast that has been typed as having nodal negative status (e.g., node-negative) or nodal positive status (e.g., node-positive). A breast cancer that is to be treated can include a tumor of the breast that has metastasized to other locations in the body. A breast cancer that is to be treated can be classified as having metastasized to a location selected from the group consisting of bone, lung, liver, or brain. A breast cancer that is to be treated can be classified according to a characteristic selected from the group consisting of metastatic, localized, regional, local-regional, locally advanced, distant, multicentric, bilateral, ipsilateral, contralateral, newly diagnosed, recurrent, and inoperable.

A compound of the invention, or a pharmaceutically acceptable salt, prodrug, metabolite, polymorph or solvate thereof, may be used to treat or prevent a cell proliferative disorder of the breast, or to treat or prevent breast cancer, in a subject having an increased risk of developing breast cancer relative to the population at large. A subject with an increased risk of developing breast cancer relative to the population at large is a female subject with a family history or personal history of breast cancer. A subject with an increased risk of developing breast cancer relative to the population at large is a female subject having a germ-line or spontaneous mutation in BRCA1 or BRCA2, or both. A subject with an increased risk of developing breast cancer relative to the population at large is a female subject with a family history of breast cancer and a germ-line or spontaneous mutation in BRCA1 or BRCA2, or both. A subject with an increased risk of developing breast cancer relative to the population at large is a female who is greater than 30 years old, greater than 40 years old, greater than 50 years old, greater than 60 years old, greater than 70 years old, greater than 80 years old, or greater than 90 years old. A subject with an increased risk of developing breast cancer relative to the population at large is a subject with atypical hyperplasia of the breast, ductal carcinoma in situ (DCIS), intraductal carcinoma, lobular carcinoma in situ (LCIS), lobular neoplasia, or a stage 0 growth or lesion of the breast (e.g., stage 0 or grade 0 breast cancer, or carcinoma in situ).

A breast cancer that is to be treated can histologically graded according to the Scarff-Bloom-Richardson system, wherein a breast tumor has been assigned a mitosis count score of 1, 2, or 3; a nuclear pleiomorphism score of 1, 2, or 3; a tubule formation score of 1, 2, or 3; and a total Scarff-Bloom-Richardson score of between 3 and 9. A breast cancer that is to be treated can be assigned a tumor grade according to the International Consensus Panel on the Treatment of Breast Cancer selected from the group consisting of grade 1, grade 1-2, grade 2, grade 2-3, or grade 3.

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can include a tumor that has been determined to be less than or equal to about 2 centimeters in diameter. A cancer that is to be treated can include a tumor that has been determined to be from about 2 to about 5 centimeters in diameter. A cancer that is to be treated can include a tumor that has been determined to be greater than or equal to about 3 centimeters in diameter. A cancer that is to be treated can include a tumor that has been determined to be greater than 5 centimeters in diameter. A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “candidate compound” refers to a compound of the invention, or a pharmaceutically acceptable salt, prodrug, metabolite, polymorph or solvate thereof, that has been or will be tested in one or more in vitro or in vivo biological assays, in order to determine if that compound is likely to elicit a desired biological or medical response in a cell, tissue, system, animal or human that is being sought by a researcher or clinician. A candidate compound is a compound of the invention, or a pharmaceutically acceptable salt, prodrug, metabolite, polymorph or solvate thereof. The biological or medical response can be the treatment of cancer. The biological or medical response can be treatment or prevention of a cell proliferative disorder. In vitro or in vivo biological assays can include, but are not limited to, enzymatic activity assays, electrophoretic mobility shift assays, reporter gene assays, in vitro cell viability assays, and the assays described herein.

As used herein, “monotherapy” refers to the administration of a single active or therapeutic compound to a subject in need thereof. In one aspect, monotherapy will involve administration of a therapeutically effective amount of an active compound. For example, cancer monotherapy with one of the compound of the invention, or a pharmaceutically acceptable salt, prodrug, metabolite, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the invention, or a pharmaceutically acceptable salt, prodrug, metabolite, polymorph or solvate thereof, is more effective than combination therapy in inducing a desired biological effect.

As used herein, “treating” or “treat” describes the management and care of a patient for the purpose of combating a disease, condition, or disorder and includes the administration of a compound of the invention, or a pharmaceutically acceptable salt, prodrug, metabolite, polymorph or solvate thereof, to alleviate the symptoms or complications of a disease, condition or disorder, or to eliminate the disease, condition or disorder.

A compound of the t invention, or a pharmaceutically acceptable salt, prodrug, metabolite, polymorph or solvate thereof, can also be used to prevent a disease, condition or disorder. As used herein, “preventing” or “prevent” describes reducing or eliminating the onset of the symptoms or complications of the disease, condition or disorder.

As used herein, the term “alleviate” is meant to describe a process by which the severity of a sign or symptom of a disorder is decreased. Importantly, a sign or symptom can be alleviated without being eliminated. In one embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. In one aspect, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. In one aspect, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. In one aspect, the specified magnification is 2×, 3×, 4×, 5×, 10×, or 50×.

Treating cancer can result in a decrease in number of metastatic lesions in other tissues or organs distant from the primary tumor site. In one aspect, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. In one aspect, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. In one aspect, the average survival time is increased by more than 30 days; by more than 60 days; by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the invention, or a pharmaceutically acceptable salt, prodrug, metabolite, analog or derivative thereof. In one aspect, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the invention, or a pharmaceutically acceptable salt, prodrug, metabolite, analog or derivative thereof. In one aspect, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. In one aspect, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. In one aspect, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. In one aspect, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. In one aspect, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. In one aspect, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

One aspect of the invention includes methods of preventing or treating a metabolic disorder comprising administering a pharmaceutical composition that includes an effective amount of one of the compounds of any of the formulae or compounds described herein. A metabolic disorder is any disorder that involves an alteration in the normal metabolism of carbohydrates, lipids, proteins, water, and nucleic acids. Examples of metabolic disorders include type 1 and type 2 diabetes mellitus, complications of diabetes (such as e.g. retinopathy, nephropathy or neuropathies, diabetic foot, ulcers, macroangiopathies), metabolic acidosis or ketosis, reactive hypoglycaemia, hyperinsulinaemia, glucose metabolic disorder, insulin resistance, metabolic syndrome, dyslipidaemias of different origins, atherosclerosis and related diseases, obesity, high blood pressure, chronic heart failure, edema and hyperuricaemia. Metabolic syndrome is one type of metabolic disorder. Metabolic syndrome is a combination of medical disorders that, when they occurr together, increase the risk of developing cardiovascular disease and diabetes. Metabolic syndrome is also known as metabolic syndrome X, cardiometabolic syndrome, syndrome X, insulin resistance syndrome, Reaven's syndrome (named for Gerald Reaven), and CHAOS (in Australia).

As used herein, the term “selectively” means tending to occur at a higher frequency in one population than in another population. The compared populations can be cell populations. In one aspect, a compound of the invention, or a pharmaceutically acceptable salt, prodrug, metabolite, polymorp In one aspect, a compound of the invention, or a pharmaceutically acceptable salt, prodrug, metabolite, polymorph or solvate thereof, acts selectively to modulate one molecular target (e.g., PKM2) but does not significantly modulate another molecular target (e.g., PKM1). The invention also provides a method for selectively inhibiting or activating the activity of an enzyme, such as a kinase (e.g., PKM2). Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

A compound of the invention, or a pharmaceutically acceptable salt, prodrug, metabolite, polymorph or solvate thereof, can modulate the activity of a molecular target (e.g., PKM2). Modulating refers to stimulating or inhibiting an activity of a molecular target. In one aspect, a compound of the invention, or a pharmaceutically acceptable salt, prodrug, metabolite, polymorph or solvate thereof, modulates the activity of a molecular target if it stimulates or inhibits the activity of the molecular target by at least 10% relative to the activity of the molecular target under the same conditions but lacking only the presence of said compound. In one aspect, a compound of the invention, or a pharmaceutically acceptable salt, prodrug, metabolite, polymorph or solvate thereof, modulates the activity of a molecular target if it stimulates or inhibits the activity of the molecular target by at least 2-fold, at least 5-fold, at least 10-fold, at least 20-fold, at least 50-fold, at least 100-fold relative to the activity of the molecular target under the same conditions but lacking only the presence of said compound. The activity of a molecular target may be measured by any reproducible means. The activity of a molecular target may be measured in vitro or in vivo. For example, the activity of a molecular target may be measured in vitro or in vivo by an enzymatic activity assay.

A compound of the invention, or a pharmaceutically acceptable salt, prodrug, metabolite, polymorph or solvate thereof, does not significantly modulate the activity of a molecular target if the addition of the compound does not stimulate or inhibit the activity of the molecular target by greater than 10% relative to the activity of the molecular target under the same conditions but lacking only the presence of said compound.

As used herein, the term “isozyme selective” means preferential inhibition or stimulation of a first isoform of an enzyme in comparison to a second isoform of an enzyme (e.g., preferential inhibition or stimulation of a kinase isozyme alpha in comparison to a kinase isozyme beta).

A change in enzymatic activity caused by a compound of the invention, or a pharmaceutically acceptable salt, prodrug, metabolite, polymorph or solvate thereof, can be measured in the disclosed assays. The change in enzymatic activity can be characterized by the change in the extent of phosphorylation of certain substrates. As used herein, “phosphorylation” refers to the addition of phosphate groups to a substrate, including proteins and organic molecules; and, plays an important role in regulating the biological activities of proteins. Preferably, the phosphorylation assayed and measured involves the addition of phosphate groups to tyrosine residues. The substrate can be a peptide or protein.

In some assays, immunological reagents, e.g., antibodies and antigens, are employed. Fluorescence can be utilized in the measurement of enzymatic activity in some assays. As used herein, “fluorescence” refers to a process through which a molecule emits a photon as a result of absorbing an incoming photon of higher energy by the same molecule. Specific methods for assessing the biological activity of the disclosed compounds are described in the examples.

Activating refers to placing a composition of matter (e.g., protein or nucleic acid) in a state suitable for carrying out a desired biological function. A composition of matter capable of being activated also has an unactivated state. An activated composition of matter may have an inhibitory or stimulatory biological function, or both.

Elevation refers to an increase in a desired biological activity of a composition of matter (e.g., a protein or a nucleic acid). Elevation may occur through an increase in concentration of a composition of matter.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci US A. 100(5): 2674-8, 2003. In an aspect, cell death occurs by apoptosis.

In one aspect, an effective amount of a compound of the invention, or a pharmaceutically acceptable salt, prodrug, metabolite, polymorph or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a compound of the invention, or a pharmaceutically acceptable salt, prodrug, metabolite, polymorph or solvate thereof, can induce or activate cell death selectively in cancer cells. Administering to a subject in need thereof a compound of the invention, or a pharmaceutically acceptable salt, prodrug, metabolite, polymorph or solvate thereof, can induce or activate cell death selectively in cancer cells. Contacting a cell with a compound of the invention, or a pharmaceutically acceptable salt, prodrug, metabolite, polymorph or solvate thereof, can induce cell death selectively in one or more cells affected by a cell proliferative disorder. In one aspect, administering to a subject in need thereof a compound of the invention, or a pharmaceutically acceptable salt, prodrug, metabolite, polymorph or solvate thereof, induces cell death selectively in one or more cells affected by a cell proliferative disorder.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18^{th} edition (1990). These texts can, of course, also be referred to in making or using an aspect of the invention.

“Combination therapy” (or “co-therapy”) includes the administration of a compound of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the invention.

“Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical.

“Combination therapy” also embraces the administration of the therapeutic agent(s) as described above in combination with other biologically active ingredients and/or non-drug therapies (e.g., surgery, immunotherapy or radiation treatment). Where the combination therapy comprises a non-drug treatment, the non-drug treatment may be conducted at any suitable time so long as a beneficial effect from the co-action of the combination of the therapeutic agent(s) and non-drug treatment is achieved. For example, in appropriate cases, the beneficial effect is still achieved when the non-drug treatment is temporally removed from the administration of the therapeutic agents, perhaps by days or even weeks.

A compound of the invention, or a pharmaceutically acceptable salt, prodrug, metabolite, analog or derivative thereof, may be administered in combination with a second anti-cancer agent. The second anti-cancer agent (also referred to as an anti-neoplastic agent or anti-proliferative agent) can be another agent that modulates cancer metabolism, an alkylating agent; an antibiotic; an anti-metabolite; a detoxifying agent; an interferon; a polyclonal or monoclonal antibody; an EGFR inhibitor; a HER₂ inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analogue drug or any chemotherapeutic, anti-neoplastic or anti-proliferative agent listed in www.cancer.org/docroot/cdg/cdg_{—}0.asp.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or vairubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine¹³¹ tositumomab (Bexxar); alemtuzumab (Campath); ibritumomab (Zevalin; In-111; Y-90 Zevalin); gemtuzumab (Mylotarg); eculizumab (Soliris) ordenosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (CI-1033); matuzumab (Emd7200) or EKB-569.

Exemplary HER2 inhibitors include, but are not limited to, trastuzumab (Herceptin); lapatinib (Tykerb) or AC-480.

Histone Deacetylase Inhibitors include, but are not limited to, vorinostat (Zolinza).

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot); exemestane (Aromasin); goserelin (Zoladex); bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BIBW 2992; E7080; Zd6474; PKC-412; motesanib; or AP24534.

Exemplary serine/threonine kinase inhibitors include, but are not limited to, ruboxistaurin; eril/easudil hydrochloride; flavopiridol; seliciclib (CYC202; Roscovitrine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WH1-P154; WH1-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxol.

Exemplary general chemotherapeutic, anti-neoplastic, anti-proliferative agents include, but are not limited to, altretamine (Hexylen); isotretinoin (Accutane; Amnesteem; Claravis; Sotret); tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur—0.4 M 5-chloro-2,4-dihydroxypyrimidine—1 M potassium oxonate) or lovastatin.

In another aspect, the second chemotherapeutic agent can be a cytokine such as G-CSF (granulocyte colony stimulating factor). In another aspect, a compound of the invention, or a pharmaceutically acceptable salt, prodrug, metabolite, analog or derivative thereof, may be administered in combination with radiation therapy. Radiation therapy can also be administered in combination with a compound of the invention and another chemotherapeutic agent described herein as part of a multiple agent therapy. In yet another aspect, a compound of the invention, or a pharmaceutically acceptable salt, prodrug, metabolite, analog or derivative thereof, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar™) or CMFP (cyclophosphamide, methotrexate, 5-fluorouracil and prednisone).

In one embodiment, a compound of the invention, or a pharmaceutically acceptable salt, prodrug, metabolite, polymorph or solvate thereof, may be administered with an inhibitor of an enzyme, such as a receptor or non-receptor kinase. Receptor and non-receptor kinases of the invention are, for example, tyrosine kinases or serine/threonine kinases. Kinase inhibitors of the invention are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevic (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1 and Erb2/Her2), GW-572016/lapatinib ditosylate (targets HER₂/Erb2), Panitumumab/Vectibix (targets EGFR), Vandetinib (targets RET/VEGFR), E7080 (multiple targets including RET and VEGFR), Herceptin (targets HER₂/Erb2), PKI-166 (targets EGFR), Canertinib/CI-1033 (targets EGFR), Sunitinib/SU-11464/Sutent (targets EGFR and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER₃), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azdl 152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

Throughout the description, where compositions are described as having, including, or comprising specific components, it is contemplated that compositions also consist essentially of, or consist of, the recited components. Similarly, where processes are described as having, including, or comprising specific process steps, the processes also consist essentially of, or consist of, the recited processing steps. Further, it should be understood that the order of steps or order for performing certain actions are immaterial so long as the invention remains operable. Moreover, two or more steps or actions may be conducted simultaneously.

The compounds, or pharmaceutically acceptable salts thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Techniques for formulation and administration of compounds of the invention can be found in Remington: the Science and Practice of Pharmacy, 19^{th} edition, Mack Publishing Co., Easton, Pa. (1995). In one aspect, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

In some embodiments, the compound is prepared for oral administration, wherein the disclosed compounds or salts thereof are combined with a suitable solid or liquid carrier or diluent to form capsules, tablets, pills, powders, syrups, solutions, suspensions and the like.

The tablets, pills, capsules, and the like contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose, saccharin, xylitol, and the like. When a dosage unit form is a capsule, it often contains, in addition to materials of the above type, a liquid carrier such as a fatty oil.

In some embodiments, various other materials are present as coatings or to modify the physical form of the dosage unit. For instance, in some embodiments, tablets are coated with shellac, sugar or both. In some embodiments, a syrup or elixir contains, in addition to the active ingredient, sucrose as a sweetening agent, methyl and propylparabens as preservatives, a dye and a flavoring such as cherry or orange flavor, and the like.

For some embodiments relating to parental administration, the compounds, or salts, solvates, tautomers or polymorphs thereof, can be combined with sterile aqueous or organic media to form injectable solutions or suspensions. In some embodiments, injectable compositions are aqueous isotonic solutions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, in another embodiment, the compositions contain about 1 to 50%, of the active ingredient.

For example, injectable solutions are produced using solvents such as sesame or peanut oil or aqueous propylene glycol, as well as aqueous solutions of water-soluble pharmaceutically-acceptable salts of the compounds. In some embodiments, dispersions are prepared in glycerol, liquid polyethylene glycols and mixtures thereof in oils. Under ordinary conditions of storage and use, these preparations contain a preservative to prevent the growth of microorganisms. The terms “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

For rectal administration, suitable pharmaceutical compositions are, for example, topical preparations, suppositories or enemas. Suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.1 to 75%, in another embodiment, compositions contain about 1 to 50%, of the active ingredient.

In some embodiments, the compounds are formulated to deliver the active agent by pulmonary administration, e.g., administration of an aerosol formulation containing the active agent from, for example, a manual pump spray, nebulizer or pressurized metered-dose inhaler. In some embodiments, suitable formulations of this type also include other agents, such as antistatic agents, to maintain the disclosed compounds as effective aerosols.

A drug delivery device for delivering aerosols comprises a suitable aerosol canister with a metering valve containing a pharmaceutical aerosol formulation as described and an actuator housing adapted to hold the canister and allow for drug delivery. The canister in the drug delivery device has a headspace representing greater than about 15% of the total volume of the canister. Often, the polymer intended for pulmonary administration is dissolved, suspended or emulsified in a mixture of a solvent, surfactant and propellant. The mixture is maintained under pressure in a canister that has been sealed with a metering valve.

For nasal administration, either a solid or a liquid carrier can be used. The solid carrier includes a coarse powder having particle size in the range of, for example, from about 20 to about 500 microns and such formulation is administered by rapid inhalation through the nasal passages. In some embodiments where the liquid carrier is used, the formulation is administered as a nasal spray or drops and includes oil or aqueous solutions of the active ingredients.

Also contemplated are formulations that are rapidly dispersing dosage forms, also known as “flash dose” forms. In particular, some embodiments of the invention are formulated as compositions that release their active ingredients within a short period of time, e.g., typically less than about five minutes, in another embodiment, less than about ninety seconds, in another embodiment, less than about thirty seconds and in another embodiment, in less than about ten or fifteen seconds. Such formulations are suitable for administration to a subject via a variety of routes, for example by insertion into a body cavity or application to a moist body surface or open wound.

Typically, a “flash dosage” is a solid dosage form that is administered orally, which rapidly disperses in the mouth, and hence does not require great effort in swallowing and allows the compound to be rapidly ingested or absorbed through the oral mucosal membranes. In some embodiments, suitable rapidly dispersing dosage forms are also used in other applications, including the treatment of wounds and other bodily insults and diseased states in which release of the medicament by externally supplied moisture is not possible.

“Flash dose” forms are known in the art; see for example, effervescent dosage forms and quick release coatings of insoluble microparticles in U.S. Pat. Nos. 5,578,322 and 5,607,697; freeze dried foams and liquids in U.S. Pat. Nos. 4,642,903 and 5,631,023; melt spinning of dosage forms in U.S. Pat. Nos. 4,855,326, 5,380,473 and 5,518,730; solid, free-form fabrication in U.S. Pat. No. 6,471,992; saccharide-based carrier matrix and a liquid binder in U.S. Pat. Nos. 5,587,172, 5,616,344, 6,277,406, and 5,622,719; and other forms known to the art.

The compounds of the invention are also formulated as “pulsed release” formulations, in which the compound is released from the pharmaceutical compositions in a series of releases (i.e., pulses). The compounds are also formulated as “sustained release” formulations in which the compound is continuously released from the pharmaceutical composition over a prolonged period.

Also contemplated are formulations, e.g., liquid formulations, including cyclic or acyclic encapsulating or solvating agents, e.g., cyclodextrins, polyethers, or polysaccharides (e.g., methylcellulose), or in another embodiment, polyanionic β-cyclodextrin derivatives with a sodium sulfonate salt group separate from the lipophilic cavity by an alkyl ether spacer group or polysaccharides. In some embodiments, the agent is methylcellulose. In another embodiment, the agent is a polyanionic β-cyclodextrin derivative with a sodium sulfonate salt separated from the lipophilic cavity by a butyl ether spacer group, e.g., CAPTISOL® (CyDex Pharmaceuticals Inc., Lenexa, Kans.). One skilled in the art can evaluate suitable agent/disclosed compound formulation ratios by preparing a solution of the agent in water, e.g., a 40% by weight solution; preparing serial dilutions, e.g. to make solutions of 20%, 10, 5%, 2.5%, 0% (control), and the like; adding an excess (compared to the amount that can be solubilized by the agent) of the disclosed compound; mixing under appropriate conditions, e.g., heating, agitation, sonication, and the like; centrifuging or filtering the resulting mixtures to obtain clear solutions; and analyzing the solutions for concentration of the disclosed compound.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

The following examples are illustrative of certain embodiments of the inventions and should not be considered to limit the scope of the invention. The reagents used can be either commercially obtained or can be prepared by standard procedures described in the literature. It is intended that the scope of this invention will cover all isomers (enantiomeric and diastereomeric) and all mixtures, including but not limited to racemic mixtures. The isomeric forms of the compounds of this invention may be separated or resolved using methods known to those skilled in the art or by synthetic methods that are stereospecific or asymmetric.

### General Methods:

All air or moisture sensitive reactions were performed under positive pressure of nitrogen with oven-dried glassware. Anhydrous solvents such as pyridine, tetrahydrofuran (THF), acetonitrile, ethanol and methanol were obtained from Bio-Lab. Hydrazine hydrate was purchased from Sigma-Aldrich and used as received. Sulfuric acid, acetic anhydride and ammonium hydroxide were purchased from Bio-Lab. Analytical HPLC experiments were carried out using Gemini-NX 3 μM C18 column (110 Å, 150×4.6 mm) on a Waters 2996 instrument equipped with a photodiode array detector. The mobile phase consisted of acetonitrile and water (each containing 0.1% trifluoroacetic acid). A gradient of 0% to 100% acetonitrile over 11 minutes was used during the purification. Fraction collection was triggered by UV detection (254 nM). Analytical analysis was performed on a Waters 2695 LC/MS (separation module). The mobile phase consisted of acetonitrile and water (each containing 0.1% formic acid). A Gemini-NX 3 μM C18 column (110 Å, 150×4.6 mm) was used. Purity determination was performed using a Waters 2996 instrument equipped with a photodiode array detector.

Preparative purification was performed with CombiFlash R_{f}, flash chromatography system. The column used was a Silica Redi sep R_{f}, flash column. Column chromatography was performed using silica gel 60 (particle size 0.04-0.06 mm) Preparative chiral separation was performed on SFC-80 (Thar, Waters) using Chiralpak AD-H column (Daicel, 5 μm, 20×250 mm) at column temperature: 40° C. The mobile phase consisted of methanol/CO₂=45/55 at flow 40 g/min and back pressure 100 Bar. ¹H NMR (300 MHz) spectra were recorded on Bruker Avance-300 spectrometer, in DMSO-d6 as an internal standard. All of the analogues for assay have purity greater than 95%.

### Example 1

### Synthesis of 2-pyrazoline derivatives

One aspect of embodiments of the invention is stereoselective preparation of 2-pyrazolines of the following general structure [4]:

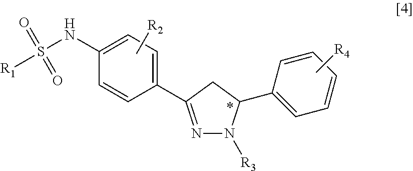

Pyrazolines are well known, nitrogen-containing 5-membered heterocyclic compounds and various methods have been worked out for their synthesis. 2-Pyrazolines are well studied pyrazoline type compounds. As a result, a number of 2-pyrazolines using different synthetic methods for their preparation have been described in the literature. A popular procedure is based on the reaction of α,β-unsaturated aldehydes and ketones with hydrazines (Bilgin, A. A. et al. (1994) Pharmazie 49, 67-69; Raiford, L. C. et al. (1937) J. Org. Chem. 1, 544-551; Raiford, L. C. et al. (1938) J. Org. Chem. 3, 265-272; Raiford, L. C. et al. (1940) J. Org. Chem. 5, 590-597; Wiley, R. H. et al. (1958) J. Org. Chem. 23, 732-738; Kabli, R. A. et al. (1991) J. Indian Chem. Soc. 68, 47-51; Mishriky, N. et al. (1996) Pharmazie 51, 544-548; Manna, F. et al. (2002) Bioorg. Med. Chem. Lett. 12, 3629-3633; Levai, A. et al. (2002) J. Heterocyclic Chem. 39, 751-758; Levai, A. (2003) Heterocycl. Commun. 9, 287-292; Chimenti, F. et al. (2004) J. Med. Chem. 47, 2071-2074; Chimenti, F. et al. (2005) Bioorg. Med. Chem. Lett. 15, 603-607; Levai, A. et al. (2005) J. Heterocyclic Chem. 42, 1231-1235; Levai, A. et al. (200) J. Heterocyclic Chem. 39, 1-13; Levai, A. et al. (2006) J. Heterocyclic Chem. 43, 111-115)

Referring to Scheme 1, the reaction of chalcones [1] and related α,β-unsaturated ketones with hydrazines [2] is the most popular procedure for the synthesis of 2-pyrazolines [5]. This reaction can be conducted under various conditions. The most commonly used method is the reaction of compounds [1] and [2] in acetic acid solution to prepare 2-pyrazoline [5].

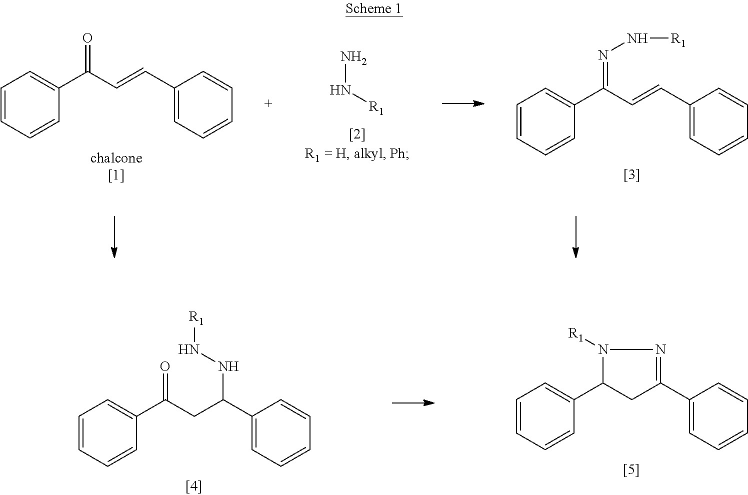

This method is used either with or without the isolation of the hydrazone intermediate [3]. Alternatively, the reaction of α,β-unsaturated ketones [1] and hydra zines [2] under acid-catalyzed conditions using hydrazine hydrochloride in hot alcoholic or dimethylformamide solution (Mishriky, N. et al. (1996) Pharmazie 51, 544; Joshi, M. G. et al. (1981) Indian J. Chem. 20B, 1090; Basaif, S. A. et al. (1995) Indian J. Heterocyclic Chem. 5, 121)

Synthesis of 2-pyrazolines [5] can also be achieved under alkaline conditions by using pyridine as catalyst in ethanolic solution (Anjaneyulu, A. S. R. et al. (1995) Indian J. Chem. 34B, 933.) or as solvent (Sammour, A. E. (1964) Tetrahedron 20, 1067.). In some cases, the two reactants were refluxed in alcoholic solution without catalyst to provide 2-pyrazolines [5]. Based on various experimental findings, it has been concluded that the reaction of α,β-enones [1] with hydrazines [2] yields 2-pyrazolines [5] via hydrazone intermediates [3] under acidic conditions. However, if piperidine was used as the catalyst, the β-hydrazinoketones [4] were formed as intermediates, the ring closure of which gives 2-pyrazolines [5] (Al-Farkh, Y. A. et al. (1979) Chem. Pharm. Bull. 27, 257).

As shown in Scheme 2, compounds according to certain embodiments of the invention were prepared as follows. In the first step, substituted p-aminoacetophenone was condensed with chlorosulphonylchloride derivative to give sulphonamidoacetophenone [6], which was then treated with arylaldehydes [7] to give 4-(substituted sulphoneamido) chalcones [8]. The obtained product, α,β-unsaturated ketones [8], was treated with substituted hydrazine hydrate [9] in ethanol and in glacial acetic acid to yield the expected 2-pyrazoline product [10].

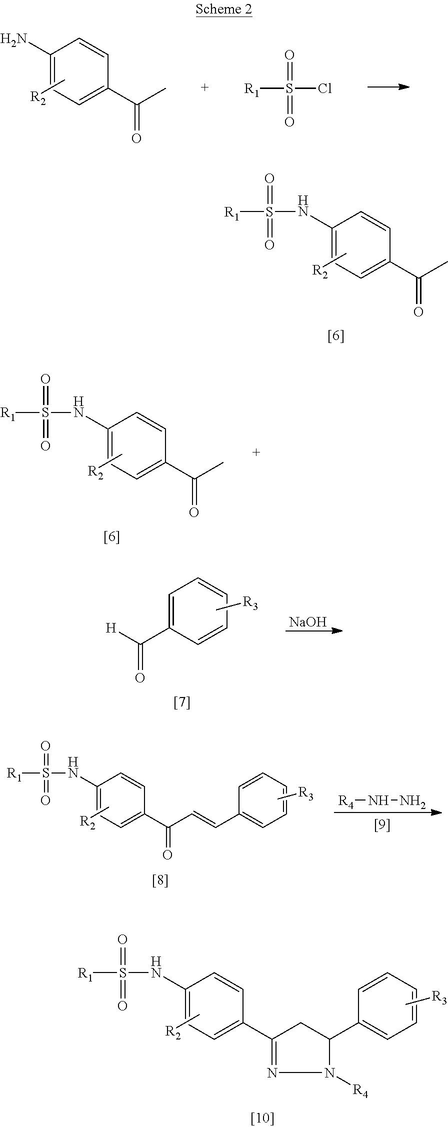

These compounds of the invention include the individual optical isomers having an asymmetric carbon at the 5-position of the 2-pyrazoline ring.

The enantiomers were resolved by known methods such as preparative HPLC using a 10 cm CHIRALPAK® AD column (US 20090042966), or deliberately prepared by enantioselective synthesis of 2-pyrazoline (Angewandte Chemie International Edition (Dec. 21, 2009), 48(52), 9975-9978). Both enantiomers were tested for their PKM2 activity.

In 2007, Kanemasa and Yanagita (Yanagita et al. (2007) Heterocycles 71, 699-709) reported an enantioselective conjugate addition cyclocondensation cascade between different aryl hydrazines and 3-phenyl-1-(2-pyridyl)-1-propanone, catalyzed by a chiral nickel complex, affording optically enriched pyrazolines in moderate enantioselectivities.

Benjamin List (Angewandte Chemie International Edition (Dec. 21, 2009), 48(52), 9975-9978) reported the catalytic asymmetric preparation of 2-pyrazolines using 3,3′-bis-(9-anthracenyl) substituted binol phosphate in high yield and high e.e. According to List's procedure the reactions were run in Ar atmosphere with enones, phenylhydrazines MS 4A and 10% mol of phosphoric acid as catalyst in chlorobenzene at 30° C.

### Example 2

### Synthesis of N-(4-(1-acetyl-5-(2,3-dihydrobenzo[b][1,4]dioxin-6-yl)-4,5-dihydro-1H-pyrazol-3-yl)phenyl)ethanesulfonamide (Compound 62A)

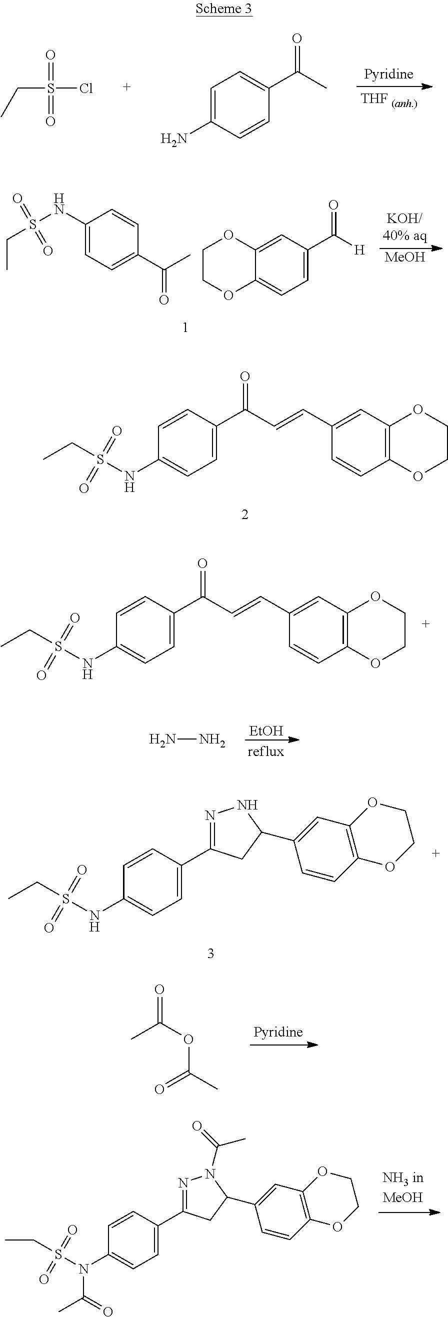

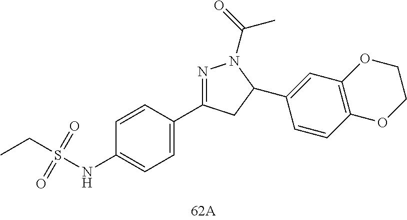

### Reagents

| | MW | | | | |
|---|---|---|---|---|---|
| Reagent/raw material | (g/mole) | Quantity | | moles | |
| ethanesulfonyl chloride | 128.58 | 3.4 | g | 26 | mmol |
| 1-(4-aminophenyl)ethanone | 135.16 | 3 | g | 22 | mmol |
| N-(4-acetylphenyl)ethanesulfonamide | 227.28 | 2 | g | 8.8 | mmol |
| 2,3-dihydrobenzo[b][1,4]dioxine-6-carbaldehyde | 164.16 | 1.44 | g | 8.8 | mmol |
| (E)-N-(4-(3-(2,3-dihydrobenzo[b][1,4]dioxin-6- | 373.42 | 0.614 | g | 1.64 | mmol |
| yl)acryloyl)phenyl)ethanesulfonamide | | | | | |
| Hydrazine | 50 | excess | | | |
| N-(4-(5-(2,3-dihydrobenzo[b][1,4]dioxin-6-yl)-4,5- | 387.45 | 0.63 | g | 1.64 | mmol |
| dihydro-1H-pyrazol-3-yl)phenyl)ethanesulfonamide | | | | | |

| | | |
|---|---|---|
| acetic anhydride | 102.09 | excess |

### Step 1:

Ethanesulfonyl chloride was added dropwise to a stirred solution of 1-(4-aminophenyl)ethanone and 1 mL of pyridine. The reaction was stirred at room temperature overnight. When complete as determined by HPLC, using the H₂O-Acetonitrile (ACN) gradient given below with a Gemini®-NX C18, μm, 110 Å, 150×4.6 mm chromatography column, as well as by LCMS, the THF was evaporated. The evaporation residue was dissolved in 50 mL ethyl acetate, washed with 100 mL of 1M KHSO₄, and then brine and dried on Na₂SO₄. The solvent was evaporated and the product used for the next step without further purification (3.57 g, yield 60.4%)

### HPLC Purification Protocol

| Time (min) | Flow | H2O (%) | ACN (%) |
|---|---|---|---|
| | 1 | 100 | 0 |
| 0.02 | 1 | 100 | 0 |
| 11.0 | 1 | 0 | 100 |
| 13.0 | 1 | 100 | 0 |
| 18.0 | 1 | 100 | 0 |
| 18.1 | 0.2 | 100 | 0 |

### Step 2:

N-(4-acetylphenyl)ethanesulfonamide and 2,3-dihydrobenzo[b][1,4]dioxine-6-carbaldehyde were refluxed in MeOH with 40% KOH/H₂O for 3 hours, and then stirred at room temperature overnight. The reaction was evaporated, redissolved in EtOAc and then washed with 1N KHSO₄ and finally with brine. The organic phase was dried, and crystallized from EtOH. The chalcone product absorbs strongly at 360 nm and eluted at 8/2 EtOAc/PE (1.2 g, 36.4% yield).

### Step 3:

(E)-N-(4-(3-(2,3-dihydrobenzo[b][1,4]dioxin-6-yl)acryloyl)phenyl)ethanesulfonamide was stirred in EtOH (20 mL) with a large excess of hydrazine at reflux for 3 hours. Reaction mixture was evaporated and used further step without purification.

### Step 4:

N-(4-(5-(2,3-dihydrobenzo[b][1,4]dioxin-6-yl)-4,5-dihydro-1H-pyrazol-3-yl)phenyl)ethanesulfonamide was stirred for 3 hours in a 1/1 mixture of acetic anhydride and pyridine at room temperature. The reaction mixture was evaporated to dryness, and was stirred into an ammonium hydroxide in MeOH solution at room temperature for 3 hours. After all the starting material was consumed, the crude reaction mixture was evaporated, and separated on CombiFlash® (EA:PE) to provide 116 mg of product (16.4% yield). HPLC: Rt=8.11 minutes, 95.5% purity, as determined by the protocol above. MS−(ES+) Calcd. for C17H17NO6S2 429.49. found 430.51 (M+H).

### Example 3

### Synthesis of N-(4-(1-acetyl-5-(4-methoxyphenyl)-4,5-dihydro-1H-pyrazol-3-yl)phenyl)-2,6-difluorobenzenesulfonamide (Compound 61A)

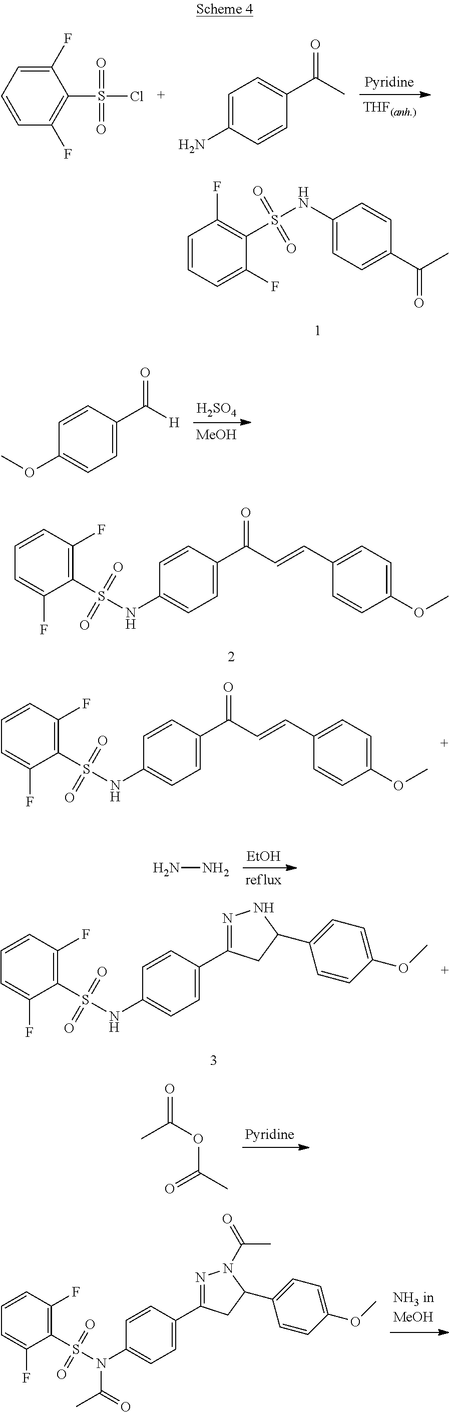

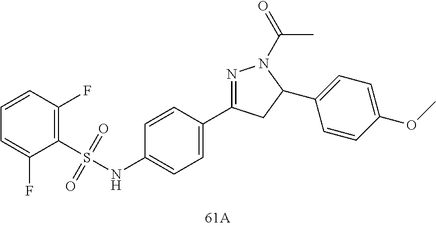

### Reagents

| | MW | | | | |
|---|---|---|---|---|---|
| Reagent/raw material | (g/mole) | Quantity | | moles | |
| 2,6-difluorobenzene-1-sulfonyl chloride | 212.60 | 2.54 | g | 12 | mmol |
| 1-(4-aminophenyl)ethanone | 135.16 | 1.35 | g | 10 | mmol |
| N-(4-acetylphenyl)-2,6-difluorobenzenesulfonamide | 311.30 | 2.4 | g | 7.7 | mmol |
| 4-methoxybenzaldehyde | 136.15 | 1.04 | g | 7.7 | mmol |
| (E)-2,6-difluoro-N-(4-(3-(4- | 429.44 | 0.292 | g | 0.68 | mmol |
| methoxyphenyl)acryloyl)phenyl)benzenesulfonamide | | | | | |
| Hydrazine | 50 | excess | | | |
| 2,6-difluoro-N-(4-(5-(4-methoxyphenyl)-4,5-dihydro- | 443.47 | 0.221 | g | 0.5 | mmol |
| 1H-pyrazol-3-yl)phenyl)benzenesulfonamide | | | | | |

| | | |
|---|---|---|
| acetic anhydride | 102.09 | excess |

### Step 1:

2,6-difluorobenzene-1-sulfonyl chloride was added dropwise to a stirred solution of 1-(4-aminophenyl)ethanone and 1 mL of pyridine. The reaction was stirred at room temperature overnight. When complete as determined by HPLC using the protocol described above, as well as by LCMS, THF was evaporated, the evaporation residue was dissolved in 50 mL ethyl acetate, washed with 100 mL of 1M KHSO₄, and then washed with brine and dried on Na2SO4. The solvent was evaporated and the residue was used for the next step without further purification (2.4 g, yield 46%).

### Step 2:

N-(4-acetylphenyl)-2,6-difluorobenzenesulfonamide and 4-methoxybenzaldehyde were refluxed in MeOH with 40% KOH/H₂O for 3 hours, and then stirred at room temperature overnight. The reaction mixture was evaporated, redissolved in EtOAc and washed with 1N KHSO4 and then brine. The organic phase was dried, evaporated and separated on CombiFlash®. The chalcone product absorbs strongly at 360 nm, eluting at 8/2 EtOAc/PE (292 mg, 8.7% yield).

### Step 3:

(E)-2,6-difluoro-N-(4-(3-(4-methoxyphenyl)acryloyl)phenyl)benzenesulfonamide was stirred in EtOH (20 mL) with a large excess of hydrazine at reflux for 3 hours. The reaction mixture was evaporated and used in the next step without further purification.

### Step 4:

2,6-difluoro-N-(4-(5-(4-methoxyphenyl)-4,5-dihydro-1H-pyrazol-3-yl)phenyl)benzenesulfonamide was stirred for 3 hours in a 1:1 mixture of acetic anhydride and pyridine, at room temperature. The reaction mixture was evaporated to dryness, and the product, N-(4-(1-acetyl-5-(4-methoxyphenyl)-4,5-dihydro-1H-pyrazol-3-yl)phenyl)-N-((2,6-difluorophenyl)sulfonyl)acetamide, was stirred in ammonium hydroxide in a MeOH solution at room temperature for 3 hours. After all the starting material was consumed, the crude reaction mixture was evaporated, and separated on CombiFlash® (EA:PE) to 6 mg of the final product (7% yield). Product Analysis: HPLC: Rt=9.085 minutes, 93.49% purity, as determined by the protocol in Example 2 above. MS−(ES+) Calcd. for C17H17NO6S2 485.5. found 486.52 (M+H).

### Example 4

### Synthesis of N-(4-(1-acetyl-5-(4-methoxyphenyl)-4,5-dihydro-1H-pyrazol-3-yl)phenyl)-4-fluorobenzenesulfonamide (Compound 6A)

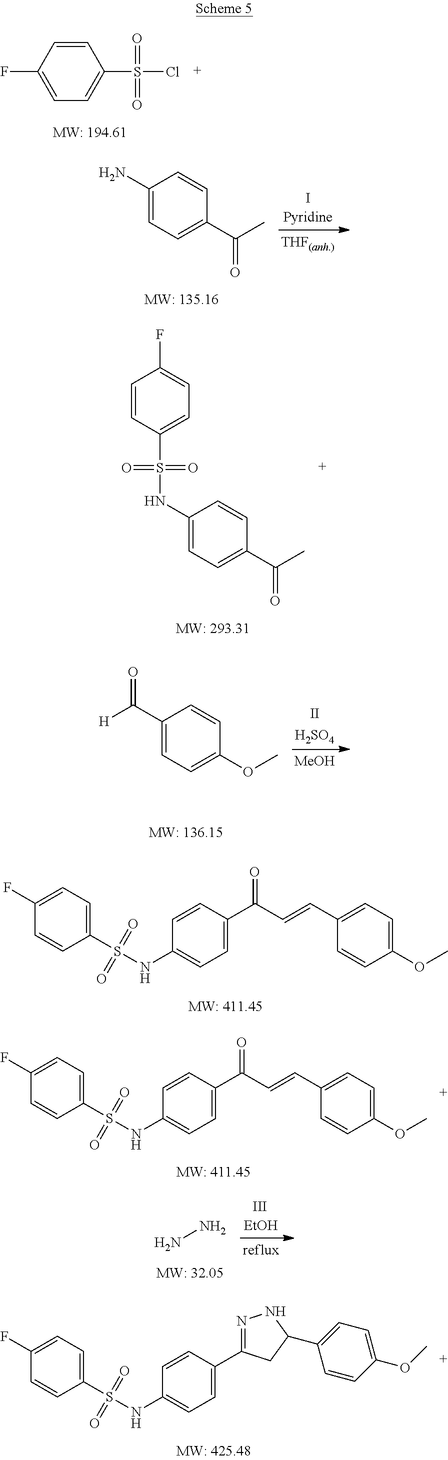

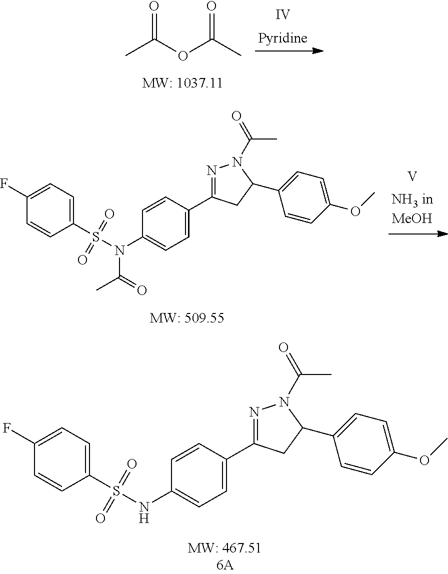

### Reagents

| | MW | | | Mole |
|---|---|---|---|---|
| Reagent/raw material | (g/mole) | Quantity | moles | ratio |
| 4-fluorobenzene-1-sulfonyl chloride | 194.61 | 1500 mg | 7.77 mmol | 1.05 eq. |
| 1-(4-aminophenyl)ethanone | 135.16 | 1000 mg | 7.40 mmol | 1.0 eq. |
| N-(4-acetylphenyl)-4-fluorobenzenesulfonamide | 293.31 | 1250 mg | 4.261 mmol | 1.0 eq. |
| 4-methoxybenzaldehyde | 136.15 | 610 mg | 4.47 mmol | 1.05 eq. |
| (E)-4-fluoro-N-(4-(3-(4- | 411.45 | 1000 mg | 2.43 mmol | 1.0 eq. |
| methoxyphenyl)acryloyl)phenyl)benzenesulfonamide | | | | |
| 4-fluoro-N-(4-(5-(4-methoxyphenyl)-4,5-dihydro-1H- | 425.48 | Approx. | | |
| pyrazol-3-yl)phenyl)benzenesulfonamide | | | | |
| acetic anhydride | 102.09 | Approx. | | |
| N-(4-(1-acetyl-5-(4-methoxyphenyl)-4,5-dihydro-1H- | 509.55 | Approx. | | |
| pyrazol-3-yl)phenyl)-N-((4- | | | | |
| fluorophenyl)sulfonyl)acetamide | | | | |
| N-(4-(1-acetyl-5-(4-methoxyphenyl)-4,5-dihydro-1H- | 467.51 | Approx. | | |
| pyrazol-3-yl)phenyl)-4-fluorobenzenesulfonamide | | | | |

A large-scale synthesis of compound 6A is described in Experiment 8a.

### Step 1:

4-fluorobenzene-1-sulfonyl chloride was added dropwise to a stirred solution of 1-(4-aminophenyl)ethanone and 1 mL of pyridine under N₂. The reaction was stirred at room temperature overnight. When complete as determined by HPLC using the protocol described in Example 2, as well as by TLC (8/2 PE/EtOAc), the THF was evaporated, the evaporation residue was dissolved in 50 mL ethyl acetate, washed with 100 mL cold 0.5M HCl and then with brine and finally dried on Na₂SO₄. The solvent was evaporated and the product was used in the next step without further purification (1250 mg, yield 58%).

### Step II:

N-(4-acetylphenyl)-4-fluorobenzenesulfonamide and 4-methoxybenzaldehyde were refluxed in MeOH with sulfuric acid (1 mL) for 3 hours, and then stirred at room temperature overnight. The reaction was evaporated, redissolved in EtOAc and then washed twice with 50 mL 0.5M NaOH and finally with brine. The organic phase was dried, evaporated and separated on CombiFlash®. The chalcone product absorbs strongly at 360 nm and eluted at 8/2 EtOAc/PE. Step II was repeated twice, until enough product accumulated (1000 mg, ˜80% pure, overall yield 20%).

### Step III:

(E)-4-fluoro-N-(4-(3-(4-methoxyphenyl)acryloyl)phenyl)benzenesulfonamide was stirred in EtOH (20 mL) with a large excess of hydrazine (70% aqueous solution) at room temperature for 3 hours. The reaction was evaporated and separated on CombiFlash®. Product-containing fractions were combined and used without further purification.

### Step IV:

4-fluoro-N-(4-(5-(4-methoxyphenyl)-4,5-dihydro-1H-pyrazol-3-yl)phenyl)benzenesulfonamide was stirred for 3 hours in 1:1 mixture of acetic anhydride and pyridine at room temperature. The reaction mixture was evaporated to dryness, and crudely separated on CombiFlash®. No complete purification was achieved as determined by MS and HPLC.

### Step V:

N-(4-(1-acetyl-5-(4-methoxyphenyl)-4,5-dihydro-1H-pyrazol-3-yl)phenyl)-N-((4-fluorophenyl)sulfonyl)acetamide, (approximately 500 mg, ˜80% pure) was stirred in ammonium in MeOH solution at room temperature for 3 hours. After all the starting material was consumed, the crude reaction mixture was evaporated, and separated twice on CombiFlash®. 140 mg of 100% pure final product was obtained (overall yield 7%). ¹H-NMR 300 MHz, (CDCl₃) δ 2.40 (CH3, s), 3.08 (1H, dd), 3.66 (1H, dd), 3.74 (3H, s), 5.56 (1H, m), 6.82 (2H, d), 7.12 (2H, d), 7.12 (2H, d), 7.13 (2H, d), 7.58 (2H, d), 7.81 (2H, d). HPLC: Rt=9.050 minutes, 100% purity, as determined by the protocol of Example 2 above. MS: (ES+) Calcd. for C24H22FN3O4S 467.51. found 468.51 (M+H).

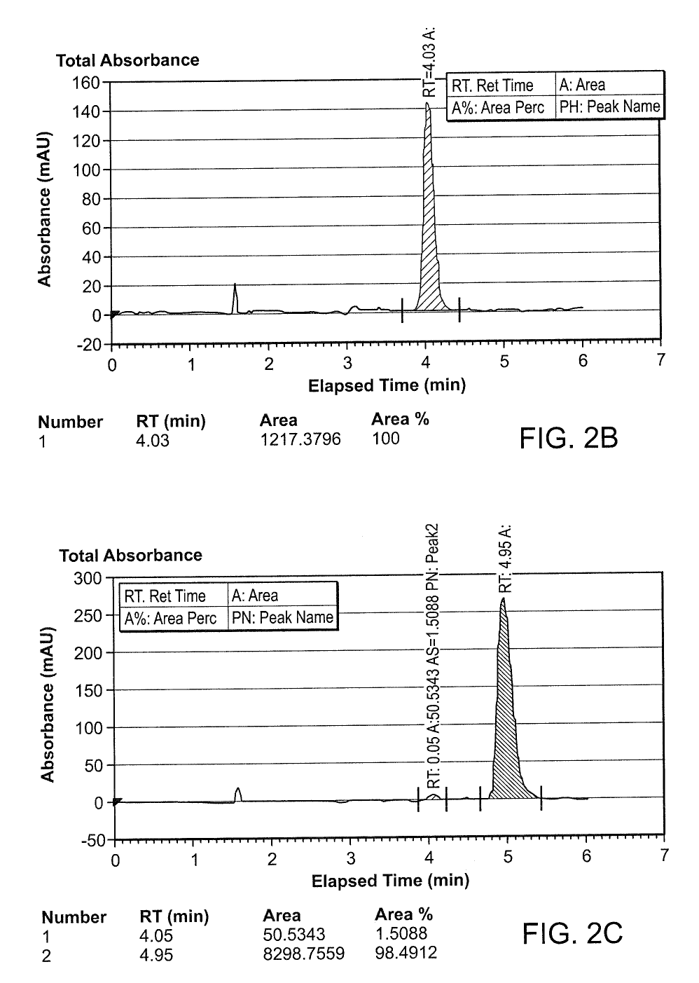

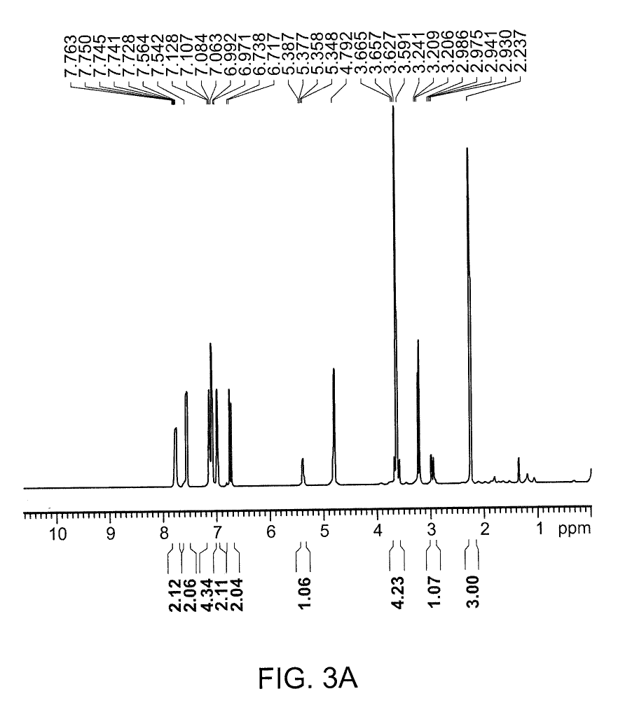

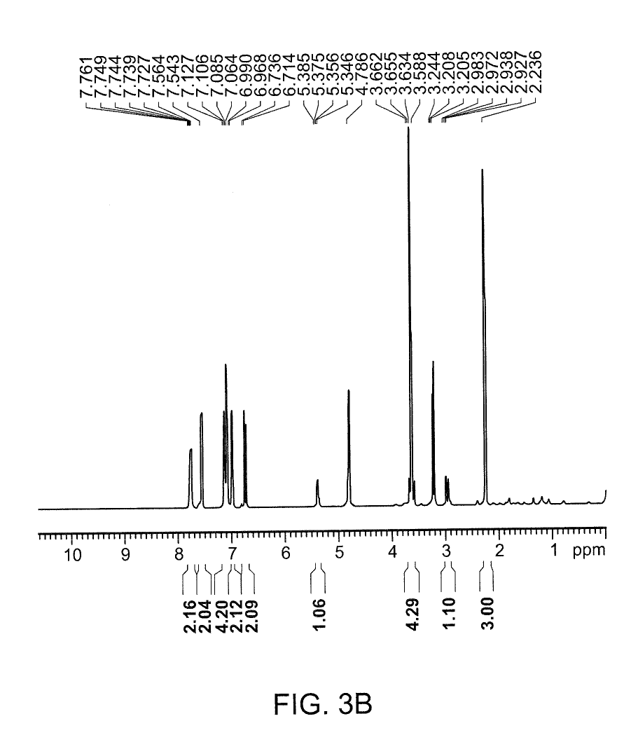

72 mg of racemic N-(4-(1-acetyl-5-(4-methoxyphenyl)-4,5-dihydro-1H-pyrazol-3-yl)phenyl)-4-fluorobenzenesulfonamide (Compound 6A) was resolved into its two individual enantiomers by preparative HPLC using a CHIRALPAK® AD-H (Daicel) 250×20 mm, 5 μm column run at 40° C. Chromatography was performed using a mobile phase of 45/55Methanol/CO2 at a flow rate of 40 g/min. and 10 g of the compound was loaded per injection. FIG. 2 shows HPLC chromatograms of the starting racemate (FIG. 2A) and each of the individual enantiomers (FIGS. 2B and 2C). A ¹H-NMR analysis for each of the individual enantiomers is shown in FIG. 3A (R-enantiomer) and FIG. 3B (S-enantiomer), respectively.

### Example 5

### Synthesis of N-(4-(1-acetyl-5-(4-methoxyphenyl)-4,5-dihydro-1H-pyrazol-3-yl)phenyl)-2,3-dihydrobenzo[b][1,4]dioxine-6-sulfonamide (Compound 7A)

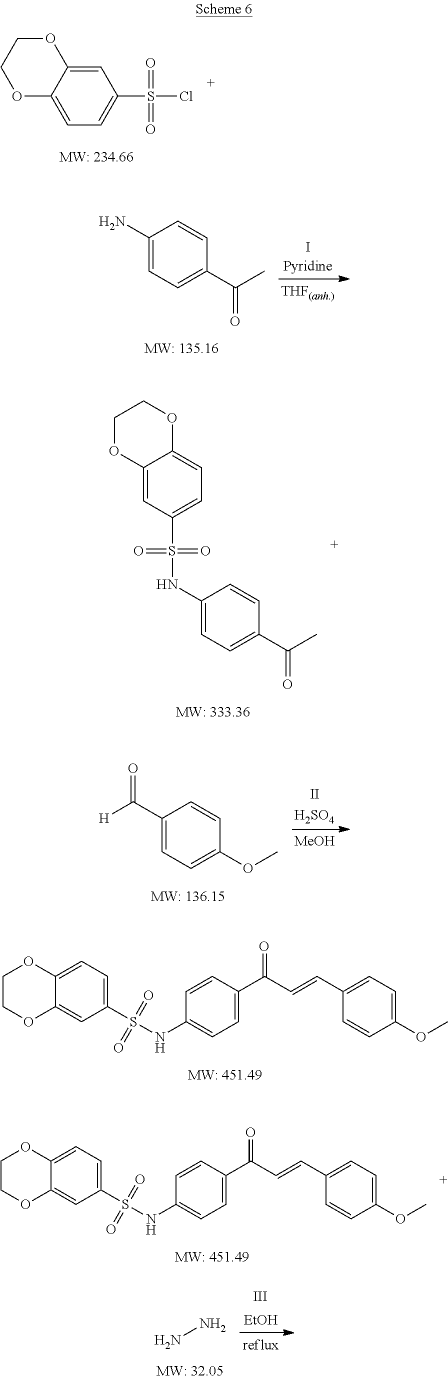

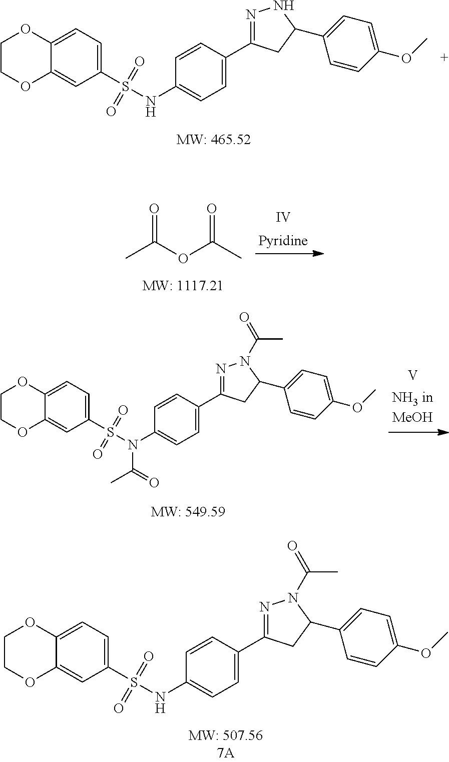

### Reagents

| | MW | | | Mole |
|---|---|---|---|---|
| Reagent/raw material | (gr/mole) | Quantity | moles | ratio |
| 2,3-dihydrobenzo[b][1,4]dioxine-6-sulfonyl chloride | 234.66 | 500 mg | 2.13 mmol | 1.00 eq. |
| 1-(4-aminophenyl)ethanone | 135.16 | 302 mg | 2.24 mmol | 1.05 eq. |
| N-(4-acetylphenyl)-2,3- | 333.36 | 605 mg | 1.82 mmol | 1.00 eq. |
| dihydrobenzo[b][1,4]dioxine-6-sulfonamide | | | | |
| 4-methoxybenzaldehyde | 136.15 | 300 mg | 2.18 mmol | 1.20 eq. |
| (E)-N-(4-(3-(4-methoxyphenyl)acryloyl)phenyl)-2,3- | 451.49 | 600 mg | 2.43 mmol | 1.0 eq. |
| dihydrobenzo[b][1,4]dioxine-6-sulfonamide | | | | |
| N-(4-(5-(4-methoxyphenyl)-4,5-dihydro-1H-pyrazol- | 465.52 | Approx. | | |
| 3-yl)phenyl)-2,3-dihydrobenzo[b][1,4]dioxine-6- | | | | |
| sulfonamide | | | | |
| acetic anhydride | 102.09 | Approx. | | |
| N-(4-(1-acetyl-5-(4-methoxyphenyl)-4,5-dihydro- | 549.59 | Approx. | | |
| 1H-pyrazol-3-yl)phenyl)-N-((2,3- | | | | |
| dihydrobenzo[b][1,4]dioxin-6-yl)sulfonyl)acetamide | | | | |
| N-(4-(1-acetyl-5-(4-methoxyphenyl)-4,5-dihydro- | 507.56 | Approx. | | |
| 1H-pyrazol-3-yl)phenyl)-2,3 - | | | | |
| dihydrobenzo[b][1,4]dioxine-6-sulfonamide | | | | |

### Step I:

2,3-dihydrobenzo[b][1,4]dioxine-6-sulfonyl chloride was added dropwise to a stirred solution of 1-(4-aminophenyl)ethanone and 1 mL of pyridine under N₂. The reaction was stirred at room temperature overnight. When complete as determined by HPLC using the protocol described in Example 2 above, as well as by TLC (8/2 PE/EtOAc), the THF was evaporated, the evaporation residue was dissolved in 50 mL ethyl acetate, washed with 100 mL cold 0.5M HCl and then with brine and finally dried on Na₂SO₄. The solvent was evaporated and the product was used in the next step without further purification (605 mg, yield 80%).

### Step II:

4-methoxybenzaldehyde and N-(4-acetylphenyl)-2,3-dihydrobenzo[b][1,4]dioxine-6-sulfonamide were refluxed in MeOH with sulfuric acid (1 mL) for 3 hours, and then stirred at room temperature overnight. The reaction was evaporated, redissolved in EtOAc and then washed twice with 50 mL 0.5M NaOH and finally with brine. The organic phase was dried, evaporated and separated on CombiFlash®. The chalcone product absorbs strongly at 360 nm and eluted at 8/2 EtOAc/PE (˜600 mg, ˜70% pure, overall yield 20%).

### Step III:

(E)-N-(4-(3-(4-methoxyphenyl)acryloyl)phenyl)-2,3-dihydrobenzo[b][1,4]dioxine-6-sulfonamide was stirred in EtOH (20 mL) with a large excess of hydrazine (70% aqueous solution) at room temperature for 3 hours. The reaction was evaporated and separated on CombiFlash®. Product-containing fractions were combined and used further without complete purification.

### Step IV:

N-(4-(5-(4-methoxyphenyl)-4,5-dihydro-1H-pyrazol-3-yl)phenyl)-2,3-dihydrobenzo[b][1,4]dioxine-6-sulfonamide was stirred for 3 hours in a 1:1 mixture of acetic anhydride and pyridine at room temperature. The reaction mixture was evaporated to dryness, and crudely separated on CombiFlash®. No complete purification was achieved as determined by MS and HPLC.

### Step V:

N-(4-(1-acetyl-5-(4-methoxyphenyl)-4,5-dihydro-1H-pyrazol-3-yl)phenyl)-N-((2,3-dihydrobenzo[b][1,4]dioxin-6-yl)sulfonyl)acetamide, (approximately 600 mg, ˜70% pure) was stirred in ammonium in MeOH solution at room temperature for 3 hours. After all the starting material was consumed, the crude reaction mixture was evaporated, and separated twice on CombiFlash® in petrol ether/ethyl acetate. 121 mg of 100% pure final product was obtained (overall yield 8%). ¹H-NMR 300 MHz, (CDCl3) δ 2.25 (CH3, s), 3.05 (1H, dd), 3.70 (3H, s), 3.78 (1H, dd), 4.28 (4H, m), 5.45 (1H, m), 6.87 (2H, d), 7.02 (1H, d), 7.08 (2H, d), 7.20 (2H, d), 7.26 (1H, d), 7.29 (1H, d), 7.67 (2H, d), 10.52 (NH, br. s); HPLC: Rt=9.002 minutes, 100% pure, as determined by the protocol of Example 2 above.

MS: (ES+) Calcd. for C26H25FN3O6S 507.56. found 508.44 (M+H).

### Example 6

### Synthesis of 1-(5-(2,3-dihydrobenzo[b][1,4]dioxin-6-yl)-3-(1-(methylsulfonyl)indolin-5-yl)-4,5-dihydro-1H-pyrazol-1-yl)ethanone (Compound 9A)

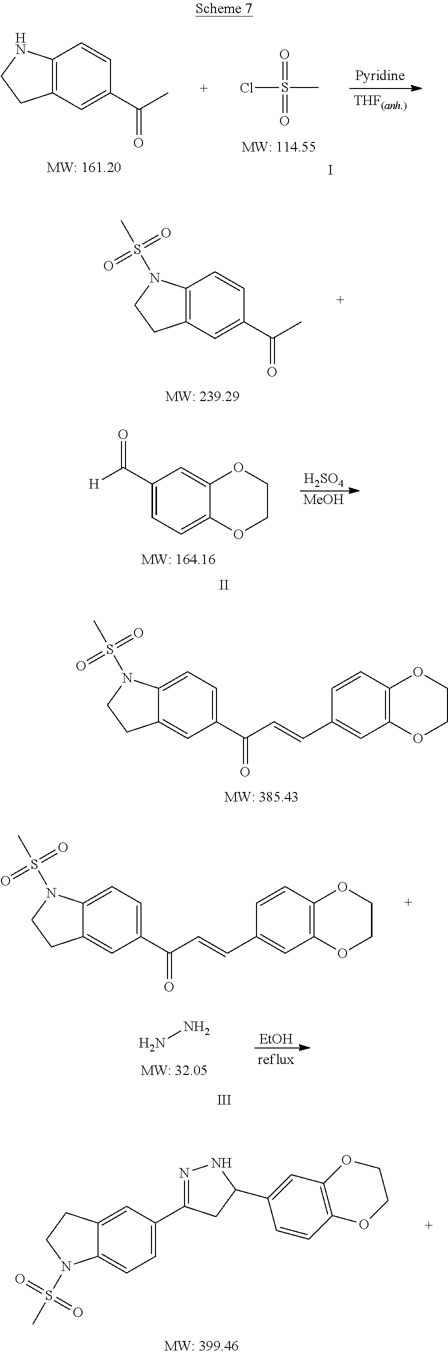

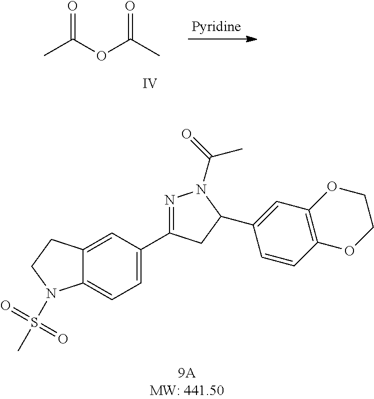

### Reagents

| | MW | | | Mole |
|---|---|---|---|---|
| Reagent/raw material | (g/mole) | Quantity | moles | ratio |
| 1-(indolin-5-yl)ethanone | 161.20 | 700 mg | 4.34 mmol | 1.0 eq. |
| methanesulfonyl chloride | 114.55 | 600 mg | 5.20 mmol | 1.2 eq. |
| 1-(1-(methylsulfonyl)indolin-5-yl)ethanone | 239.29 | 400 mg | 1.67 mmol | 1.0 eq. |
| 2,3-dihydrobenzo[b][1,4]dioxine-6-carbaldehyde | 164.16 | 280 mg | 1.67 mmol | 1.0 eq. |
| (E)-3-(2,3-dihydrobenzo[b][1,4]dioxin-6-yl)-1-(1- | 385.43 | 400 mg | 1.04 mmol | 1.0 eq. |
| (methylsulfonyl)indolin-5-yl)prop-2-en-1-one | | | | |
| 5-(5-(2,3-dihydrobenzo[b][1,4]dioxin-6-yl)-4,5- | 399.46 | 400 mg. | 1.00 mmol | 1.00 |
| dihydro-1H-pyrazol-3-yl)-1- | | | | |
| (methylsulfonyl)indoline | | | | |
| acetic anhydride | 102.09 | Approx. | excess | excess |
| | | 5 mL | | |
| 1-(5-(2,3-dihydrobenzo[b][1,4]dioxin-6-yl)-3-(1- | 413.49 | | | |
| (methylsulfonyl)indolin-5-yl)-4,5-dihydro-1H- | | | | |
| pyrazol-1-yl)ethanone | | | | |

### Step I:

Methanesulfonyl chloride was added dropwise to a stirred solution of 1-(indolin-5-yl)ethanone and 5 mL of pyridine under N₂. The reaction was stirred at room temperature overnight. When complete as determined by HPLC using the protocol described in Example 2 above, as well as by TLC (8/2 PE/EtOAc), the solvent was evaporated, the evaporation residue was dissolved in 50 mL ethyl acetate, washed with 100 mL cold 0.5M HCl and then with brine and finally dried on Na₂SO₄. The solvent was evaporated and the product was used in the next step without further purification (400 mg, yield 38%).

### Step II:

1-(1-(methylsulfonyl)indolin-5-yl)ethanone and 2,3-dihydrobenzo[b][1,4]dioxine-6-carbaldehyde were refluxed in MeOH with sulfuric acid (1 mL) for 3 hours, and then stirred at room temperature overnight. The reaction was evaporated, redissolved in EtOAc and then washed twice with 50 mL 0.5M NaOH and then brine. The organic phase was dried, evaporated and separated on CombiFlash® (yield 60%).

### Step III:

(E)-3-(2,3-dihydrobenzo[b][1,4]dioxin-6-yl)-1-(1-(methylsulfonyl)indolin-5-yl)prop-2-en-1-one was stirred in EtOH (20 mL) with a large excess of hydrazine (70% aqueous solution) at room temperature for 3 hours. The reaction was evaporated and separated on CombiFlash®. Product-containing fractions were combined and used without further purification.

### Step IV:

5-(5-(2,3-dihydrobenzo[b][1,4]dioxin-6-yl)-4,5-dihydro-1H-pyrazol-3-yl)-1-(methylsulfonyl)indoline was stirred for 3 hours in a 1:1 mixture of acetic anhydride and pyridine at room temperature. The reaction mixture was evaporated to dryness, and separated on CombiFlash® twice. No complete purification was achieved as determined by MS and HPLC. 92 mg of 100% pure final product was obtained (overall yield 6%). ¹H-NMR 300 MHz, (CDCl3) δ 2.27 (CH3, s), 3.16 (2H, t), 3.76 (1H, m), 3.98 (2H, t), 4.18 (3H, s), 4.19 (4H, s), 5.40 (1H, dd), 5.44 (1H, dd), 6.63 (1H, m), 6.64 (1H, m), 6.80 (1H, d), 7.31 (1H, d), 7.60 (1H, d), 7.72 (2H, d). HPLC: Rt=8.683 minutes, 100% pure, as determined by the protocol in Example 2 above. MS: (ES+) Calcd. for C22H23N3O5S 441.50. found 442.62 (M+H).

### Example 7

### Synthesis of 4-(1-acetyl-5-(4-methoxyphenyl)-4,5-dihydro-1H-pyrazol-3-yl)-N-ethylbenzenesulfonamide (Compound 10A)

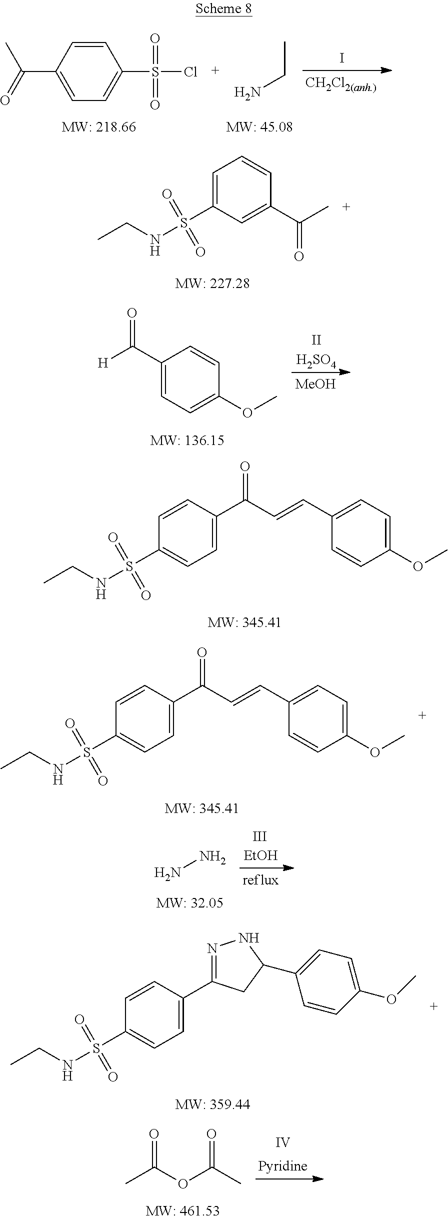

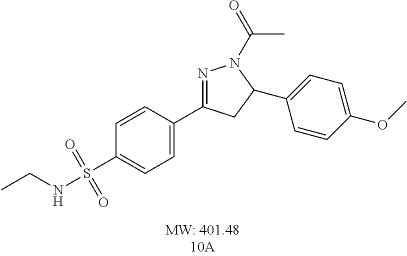

### Reagents

| | MW | | | Mole |
|---|---|---|---|---|
| Reagent/raw material | (gr/mole) | Quantity | moles | ratio |
| 4-acetylbenzene-1-sulfonyl chloride | 218.66 | 514 mg | 2.35 mmol | 1.00 eq. |
| Ethanamine | 45.08 | 302 mg | 2.82 mmol | 1.20 eq. |
| 3-acetyl-N-ethylbenzenesulfonamide | 227.28 | 300 mg | 1.32 mmol | 1.00 eq. |
| 4-methoxybenzaldehyde | 136.15 | 270 mg | 2.00 mmol | 1.50 eq. |
| (E)-N-ethyl-4-(3-(4- | 345.41 | 284 mg | 1.21 mmol | 1.0 eq. |
| methoxyphenyl)acryloyl)benzenesulfonamide | | | | |
| N-(4-(5-(4-methoxyphenyl)-4,5-dihydro-1H- | 359.44 | Approx. | | |
| pyrazol-3-yl)phenyl)-2,3- | | 300 mg | | |
| dihydrobenzo[b][1,4]dioxine-6-sulfonamide | | | | |
| acetic anhydride | 102.09 | excess | Excess | |
| 4-(1-acetyl-5-(4-methoxyphenyl)-4,5-dihydro- | 549.59 | | | |
| 1H-pyrazol-3-yl)-N-ethylbenzenesulfonamide | | | | |

### Step I:

4-acetylbenzene-1-sulfonyl chloride was added dropwise to a stirred solution of excess ethanamine and 1 mL of pyridine under N2. The reaction was stirred at room temperature overnight. When complete as determined by HPLC using the protocol described in Example 2 above, as well as by TLC (8/2 PE/EtOAc), the pyridine was evaporated, the evaporation residue was dissolved in 50 mL ethyl acetate, and then washed with 100 mL cold 0.5M HCl and then with brine and finally dried on Na2SO4. The solvent was evaporated and the product was used for the next step without further purification (500 mg, yield 91%, HPLC Rt=7.529 min).

### Step II:

3-acetyl-N-ethylbenzenesulfonamide and 4-methoxybenzaldehyde were refluxed in MeOH with sulfuric acid (1 mL) for 3 hours, and then stirred at room temperature overnight. The reaction was evaporated, redissolved in EtOAc and then washed twice with 50 mL 0.5M NaOH and then with brine. The organic phase was dried, evaporated and separated on CombiFlash®. The chalcone product absorbs strongly at 360 nm and eluted at 8/2 EtOAc/PE (˜300 mg isolated, 98% pure, Rt=9.025 minutes).

### Step III:

(E)-N-ethyl-4-(3-(4-methoxyphenyl)acryloyl)benzenesulfonamide was stirred in EtOH (20 mL) with a large excess of hydrazine (70% aqueous solution) at room temperature for 3 hours. The reaction was evaporated and separated on CombiFlash®. Product-containing fractions were combined and used without further purification.

### Step IV:

N-(4-(5-(4-methoxyphenyl)-4,5-dihydro-1H-pyrazol-3-yl)phenyl)-2,3-dihydrobenzo[b][1,4]dioxine-6-sulfonamide was stirred for 3 hours in a 1:1 mixture of acetic anhydride and pyridine at room temperature. The reaction mixture was evaporated to dryness, and crudely separated on CombiFlash®. The resulting product was identified as the desired monoacylation product by MS and HPLC. 122 mg of 96% pure final product was obtained (overall yield 5%). ¹H-NMR 300 MHz, (CDCl₃) δ 1.11 (CH3, t), 2.42 (CH3, s), 3.04 (2H, q), 3.19 (1H, dd), 3.75 (1H, dd), 3.77 (3H, s), 4.59 (NH, t), 5.61 (1H, m), 6.85 (2H, d), 7.17 (2H, d), 7.87 (2H, d), 7.93 (2H, d); HPLC: Rt=8.247 minutes, 96.9% pure, as determined by the protocol in Example 2 above. MS−(ES+) Calcd. for C20H23FN3O4S 401.48. found 402.56 (M+H).

### Example 8

### Synthesis of substituted 3-(4-substitutedsulphonamidophenyl)-5-aryl-2-pyrazolines activators of the tumor cell specific M2 isoform of pyruvate kinase

The preparation of compounds of the invention was based on known procedures (Patel, P. et al. (1996) IL FARMACO 51(1), 59-63 and Jamode, V. S. et al. (2003) Indian J. Heterocyclic Chem. 12, 323-326).

### Example 8-1

### Compound 6A

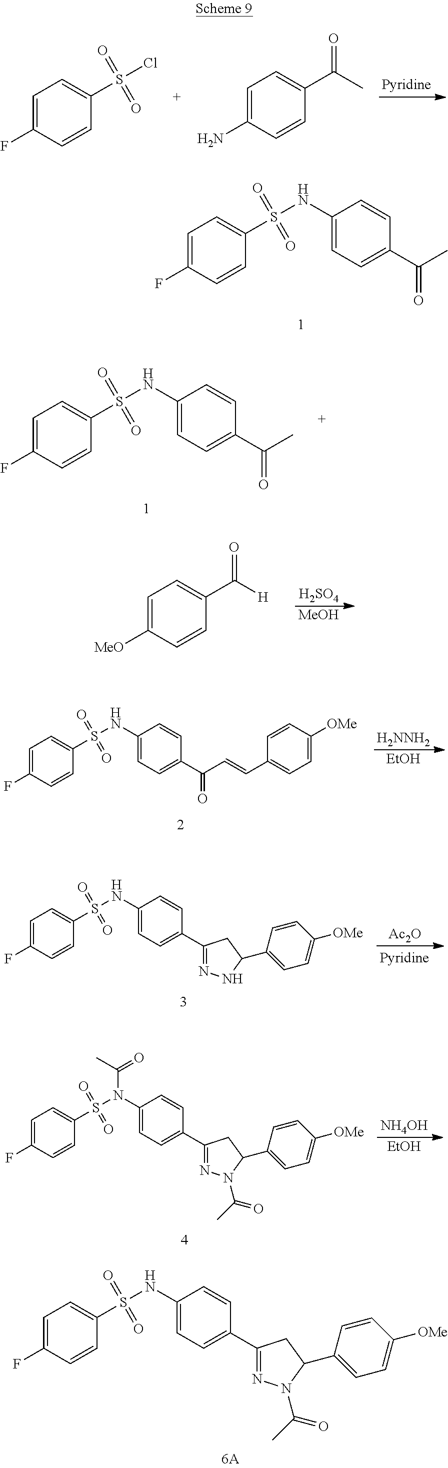

### N-(4-Acetylphenyl)-4-fluorobenzenesulfonamide (1)

4-Fluorobenzene-1-sulfonyl chloride (20.0 g, 103.0 mmol, 1.1 equiv) was added dropwise to a stirred solution of 4′-Aminoacetophenone (12.6 g, 93.6 mmol, 1.0 equiv) in pyridine (500 mL). The reaction was stirred overnight at rt. Monitoring by HPLC and LCMS indicated that the reaction was completed. The solvent was evaporated under reduced pressure. A solution of HCl 1N (200 mL) and EtOAc (200 mL) were added. The organic layer was separated and the aqueous phase was extracted twice with EtOAc (2×200 mL). The combined organic phase was dried over Na₂SO₄ and the solvent was evaporated under reduced pressure to yield the pure product: 26.2 gr, 95.5% yield.

### (E)-4-Fluoro-N-(4-(3-(4-methoxyphenyl)acryloyl)phenyl)benzenesulfonamide (2)

N-(4-Acetylphenyl)-4-fluorobenzenesulfonamide (26.2 g, 89.3 mmol, 1.0 equiv) and 4-Methoxybenzaldehyde (10.8 mL, 89.3 mmol, 1.0 equiv) were refluxed overnight in MeOH (500 mL) with sulfuric acid (2 ml). After cooling the reaction, the precipitate formed was filtered to obtain the pure product as an off white solid: 26.2 gr, 71.3% yield.

### 4-Fluoro-N-(4-(5-(4-methoxyphenyl)-4,5-dihydro-1H-pyrazol-3-yl)phenyl)benzene sulfonamide (3)

(E)-4-fluoro-N-(4-(3-(4-methoxyphenyl)acryloyl)phenyl)benzene sulfonamide (26.2 g, 63.6 mmol, 1.0 equiv) and pyridine (5 ml) were stirred in EtOH (200 mL) with large excess of hydrazine (10 mL) at reflux for 3 hours. The solvent was evaporated and the mixture was transferred to the next step without further treatment: 22.9 gr, 85% yield.

### N-(4-(1-Acetyl-5-(4-methoxyphenyl)-4,5-dihydro-1H-pyrazol-3-yl)phenyl)-N-((4-fluorophenyl)sulfonyl)acetamide (4)

4-fluoro-N-(4-(5-(4-methoxyphenyl)-4,5-dihydro-1H-pyrazol-3-yl)phenyl)benzenesulfonamide (22.9 g, 53.8 mmol, 1.0 equiv) was dissolved in pyridine (50 mL). Acetic anhydride (20 mL) was added and the reaction mixture was stirred for 2 h at rt. Monitoring by HPLC and LCMS indicated that the reaction was completed. The solvent was evaporated and the diacetylation product was transferred to the next step without further treatment: 24.0 gr, 87.5% yield.

### N-(4-(1-Acetyl-5-(4-methoxyphenyl)-4,5-dihydro-1H-pyrazol-3-yl)phenyl)-4-fluoro benzenesulfonamide (6A)

N-(4-(1-acetyl-5-(4-methoxyphenyl)-4,5-dihydro-1H-pyrazol-3-yl)phenyl)-N-((4-fluorophenyl)sulfonyl)acetamide (24.0 g, 47.1 mmol, 1.0 equiv) was stirred with ammonium hydroxide (15 mL) in EtOH (200 mL) solution at room temperature for 2 h. Monitoring by HPLC and LCMS indicated that the reaction was completed. The solvent was evaporated under reduced pressure. H₂O (200 mL) and EtOAc (200 mL) were added. The organic layer was separated and the aqueous phase was extracted twice with EtOAc. The combined organic phase was dried over Na₂SO₄ and the solvent was evaporated under reduced pressure. Recrystallization from EtOH afforded the pure product as a white solid: 11.4 gr, 51.8% yield. ¹H NMR (300 MHz, DMSO) δ 10.65 (brs, 1H, NH), 7.88 (d, 1H, J=8.7 Hz), 7.83 (d, 1H, J=8.7 Hz), 7.63 (d, 2H, J=8.7 Hz), 7.40 (t, 2H, J=8.7 Hz), 7.19 (d, 2H, J 8.7 Hz), 7.05 (d, 2H, J=8.7 Hz), 6.86 (d, 2H, J=8.4 Hz), 5.43 (dd, 1H, J=11.4 Hz, J=4.2 Hz), 3.74 (dd, 1H, J=18.0 Hz, J=11.7 Hz), 3.70 (s, 3H), 2.99 (dd, 1H, J=18.0 Hz, J=4.2 Hz), 2.24 (s, 3H); LC/MS: retention time: 7.531 min; m/z (M+H⁺)=468.70 (Calculated for C₂₄H₂₂FN₃O₄S=467.51); HPLC: 98% purity, retention time: 8.590 min.

### Example 8-2

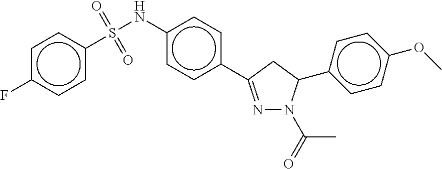

### Compound 12A: Enantiomer A of Compound 6A.

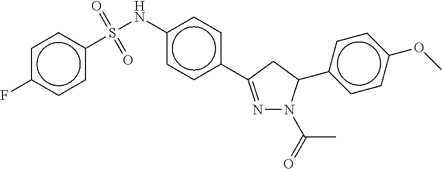

### Compound 13A: Enantiomer B of Compound 6A.

### HPLC Method for the Above Synthetic Steps:

### Column: Gemini-NX 3 μm C₁₈ 110 Å, 150×4.6 mm

| Time (min) | Flow | H₂O (%) | ACN (%) |
|---|---|---|---|
| | 1 | 100 | 0 |
| 0.02 | 1 | 100 | 0 |
| 11.0 | 1 | 0 | 100 |
| 13.0 | 1 | 100 | 0 |
| 18.0 | 1 | 100 | 0 |
| 18.1 | 0.2 | 100 | 0 |

The racemic compound Compound 6A was separated to its individual enantiomers using SFC separation technique:

### Analytical—SFC Conditions

### Instrument: SFC Method Station (Thar, Waters)

### Column: CHIRALPAK AD-H 4.6*250 mm 5 um (Daicel)

Column temperature: 40° C.
Mobile phase: Methanol/CO2=50/50
Flow: 3.0 mL/min

### Back Pressure: 150 Bar

Injection volume: 3 uL

### Preparative-SFC Conditions

### Instrument: SFC-80 (Thar, Waters)

Column: CHIRALCEL AD-H 50*250 mm 5 um (Daicel); column temperature: 40° C.
Mobile phase: Methanol/CO2=50/50
Flow: 80 g/min
Back pressure: 100 Bar
Cycle time of stack injection: 10 min
Load per injection: 16 mg

### Representative Chromatograms

1. SFC analysis of compound Compound 6A.
2. Purity check of enantiomer-A Compound 12A and enantiomer-B Compound 13A after purification.
3. 1H-NMR analysis of Compound 13A (MeOD)

### Example 8-3

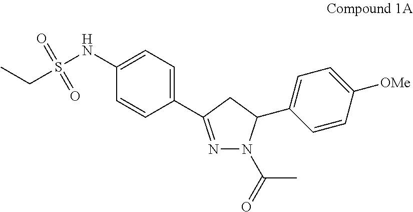

### N-(4-(1-Acetyl-5-(4-methoxyphenyl)-4,5-dihydro-1H-pyrazol-3-yl)phenyl)ethane sulfonamide (Compound 1A)

Analogue Compound 1A was prepared in the same procedure as analogue Compound 6A. ¹H NMR (300 MHz, DMSO) δ 10.11 (brs, 1H, NH), 7.72 (d, 2H, J=8.4 Hz), 7.29 (d, 2H, J=8.4 Hz), 7.08 (d, 2H, J=8.1 Hz), 6.88 (d, 2H, J=8.4 Hz), 5.46 (dd, 1H, J=12.0 Hz, J=4.5 Hz), 3.77 (dd, 1H, J=18.0 Hz, J=12.0 Hz), 3.71 (s, 3H), 3.13 (q, 2H, J=7.5 Hz), 3.03 (dd, 1H, J=18.0 Hz, J=4.5 Hz), 2.27 (s, 3H), 1.18 (t, 3H, J=7.2 Hz); LC/MS: retention time: 6.623 min; m/z (M+H⁺)=402.43 (Calculated for C₂₀H₂₃N₃O₄S=401.48); HPLC: 100% purity, retention time: 8.502 min.

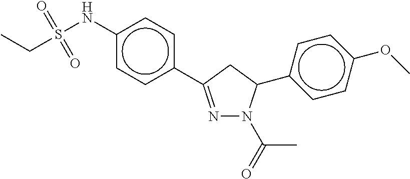

### Compound 2A: Enantiomer A of Compound 1A.

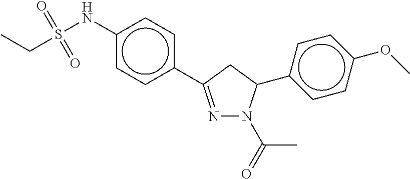

### Compound 3A: Enantiomer B of Compound 1A.

### Example 8-4

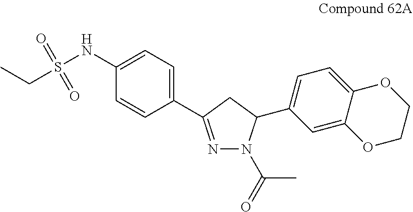

### N-(4-(1-Acetyl-5-(2,3-dihydrobenzo[b][1,4]dioxin-6-yl)-4,5-dihydro-1H-pyrazol-3-yl)phenyl)ethanesulfonamide (Compound 62A)

Analogue Compound 62A was prepared in the same procedure as analogue Compound 6A. ¹H NMR (300 MHz, DMSO) δ 10.11 (brs, 1H, NH), 7.71 (d, 2H, J=8.7 Hz), 7.29 (d, 2H, J=8.7 Hz), 6.77 (d, 1H, J=9.0 Hz), 6.60-6.64 (m, 2H), 5.40 (dd, 1H, J=11.7 Hz, J=4.2 Hz), 4.19 (s, 4H), 3.70 (dd, 1H, J=18.0 Hz, J=11.7 Hz), 3.13 (q, 2H, J=7.5 Hz), 3.04 (dd, 1H, J=18.0 Hz, J=4.2 Hz), 2.27 (s, 3H), 1.19 (t, 3H, J=7.2 Hz); LC/MS: retention time: 6.731 min; m/z (M+H⁺)=430.51 (Calculated for C₂₁H₂₃N₃O₅S=429.49); HPLC: 95.5% purity, retention time: 8.119 min.

### Example 8-5

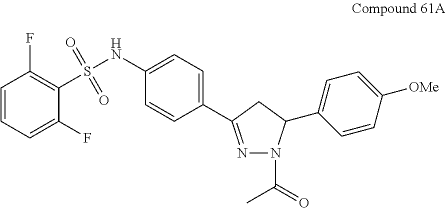

### N-(4-(1-Acetyl-5-(4-methoxyphenyl)-4,5-dihydro-1H-pyrazol-3-yl)phenyl)-2,6-difluorobenzenesulfonamide (Compound 61A)

Analogue Compound 61A was prepared in the same procedure as analogue Compound 6A. ¹H NMR (300 MHz, MeOD) δ 11.23 (brs, 1H, NH), 7.64-7.74 (m, 3H), 7.17-7.31 (m, 4H), 7.04 (d, 2H, J=8.7 Hz), 6.86 (d, 2H, J=8.7 Hz), 5.41 (dd, 1H, J=11.7 Hz, J=4.2 Hz), 3.70 (s, 3H), 3.68 (dd, 1H, J=17.7 Hz, J=11.7 Hz), 3.00 (dd, 1H, J=17.7 Hz, J=4.2 Hz), 2.24 (s, 3H); LC/MS: retention time: 7.195 min; m/z (M+H⁺)=486.52 (Calculated for C₂₄H₂₁F₂N₃O₄S=485.50); HPLC: 93.5% purity, retention time: 9.085 min.

### Example 8-6

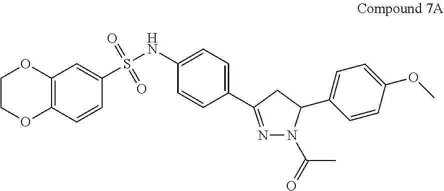

### N-(4-(1-Acetyl-5-(4-methoxyphenyl)-4,5-dihydro-1H-pyrazol-3-yl)phenyl)-2,3-dihydrobenzo[b][1,4]dioxine-6-sulfonamide (Compound 7A)

Analogue Compound 7A was prepared in the same procedure as analogue Compound 6A. ¹H NMR (300 MHz, DMSO) δ 10.52 (brs, 1H, NH), 7.63 (d, 2H, J=8.7 Hz), 7.28 (dd, 1H, J=8.7 Hz, J=2.1 Hz), 7.26 (s, 1H), 7.20 (d, 2H, J=8.7 Hz), 7.05 (d, 2H, J=8.4 Hz), 6.98 (dd, 1H, J=7.8 Hz, J=1.2 Hz), 6.86 (d, 2H, J=8.7 Hz), 5.43 (dd, 1H, J=11.4 Hz, J=4.2 Hz), 4.27-4.28 (m, 4H), 3.72 (dd, 1H, J=18.0 Hz, J=11.4 Hz), 3.70 (s, 3H), 3.00 (dd, 1H, J=18.0 Hz, J=4.5 Hz), 2.25 (s, 3H); LC/MS: retention time: 7.195 min; m/z (M+H⁺)=508.44 (Calculated for C₂₆H₂₅N₃O₆S=507.56); HPLC: 100% purity, retention time: 9.002 min.

### Example 8-7

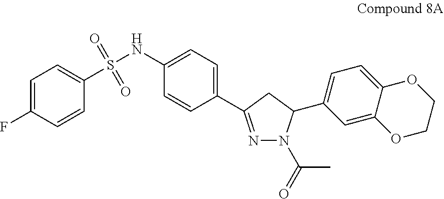

### N-(4-(1-Acetyl-5-(2,3-dihydrobenzo[b][1,4]dioxin-6-yl)-4,5-dihydro-1H-pyrazol-3-yl)phenyl)-4-fluorobenzenesulfonamide (Compound 8A)

Analogue Compound 8A was prepared in the same procedure as analogue Compound 6A. ¹H NMR (300 MHz, DMSO) δ 10.64 (brs, 1H, NH), 7.85 (d, 1H, J=9.0 Hz), 7.83 (d, 1H, J=9.0 Hz), 7.62 (d, 2H, J=8.7 Hz), 7.40 (t, 2H, J=8.7 Hz), 7.19 (d, 2H, J=8.7 Hz), 6.75 (d, 1H, J=8.7 Hz), 6.58-6.61 (m, 2H), 5.37 (dd, 1H, J=11.4 Hz, J=4.2 Hz), 4.19 (s, 4H), 3.69 (dd, 1H, J=18.0 Hz, J=11.7 Hz), 2.98 (dd, 1H, J=18.0 Hz, J=4.2 Hz), 2.25 (s, 3H); LC/MS: retention time: 7.427 min; m/z (M+H⁺)=496.52 (Calculated for C₂₅H₂₂FN₃O₅S=495.52); HPLC: 99% purity, retention time: 9.108 min.

### Example 8-8

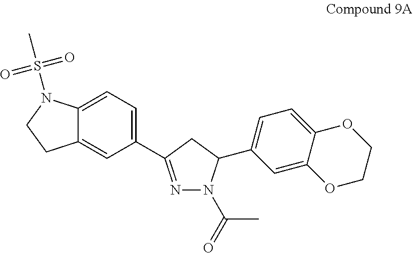

### 1-(5-(2,3-dihydrobenzo[b][1,4]dioxin-6-yl)-3-(1-(methylsulfonyl)indolin-5-yl)-4,5-dihydro-1H-pyrazol-1-yl)ethanone (Compound 9A)

Analogue Compound 9A was prepared in the same procedure as analogue Compound 6A. ¹H NMR (300 MHz, DMSO)S 7.72 (s, 1H), 7.57 (d, 1H, J=8.4 Hz), 7.31 (d, 1H, J=8.4 Hz), 6.77 (d, 1H, J=8.7 Hz), 6.62-6.63 (m, 2H), 5.40 (dd, 1H, J=11.7 Hz, J=4.2 Hz), 4.19 (s, 4H), 3.98 (t, 2H, J=8.4 Hz), 3.70 (dd, 1H, J=18.0 Hz, J=11.7 Hz), 3.16 (t, 2H, J=8.4 Hz), 3.09 (dd, 1H, J=18.0 Hz, J=4.2 Hz), 3.04 (s, 3H), 2.27 (s, 3H); LC/MS: retention time: 7.088 min; m/z (M+H⁺)=442.62 (Calculated for C₂₂H₂₃N₃O₅S=441.50); HPLC: 100% purity, retention time: 8.683 min.

### Example 8-9

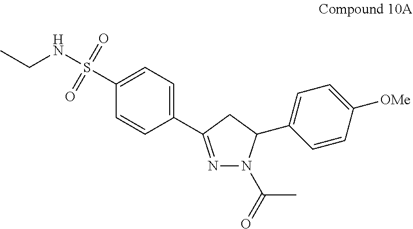

### 4-(1-Acetyl-5-(4-methoxyphenyl)-4,5-dihydro-1H-pyrazol-3-yl)-N-ethylbenzene sulfonamide (Compound 10A)

Analogue Compound 10A was prepared in the same procedure as analogue Compound 6A. ¹H NMR (300 MHz, CDCl₃) δ 7.90 (q, 4H, J=8.7 Hz), 7.14 (d, 2H, J=8.7 Hz), 6.86 (d, 2H, J=8.7 Hz), 5.58 (dd, 1H, J=12.0 Hz, J=4.8 Hz), 4.60 (t, 1H, J=6.0 Hz, NH), 3.77 (s, 3H), 3.70 (dd, 1H, J=17.7 Hz, J=12.0 Hz), 3.13 (dd, 1H, J=17.7 Hz, J=4.8 Hz), 3.04 (quin, 2H, J=7.2 Hz), 2.42 (s, 3H), 1.11 (t, 3H, J=7.2 Hz); LC/MS: retention time: 6.793 min; m/z (M+H⁺)=402.56 (Calculated for C₂₀H₂₃N₃O₄S=401.48); HPLC: 98% purity, retention time: 8.246 min.

### Example 8-10

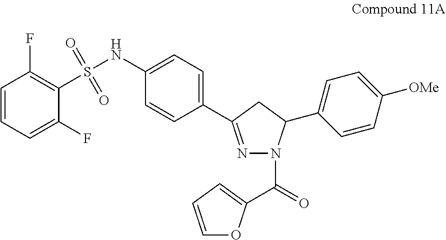

### 2,6-Difluoro-N-(4-(1-(furan-2-carbonyl)-5-(4-methoxyphenyl)-4,5-dihydro-1H-pyrazol-3-yl)phenyl)benzenesulfonamide (Compound 11A)

Analogue Compound 11A was prepared in the same procedure as analogue Compound 6A. ¹H NMR (300 MHz, MeOD) S 7.69-7.76 (m, 4H), 7.54-7.64 (m, 1H), 7.31 (d, 2H, J=8.7 Hz), 7.06-7.16 (m, 4H), 6.86 (d, 2H, J=8.7 Hz), 6.64-6.65 (m, 1H), 5.65-5.70 (m, 1H), 3.75 (dd, 1H, J=18.0 Hz, J=11.7 Hz), 3.73 (s, 3H), 3.11 (dd, 1H, J=18.0 Hz, J=4.5 Hz); LC/MS: retention time: 7.645 min; m/z (M+H⁺)=538.52 (Calculated for C₂₇H₂₁F₂N₃O₅S=537.53); HPLC: 100% purity, retention time: 9.132 min.

### Example 8-11

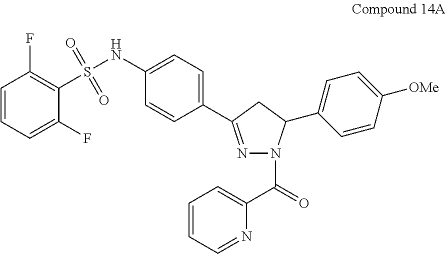

### 2,6-Difluoro-N-(4-(5-(4-methoxyphenyl)-1-picolinoyl-4,5-dihydro-1H-pyrazol-3-yl)phenyl)benzenesulfonamide (Compound 14A)

Analogue Compound 14A was prepared in the same procedure as analogue Compound 6A. ¹H NMR (300 MHz, DMSO) δ 8.62-8.63 (m, 1H), 7.91 (t, 1H, J=7.8 Hz), 7.67-7.73 (m, 1H), 7.61 (d, 1H, J=7.8 Hz), 7.49 (d, 3H, J=8.1 Hz), 7.22-7.29 (m, 4H), 7.16 (d, 2H, J=8.4 Hz), 6.93 (d, 2H, J=8.4 Hz), 5.66 (dd, 1H, J=11.4 Hz, J=4.5 Hz), 3.77 (dd, 1H, J=18.0 Hz, J=11.4 Hz), 3.73 (s, 3H), 3.08 (dd, 1H, J=18.0 Hz, J=4.5 Hz); LC/MS: retention time: 7.268 min; m/z (M+H⁺)=549.45 (Calculated for C₂₈H₂₂F₂N₄O₄S=548.56); HPLC: 97% purity, retention time: 7.858 min

### Example 8-12

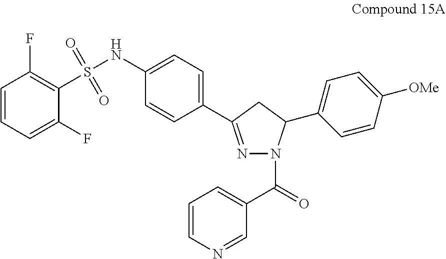

### 2,6-difluoro-N-(4-(5-(4-methoxyphenyl)-1-nicotinoyl-4,5-dihydro-1H-pyrazol-3-yl)phenyl)benzenesulfonamide (Compound 15A)

Analogue Compound 15A was prepared in the same procedure as analogue Compound 6A. LC/MS: retention time: 5.769 min; m/z (M+H⁺)=549.03 (Calculated for C₂₈H₂₂F₂N₄O₄S=548.56); HPLC: 94% purity, retention time: 7.571 min.

### Example 8-13

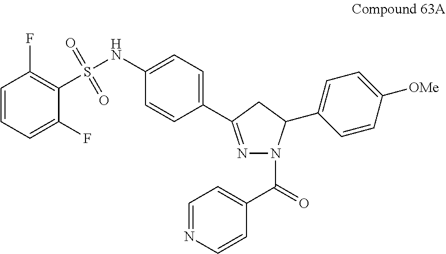

### 2,6-Difluoro-N-(4-(1-isonicotinoyl-5-(4-methoxyphenyl)-4,5-dihydro-1H-pyrazol-3-yl)phenyl)benzenesulfonamide (Compound 63A)

Analogue Compound 63A was prepared in the same procedure as analogue Compound 6A. ¹H NMR (300 MHz, DMSO) δ 11.30 (brs, 1H, NH), 8.70 (d, 2H, J=5.4 Hz), 7.67-7.73 (m, 3H), 7.62 (d, 2H, J=8.7 Hz), 7.27 (t, 2H, J=9.0 Hz), 7.19-7.22 (m, 4H), 6.91 (d, 2H, J=7.8 Hz), 5.66 (dd, 1H, J=11.1 Hz, J=4.2 Hz), 3.77 (dd, 1H, J=18.0 Hz, J=11.4 Hz), 3.72 (s, 3H), 3.08 (dd, 1H, J=18.0 Hz, J=4.5 Hz); LC/MS: retention time: 6.573 min; m/z (M+H)=549.45 (Calculated for C₂₈H₂₂F₂N₄O₄S=548.56); HPLC: 94% purity, retention time: 7.477 min.

### Example 8-14

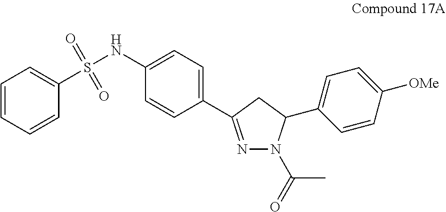

### N-(4-(1-Acetyl-5-(4-methoxyphenyl)-4,5-dihydro-1H-pyrazol-3-yl)phenyl)benzene sulfonamide (Compound 17A)

Analogue Compound 17A was prepared in the same procedure as analogue Compound 6A. ¹H NMR (300 MHz, DMSO) δ 10.63 (brs, 1H, NH), 7.79 (d, 2H, J=9.0 Hz), 7.55-7.65 (m, 5H), 7.19 (d, 2H, J=8.7 Hz), 7.05 (d, 2H, J=8.7 Hz), 6.86 (d, 2H, J=8.4 Hz), 5.42 (dd, 1H, J=11.7 Hz, J=4.2 Hz), 3.70 (s, 4H), 3.66 (dd, 1H, J=18.0 Hz, J=11.7 Hz), 2.97 (dd, 1H, J=18.0 Hz, J=4.5 Hz), 2.24 (s, 3H); LC/MS: retention time: 7.279 min; m/z (M+H)=450.50 (Calculated for C₂₄H₂₃N₃O₄S=449.52); HPLC: 100% purity, retention time: 8.571 min.

### Example 8-15

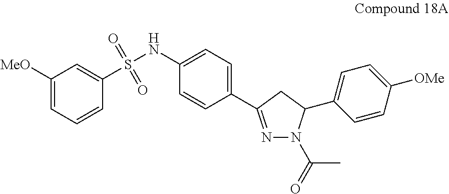

### N-(4-(1-Acetyl-5-(4-methoxyphenyl)-4,5-dihydro-1H-pyrazol-3-yl)phenyl)-3-methoxy benzenesulfonamide (Compound 18A)

Analogue Compound 18A was prepared in the same procedure as analogue Compound 6A. ¹H NMR (300 MHz, DMSO) δ 10.63 (brs, 1H, NH), 7.63 (d, 2H, J=8.4 Hz), 7.47 (t, 1H, J=8.1 Hz), 7.29-7.36 (m, 2H), 7.20 (d, 3H, J=8.7 Hz), 7.05 (d, 2H, J=8.4 Hz), 6.86 (d, 2H, J=8.4 Hz), 5.42 (dd, 1H, J=11.7 Hz, J=4.2 Hz), 3.77 (s, 3H), 3.70 (s, 3H), 3.67 (dd, 1H, J=18.0 Hz, J=11.7 Hz), 2.97 (dd, 1H, J=18.0 Hz, J=4.5 Hz), 2.24 (s, 3H); LC/MS: retention time: 7.551 min; m/z (M+H⁺)=480.56 (Calculated for C₂₅H₂₅N₃O₅S=479.55); HPLC: 100% purity, retention time: 8.639 min.

### Example 8-16

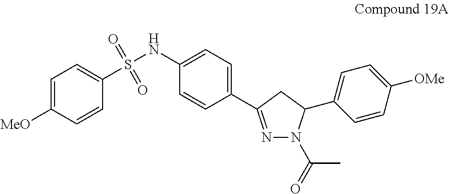

### N-(4-(1-Acetyl-5-(4-methoxyphenyl)-4,5-dihydro-1H-pyrazol-3-yl)phenyl)-4-methoxy benzenesulfonamide (Compound 19A)

Analogue Compound 19A was prepared in the same procedure as analogue Compound 6A. ¹H NMR (300 MHz, DMSO) δ 10.45 (brs, 1H, NH), 7.71 (d, 2H, J=9.0 Hz), 7.61 (d, 2H, J=8.7 Hz), 7.15 (d, 2H, J=8.7 Hz), 7.07 (d, 2H, J=9.0 Hz), 7.05 (d, 2H, J=8.7 Hz), 6.86 (d, 2H, J=8.7 Hz), 5.42 (dd, 1H, J=11.7 Hz, J=4.5 Hz), 3.79 (s, 3H), 3.70 (s, 3H), 3.67 (dd, 1H, J=18.0 Hz, J=11.7 Hz), 2.97 (dd, 1H, J=18.0 Hz, J=4.5 Hz), 2.24 (s, 3H); LC/MS: retention time: 7.404 min; m/z (M+H⁺)=480.62 (Calculated for C₂₅H₂₅N₃O₅S=479.55); HPLC: 100% purity, retention time: 8.504 min.

### Example 8-17

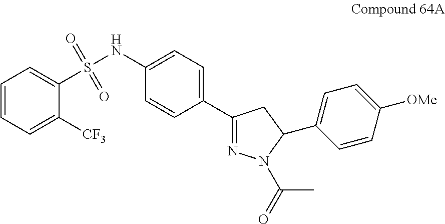

### N-(4-(1-Acetyl-5-(4-methoxyphenyl)-4,5-dihydro-1H-pyrazol-3-yl)phenyl)-2-(trifluoromethyl)benzenesulfonamide (Compound 64A)

Analogue Compound 64A was prepared in the same procedure as analogue Compound 6A. ¹H NMR (300 MHz, DMSO) δ 8.10-8.13 (m, 1H), 7.99-8.02 (m, 1H), 7.81-7.90 (m, 2H), 7.65 (d, 2H, J=8.7 Hz), 7.19 (d, 2H, J=8.7 Hz), 7.05 (d, 2H, J=8.7 Hz), 6.86 (d, 2H, J=8.7 Hz), 5.41 (dd, 1H, J=11.7 Hz, J=4.2 Hz), 3.70 (s, 3H), 3.67 (dd, 1H, J=18.0 Hz, J=11.7 Hz), 2.98 (dd, 1H, J=18.0 Hz, J=4.5 Hz), 2.24 (s, 3H); LC/MS: retention time: 11.390 min; m/z (M+H⁺)=518.32 (Calculated for C₂₅H₂₂F₃N₃O₄S=517.52); HPLC: 96% purity, retention time: 8.995 min.

### Example 8-18

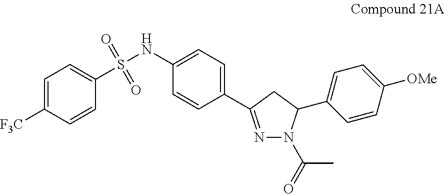

### N-(4-(1-Acetyl-5-(4-methoxyphenyl)-4,5-dihydro-1H-pyrazol-3-yl)phenyl)-4-(trifluoromethyl)benzenesulfonamide (Compound 21A)

Analogue Compound 21A was prepared in the same procedure as analogue Compound 6A. ¹H NMR (300 MHz, DMSO) δ 10.84 (brs, 1H, NH), 7.98 (m, 4H), 7.65 (d, 2H, J=8.7 Hz), 7.20 (d, 2H, J=8.7 Hz), 7.05 (d, 2H, J=8.7 Hz), 6.86 (d, 2H, J=8.7 Hz), 5.41 (dd, 1H, J=11.7 Hz, J=4.5 Hz), 3.70 (s, 3H), 3.68 (dd, 1H, J=18.0 Hz, J=11.7 Hz), 2.98 (dd, 1H, J=18.0 Hz, J=4.5 Hz), 2.24 (s, 3H); LC/MS: retention time: 7.932 min; m/z (M+H⁺)=518.69 (Calculated for C₂₅H₂₂F₃N₃O₄S=517.52); HPLC: 100% purity, retention time: 9.282 min.

### Example 8-19

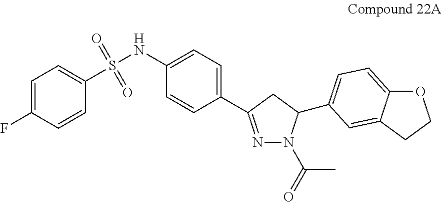

### N-(4-(1-Acetyl-5-(2,3-dihydrobenzofuran-5-yl)-4,5-dihydro-1H-pyrazol-3-yl)phenyl)-4-fluorobenzenesulfonamide (Compound 22A)

Analogue Compound 22A was prepared in the same procedure as analogue Compound 6A. ¹HNMR (300 MHz, DMSO) δ 10.60 (brs, 1H, NH), 7.82 (d, 1H, J=9.0 Hz), 7.80 (d, 1H, J=8.7 Hz), 7.60 (d, 2H, J=8.7 Hz), 7.38 (t, 2H, J=8.7 Hz), 7.13 (d, 2H, J=8.4 Hz), 6.96 (s, 1H), 6.84 (d, 1H, J=8.1 Hz), 6.65 (d, 1H, J=8.1 Hz), 5.38 (dd, 1H, J=11.4 Hz, J=4.2 Hz), 4.45 (t, 2H, J=8.4 Hz), 3.69 (dd, 1H, J=18.0 Hz, J=11.7 Hz), 3.08 (t, 2H, J=8.4 Hz), 2.97 (dd, 1H, J=18.0 Hz, J=4.2 Hz), 2.22 (s, 3H); LC/MS: retention time: 7.510 min; m/z (M+H⁺)=480.37 (Calculated for C₂₅H₂₂FN₃O₄S=479.52); HPLC: 95% purity, retention time: 8.602 min.

### Example 8-20

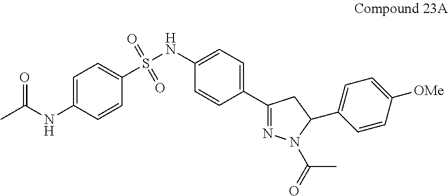

### N-(4-(N-(4-(1-Acetyl-5-(4-methoxyphenyl)-4,5-dihydro-1H-pyrazol-3-yl)phenyl)sulfamoyl)phenyl)acetamide (Compound 23A)

Analogue Compound 23A was prepared in the same procedure as analogue Compound 6A. ¹H NMR (300 MHz, DMSO) δ 10.50 (brs, 1H, NH), 10.30 (s, 1H, NH), 7.71 (m, 4H), 7.61 (d, 2H, J=8.7 Hz), 7.17 (d, 2H, J=8.7 Hz), 7.05 (d, 2H, J=8.7 Hz), 6.86 (d, 2H, J=8.7 Hz), 5.42 (dd, 1H, J=11.7 Hz, J=4.2 Hz), 3.70 (s, 3H), 3.67 (dd, 1H, J=18.0 Hz, J=11.7 Hz), 2.99 (dd, 1H, J=18.0 Hz, J=4.2 Hz), 2.24 (s, 3H), 2.05 (s, 3H); LC/MS: retention time: 6.943 min; m/z (M+H⁺)=507.45 (Calculated for C₂₆H₂₆N₄O₅S=506.57); HPLC: 99% purity, retention time: 7.539 min.

### Example 8-21

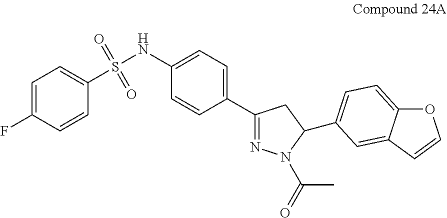

### N-(4-(1-Acetyl-5-(benzofuran-5-yl)-4,5-dihydro-1H-pyrazol-3-yl)phenyl)-4-fluorobenzenesulfonamide (Compound 24A)

Analogue Compound 24A was prepared in the same procedure as analogue Compound 6A. ¹H NMR (300 MHz, DMSO) δ 11.00 (brs, 1H, NH), 8.10-8.13 (m, 1H), 7.98-8.01 (m, 1H), 7.83-7.86 (m, 3H), 7.64 (d, 2H, J=8.4 Hz), 7.17 (d, 2H, J=8.4 Hz), 7.05 (d, 2H, 8.4 Hz), 6.86 (d, 2H, J=8.7 Hz), 5.42 (dd, 1H, J=11.7 Hz, 4.2 Hz), 3.67 (dd, 1H, J=18.0 Hz, J=11.7 Hz), 2.99 (dd, 1H, J=18.0 Hz, J=4.2 Hz), 2.24 (s, 3H); LC/MS: retention time: 6.943 min; m/z (M+H⁺)=507.45 (Calculated for C₂₅H₂₂FN₃O₄S=477.51); HPLC: 98% purity, retention time: 8.835 min.

### Example 8-22

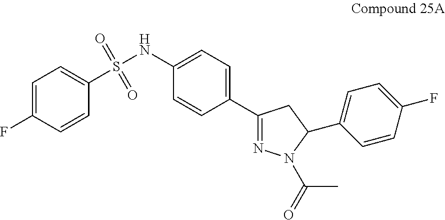

### N-(4-(1-Acetyl-5-(4-fluorophenyl)-4,5-dihydro-1H-pyrazol-3-yl)phenyl)-4-fluoro benzenesulfonamide (Compound 25A)

Analogue Compound 25A was prepared in the same procedure as analogue Compound 6A. ¹H NMR (300 MHz, DMSO) δ 10.62 (brs, 1H, NH), 7.86 (d, 1H, J=9.0 Hz), 7.84 (d, 1H, J=8.7 Hz), 7.61 (d, 2H, J=8.7 Hz), 7.38 (t, 2H, J=8.7 Hz), 7.10-7.17 (m, 6H), 5.45 (dd, 1H, J=12.0 Hz, J=4.5 Hz), 3.69 (dd, 1H, J=18.3 Hz, J=12.0 Hz), 3.00 (dd, 1H, J=18.0 Hz, J=4.5 Hz), 2.24 (s, 3H); LC/MS: retention time: 7.514 min; m/z (M+H⁺)=456.46 (Calculated for C₂₃H₁₉F₂N₃O₃S=455.48); HPLC: 96.5% purity, retention time: 8.461 min.

### Example 8-23

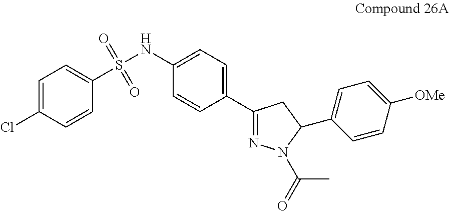

### N-(4-(1-Acetyl-5-(4-methoxyphenyl)-4,5-dihydro-1H-pyrazol-3-yl)phenyl)-4-chloro benzenesulfonamide (Compound 26A)

Analogue Compound 26A was prepared in the same procedure as analogue Compound 6A. ¹H NMR (300 MHz, DMSO) δ 10.67 (brs, 1H, NH), 7.75 (d, 2H, J=8.1 Hz), 7.60-7.65 (m, 4H), 7.16 (d, 2H, J=8.7 Hz), 7.03 (d, 2H, J=8.7 Hz), 6.84 (d, 2H, J=8.7 Hz), 5.41 (dd, 1H, J=11.7 Hz, J=4.2 Hz), 3.68 (s, 3H), 3.65 (dd, 1H, J=18.0 Hz, J=11.7 Hz), 2.98 (dd, 1H, J=18.0 Hz, J=4.5 Hz), 2.22 (s, 3H); LC/MS: retention time: 7.998 min; m/z (M+H⁺)=484.41 (Calculated for C₂₄H₂₂ClN₃O₄S=483.97); HPLC: 100% purity, retention time: 8.926 min.

### Example 8-24

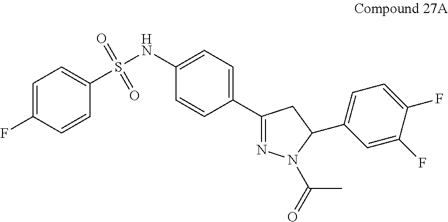

### N-(4-(1-Acetyl-5-(3,4-difluorophenyl)-4,5-dihydro-1H-pyrazol-3-yl)phenyl)-4-fluoro benzenesulfonamide (Compound 27A)

Analogue Compound 27A was prepared in the same procedure as analogue Compound 6A. ¹H NMR (300 MHz, DMSO) δ 10.65 (brs, 1H, NH), 7.86 (d, 1H, J=9.0 Hz), 7.83 (d, 1H, J=9.0 Hz), 7.63 (d, 2H, J=8.7 Hz), 7.41 (t, 2H, J=8.7 Hz), 7.20-7.35 (m, 2H), 7.19 (d, 2H, J=8.7 Hz), 6.98-7.05 (m, 1H), 5.47 (dd, 1H, J=11.7 Hz, J=4.2 Hz), 3.75 (dd, 1H, J=18.0 Hz, J=11.7 Hz), 3.06 (dd, 1H, J=18.0 Hz, J=4.2 Hz), 2.27 (s, 3H); LC/MS: retention time: 7.007 min; m/z (M+H⁺)=474.10 (Calculated for C₂₃H₁₈F₃N₃O₃S 473.47); HPLC: 100% purity, retention time: 8.817 min.

### Example 8-25

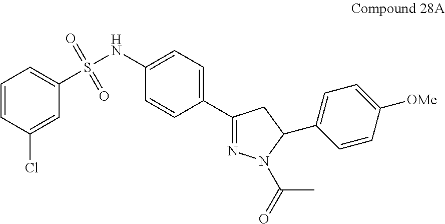

### N-(4-(1-Acetyl-5-(4-methoxyphenyl)-4,5-dihydro-1H-pyrazol-3-yl)phenyl)-3-chloro benzenesulfonamide (Compound 28A)

Analogue Compound 28A was prepared in the same procedure as analogue Compound 6A. ¹H NMR (300 MHz, DMSO) δ 10.70 (brs, 1H, NH), 7.57-7.81 (m, 6H), 7.20 (d, 2H, J=8.7 Hz), 7.05 (d, 2H, J=8.7 Hz), 6.86 (d, 211, J=8.4 Hz), 5.42 (dd, 111, J=11.7 Hz, J=4.2 Hz), 3.70 (s, 3H), 3.69 (dd, 1H, J=18.0 Hz, J=11.7 Hz), 3.00 (dd, 1H, J=18.0 Hz, J=4.2 Hz), 2.24 (s, 3H); LC/MS: retention time: 7.766 min; m/z (M+H⁺)=484.53 (Calculated for C₂₄H₂₂ClN₃O₄S=483.97); HPLC: 100% purity, retention time: 8.911 min.

### Example 8-26

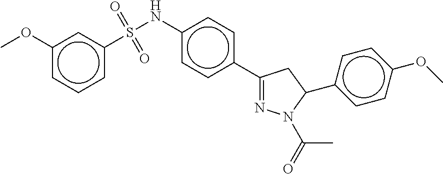

Compound 29A: Enantiomer A of Compound 18A.

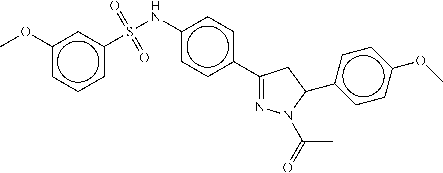

Compound 30A: Enantiomer B of Compound 18A.

### Example 8-27

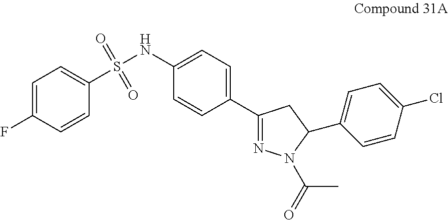

### N-(4-(1-Acetyl-5-(4-chlorophenyl)-4,5-dihydro-1H-pyrazol-3-yl)phenyl)-4-fluoro benzenesulfonamide (Compound 31A)

Analogue Compound 31A was prepared in the same procedure as analogue Compound 6A. ¹H NMR (300 MHz, DMSO) δ 10.61 (brs, 1H, NH), 7.88 (d, 1H, 9.0 Hz), 7.86 (d, 1H, J=9.0 Hz), 7.63 (d, 2H, J=8.7 Hz), 7.34-7.43 (m, 4H), 7.19 (d, 4H, J=8.7 Hz), 5.47 (dd, 1H, J=11.7 Hz, J=4.5 Hz), 3.72 (dd, 1H, J=18.0 Hz, J=12.0 Hz), 3.00 (dd, 1H, J=18.0 Hz, J=4.5 Hz), 2.26 (s, 3H); LC/MS: retention time: 6.480 min; m/z (M+H⁺)=472.18 (Calculated for C₂₃H₁₉ClFN₃O₃S=471.93); HPLC: 97% purity, retention time: 8.979 min

### Example 8-28

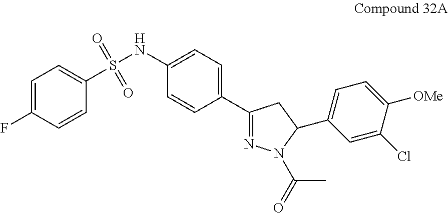

### N-(4-(1-Acetyl-5-(3-chloro-4-methoxyphenyl)-4,5-dihydro-1H-pyrazol-3-yl)phenyl)-4-fluorobenzenesulfonamide (Compound 32A)

Analogue Compound 32A was prepared in the same procedure as analogue Compound 6A. ¹H NMR (300 MHz, DMSO) δ 10.58 (brs, 1H, NH), 7.85 (d, 1H, J=9.0 Hz), 7.83 (d, 1H, J=9.0 Hz), 7.63 (d, 2H, J=8.7 Hz), 7.40 (t, 2H, J=8.7 Hz), 7.20 (s, 1H), 7.19 (d, 2H, J=8.7 Hz), 7.07 (s, 2H), 5.43 (dd, 1H, J=11.7 Hz, J=4.8 Hz), 3.81 (s, 3H), 3.72 (dd, 1H, J=18.0 Hz, J=11.7 Hz), 3.00 (dd, 1H, J=18.0 Hz, J=4.8 Hz), 2.25 (s, 3H); LC/MS: retention time: 8.189 min; m/z (M+H⁺)=502.54 (Calculated for C₂₄H₂₁ClFN₃O₄S=501.96); HPLC: 95% purity, retention time: 8.748 min.

### Example 8-29

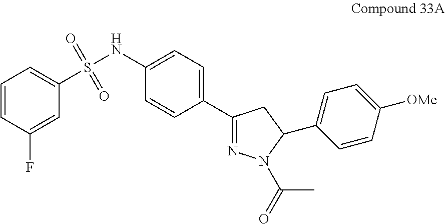

### N-(4-(1-Acetyl-5-(4-methoxyphenyl)-4,5-dihydro-1H-pyrazol-3-yl)phenyl)-3-fluoro benzenesulfonamide (Compound 33A)

Analogue Compound 33A was prepared in the same procedure as analogue Compound 6A. ¹H NMR (300 MHz, DMSO) δ 10.68 (brs, 1H, NH), 7.58-7.67 (m, 5H), 7.47-7.56 (m, 1H), 7.20 (d, 2H, J=8.7 Hz), 7.05 (d, 2H, J=8.7 Hz), 6.86 (d, 2H, J=8.7 Hz), 5.43 (dd, 1H, J=11.7 Hz, J=4.5 Hz), 3.70 (s, 3H), 3.68 (dd, 1H, J=18.0 Hz, J=11.7 Hz), 3.00 (dd, 1H, J=18.0 Hz, J=4.5 Hz), 2.24 (s, 3H); LC/MS: retention time: 7.725 min; m/z (M+H⁺)=468.57 (Calculated for C₂₄H₂₂FN₃O₄S=467.51); HPLC: 100% purity, retention time: 8.675 min.

### Example 8-30

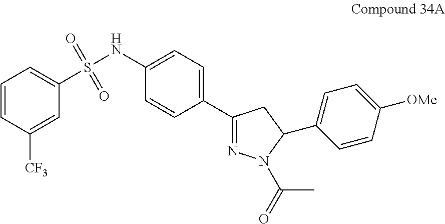

### N-(4-(1-Acetyl-5-(4-methoxyphenyl)-4,5-dihydro-1H-pyrazol-3-yl)phenyl)-3-(trifluoromethyl)benzenesulfonamide (Compound 34A)

Analogue Compound 34A was prepared in the same procedure as analogue Compound 6A. ¹H NMR (300 MHz, DMSO) δ 10.73 (brs, 1H, NH), 8.01-8.07 (m, 3H), 7.81 (t, 1H, J=8.7 Hz), 7.64 (d, 2H, J=8.7 Hz), 7.20 (d, 2H, J=9.0 Hz), 7.05 (d, 2H, J=8.7 Hz), 6.86 (d, 2H, J=8.7 Hz), 5.43 (dd, 1H, J=11.4 Hz, J=4.2 Hz), 3.71 (dd, 1H, J=18.0 Hz, J=11.4 Hz), 3.70 (s, 3H), 2.99 (dd, 1H, J=18.0 Hz, J=4.2 Hz), 2.24 (s, 3H); LC/MS: retention time: 7.920 min; m/z (M+H⁺)=518.00 (Calculated for C₂₅H₂₂F₃N₃O₄S=517.52); HPLC: 97% purity, retention time: 9.135 min.

### Example 8-31

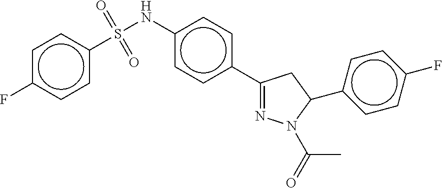

Compound 35A: Enantiomer A of Compound 25A.

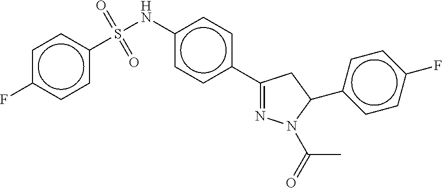

Compound 36A: Enantiomer B of Compound 25A.

### Example 8-32

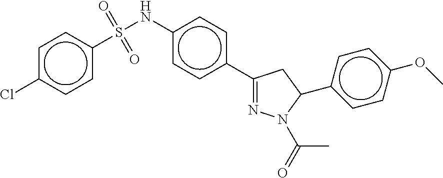

Compound 37A: Enantiomer A of Compound 26A.

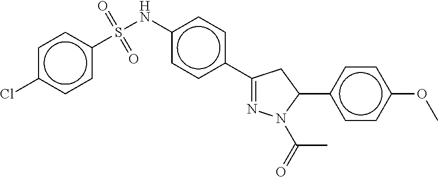

Compound 38A: Enantiomer B of Compound 26A.

### Example 8-33

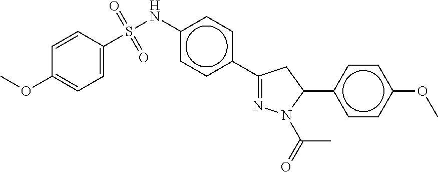

Compound 39A: Enantiomer A of Compound 19A.

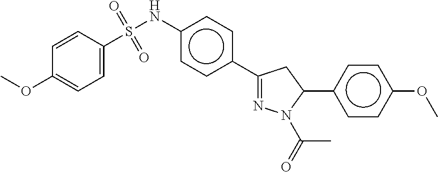

Compound 40A: Enantiomer B of Compound 19A.

### Example 8-34

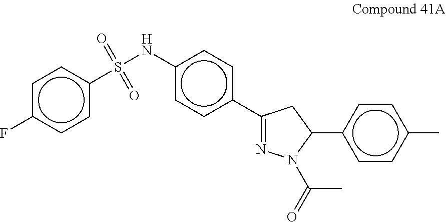

### (R)—N-(4-(1-Acetyl-5-(p-tolyl)-4,5-dihydro-1H-pyrazol-3-yl)phenyl)-4-fluorobenzene sulfonamide (Compound 41A)

Analogue Compound 41A was prepared in the same procedure as analogue Compound 6A. LC/MS: retention time: 7.562 min; m/z (M+H⁺)=452.0 (Calculated for C₂₄H₂₂FN₃O₃S=451.51); HPLC: 98% purity, retention time: 8.736 min.

### Example 8-35

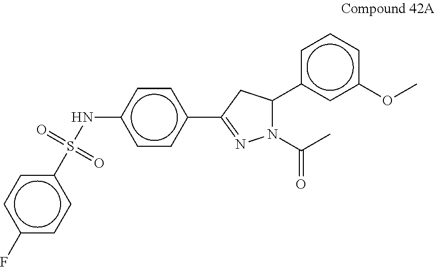

### (R)—N-(4-(1-Acetyl-5-(3-methoxyphenyl)-4,5-dihydro-1H-pyrazol-3-yl)phenyl)-4-fluoro benzenesulfonamide (Compound 42A)

Analogue Compound 42A was prepared in the same procedure as analogue Compound 6A. LC/MS: retention time: 7.080 min; m/z (M+H⁺)=468.2 (Calculated for C₂₄H₂₂FN₃O₄S=467.51); HPLC: 99% purity, retention time: 8.572 min.

### Example 8-36

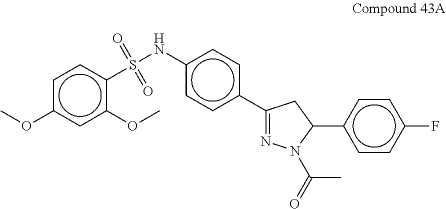

### (S)—N-(4-(1-Acetyl-5-(4-fluorophenyl)-4,5-dihydro-1H-pyrazol-3-yl)phenyl)-2,4-dimethoxybenzenesulfonamide (Compound 43A)

Analogue Compound 43A was prepared in the same procedure as analogue Compound 6A. LC/MS: retention time: 7.375 min; m/z (M+H⁺)=498.0 (Calculated for C₂₅H₂₄FN₃O₅S=497.54); HPLC: 95% purity, retention time: 8.255 min.

### Example 8-37

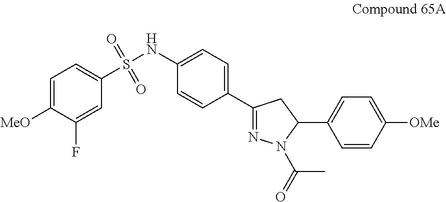

### N-(4-(1-Acetyl-5-(4-methoxyphenyl)-4,5-dihydro-1H-pyrazol-3-yl)phenyl)-3-fluoro-4-methoxybenzenesulfonamide (Compound 65A)

Analogue Compound 65A was prepared in the same procedure as analogue Compound 6A. ¹H NMR (300 MHz, DMSO) δ 10.60 (brs, 1H, NH), 7.63 (d, 2H, J=8.7 Hz), 7.61 (d, 2H, J=8.7 Hz), 7.31 (t, 1H, J=8.1 Hz), 7.19 (d, 2H, J=8.7 Hz), 7.05 (d, 2H, J=8.7 Hz), 6.86 (d, 2H, J=8.7 Hz), 5.43 (dd, 1H, J=11.4 Hz, J=4.2 Hz), 3.88 (s, 3H), 3.71 (s, 3H), 3.68 (dd, 1H, J=18.0 Hz, J=11.4 Hz), 2.98 (dd, 1H, J=17.7 Hz, J=4.2 Hz), 2.25 (s, 3H); LC/MS: retention time: 7.745 min; m/z (M+H⁺)=498.00 (Calculated for C₂₅H₂₄FN₃O₅S=497.54); HPLC: 97% purity, retention time: 8.251 min

### Example 8-38

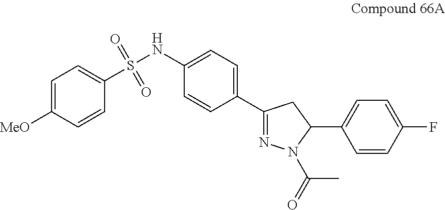

### N-(4-(1-Acetyl-5-(4-fluorophenyl)-4,5-dihydro-1H-pyrazol-3-yl)phenyl)-4-methoxy benzenesulfonamide (Compound 66A)

Analogue Compound 66A was prepared in the same procedure as analogue Compound 6A. ¹H NMR (300 MHz, DMSO) δ 10.48 (brs, 1H, NH), 7.72 (d, 2H, J=9.0 Hz), 7.62 (d, 2H, J=8.7 Hz), 7.05-7.21 (m, 8H), 5.48 (dd, 1H, J=11.7 Hz, J=4.5 Hz), 3.79 (s, 3H), 3.70 (dd, 1H, J=18.0 Hz, J=11.7 Hz), 3.00 (dd, 1H, J=18.0 Hz, J=4.5 Hz), 2.26 (s, 3H); LC/MS: retention time: 7.241 min; m/z (M+H⁺)=468.00 (Calculated for C₂₄H₂₂FN₃O₄S=467.51); HPLC: 100% purity, retention time: 8.433 min.

### Example 8-39

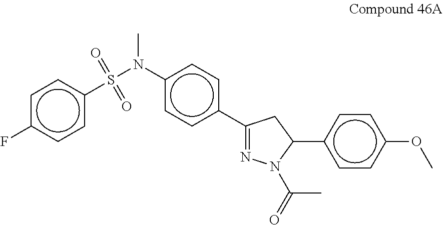

### N-(4-(1-Acetyl-5-(4-methoxyphenyl)-4,5-dihydro-1H-pyrazol-3-yl)phenyl)-4-fluoro-N-methylbenzenesulfonamide (Compound 46A)

Analogue Compound 46A was prepared in the same procedure as analogue Compound 6A. LC/MS: retention time: 8.341 min; m/z (M+H⁺)=481.9 (Calculated for C₂₅H₂₄FN₃O₄S=481.54); HPLC: 99% purity, retention time: 8.771 min.

### Example 8-40

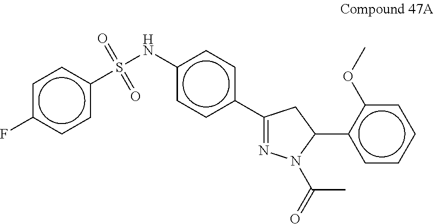

### N-(4-(1-Acetyl-5-(2-methoxyphenyl)-4,5-dihydro-1H-pyrazol-3-yl)phenyl)-4-fluoro benzenesulfonamide (Compound 47A)

Analogue Compound 47A was prepared in the same procedure as analogue Compound 6A. LC/MS: retention time: 7.859 min; m/z (M+H⁺)=467.6 (Calculated for C₂₄H₂₂FN₃O₄S=467.51); HPLC: 95% purity, retention time: 8.343 min.

### Example 8-41

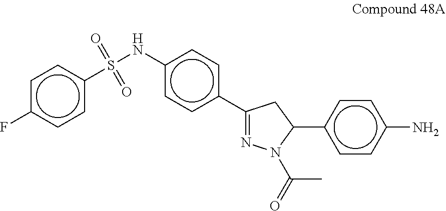

### (S)—N-(4-(1-Acetyl-5-(4-aminophenyl)-4,5-dihydro-1H-pyrazol-3-yl)phenyl)-4-fluoro benzenesulfonamide (Compound 48A).

Analogue Compound 48A was prepared in the same procedure as analogue Compound 6A. LC/MS: retention time: 2.548 min; m/z (M+H⁺)=453.0 (Calculated for C₂₃H₂₁FN₄O₃S=452.50); HPLC: 99% purity, retention time: 6.077 min.

### Example 8-42

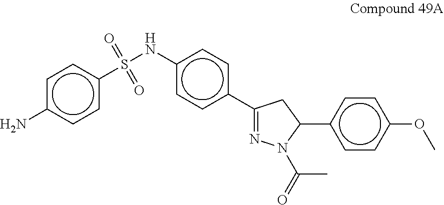

### (S)—N-(4-(1-Acetyl-5-(4-methoxyphenyl)-4,5-dihydro-1H-pyrazol-3-yl)phenyl)-4-amino benzenesulfonamide (Compound 49A)

Analogue Compound 49A was prepared in the same procedure as analogue Compound 6A. LC/MS: retention time: 7.858 min; m/z (M+H⁺)=465.0 (Calculated for C₂₄H₂₄N₄O₄S=464.54); HPLC: 95% purity, retention time: 7.328 min.

### Example 8-43

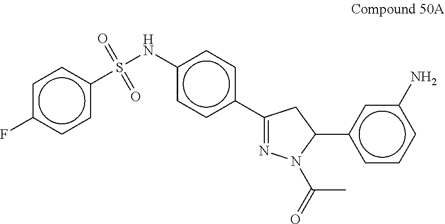

### (S)—N-(4-(1-Acetyl-5-(3-aminophenyl)-4,5-dihydro-1H-pyrazol-3-yl)phenyl)-4-fluoro benzenesulfonamide (Compound 50A)

Analogue Compound 50A was prepared in the same procedure as analogue Compound 6A. LC/MS: retention time: 6.648 min; m/z (M+H⁺)=453.4 (Calculated for C₂₃H₂₁FN₄O₃S=452.50); HPLC: 94% purity, retention time: 7.396 min.

### Example 8-44

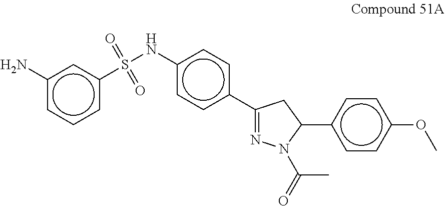

### (S)—N-(4-(1-Acetyl-5-(4-methoxyphenyl)-4,5-dihydro-1H-pyrazol-3-yl)phenyl)-3-amino benzenesulfonamide (Compound 51A)

Analogue Compound 51A was prepared in the same procedure as analogue Compound 6A. LC/MS: retention time: 8.498 min; m/z (M+H⁺)=465.18 (Calculated for C₂₄H₂₄N₄O₄S=464.54); HPLC: 98% purity, retention time: 7.025 min.

### Example 9

### PKM2 Enzymatic Activity Assays

### Assay Protocol

The ability of several test compounds to activate PKM2 was determined and the results are presented in Table 1 below. PKM2 performs the following enzymatic reaction, where the PKM2 substrates, PEP (phosphoenolpyruvate) and ADP, are converted to pyruvate and ATP as follows:

PEP+ADP→Pyruvate+ATP.

All of the enzymatic reactions were conducted in duplicates at room temperature for 1 hour in a 50 μL mixture containing:
PKM2 assay buffer (50 mM Imidazole, pH 7.2; 7 mM MgCl₂; 50 mM KCl; 0.01% Tween;
and 0.05% BSA), 0.1 mM ADP, 0.5 mM PEP, 0.1 nM PKM2 enzyme (PKM2, human recombinant (BPS catalog number 50295)) and the indicated test compound.
   Fructose 1,6 biphosphat e, (FBP (Sigma)) was used as a positive control for activation at concentration of 0.5 mM. After enzymatic reactions, 50 μL of Kinase-Glo® Plus reagent (Promega kit) was added to each well and luminescence was measured using a BioTek Synergy™ 2 microplate reader.

### Data Analysis

PKM2 activity assays were performed in duplicates at each concentration. The luminescence data were analyzed using the computer software, GraphPad PRISM®. In the absence of the compound, the luminescence was defined as 0% activity. The percent activity in the presence of each compound was calculated according to the following equation:

% Activity=[(L−L_{b})/(Lₜ−L_{b})−1]×100%

where L=the luminescence in the presence of the test compound, L_{b}=the luminescence in the absence of PKM2, and Lₜ=the luminescence intensity in the absence of the test compound. The % activity values versus a series of compound concentrations were plotted using non-linear regression analysis of Sigmoidal dose-response curve generated with the equation Y=B+(T−B)/1+10^{((LogEC50-X)×Hill Slope)}, where Y=percent activity, B=minimum percent activity, T=maximum percent activity, X=logarithm of compound and Hill Slope=slope factor or Hill coefficient. The AC₅₀ values, shown in Table 2 were determined for the indicated test compounds as the concentration causing a half-maximal percent activity.

TABLE 2

| | | | Max | | Ret. | |
|---|---|---|---|---|---|---|
| Comp. | | AC₅₀ | activity | | Time | Optical |
| # | Structure | group¹ | Group² | Enantiomer | (Min.) | Rotation |
| 1A | 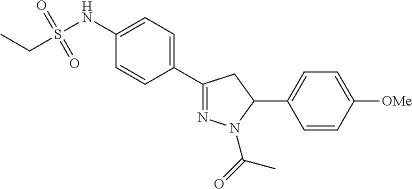 | A | B | | | |
| 2A | 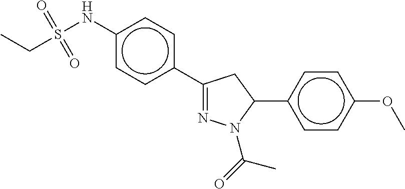 | A | B | Enantiomer A | 3.37 min. | [α]_{D}²⁰ = +18.9 In DCM |
| 3A | 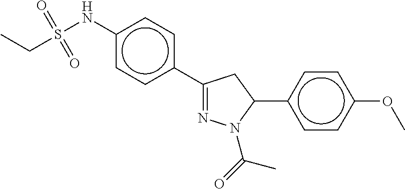 | A | B | Enantiomer B | 6.07 min. | [α]_{D}²⁰ = −19.8 In DCM |
| 4A | 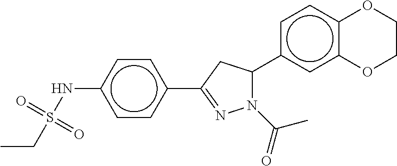 | A | B | | | |
| 5A | 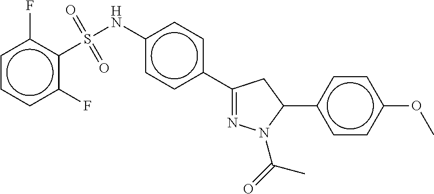 | A | A | | | |
| 6A | 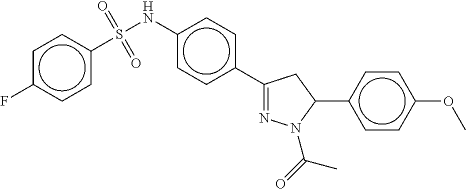 | A | B | | | |
| 7A | 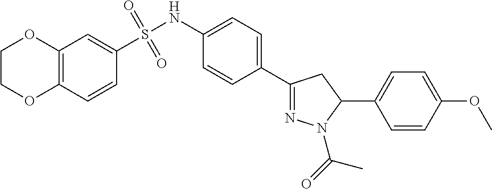 | B | B | | | |
| 8A | 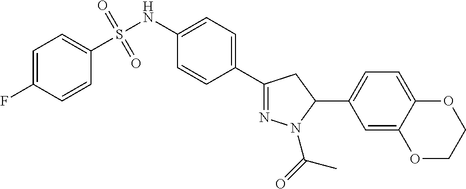 | B | B | | | |
| 9A | 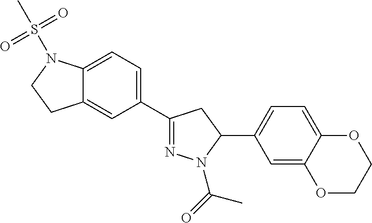 | C | C | | | |
| 10A | 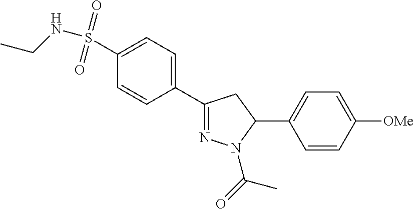 | A | B | | | |
| 11A | 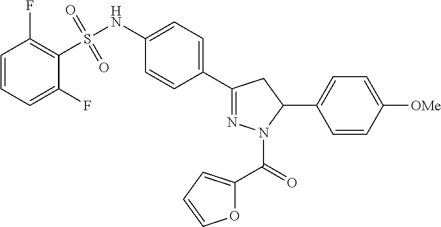 | B | A | | | |
| 12A | 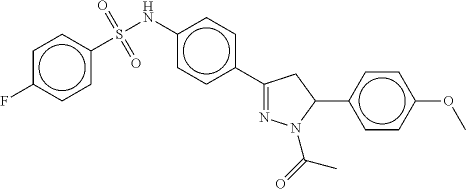 | C | C | Enantiomer A | 4.03 min. | [α]_{D}²⁰ = +7.95 In MeOH |
| 13A | 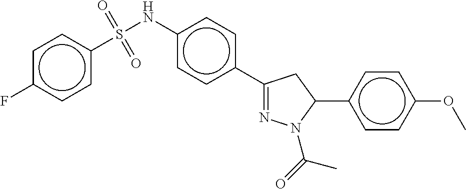 | A | B | Enantiomer B | 4.95 min | [α]_{D}²⁰ = −7.26 In MeOH |
| 14A | 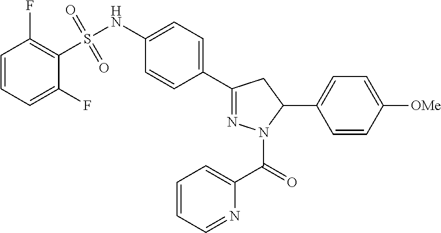 | B | C | | | |
| 15A | 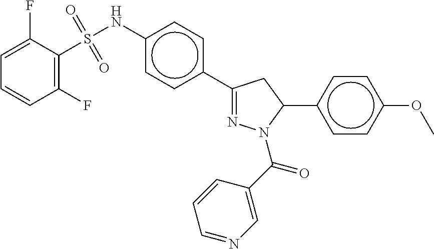 | B | A | | | |
| 16A | 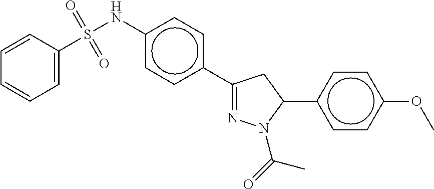 | B | B | | | |
| 17A | 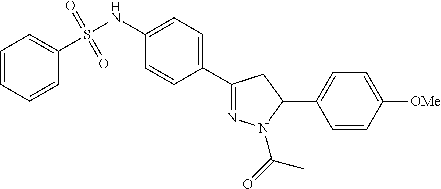 | B | C | | | |
| 18A | 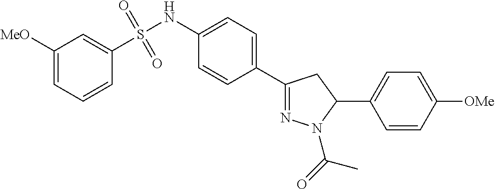 | A | B | | | |
| 19A | 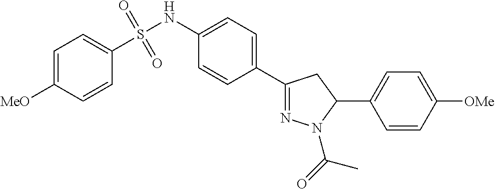 | A | A | | | |
| 20A | 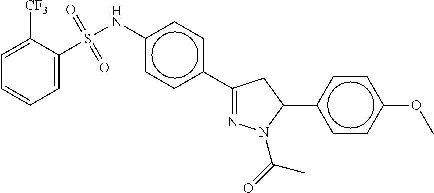 | A | B | | | |
| 21A | 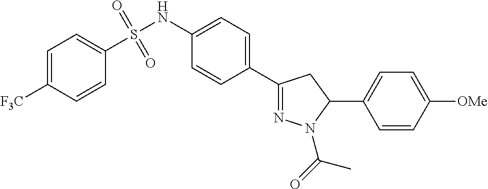 | A | B | | | |
| 22A | 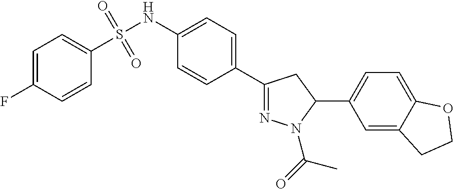 | A | B | | | |
| 25A | 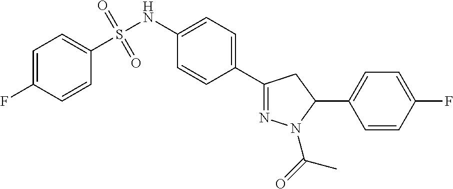 | A | C | | | |
| 26A | 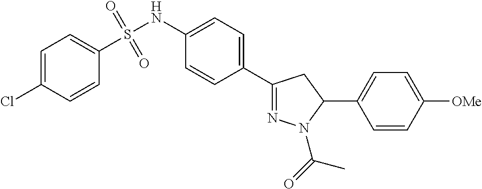 | A | C | | | |
| 27A | 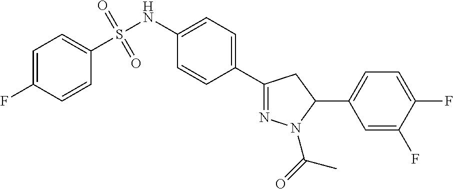 | B | C | | | |
| 28A | 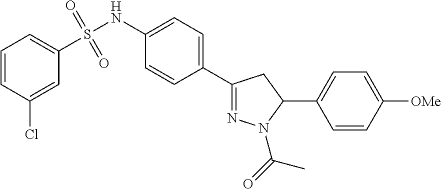 | B | C | | | |
| 29A | 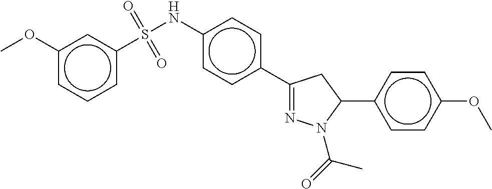 | ND | ND | Enantiomer A | 2.51 min | [α]_{D}²⁰ = +8.46 In DCM |
| 30A | 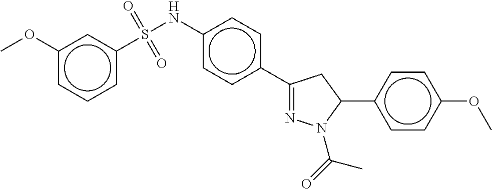 | ND | ND | Enantiomer B | 3.51 min | [α]_{D}²⁰ = −10.77 In DCM |
| 33A | 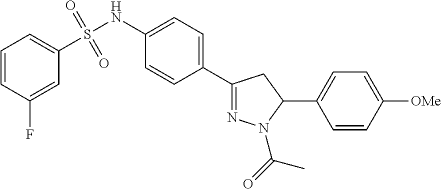 | A | B | | | |
| 35A | 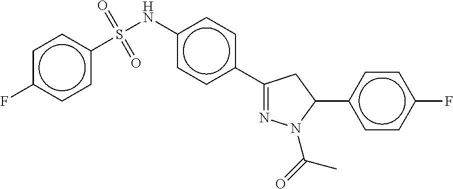 | A | C | Enantiomer A | 2.35 min | [α]_{D}²⁰ = +42.86 In DCM |
| 36A | 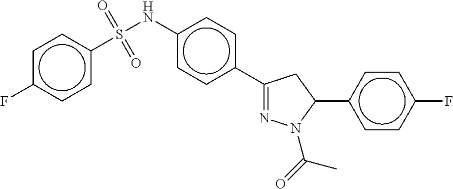 | A | C | Enantiomer B | 3.36 min | [α]_{D}²⁰ = −46.94 In DCM |
| 37A | 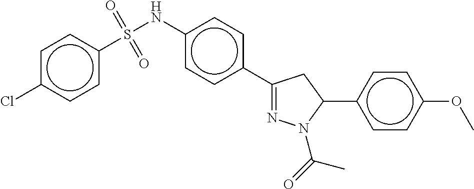 | ND | ND | Enantiomer A | 3.78 min | [α]_{D}²⁰ = +14.97 In DCM |
| 38A | 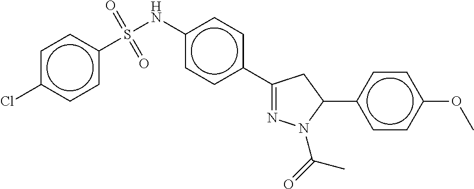 | ND | ND | Enantiomer B | 5.13 min | [α]_{D}²⁰ = −13.61 In DCM |
| 39A | 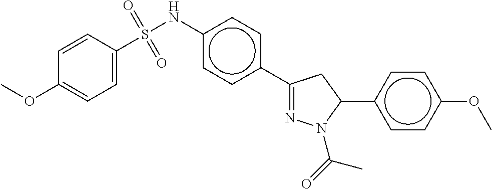 | A | C | Enantiomer A | 2.77 min | [α]_{D}²⁰ = +13.53 In DCM |
| 40A | 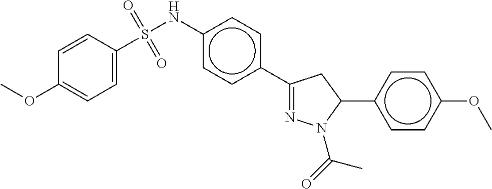 | A | A | Enantiomer B | 3.79 min | [α]_{D}²⁰ = −10.51 In DCM |
| 44A | 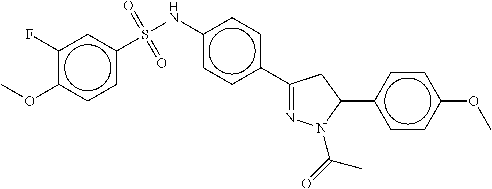 | A | B | | | |
| 45A | 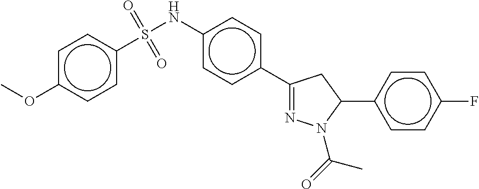 | A | C | | | |
| 52A | 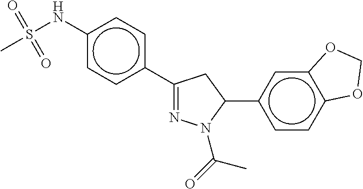 | A | C | | | |
| 53A | 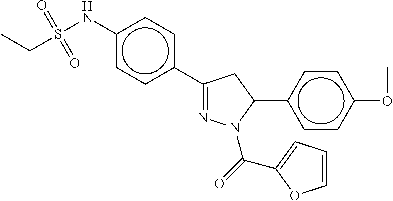 | B | A | | | |
| 54A | 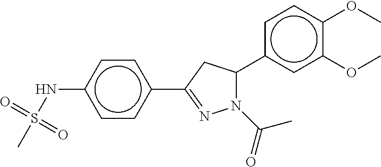 | ND | 6% (30 μM) | | | |
| 55A | 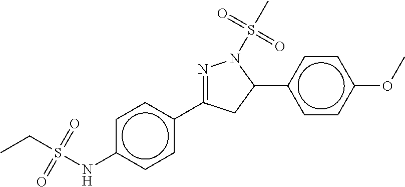 | ND | −5% (30 μM) | | | |
| 56A | 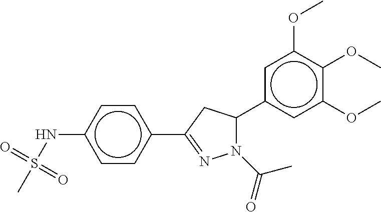 | ND | −6% (30 μM) | | | |
| 57A | 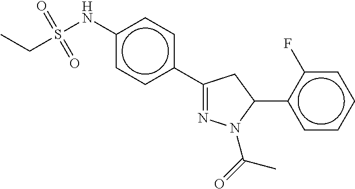 | A | C | | | |
| 59A | 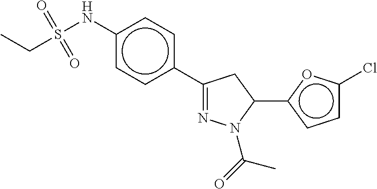 | ND | 47% (30 μM) | | | |
| 60A | 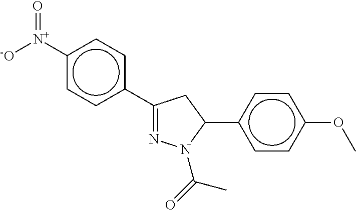 | ND | 88% (30 μM) | | | |
| 67A | 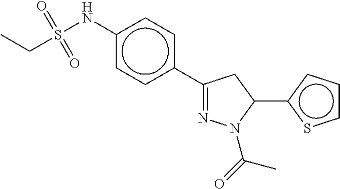 | C | B | | | |

¹A: ≦1 μM; B: 1-10 μm; C: 10-100 μM
²A: ≧200%; B: 130-200%; C: ≦130%

### Example 10

### In Vitro Cell Proliferation Assays

### Example 10-1

### In Vitro Cell Proliferation Assays of Compounds 1A, 6A, and 61A

### Assay Protocol I

Growth inhibitory activity against the human tumor cell lines was determined using Promega's CellTiter-Glo® assay. The cell lines of interest were placed in a 96-well microculture plate (Costar® white, flat bottom #3917) in a total volume of 90 μL/well. After 24 hours of incubation in a humidified incubator at 37° C. with 5% CO₂ and 95% air, 10 μL of serially diluted test compounds in growth medium were added to each well. After 96 total hours of culture in a CO₂ incubator, the plated cells and CellTiter-Glo® (Promega # G7571) reagents were brought to room temperature to equilibrate for 30 minutes. 100 μL of CellTiter-Glo® reagent was added to each well. The plate was shaken for 2 minutes and then left to equilibrate for 10 minutes before reading luminescence on the Tecan GENios microplate reader.

Percent inhibition of cell growth was calculated relative to untreated control wells. All tests were performed in duplicate for each concentration level. The IC₅₀ value for the test compounds was estimated using Prism 3.03 by curve-fitting the data.

### Assay Protocol II

In a second assay to determine the inhibition of cell proliferation by a test compound, cell proliferation of the cell line of interest was measured by a cell proliferation index assay using Fluofarma (www.fluofarma.com) based technology. This assay is based on the dilution rate of a fluorescent membrane marker, which is a direct function of the number of cell divisions. Briefly, the assay is performed by loading the cells with a non-toxic fluorescent phospholipid analog before the seeding. The probe inserts stably into the cell membrane, after which the probe does not exchange with neighboring cells or the surrounding medium; however, the probe will be distributed between daughter cells after division. Flow cytometry analysis of the fluorescent probe is performed after the loading of the cells both prior to, as well as after treatment with various concentrations of the test compound. The dilution rate of the fluorescent probe at the single cell level is directly correlated to the number of cell divisions.

### Results

The results for the indicated tumor cells lines of inhibition of cellular proliferation by the indicated test compounds, following the numbering of Table 1, are provided below (all results are as measured using Assay Protocol I, except as otherwise noted):

### N-(4-(1-acetyl-5-(4-methoxyphenyl)-4,5-dihydro-1H-pyrazol-3-yl)phenyl)ethanesulfonamide

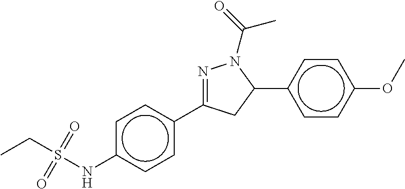

Test Compound 1A

| | IC₅₀ |
|---|---|
| Cell line | Test Compound 1A |
| H1299 (Non-small cell lung carcinoma cell | 40 μM-100 μM/ |
| line) | 40 μM-100 μM^{†} |
| A549 (Non-small cell lung carcinoma cell line) | <40 μM |
| SW480 (Colorectal cancer cell line) | 40 μM-100 μM |
| HT-29 (Colorectal cancer cell line) | >100 μM |
| HepG2 (Hepatocellular cancer cell line) | 40 μM-100 μM |

^{†}As measured by Assay Protocol II.

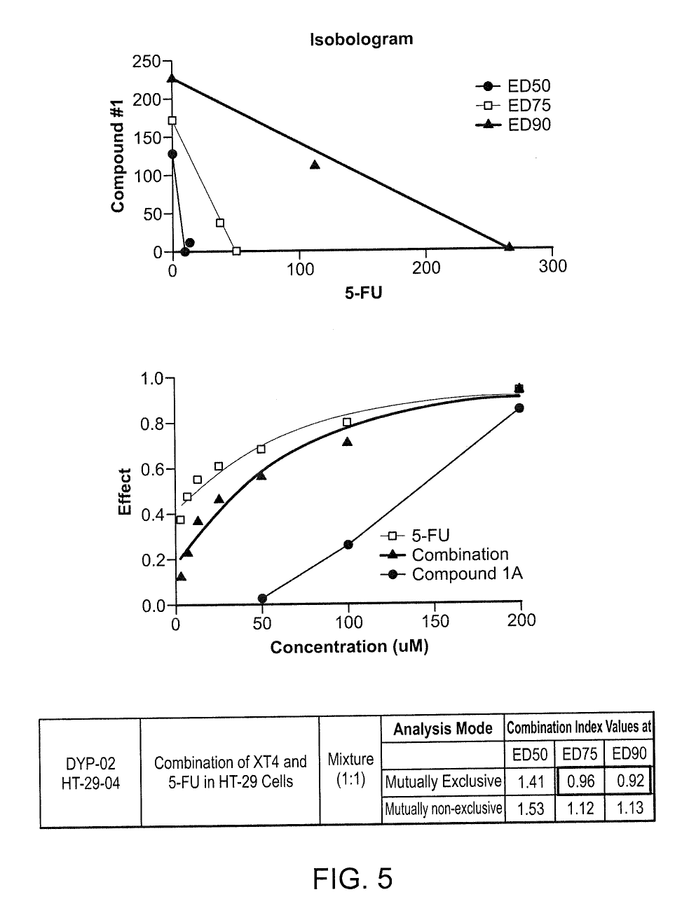

Furthermore, as shown in FIG. 5, when Compound 1A was used in combination with 5-FU, a standard of care first line chemotherapy for colorectal cancer, on HT-29-cell, a colorectal cancer cell line, an effect that was at least additive was observed.

### N-(4-(1-acetyl-5-(4-methoxyphenyl)-4,5-dihydro-1H-pyrazol-3-yl)phenyl)-4-fluorobenzenesulfonamide

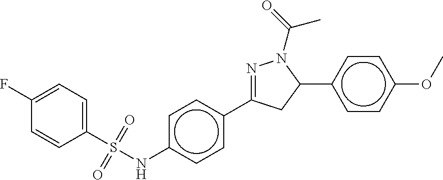

Test Compound 6A

| | IC₅₀ |
|---|---|
| Cell line | Test Compound 6A |
| H1299 (Non-small cell lung carcinoma cell line) | <40 μM^{†} |

^{†}As measured by Assay Protocol II.

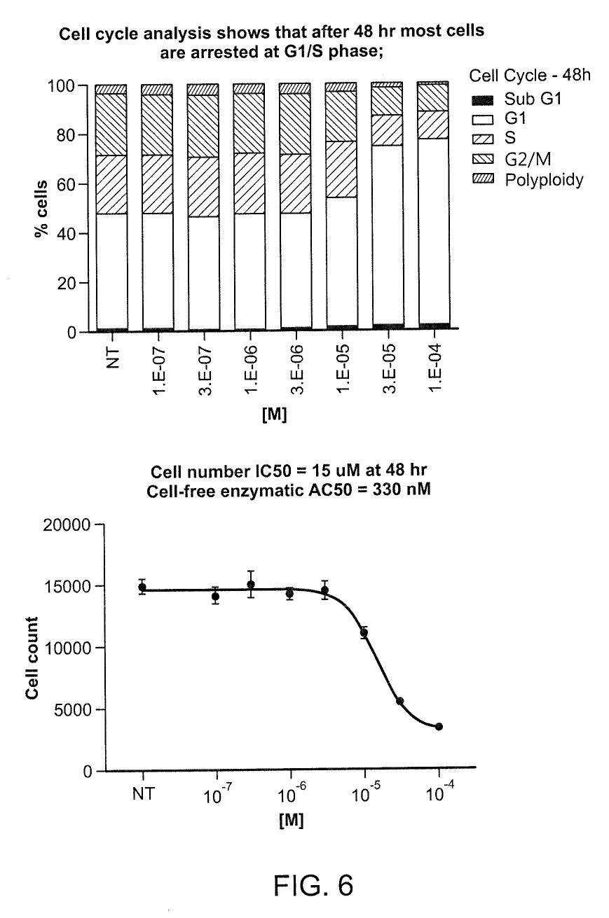

Furthermore, as shown in FIG. 6, cell cycle analysis of Compound 13A—i.e., the S-enantiomer of Compound 6A, both following the numbering of Table 1, was performed on H1299 cells. After 48 hours, most cells were arrested at the G1/S phase, in other words, the compound induces growth arrest and stops the cell cycle at the G1 phase, just before DNA synthesis, consistent with a mechanism of action where the cell is deprived of the building blocks necessary for biosynthesis.

### N-(4-(1-acetyl-5-(4-methoxyphenyl)-4,5-dihydro-1H-pyrazol-3-yl)phenyl)-2,6-difluorobenzenesulfonamide

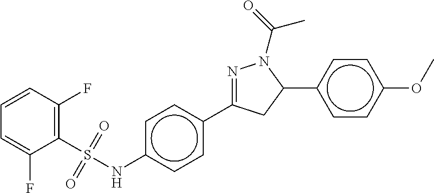

Compound 61A

| | IC₅₀ |
|---|---|
| Cell line | Test Compound 61A |
| H1299 (Non-small cell lung carcinoma cell line) | <40 μM^{†} |

^{†}As measured by Assay Protocol II.

### Example 10-2

### In Vitro Cell Proliferation Assays of Compounds 13A, 35A, 39A, 40A, 25A and 19A

The aim of the current study was to evaluate the effect of compounds of the invention on cell proliferation. The following compounds were tested:
1. Compound 13A at 10 and 100 μM
2. Compound 35A at 10 μM
3. Compound 39A at 10 μM
4. Compound 40A at 10 μM
5. Compound 25A at 10 μM
6. Compound 19A at 10 μM

Tests were performed under following conditions:
1) Full media (DMEM, 4.5 g/L glucose [25 mM], 10% FBS, pen/strep, L-glutamine)
2) 1 mM glucose media (DMEM, 1 mM D-glucose, 10% FBS, pen/strep, L-glutamine)
3) BME media (basic media lacking non-essential amino acids [NEAA], 5 mM glucose [1 g/L], 10% FBS, pen/strep, L-glutamine)
4) BME media plus NEAA
5) Lipoprotein-free media (DMEM, 4.5 g/L glucose [25 mM], 10% lipoprotein-free FBS, pen/strep, L-glutamine)
6) Glutamine-free media (DMEM, 4.5 g/L glucose [25 mM], 10% FBS, pen/strep, w/o L-glutamine)

These conditions were applied to 2 cell lines:
NCI-H460 (ATCC #HTB-177), human large cell lung carcinoma
HT-29 (ATCC #HTB-38), human colorectal adenocarcinoma

### Materials

NCI-H460 cell line was acquired from Da-Ta Biotech (Rehovot, Israel) and maintained in full media (DMEM, 4.5 g/L glucose, 10% FBS, pen/strep, L-glutamine). HT-29 cell line was purchased from ATCC and maintained in full media (DMEM, 4.5 g/L glucose, 10% FBS, pen/strep, L-glutamine). 20% D-glucose, BME, NEAA solution and XTT proliferation kit were from Biological Industries (Beit Ha-Emek, Israel). Lipoprotein-free serum was purchased from Sigma (Lot #S5394).

### Protocol

Day 0 Cells were seeded at 3000 cells/well in 100 μl full media in 96-well culture plates (60 wells/plate, external wells filled with 100 μl PBS) and incubated overnight.
Day 1 On the following day, full media was changed to the tested media and compounds were added in 0.5% DMSO final.
Day 2-5 Cells were incubated over 72 hr.
Day 5 Cell proliferation was assessed using XTT proliferation assay as follows:

### Assay Principles

The assay is based on the ability of metabolic active cells to reduce the tetrazolium salt XTT to orange colored compounds of formazan. The dye formed is water soluble and the dye intensity can be read at a given wavelength with a spectrophotometer. The intensity of the dye is proportional to the number of metabolic active cells. The use of multiwell plates and an ELISA reader enables testing a large number of samples and obtaining easy and rapid results. The test procedure includes cultivation of cells in a 96-well plate, adding the XTT reagent and incubation for 2-24 hours. During incubation an orange color is formed, the intensity of which can be measured with a spectrophotometer, in this instance with an ELISA reader. The greater the number of active cells in the well, the greater the activity of mitochondria enzymes, and the higher the concentration of the dye formed, which can then be measured and quantitated.

### Procedure

1. As described in the above protocol, the cells were cultivated in a flat 96-well plate. To each well was added 100 μl of growth media. The cells were incubated in a CO₂ incubator at 37° C. In most cases cells were used to assay proliferation after 24-96 hours. Each test included a blank containing complete medium without cells (see 7: background control).
2. The XTT reagent solution and the activation solution were defrosted immediately prior to use in a 37° C. bath. The solution was swirled gently until clear solutions were obtained.
3. For each plate (96 wells), a reaction solution was prepared by adding 0.1 ml activation solution to 5 ml XTT reagent.
4. 50 μl of the reaction solution was added to each well and the plate was incubated in an incubator for 2-24 hours (usually, 2-5 hours are sufficient).
5. The plate was shaken gently to evenly distribute the dye in the wells.
6. The absorbance of the samples was measured against a background control as a blank (see 7) with a spectrophotometer (ELISA reader) at a wavelength of 450-500 nanometer. In order to measure reference absorbance (to measure non-specific readings), a wavelength of 630-690 nanometer was used and subtracted from the 450-500 nanometer measurement.
7. Background control (blank): Slight spontaneous absorbance around 450-500 nanometer occurs in the culture medium incubated with the XTT reagent. This background absorbance depends on the culture medium, pH, incubation time and length of exposure to light. One or more blank control wells without cells were prepared by adding the same volume of culture medium and XTT Reagent solution as used in the experiment. The average absorbance of the blank control wells was subtracted from that of the other wells.

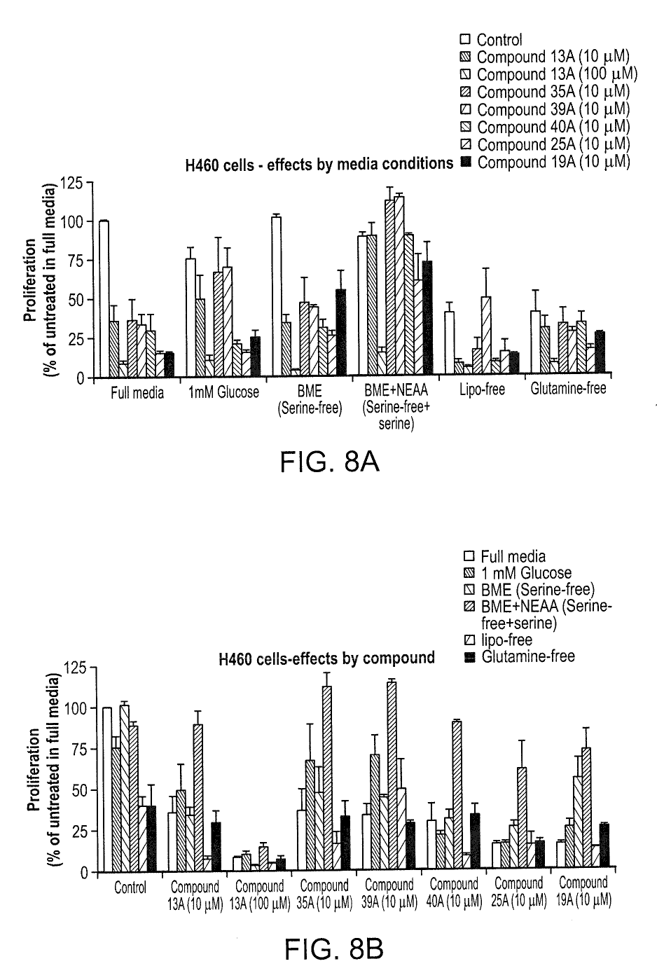

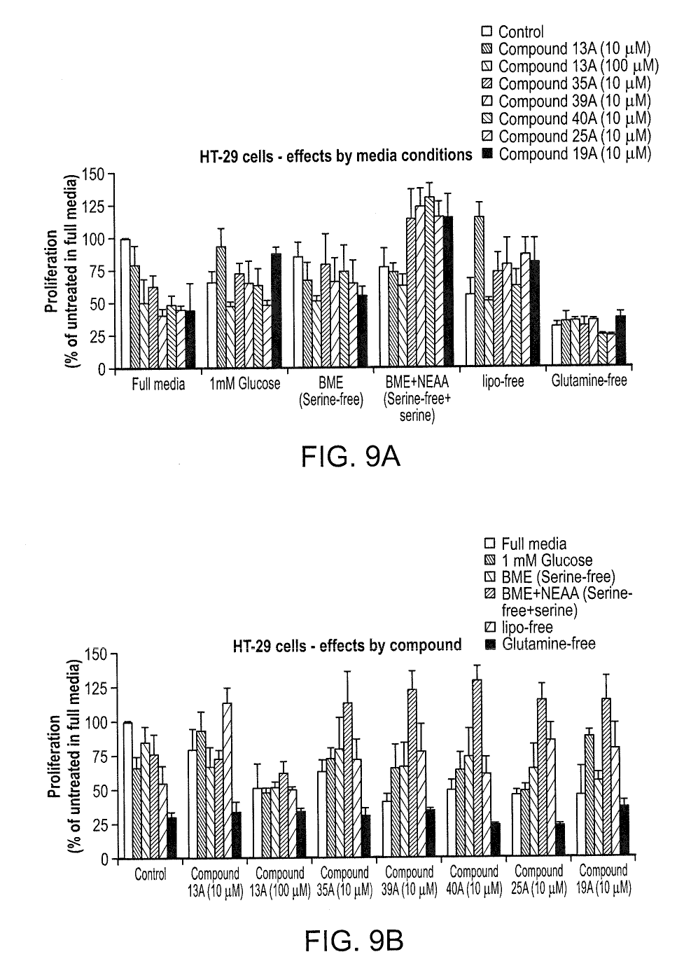

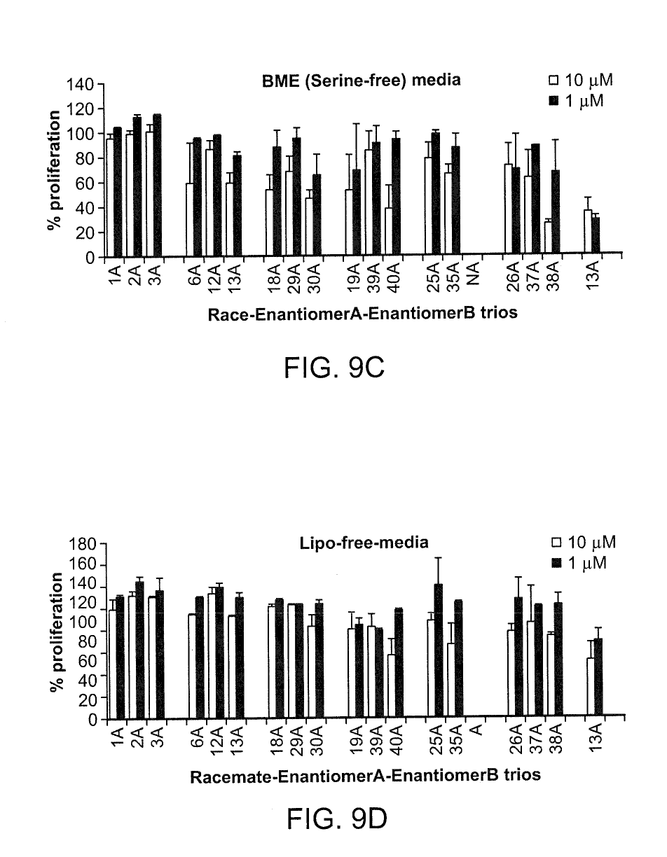

Data was analyzed using GraphPad Prism software. The proliferation changes of H460 cells and HT-29 cells under effects of media conditions or test compounds are presented in FIGS. 8 and 9.

### Example 10-2A

### In Vitro Cell Proliferation Assays of Racemates and Enantiomers

The aim of the current study was to evaluate the effect of some compounds of the invention on H460 cell proliferation under serine-free and lipoprotein-free media conditions.

The following racemate-enantiomerA-enantiomerB “trios” were tested:

### Racemate Compound 1A

### Enantiomer A Compound 2A

### Enantiomer B Compound 3A

### Racemate Compound 6A

### Enantiomer A Compound 12A

### Enantiomer B Compound 13A

### Racemate Compound 18A

### Enantiomer A Compound 29A

### Enantiomer B Compound 30A

### Racemate Compound 19A

### Enantiomer A Compound 39A

### Enantiomer B Compound 40A

### Racemate Compound 25A

### Enantiomer A Compound 35A

### Enantiomer B N/A

### Racemate Compound 26A

### Enantiomer A Compound 37A

### Enantiomer B Compound 38A

Control compound Compound 13A

Each compound was tested in 2 concentrations (10 and 1 μM) in duplicates.

Tests were performed under following conditions:
1) BME media (basic media lacking non-essential amino acids [NEAA], 5 mM glucose [1 g/L], 10% FBS, pen/strep, L-glutamine)
2) Lipoprotein-free media (DMEM, 4.5 g/L glucose [25 mM], 10% lipoprotein-free FBS, pen/strep, L-glutamine)
These conditions were applied to 1 cell line:

NCI-H460 (ATCC #HTB-177), human large cell lung carcinoma

### Materials

NCI-H460 cell line was acquired from Da-Ta Biotech (Rehovot, Israel) and maintained in full media (DMEM, 4.5 g/L glucose, 10% FBS, pen/strep, L-glutamine). DMEM and BME media, XTT proliferation kit were from Biological Industries (Beit Ha-Emek, Israel). Lipoprotein-free serum was purchased from Sigma (Lot #S5394).

### Protocol

At Day 0, cells were seeded at 3000 cells/well in 100 μl full media in 96-well culture plates (60

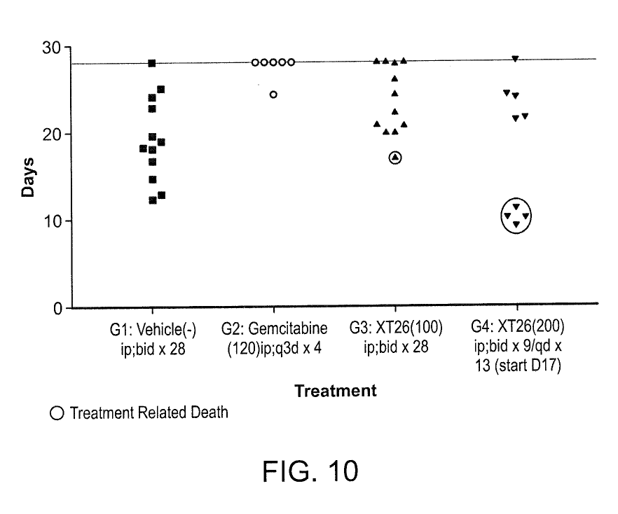

wells/plate, external wells filled with 120 μl PBS) and incubated overnight.
The rest of protocols, including XTT proliferation assay and data analysis, were the same as those in Example 10-2.
Results are shown in FIGS. 10C and 10D.

### Example 10-3

### In Vitro Cell Proliferation Assays of Compounds 1A, 13A, 18A, 21A, 26A, 29A, 30A, 32A, 33A, 34A, 36A-42A, 45A and 47A

### Assay Procedure

Cell lines H1299 (ATCC CRL-5803)

1. Cells were grown according to the ATCC conditions.
2. Cell feeding (passaging) was timed such that the cells were in exponential growth phase at the time of the assay.

### Proliferation Assay Using the Celltiter-Glo Luminescent Assay

3. On day 1, H1299 cells were counted. Cell numbers were adjusted to the desired cell concentration in Media 1: RPMI 1640 Invitrogen 11879020 (that contained no glucose or pyruvate) with 10% FBS, and 1 mM D-Glucose (4 μl of 2.5 M D-glucose G8769 was added in preparing 10 ml RPMI media with 1 mM glucose).
A 200 μl of H1299 cell suspension was added to appropriate wells in the 96-well plates, and incubated at 37° C. in the CO₂ cell incubator under normal growing conditions for overnight.
4. On day 2, a 100 mM stock concentration of test compounds in 100% DMSO was prepared and serially diluted with 100% DMSO to 33.33 mM, 11.11 mM, and 3.7 mM. A 1:1000 dilution was made to each concentration in media 1 (yielding 0.1% final DMSO concentration). The media was aspirated completely from the cell plate. Each 100 μl of the following solutions (100 μM, 33.33 μM, 11.11 μM, and 3.7 μM) was added to the cells. DMSO in a final concentration of 0.1%, and media without DMSO, were added to the cells and used as a negative control. Empty wells around the experimental wells were filled with PBS. The plate was sealed with parafilm to reduce evaporation.
5. The plate was incubated under normal conditions (5% CO₂, and 95% air), for 96 hours at 37° C.
6. On day 6 (after 96 hr incubation), the plate was equilibrated to room temperature for approximately 30 minutes. A 100 μl of CellTiter-Glo Reagent was added in each well of the plate. Contents were mixed for 2 minutes on an orbital shaker to induce cell lysis. The plate was incubated at room temperature for 10 minutes to stabilize luminescent signal. Luminescence was recorded with Flexstation 3.

### Results

Unless noted otherwise, proliferation of control or compound treated H1299 cells was measured using CellTiter-Glo Luminescent assay following 96 hr incubation of the cells with and without test compounds. The AC₅₀ values, shown in Table 3, were coded according to follows: A: <3 μM; B: 3-10 μM; C: 10-20 μM; or D: >20 μM. Table 4 shows AC₅₀ values of Compound 13A to pyruvate kinase isoforms.

TABLE 3
AC₅₀ values* measured in H1299 cell proliferation study.

| | | | | DU145 | | | | | | | | | | |
|---|---|---|---|---|---|---|---|---|---|---|---|---|---|---|
| | | BT-474 | BT-549 | (pros- | | | | H-460 (lung) | | | HCT- | HepG2 | | HT-29 |
| | 786-O | (Breast) | (breast) | tate) | H1299 (lung) | | | Lipo- | | | 116 | (liver) | | (colon) |
| Cell line | (kidney) | Com- | Com- | Com- | | | Com- | protein- | | Com- | (colon) | Com- | | Com- |
| Media | 1 mM | plete | plete | plete | 1 mM | 5 mM | plete | free | 1 mM | plete | 1 mM | plete | 1 mM | plete |
| conditions | glucose | media | media | media | glucose | glucose | media | media | glucose | media | glucose | media | glucose | media |
| Compound | A | C | C | C | C | C | C | A² | C² | D² | C | D² | B² | C |
| 13A | | | | | | | | | | | | | | |
| Compound | | | D¹ | | C | | | | | | | | | |
| 1A | | | | | | | | | | | | | | |
| Compound | | | | | D | | | | | | | | | |
| 18A | | | | | | | | | | | | | | |
| Compound | B | | | | C | | | | | | | | | |
| 21A | | | | | | | | | | | | | | |
| Compound | B | | | | D | | | | | | | | | |
| 26A | | | | | | | | | | | | | | |
| Compound | | | | | D | | | | | | | | | |
| 29A | | | | | | | | | | | | | | |
| Compound | | | | | D | | | | | | | | | |
| 30A | | | | | | | | | | | | | | |
| Compound | B | | | | D | | | | | | | | | |
| 32A | | | | | | | | | | | | | | |
| Compound | | | | | D | | | | | | | | | |
| 33A | | | | | | | | | | | | | | |
| Compound | B | | | | D | | | | | | | | | |
| 34A | | | | | | | | | | | | | | |
| Compound | B | | | | D | | | | | | | | | |
| 36A | | | | | | | | | | | | | | |
| Compound | | | | | D | | | | | | | | | |
| 37A | | | | | | | | | | | | | | |
| Compound | | | | | D | | | | | | | | | |
| 38A | | | | | | | | | | | | | | |
| Compound | | | | | D | | | | | | | | | |
| 39A | | | | | | | | | | | | | | |
| Compound | | | | | D | | D | | | | | | | |
| 40A | | | | | | | | | | | | | | |
| Compound | B | | | | C | | | | | | | | | |
| 41A | | | | | | | | | | | | | | |
| Compound | C | | | | | | | | | | | | | |
| 42A | | | | | | | | | | | | | | |
| Compound | B | | | | D | | | | | | | | | |
| 45A | | | | | | | | | | | | | | |
| Compound | B | | | | D | | | | | | | | | |
| 47A | | | | | | | | | | | | | | |

*coded according to A: <3 μM; B: 3-10 μM; C: 10-20 μM; or D: >20 μM.
¹Assay duration: 72 hours; Readout: CellTiter Glo (Promega)
²Assay duration: 72 hours; Readout: XTT

TABLE 4
Isoform selectivity of pyruvate kinases

| Compound | Enzyme | AC₅₀ |
|---|---|---|
| 13A | PKM1 | Not active |
| 13A | PKL | >11 μM |
| 13A | PKR | >30 μM |

### Example 11

### Efficacy and Safety of N-(4-(1-acetyl-5-(4-methoxyphenyl)-4,5-dihydro-1H-pyrazol-3-yl)phenyl)ethanesulfonamide in a HT-29 Xenograft Mouse Model

This example illustrates the efficacy and safety of a compound according to embodiments of the present invention, i.e., Compound 1A, using a HT-29 xenograft mouse model (a colorectal cancer model).

### Animals and Animal Housing:

Animals

Species: Mus Musculus

Strain: BALB/c nude

Age: 6-8 weeks

Sex: female

Body weight: 18-22 g

Housing Conditions

The mice were kept in Individual Ventilation Cages at constant temperature and humidity with 3 animals in each cage.

Temperature: 20-26° C.

Humidity 40-70%.

Cages: Made of polycarbonate. The size is 300 mm×180 mm×150 mm. The bedding material is corn cob, which was changed twice per week.

Diet: Animals had free access to irradiation sterilized dry granule food during the entire study period.

Water: Animals had free access to sterile drinking water.

Cage identification: the identification labels for each cage contain the following information: number of animals, sex, strain, date received, treatment, study number, group number, and the starting date of the treatment.

Animal identification: Animals were marked by ear coding.

### Experimental Methods and Procedures:

Cell Culture

The HT-29 cells were maintained in vitro as a monolayer culture in McCoy's 5A medium supplemented with 10% heat inactivated fetal bovine serum, 100 U/ml penicillin and 100 μg/ml streptomycin at 37° C. in an atmosphere of 5% CO₂ in air. The tumor cells were routinely subcultured twice weekly by trypsin-EDTA treatment. The cells growing in an exponential growth phase were harvested and counted for tumor inoculation.

### Tumor Inoculation

65 mice were inoculated subcutaneously at the right flank with 3.0×10⁶ HT-29 tumor cells in 0.1 ml of serum-free medium for tumor development. The treatments were started when the tumor size reached approximately 75 (50-100) mm³. The test article administration and the animal numbers in each group are shown in the following experiment design Table 5.

TABLE 5
Groups and Treatments for Tumor Efficacy Study in HT-29 model

| | | | Dose | Dosing | Dosing | |
|---|---|---|---|---|---|---|
| Group | n | Treatment | (mg/kg) | Volume | Route | Schedule |
| 1 | 6 | Vehicle | — | 10 μl/g | i.p. | QD × 23 |
| | | Control | | | | |
| 2 | 6 | Irino- | 100 | 10 μl/g | i.p. | Q5D × 4 |
| | | tecan | | | | |
| 3 | 6 + 2* | Com- | 100 | 10 μl/g | i.p. | Q2D × 12 |
| | | pound 1A | | | | |
| 4 | 6 + 2 | Com- | 100 | 10 μl/g | i.p. | QD × 23 |
| | | pound 1A | | | | |
| 5 | 6 + 2 | Com- | 200 | 10 μl/g | i.p. | Q2D × 12 |
| | | pound 1A | | | | |
| 6 | 6 + 2 | Com- | 200 | 10 μl/g | i.p. | QD × 23 |
| | | pound 1A | | | | |
| 7 | 6 + 2 | Com- | 400 | 10 μl/g | i.p. | QD × 23 |
| | | pound 1A | | | | |

Note:
6 + 2: In these groups, PK analysis on days 1 & 23 were carried out only on the 2 extra mice.

The mice body weight, tumor volume and the tumor weight at the end of the study for the PK mice were followed.

TABLE 6
Detailed Formulation Description

| | | | Concentration | |
|---|---|---|---|---|
| Compounds | Package | Preparation | (mg/mL) | Storage |
| Vehicle (0.5% | — | Dissolved 1.25 g CMC- | — | 4° C. |
| CMC + | | Na and 2.5 mL | | |
| 1% Tween80) | | Tween80 in 247.5 mL | | |
| | | sterile water. | | |
| Irinotecan | 250 mg/vial | Weighed 18 mg | 10 | 4° C. |
| (Positive control) | | Irinotecan hydrochloride | | |
| | | directly in a brown glass | | |
| | | vial. Added 0.18 mL | | |
| | | DMSO to completely | | |
| | | dissolve. Diluted to final | | |
| | | concentration as | | |
| | | 10 mg/ml with sterile | | |
| | | water just before dosing. | | |
| Compound 1A | 10 g/vial | Weigh 299 mg | 40 | 4° C. |
| | | compound directly in a | | |
| | | brown glass vial. Add | | |
| | | 7.475 mL vehicle into | | |
| | | the vial. Vortex the | | |
| | | mixture for 3-4 min and | | |
| | | sonicate for 15 min. | | |
| | | Stirred for one hour. | | |
| | | The formulation is | | |
| | | homogeneous | | |
| | | suspension. Light- | | |
| | | shielded. | | |
| Compound 1A | — | Dilute 5.2 ml of | 20 | 4° C. |
| | | 40 mg/ml Compound 1A | | |
| | | with 5.2 mL vehicle. | | |
| | | The formulation is | | |
| | | homogeneous | | |
| | | suspension. Light- | | |
| | | shielded. | | |
| Compound 1A | — | Dilute 2.3 ml of | 10 | 4° C. |
| | | 20 mg/ml Compound 1A | | |
| | | with 2.3 mL vehicle. | | |
| | | The formulation is | | |
| | | homogeneous | | |
| | | suspension. Light- | | |
| | | shielded. | | |

Ensured that formulation was homogenous immediately before use by gently turning the tube up and down (The test article formulation was prepared before each dosing).

Observations

All the procedures related to animal handling, care, and the treatment in this study were performed according to approved guidelines a following the guidance of the Association for Assessment and Accreditation of Laboratory Animal Care (AAALAC). At the time of routine monitoring, the animals were checked for any effects of tumor growth and treatments on normal behavior such as mobility, food and water consumption (by looking only), body weight gain/loss (body weights were measured thrice weekly), eye/hair matting and any other abnormal effect. Death and observed clinical signs were recorded on the basis of the numbers of animals within each subset.

Tumor Measurements and Endpoints

The major endpoint was to see if the tumor growth can be delayed or mice can be cured. Tumor size was measured three times weekly in two dimensions using a caliper, and the volume was expressed in mm³ using the formula: V=0.5 a×b² where a and b are the long and short diameters of the tumor, respectively. The tumor size was then used for calculations of both T-C and TGI values. T-C was calculated with T as the median time (in days) required for the treatment group tumors to reach a predetermined size (e.g., 1,000 mm³), and C as the median time (in days) for the control group tumors to reach the same size. The TGI (in percent)=(T−C)/C*100% represents the tumor growth inhibition rate, T and C were the mean volume of the treated and control groups, respectively, on a given day. Tumor weight was measured at the study termination. The T/C value (in percent) was calculated where T and C are the mean tumor weights of the treated and control groups, respectively.

### Blood Collection (Via Tail Vein) for the PK Mice

a. Blood samples were collected after the 1st dose and the 23rd dose. Collected blood samples from 2 animals in each group at the designated timepoints post dose. The detailed timepoints are as below:
   For the groups with QD dosing schedule: 1, 4, 8 and 24 hr post dose.
   For the groups with Q2D dosing schedule: 1, 8, 24 and 48 hr post dose.
b. 20 μl blood was collected via tail vein in EDTA-2K tube (0.5 ml tube containing 0.4 μl 15% EDTA-2K. 60u1 double distilled water was added into the tube and mixed well.

Samples were stored at −70° C. until PK analysis.

### Results:

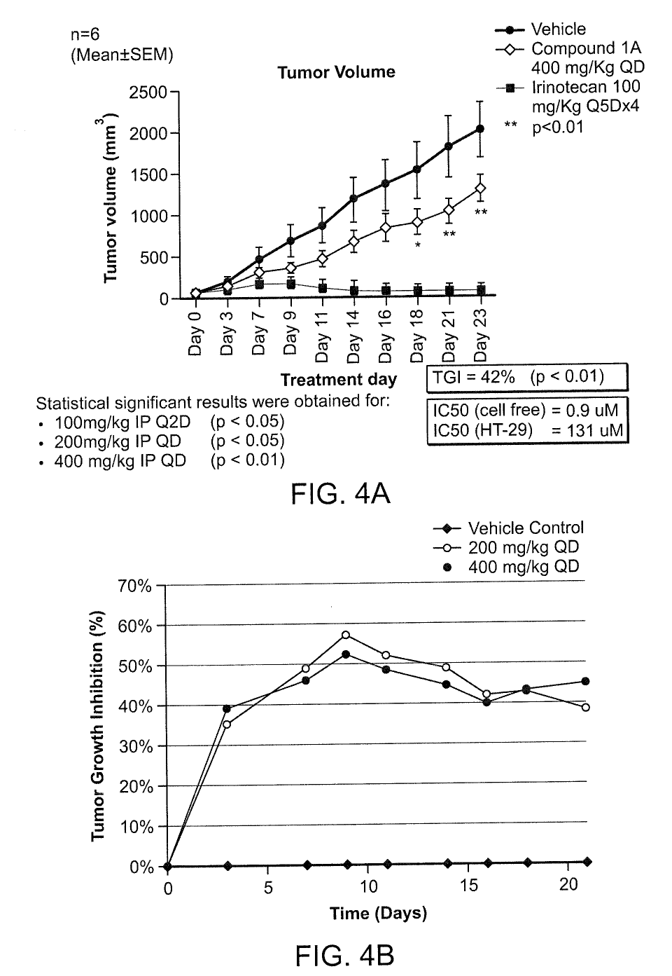

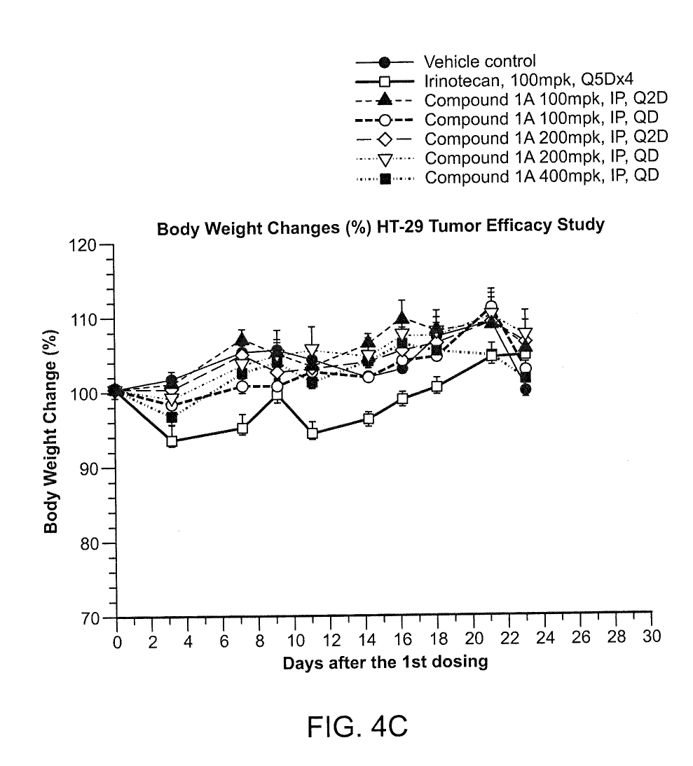

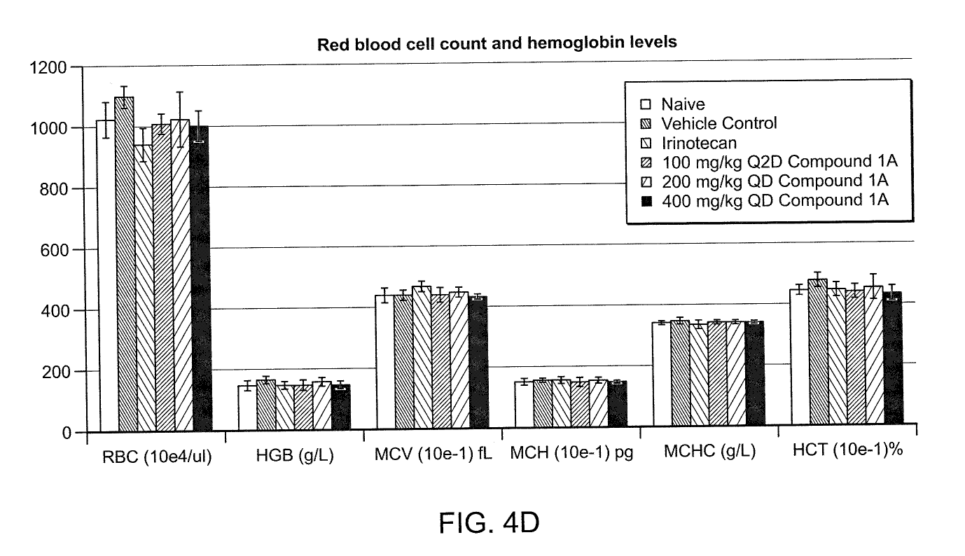

In this study, the therapeutic efficacy of Compound 1A as a single agent in the treatment of HT-29 xenograft models was evaluated. No significant body weight loss was observed in all the treatment groups. The results of tumor sizes in different groups at different time points after tumor inoculation are shown in FIG. 4A, for a dosing schedule of 400 mg/kg QD. The (Tumor Growth Inhibition) TGI for this dosing schedule was 42%. The dosing schedules of 200 mg/kg QD and 100 mg/kg Q2D also yielded statistically significantly anti-tumor activity. In particular, >50% TGI was reached by day ten for two dosing schedules, i.e., 400 mg/kg QD and 200 mg/kg QD (See FIG. 4B).

In addition, even at the highest dosages levels of 400 mg/kg QD, Compound 1A a good in vivo safety profile was observed. No significant body weight loss was observed in all the treatment groups (See FIG. 4C), and pharmacokinetic analysis of the blood samples also demonstrate good safety profiles (See FIG. 4D). This data suggests that as a PKM2 activator, Compound 1A, effects mainly cancer cells, while leaving the metabolism of normal cells relatively unaltered.

### Experiment 12: Evaluation of Compound 13A (XT26) in the H1299 Human Non-Small Cell Lung Carcinoma Nude Mouse Xenograft Model.

### Methods and Materials

### Mice

Female nude mice (nu/nu, Harlan) were 8 weeks old, and had a BW (body weight) range of 17.2-24.0 g, on D1 (day one) of the study. The animals were fed ad libitum water (reverse osmosis, 1 ppm Cl) and NIH 31 Modified and Irradiated Lab Diet® consisting of 18.0% crude protein, 5.0% crude fat, and 5.0% crude fiber. The mice were housed on irradiated Enrich-o′ Cobs™ Laboratory Animal Bedding in static microisolators on a 12-hour light cycle at 20-22° C. (68-72° F.) and 40-60% humidity.

### Tumor Cell Culture

H1299 (NCI-H1299) NSCLC cells were obtained from the American Type Culture Collection, which reports the cell line to have originated from a lymph node metastasis and to lack p53 expression. The cell line was maintained as exponentially growing cultures in RPMI-1640 medium supplemented with 10% fetal bovine serum, 2 mM glutamine, 100 units/mL penicillin G sodium, 100 μg/mL streptomycin sulfate, 25 μg/mL gentamicin, 10 mM HEPES, and 0.075% sodium bicarbonate. The tumor cells were cultured in tissue culture flasks in a humidified incubator at 37° C., in an atmosphere of 5% CO₂ and 95% air.

### Tumor Implantation and Measurement

H1299 tumor cells were harvested during log phase growth and suspended in cold PBS. Each nude mouse was inoculated subcutaneously in the right flank with 0.2 mL of the cell suspension (1×10⁷ cells). The tumors were periodically calipered in two dimensions to monitor size as the mean volume approached the desired 90-130 mm³ range. Tumor size was calculated by the following formula:

$$Tumor Volume ( {mm}^{3} ) = \frac{{w}^{2} \times l}{2}$$

where w=width and l=length, in mm, of the tumor. Tumor weight may be estimated with the assumption that 1 mg is equivalent to 1 mm³ of tumor volume. Thirteen days after tumor implantation, on D1 of the study, mice with individual tumor volumes ranging from 75 to 162 mm³ were sorted into four treatment groups with group mean tumor volumes of 104 mm³.

### Test Articles

Compound 13A (MW 467.51, code-named XT26) was provided as a powder stored at −20° C., and protected from light during storage and handling. Dosing suspensions, in 0.5% carboxymethyl cellulose and 1% Tween® 80 in deionized water (Vehicle), were prepared fresh for each dose, and kept at 4° C. post formulation. Gemcitabine (Gemzar®, Lilly, 38 mg/mL, Lot # A866884A) was stored at room temperature. A fresh 12 mg/mL gemcitabine dosing solution was prepared on each treatment day by diluting an aliquot of the stock solution with saline.

### Treatment Plan

Mice were treated in accordance with the protocol in Table 7, except for the footnoted schedule modifications in Group 4.

TABLE 7
Protocol Design for the H1299-e281 Study

| | | Treatment Regimen 1 | | | |
|---|---|---|---|---|---|
| Group | n | Agent | mg/kg | Route | Schedule |
| 1 | 12 | Vehicle | — | ip | bid × 28 first day 1 |
| | | | | | dose |
| 2 | 6 | Gemcitabine | 120 | ip | q3d × 4 |
| 3 | 12 | XT26 | 100 | ip | bid × 28 first day 1 |
| | | | | | dose |
| 4 | 12 | XT26 | 200 | ip | bid × 28 first day 1 |
| | | | | | dose |

Annotation: Vehicle = 0.5% carboxymethyl cellulose and 1% Tween ® 80 in deionized water
Protocol changes:
Group 1 tumor volume endpoint was reached on Day 21; at the client's request, the study was converted from a TGI assay to a TGD assay and dosing continued. Group 4 dosing stopped after b.i.d. × 8 treatment because of mortality and continued weight loss. Following improved health and weight gain, dosing at 200 mg/kg resumed on Day 17 on a qd schedule; however, the Day 19 dose was omitted by error. Sampling protocols for Groups 1 and 4 were added while the study was in progress.

Dosing began on D1 in four groups of mice (n=12/group in Groups 1, 3, and 4; n=6 in Group 2). XT26 and its vehicle were administered intraperitoneally (i.p.) twice daily for 28 days (b.i.d.×28), starting with the p.m. dose on D1 and ending with the a.m. dose on D29. Gemcitabine was administered i.p. once daily at three-day intervals for four doses (q3d×4). In all groups, the dosing volume of 10 ml/kg (0.2 ml/20 g mouse) was scaled to the weight of each individual animal, as measured twice weekly.

Group 1 mice received the XT26 vehicle b.i.d.×28, and served as the controls for the study.
Group 2 received gemcitabine at 120 mg/kg. Group 3 was to receive XT26 at 100 mg/kg b.i.d.×28; on D21, four mice (Animals 1, 3, 4, and 5) erroneously received 200 mg/kg XT26 for their a.m. dose. Group 4 was to receive XT26 at 200 mg/kg b.i.d.×28, but dosing stopped on D9 (b.i.d.×8) because of toxicity. Group 4 dosing at 200 mg/kg resumed on D17 on a qd (once daily)×13 dosing schedule. Because of a scheduling error, the D19 dose for Group 4 mice was omitted; qd dosing occurred on Days 17, 18, and 20-28.

### Endpoint and Tumor Growth Delay (TGD) Analysis

Tumors were calipered twice weekly for the duration of the study, and each animal was euthanized when its neoplasm reached the predetermined endpoint volume (1200 mm³) or at the end of the study, whichever came first. Animals that exited the study for tumor growth were documented as euthanized for tumor progression (TP), with the date of euthanasia. The time to endpoint (TTE) for each mouse was calculated by the following equation:

$$T T E = \frac{{log}_{10} ( endpoint volume ) - b}{m}$$

where TTE is expressed in days, endpoint volume is expressed in mm³, b is the intercept, and m is the slope of the line obtained by linear regression of a log-transformed tumor growth data set. The data set consists of the first observation that exceeded the endpoint volume used in analysis and the three consecutive observations that immediately preceded the attainment of this endpoint volume. The calculated TTE is usually less than the TP date, the day on which an animal was euthanized for tumor size. Animals that did not reach the endpoint volume were assigned a TTE value equal to the last day for which measurements were available (D28). Any animal classified as dead from treatment-related (TR) causes was assigned a TTE value equal to the day of death. Any animal that was euthanized for sampling (ES) was excluded from the analysis. Any animal dead from non-treatment-related (NTR) causes was to be excluded from TTE calculations. Treatment efficacy was determined from tumor growth delay (TGD), which is defined as the increase in the median TTE, in days, for a treatment group compared to the control group:

TGD=T−C

The percent increase in the median TTE, relative to the control group, is

$$\% TGD = \frac{T - C}{C} \times 100$$

where:

T=median TTE for a treatment group, and

C=median TTE for the designated control group.

### MTV and Criteria for Regression Responses

Treatment efficacy in each group was also determined from the median tumor volume, MTV(n), which was defined as the median tumor volume on the last day of the study (D28) in the number of animals remaining (n) whose tumors had not attained the endpoint volume.

Treatment efficacy may also be determined from the incidence and magnitude of regression responses observed during the study. Treatment may cause partial regression (PR) or complete regression (CR) of the tumor in an animal. In a PR response, the tumor volume is 50% or less of its D1 volume for three consecutive measurements during the course of the study, and equal to or greater than 13.5 mm³ for one or more of these three measurements. In a CR response, the tumor volume is less than 13.5 mm³ for three consecutive measurements during the course of the study.

### Toxicity

Animals were weighed daily on D1-5, then twice weekly until the completion of the study. The mice were observed frequently for overt signs of any adverse, treatment-related side effects.

### Sampling

On D10, three mice in Group 1 (Animals 4, 8, and 12) were sampled for blood (0.3 mL) from the mandibular vein without anesthesia. A portion of each blood sample (80 μL) was diluted 1:3 (with K-EDTA as anticoagulant) for CBC analysis, as described below. The remainder of each sample was processed for serum and stored at 4° C. until it was transmitted at ambient temperature to ANTECH for clinical chemistry analysis. On D10, three mice in Group 4 (Animals 2, 6, and 10) were euthanized by terminal cardiac puncture under CO₂ anesthesia and the full blood volume was collected. A portion of this blood sample (80 μL) was utilized for CBC analysis with differential; another portion (20 μL) was diluted with three volumes of deionized water, frozen at −80° C., and shipped on dry ice to Dynamix; and the remainder was processed for serum which was transmitted to ANTECH for clinical chemistry analysis.

On D16, blood was collected from the mandibular veins of three Group 4 mice (Animals 1, 8, and 12) for CBC analyses. D22 blood samples were not collected because the specified Group 1 mice (Animals 2, 7, and 9) had previously exited the study for tumor progression.

On D10, both kidneys and the liver were harvested from each euthanized Group 4 animal. Each organ was weighed separately, fixed overnight in 10% neutral buffered formalin, and transferred to 70% ethanol. The list of organ weights is provided in Table 11.

TABLE 11
Organ Weights
Organ Weight
Group 4: XT26 (200 mg/kg, ip, bid × 8), XT26
(200 mg/kg, ip, qd × 13 (start on day 17))
Day of Study 10

| A# | Kidney (R) (mg) | Kidney (L) (mg) | Liver (mg) |
|---|---|---|---|
| 2 | 135.3 | 125.2 | 833.1 |
| 6 | 110.4 | 103.2 | 791.9 |
| 10 | 124.1 | 119.6 | 684.4 |
| n | 3 | 3 | 3 |

### CBC Analysis

CBCs with differentials were determined using a Cell-Dyn® 3700 System (Abbott Diagnostics) for automated hematology analyses. Red blood cells and platelets were measured by electrical impedance. White blood cells were counted and differentiated into granulated (neutrophils, basophils, and eosinophils) or agranulated (lymphocytes and monocytes) cell types by flow cytometry, and hemoglobin concentrations were determined spectrophotometrically.

### Statistical and Graphical Analyses

All statistical and graphical analyses and were performed with Prism 3.03 (GraphPad) for Windows. Animals that exited the study for sampling purposes were excluded from these analyses. Survival was analyzed by the Kaplan-Meier method, based on TTE values. The logrank test compared the survival experiences (survival curves) of two groups. The two-tailed statistical analyses were conducted at P=0.05. Prism reports results as non-significant (ns) at P>0.05, significant (symbolized by “*”) at 0.01<P≦0.05, very significant (“*”) at 0.001<P≦0.01 and extremely significant (“***”) at P≦0.001.

A scatter plot was constructed to show TTE values for individual animals, by group. Group median tumor volumes were graphed on a semi-log plot as functions of time. When an animal exited the study because of tumor size or TR death, the final tumor volume recorded for the animal was included with the data used to calculate the median volume at subsequent time points. The tumor growth curve was truncated when TR mortality exceeded 10%. The percentage of animals in each group remaining in the study versus time was presented in a Kaplan-Meier survival plot. Group mean BW changes were graphed as percent change, ±SEM, from D1. Tumor growth and BW change curves were truncated when the tumors in more than 50% of the assessable animals in a group had progressed to the volume endpoint.

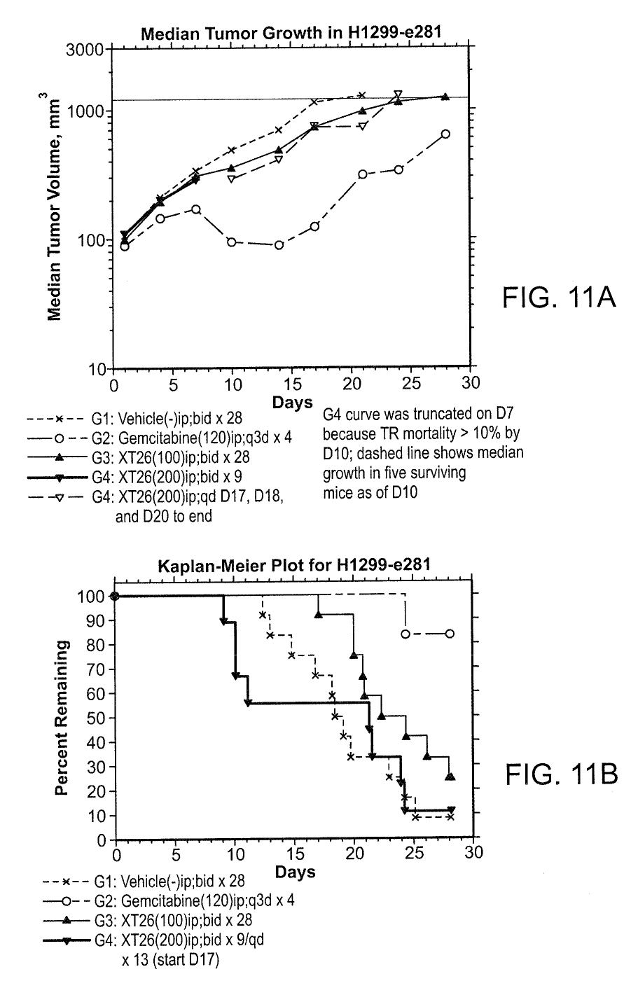

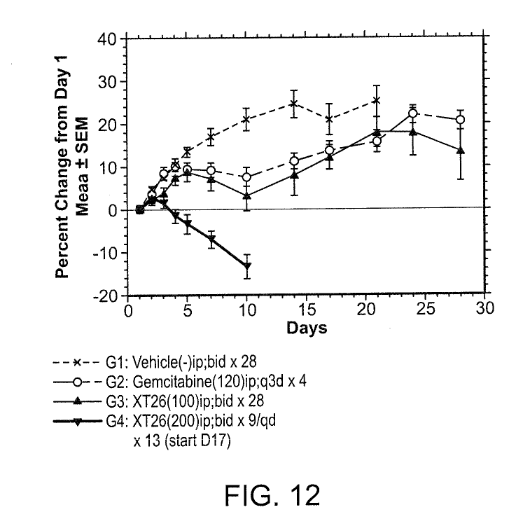

Table 12 summarizes group treatment responses and reports the logrank test significance of survival extensions. No tumors regressed during this study. FIG. 10 is a scatter plot of individual TTE values, by group. FIGS. 11A and 11B show median tumor growth and the Kaplan-Meier survival curves.

TABLE 12
Response Summary for the H1299-e281 Study

| | | | | | | | | | Statistical | MTV | | | | Mean | | |
|---|---|---|---|---|---|---|---|---|---|---|---|---|---|---|---|---|
| | | Treatment Regimen | | | | Median | T − | % | Signifi- | (n) | Regressions | | | BW | Deaths | |
| Group | n | Agent | mg/kg | Route | Schedule | TTE | C | TGD | cance | Day 28 | PR | CR | TFS | Nadir | TR | NTR |
| 1 | 12 | Vehicle | — | ip | bid × 28 | 18.7 | — | — | — | 847 (1) | 0 | 0 | 0 | — | 0 | 0 |
| | | | | | first day | | | | | | | | | | | |
| | | | | | 1 dose | | | | | | | | | | | |
| 2 | 6 | Gemcitabine | 120 | ip | q3d × 4 | 28.0 | 9.3 | 50 | ** | 600 (5) | 0 | 0 | 0 | — | 0 | 0 |
| 3 | 12 | XT26 | 100^{a} | ip | bid × 28 | 23.2 | 4.5 | 24 | * | 1080 (3) | 0 | 0 | 0 | — | 1 | 0 |
| | | | | | first day | | | | | | | | | | | |
| | | | | | 1 dose | | | | | | | | | | | |
| 4 | 9 | XT26 | 200 | ip | bid × 8; | 21.2 | 2.5 | 13 | ne | 527 (1) | 0 | 0 | 0 | −13.6% | 4 | 0 |
| | | XT26 | 200 | ip | then qd | | | | | | | | | Day 10 | | |
| | | | | | on Days 17, | | | | | | | | | | | |
| | | | | | 18, 20-28^{b} | | | | | | | | | | | |

^{a}Group 3 Animals 1, 3, 4, and 5 erroneously received 200 mg/kg XT26 for the D 21 a.m. dose.
^{b}Group 4 dosing was discontinued after a b.i.d. × 8 treatment because of mortality and weight loss. Following improved health and weight gain, dosing at 200 mg/kg resumed on Day 17 on a qd schedule; however, the Day 19 dose was omitted by error.
Study Endpoint = 1200 mm³, Days in Progress = 28
n = number of animals in a group not dead from accidental or unknown causes, or euthanized for sampling
TTE = time to endpoint, T − C = difference between median TTE (days) of treated versus control group, % TGD = [(T − C)/C] × 100. The maximum T − C in this study is 9.3 days (50%), compared with Group 1.
Statistical Significance (Logrank test):
ne = not evaluable,
ns = not significant,
*= P < 0.05,
**= P < 0.01,
***= P < 0.001, compared to Group 1 MTV
(n) = median tumor volume (mm³) for the number of animals on the day of TGD analysis (excludes animals with tumor volume at endpoint) PR = partial regressions; CR = total number complete regressions; TFS = tumor free survivors, i.e., CRs at end of study
Mean BW Nadir = lowest group mean body weight, as % change from Day 1; — indicates no decrease in mean body weight was observed
TR = treatment-related death; NTR = non-treatment-related death

TABLE 13
Statistical Analysis of H1299-e281

| Groups | Vehicle; ip; | Vehicle; ip; | Vehicle; ip; |
|---|---|---|---|
| Compared | bid × 28 | bid × 28 | bid × 28 |
| | first day 1 | first day 1 | first day 1 |
| | dose | dose | dose |
| | — | — | — |
| | Group 1 vs 2 | Group 1 vs 3 | Group 1 vs 4 |
| | Gemcitabine; ip; | XT26; ip; bid × 28 | XT26; ip; bid × 9/ |
| | q3d × 4 | first day 1 dose | XT26; ip; qd × 13 |
| | 120 | 100 | (start on day 17) |
| | | | 200/200 |
| Logrank Test | | | |
| Chi square | 9.492 | 3.854 | ne |
| df | 1 | 1 | 40% |
| P value | 0.0021 | 0.0496 | mortality |
| P value summary | ** | * | |
| Are the survival | Yes | Yes | |
| curves sig different? | | | |
| Median survival | | | |
| Column A | 18.65 | 18.65 | |
| Column B | Undefined | 23.25 | |
| Ratio | | 0.8022 | |
| 95% CI of ratio | | 0.3878 to 1.217 | |
| Hazard Ratio | | | |
| Ratio | 11.48 | 2.315 | |
| 95% CI of ratio | 1.961 to 20.67 | 1.002 to 6.575 | |

ns = non significant
* = P ≦ 0.05
** = P ≦ 0.01
*** = P ≦ 0.001

### Efficacy

### Growth of H1299 Non-small Cell Lung Tumors in Control Mice (Group 1)

Group 1 mice (n=12) received the XT26 vehicle (0.5% carboxymethyl cellulose and 1% Tween® 80 in deionized water) i.p. b.i.d.×28 and served as controls for all analyses. The median TTE for Group 1 mice, 18.7 days, established a maximum possible TGD of 9.3 days (50%) in this 28-day study (Table 12). Eleven Group 1 tumors attained the 1200 mm³ volume endpoint, and one animal remained on D28 with an 847 mm³ tumor. Group 1 TTEs ranged from 12.3 to 28.0 days (FIG. 10).

### Response to Gemcitabine Therapy (Group 2)

In Group 2 (n=6), treatment with 120 mg/kg gemcitabine i.p. q3d×4 resulted in an assigned median TTE of 28.0 days, corresponding to the maximum possible TGD of 9.3 days (50%), and significant survival extension (P<0.01; logrank test, Table 12). Five (5/6) Group 2 mice remained on study on D28 with an MTV of 600 mm³. The Group 2 median tumor volume decreased between D7 and D14, but subsequent median tumor growth resembled that observed in the control group (FIG. 11A).

### Response to XT26Therapies (Groups 3 and 4)

In Group 3 (n=12), mice were to receive 100 mg/kg XT26 i.p. b.i.d.×28. On D21, four mice (Animals 1, 3, 4, and 5) erroneously received 200 mg/kg for the a.m. dose. It was concluded that the dosing error had no substantial impact on the overall response. The Group 3 treatment resulted in a median TTE of 23.2 days, corresponding to a 4.5-day TGD (24%), and significant survival extension (P<0.05, Table 12). Three (3/12) Group 3 mice survived to D28 with an MTV of 1080 mm³. One TR death occurred in Group 3 on D17, prior to the D21 dosing error.

In Group 4 (n=12), treatment with 200 mg/kg XT26 i.p. b.i.d. resulted in four TR deaths on Days 9-11; the b.i.d. treatment was discontinued after the a.m. dose on D9. The median tumor growth curve for Group 4 (solid line) was curtailed on D7 because mortality exceeded 10% by the subsequent measurement day (FIG. 11A). The curtailed median tumor growth curve for Group 4 is similar to the curve for Group 3. After three Group 4 mice were euthanized for sampling on D10, five mice remained on study in this group. As there were no further deaths, and these animals regained weight by D17, dosing resumed, but on a qd treatment schedule. The animals were actually dosed qd on Days 17, 18, and 20-28; omission of the D19 dose was due to a scheduling error. After exclusion of the three sampled animals, the median TTE for nine Group 1 mice was 21.2 days, corresponding to a 2.5-day TGD (13%). For informational purposes, median tumor growth in the five mice that remained after D10 was represented in FIG. 11A by a dotted line, which was similar to the curve for Group 3. According to the survival curve for these five mice, the discontinuous 200 mg/kg XT26 therapy afforded little or no survival advantages relative to the vehicle treatment (FIG. 11B). One Group 4 mouse survived to D28 with a 527 mm³ tumor.

In summary, compound 13A (XT26) at 100 mg/kg b.i.d.×28 resulted in modest (24%), statistically tumor growth reduction (TGD) and one tumor related (TR) death in the H1299 NSCLC xenograph model. Compound 13A at 200 mg/kg b.i.d.×8 caused 33% mortality. All mice treated with compound 13A had intrapertoneal deposits of white material as of day 3. The tumor model responded moderately well to standard 120 mg/kg gemcitabine therapy.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims. It will be understood by those skilled in the art that various changes in form and details may be made therein without departing from the scope of the invention encompassed by the appended claims.

What is claimed is:

1. A compound according to Formula I: 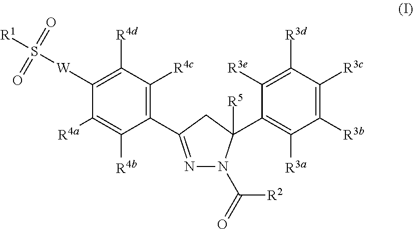
or a pharmaceutically acceptable salt thereof,
wherein:
W is NR^{a} or is absent,
wherein R^{a} is
(i) a group selected from the group consisting of a hydrogen, a linear or branched, saturated or unsaturated C₁-C₆ alkyl, C(O)R^{c}, and a linear or branched, saturated or unsaturated C₁-C₆ alkoxy; or
(ii) R^{a} and R^{4a} together with the atoms to which they are attached, form a five to seven membered heterocycloalkyl or heteroaromatic ring;
R¹ is selected from the group consisting of a hydrogen a linear or branched, saturated or unsaturated C₁-C₆ alkyl or heteroalkyl, a saturated or unsaturated C₃-C₈ cycloalkyl or heterocycloalkyl, an aryl and a heteroaryl,
wherein said alkyl, heteroalkyl, cycloalkyl, heterocycloalkyl, aryl or heteroaryl is unsubstituted or substituted with one or more R^{b},
wherein R^{b} is independently at each occurrence selected from the group consisting of:
u) a linear or branched, saturated or unsaturated C₁-C₆ alkyl,
v) a linear or branched, saturated or unsaturated C₁-C₆ haloalkyl,
w) a linear or branched, saturated or unsaturated C₁-C₆ alkoxy or aryloxy,
x) a linear or branched, saturated or unsaturated C₁-C₆ haloalkoxy,
y) a linear or branched, saturated or unsaturated C₁-C₆ alkylsulfonyl,
z) a linear or branched, saturated or unsaturated C₁-C₆ thioalkyl or thioaryl,
aa) a saturated or unsaturated C₃-C₈ cycloalkyl,
bb) an aryl,
cc) a heteroaryl,
dd) a heterocycloalkyl,
ee) hydroxyl,
ff) cyano,
gg) amino,
hh) nitro,
ii) halogen,
jj) COR^{c},
kk) COOR^{c},
ll) CONR^{c}R^{d},
m) NHCOR^{c}, and
nn) NR^{c}R^{d}
wherein R^{c} and R^{d} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
or wherein two R^{b} on adjacent carbon atoms form a heteroaryl ring or heterocycloalkyl ring;
R² and R⁵ are, each independently, selected from hydrogen, a linear or branched, saturated or unsaturated C₁-C₆ alkyl or heteroalkyl, heteroaryl, or aryl,
wherein said alkyl, heteroalkyl, heteroaryl, or aryl is unsubstituted or substituted with one or more R^{e},
wherein R^{e} is independently at each occurrence selected from the group consisting of COR^{f}, COOR^{f}, CONR^{f}R^{g}, NHCOR^{f}, and NR^{f}R^{g},
wherein R^{f} and R^{g} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
R^{3a}, R^{3b}, R^{3c}, R^{3d} and R^{3e} are, each independently, selected from:
v) hydrogen
w) a linear or branched, saturated or unsaturated C₁-C₆ alkyl,
x) a linear or branched, saturated or unsaturated C₁-C₆ haloalkyl,
y) a linear or branched, saturated or unsaturated C₁-C₆ alkoxy or aryloxy,
z) a linear or branched, saturated or unsaturated C₁-C₆ haloalkoxy,
aa) a linear or branched, saturated or unsaturated C₁-C₆ alkylsulfonyl,
bb) a linear or branched, saturated or unsaturated C₁-C₆ thioalkyl or thioaryl,
cc) a saturated or unsaturated C₃-C₈ cycloalkyl,
dd) an aryl,
ee) a heteroaryl,
ff) a heterocycloalkyl,
gg) hydroxyl,
hh) cyano,
ii) amino,
jj) nitro,
kk) halogen,
ll) COR^{h},
m) COOR^{h},
m) CONR^{h}Rⁱ,
oo) NHCOR^{h}, or
pp) NR^{h}Rⁱ
wherein R^{h} and Rⁱ are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
or
two adjacent of R^{3a}, R^{3b}, R^{3c}, R^{3d} and R^{3e}, together with the atoms to which they are attached, form a C₅-C₇ cycloalkyl, heterocycloalkyl, aromatic or heteroaromatic ring;
R^{4b}, R_{4c} and R^{4d}, and a when R^{4a} does not form a five to seven membered ring with R^{a}, R^{4a}, are, each independently, selected from:
v) hydrogen
w) a linear or branched, saturated or unsaturated C₁-C₆ alkyl,
x) a linear or branched, saturated or unsaturated C₁-C₆ haloalkyl,
y) a linear or branched, saturated or unsaturated C₁-C₆ alkoxy or aryloxy,
z) a linear or branched, saturated or unsaturated C₁-C₆ haloalkoxy,
aa) a linear or branched, saturated or unsaturated C₁-C₆ alkylsulfonyl,
bb) a linear or branched, saturated or unsaturated C₁-C₆ thioalkyl or thioaryl,
cc) a saturated or unsaturated C₃-C₈ cycloalkyl,
dd) an aryl,
ee) a heteroaryl,
ff) a heterocycloalkyl,
gg) hydroxyl,
hh) cyano,
ii) amino,
jj) nitro,
kk) halogen,
ll) COR^{j},
m) COORⱼ,
m) CONR^{j}R^{k},
oo) NHCOR^{j}, or
pp) NR^{j}R^{k}
wherein R^{j} and R^{k} are, each independently, hydrogen or a group selected from a linear or branched, saturated or unsaturated C₁-C₆ alkyl, a saturated or unsaturated C₃-C₈ cycloalkyl or heterocycloalkyl, an aryl and a heteroaryl;
or
two adjacent of R^{4a}, R^{4b}, R^{4c} and R^{4d}, together with the atoms to which they are attached, form a C₅-C₇ cycloalkyl, heterocycloalkyl, aromatic or heteroaromatic ring.

2. The compound according to claim 1, wherein R¹ is a C₁-C₃ alkyl.

3. The compound according to claim 1, wherein R¹ is a substituted or unsubstituted aryl.

4. The compound according to claim 1, wherein R¹ is a phenyl substituted with one or more R^{b}, further wherein R^{b} is selected from fluorine, chlorine, methoxy, CF₃, NHC(O)CH₃, and NH₂ or two R^{b} on adjacent carbon atoms form a heterocycloalkyl ring.

5. The compound according to claim 1, wherein R¹ is a phenyl substituted with one or more R^{b} and R^{b} is selected from fluorine, CF₃, NHC(O)CH₃, and NH₂ or two R^{b} on adjacent carbon atoms form a heterocycloalkyl ring.

6. The compound according to claim 1, wherein R² is selected from C₁-C₃ alkyl, heteroaryl, and aryl.

7. The compound according to claim 1, wherein one or more of R^{3a}, R^{3b}, R^{3c}, R^{3d} and R^{3e} is C₁-C₃ alkoxy, halogen, C₁-C₃ alkyl, or NR^{h}Rⁱ, wherein R^{h} and Rⁱ are each independently, hydrogen or C₁-C₃ alkyl or two adjacent of R^{3a}, R^{3b}, R^{3c}, R^{3d}, and R^{3e}, together with the atoms to which they are attached, form a heterocycloalkyl or heteroaromatic ring.

8. The compound according to claim 1, wherein one of R^{3a}, R^{3b}, R^{3c}, R^{3d} and R^{3e} is methoxy and the remaining R^{3a}, R^{3b}, R^{3c}, R^{3d} and R^{3e} are hydrogen.

9. The compound according to claim 1, wherein R^{4a}, R^{4b}, R^{4c}, and R^{4d} are all hydrogen.

10. The compound according to claim 1, wherein R⁵ is hydrogen.

11. The compound according to claim 1, wherein the compound is a single enantiomer.

12. A compound selected from a compound listed in Table 1 or a pharmaceutically acceptable salt thereof.

13. A pharmaceutical composition comprising a compound claim 1 and at least one pharmaceutically acceptable carrier or excipient.

14. A method of preventing or treating a cancer and/or a cell proliferation disorder in a subject, wherein said method comprises the step of administering to the subject in need thereof, an effective amount of a compound of claim 1 or a pharmaceutically acceptable salt thereof or a compound selected from a compound in Table 1, 1A, or 1B or a pharmaceutically acceptable salt thereof.

15. The method according to claim 14, wherein the cancer and/or cell proliferation disorder is selected from a disorder of the hematologic system, lung, colon, pancreas, prostate, skin, ovary, and breast.

16. The method according to claim 15, wherein the medicament is administered as part of a combination therapy comprising an additional therapy.

17. A method of preventing or treating a disorder selected from immune system dysfunction, autoimmune disease, transplant rejection, inflammatory disorder or disease, and metabolic disorder in a subject, wherein said method comprises the step of administering to the subject in need thereof, an effective amount of a compound of claim 1 or a pharmaceutically acceptable salt thereof or a compound selected from a compound in Table 1, 1A, or 1B or a pharmaceutically acceptable salt thereof.

18. The method according to claim 14, wherein the medicament is to be administered orally, topically or intravenously.

19. The method according to claim 17, wherein the medicament is to be administered orally, topically or intravenously.
